(19) United States
(12) Patent Application Publication
(10) Pub. No.: US 2026/0014092 A1
(43) Pub. Date: Jan. 15, 2026

(54) EXTRACELLULAR VESICLES FROM MICROALGAE, THEIR USE FOR VACCINES AND FOR IMMUNOMODULATION

(71) Applicant: AGS THERAPEUTICS SAS, Paris (FR)

(72) Inventors: Lila DRITTANTI, Paris (FR); Manuel VEGA, Paris (FR); Rana LEBDY, Antony (FR)

(21) Appl. No.: 19/334,304

(22) Filed: Sep. 19, 2025

Related U.S. Application Data
(63) Continuation of application No. PCT/EP2024/057645, filed on Mar. 21, 2024.
(60) Provisional application No. 63/491,920, filed on Mar. 23, 2023.
(60) Provisional application No. 63/517,083, filed on Aug. 1, 2023.

(51) Int. Cl. A61K 9/51; A61K 31/713; A61K 39/00; A61P 37/04

(52) U.S. Cl. CPC A61K 9/5176; A61K 31/713; A61P 37/04; A61K 2039/55555

(57) ABSTRACT

Provided are vaccines and immunomodulatory compositions containing extracellular vesicles from microalgae (MEVs) that are loaded with bioactive cargo, that includes antigens and/or immunomodulatory proteins, nucleic acids, and nucleic acid encoding the proteins. The MEVs are formulated and administered by a variety of routes of administration that are advantageous for modulating the immune systems. Vaccines include those that are therapeutic for treating a disease, disorder, or condition, those that elicit an immunoprotective response, and/or otherwise modulate the immune system. The compositions include MEVs containing cargos for modulating intracellular receptors.

### RELATED APPLICATIONS

This application is a continuation of International PCT application No. PCT/EP2024/057645, filed Mar. 21, 2024, published as International Publication No. WO 2024/194423 on Sep. 26, 2024, entitled “Extracellular Vesicles from Microalgae, Their Use for Vaccines and for Immunomodulation,” to inventors Lila Drittanti, Manuel Vega, and Rana Lebdy, and to Applicant AGS Therapeutics SAS, which claims benefit of priority to U.S. provisional application Ser. No. 63/517,083, filed Aug. 1, 2023, entitled “Extracellular Vesicles From Microalgae, Their Biodistribution Upon Intranasal Administration, and Uses Thereof,” to inventors Lila Drittanti and Manuel Vega, and to Applicant AGS Therapeutics SAS; and to U.S. provisional application Ser. No. 63/491,920, filed Mar. 23, 2023, entitled “Extracellular Vesicles from Microalgae, Their Use for Vaccines and for Immunomodulation,” to inventors Lila Drittanti, Rana Lebdy, and Manuel Vega, and to Applicant AGS Therapeutics SAS.

Benefit of priority is claimed to U.S. provisional application Ser. No. 63/491,920, filed Mar. 23, 2023, entitled “Extracellular Vesicles From Microalgae, Their Use for Vaccines and for Immunomodulation,” to inventors Lila Drittanti, Rana Lebdy, and Manuel Vega, and to Applicant AGS Therapeutics SAS.

Benefit of priority is claimed to U.S. provisional application Ser. No. 63/517,083, filed Aug. 1, 2023, entitled “Extracellular Vesicles from Microalgae, Their Biodistribution Upon Intranasal Administration, and Uses Thereof,” to inventors Lila Drittanti and Manuel Vega, and to Applicant AGS Therapeutics SAS.

This application is related to International PCT application No. PCT/EP2023/051650, filed Jan. 24, 2023, published as International PCT publication No. WO 2023/144127, on Aug. 3, 2023, entitled “Extracellular Vesicles from Microalgae, Their Biodistribution Upon Administration, and Uses,” to inventors Lila Drittanti and Manuel Vega, and to Applicant AGS Therapeutics SAS.

This application is related to International PCT application No. PCT/EP2022/070371, filed Jul. 20, 2022, published as International PCT publication No. WO2023/001894, on Jan. 26, 2023, entitled “Extracellular Vesicles from Microalgae, Their Preparation, and Uses,” to inventors Lila Drittanti, Juan Pablo Vega, Jeremy Pruvost, and Manuel Vega, and to Applicants: AGS Therapeutics SAS, 10 rue Greneta, 75003 Paris, France; AGS-M SAS, 41-43 Quai de Malakoff, 44000 Nantes, France; and Nantes Universite, 1 Quai de Tourville, 44035 Nantes, France.

Where permitted, the subject matter of each of these applications is incorporated by reference in its entirety.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file was created on Sep. 17, 2025, is 1,231,339 bytes in size, and is titled 5508SEQ001.xml.

### FIELD

Provided are compositions containing extracellular vesicles from microalgae (MEVs) that are loaded with bioactive cargo (payloads) and for use as vaccines to induce an immune response for prevention or treatment of a disease or disorder, and for delivery of immune modulators to modulate immune responses.

### BACKGROUND

Extracellular vesicles (EVs) are natural particles produced by most cells. EVs include exosomes (generally about 30-150 nm in size), which are released to the extracellular environment upon fusion of multivesicular endosomes with the plasma membrane, and include microvesicles (about 50-1000 nm), which are produced by the outward budding of membrane vesicles from the cell surface. Exosomes and microvesicles have similar properties, and in general are referred to as EVs.

EVs facilitate intercellular communication via cell-cell transfer of proteins and nucleic acids, such as microRNAs (miRNAs), long noncoding RNAs (lncRNAs), and mRNAs. By virtue of this, EVs derived from mammals and plants have been used as carriers for short interfering RNA (siRNA) delivery, microRNA (miRNA), and small molecule drugs. They are a promising delivery vehicle. There is a need for conveniently produced EVs that are readily delivered to cells and tissues. It is an object herein to provide such EVs.

### SUMMARY

Provided are cargo-loaded extracellular vesicles (EVs) for use for administration to subjects in vivo and cells and cell lines in vitro. EVs are loaded with cargo that includes bioactive molecules, including biomolecules and small molecules, such as diagnostic and/or therapeutic molecules. The EVs herein are from microalgae. Microalgae are unicellular green algae. The EVs herein are from microalgae and are referred to as MEVs. Microalgae are unicellular green algae, and include those that belong to the order Chlorellales, in particular, the Chlorellaceae family, and in particular those that belong to the Chlorella genus, such as Chlorella vulgaris. The MEVs, thus, are from microalgae that is a species of the family Chlorellaceae. Such microalgae include members of the genus Chlorella or Parachlorella. Exemplary thereof are: Chlorella ellipsoidea, Chlorella pyrenoidosa, Chlorella sorokiniana, Chlorella vulgaris, and Chlorella variabilis, a species of Parachlorella, such as Parachlorella kessleri, Parachlorella beijerinckii, and Parachlorella hussii.

The MEVs and compositions provided herein are for immunomodulation and/or vaccination. The MEVs comprise cargo that is immunomodulatory or that can vaccinate or treat or prevent a disease or condition by virtue of an immune response induced by or responsive to cargo in the MEVs. The MEVs can comprise antigen or immunogen and/or an immunomodulator, or nucleic acid encoding the antigen, immunogen, and/or immunomodulator. The vaccine is for treating, preventing, or reducing the severity of a disease, disorder, or condition; and the immunomodulator is an agent that acts on the immune system directly or indirectly; and the composition containing the MEVs is formulated for administration by a route whereby the MEVs traffic to a cell, tissue, or organ of the immune system. Exemplary of routes of administration is oral, intramuscular, and inhalation into the lung. It is shown herein that upon such administration the MEVs can deliver their cargo to targets, including intracellular receptors, such as TLRs and immune cells, that modulate or induce an innate or acquired or humoral immune response.

Microalgae extracellular vesicles (MEVs) can be manufactured on a large scale. MEVs are exogenously loaded (exo-loaded) with the bioactive molecule cargo. The MEVs can be endogenously loaded (endo-loaded) by producing them in genetically-modified microalgae that encode or express proteins, polypeptides, small peptides, various RNA molecules and/or other biomolecules that the microalgae can be genetically programmed to express and thereby package in MEVs. The MEVs provided herein are vaccines and/or are for delivery of antigens, immunogens, immunomodulators, and combinations thereof. The cargo can be polypeptides, proteins, or peptides, and immunogenic or antigenic fragments, or can comprise nucleic acid, DNA or RNA, encoding the products. The MEVs are in compositions formulated, for example, for oral administration, or mucosal administration, or for subcutaneous administration, or intramuscular administration, or for inhalation, and can be for delivery to mucosal tissues. Further description is provided below and in the sections that follow. Routes of administration and trafficking patterns are described. Of interest for the MEV vaccines is oral administration. As shown and described herein, MEVS can be orally administered, and they traffic through the gut-associated lymphoid tissue (GALT) and ultimately to the spleen and immune system.

The MEVs can be used to deliver antigenic payloads for use as vaccines for administration orally or intramuscularly, or other routes. They also can be used to modulate innate immune responses via targeted activation or inactivation of particular toll-like receptors (TLRs).

As provided and shown herein, the MEVs can be administered by various routes, including orally, by inhalation into the lungs, and/or intramuscularly, to deliver payloads to modulate the immune system, such as for the delivery of vaccines, such as antigens, and immunomodulatory agents that affect the immune system. For example, MEVs can be delivered into the lungs by inhalation, and into the gut, particularly into gut-associated lymphoid tissue GALT, by oral administration. GALT immune cells traffic the internalized MEVs to the spleen. From the GALT, the MEVs trigger a humoral and cellular response against antigenic payloads. MEVS do not traffic to the liver following oral administration.

In the intestine, MEVs are internalized by dendritic cells and resident macrophages. By oral-intestinal administration, antigen-loaded MEVs elicit an immune response against the antigenic payload. The humoral response includes strong class switching from IgG to IgA, indicating generation of antigen-specific mucosal immunity. There was no observed neutralizing response to MEVs that were administered orally.

It is shown herein, that, when administered by intramuscular (IM) administration, the MEVs elicit a humoral immune response against an antigenic payload. A significant isotype class switching to IgA antibodies, from IgG, occurs. The examples show an antigen-mediated antigen-specific humoral response (see, e.g., FIGS. 14A-14C, and accompanying description).

As described and shown herein that MEVs, not only can be used to deliver payloads into cells, they also have the, heretofore rare, capacity to reach endosomes, including endosomes that host toll-like receptors (TLRs). They can deliver to intracellular TLRs modulators of TLRs, to thereby modulate pathways that involve TLRs. The MEVs, for example, can be loaded with agonists and/or antagonists of endosomal TLRs, such as TLR3 and TLR9. MEVs loaded with modulators of endosomal TLRs can trigger (or inhibit) TLR-dependent signaling pathways in epithelial cells, including those in the lungs and intestine, and in immune cells, such as in macrophages. MEVs can be used to activate/inactivate intracellular TLRs in cells, including intestinal epithelial cells, lung epithelial cells, and monocytes.

It is shown herein that MEVs can effectively reach and modulate endosomal/intracellular TLRs, as exemplified by modulation of TLR9 and TLR3. This can be effected in vivo by oral administration of the MEVs. It is shown herein that: (1) the modulation of TLRs by the payload (cargo) carried by the MEVs, leads to the triggering of signaling pathways downstream from the TLRs; and (2) the signaling pathways lead to the (up and down) regulation of immune mediators, such as inflammatory and non-inflammatory cytokines.

It is shown herein that through the specific payloads carried by the MEVs, the inflammatory and/or anti-inflammatory response or state can be modulated, such as by oral administration or delivery of ligands by MEVs into cells, such as by MEVs containing or encoding agonists and antagonists of TLRs. Through the delivery of the payloads carried by the MEVs, an innate immune response can be elicited. Additionally, through delivery of specific payloads carried by the MEVs, a humoral and a cellular response, an “acquired response” can be elicited.

The MEVs can be exogenously loaded following isolation or partial purification/isolation of the MEVs from microalgae by contacting the MEVs with the cargo to produce the compositions in which substantially all of the MEVs have substantially the same exogenously-loaded heterologous cargo. The biodistribution pattern does not depend upon the manner in which the MEVs are loaded (see e.g., Example 14, in which exogenously and endogenously loaded MEVs (as a control) deliver biologically active cargo).

The MEVs provided herein have unique biodistribution patterns, which are a function of the route of administration. Biodistribution of the MEVs is different from mammalian EVs and other EVs and/or nanoparticles. For example, systemically delivered mammalian EVs accumulate in the liver, kidneys, and spleen. Some mammalian-derived secreted EVs have limited pharmaceutical acceptability (see, e.g., International PCT Publication No. WO2021/122880). While others have shown that certain photosynthetic microalgae release EVs into growth medium, there is no description or understanding of the use of such EVs as drugs or as drug delivery vehicles; there is no description of or understanding of their fate upon administration. It is shown herein that MEVs upon administration via various routes are distributed to organs and tissues differently from mammalian EVs. As one example, while mammalian EVs, with the exception of bovine milk EVs, cannot be administered orally because they do not survive the harsh environment of the stomach, MEVs can be orally administered and delivered to the intestine, from where they traffic to the spleen, including the white spleen.

The MEVs are loaded with a variety of cargos (also referred to as “payloads”), including, but not limited to, RNA, such as inhibitory RNAs and other RNA products, oligonucleotides, plasmids, peptides, proteins, and/or small molecules. As shown herein, the MEVs can deliver the cargo to organs, tissues, and cells, and can be targeted by the route of delivery, where they can be delivered. It is shown herein that the MEVs, including those from the order Chlorellales, in particular, the Chlorellaceae family, and in particular those that belong to the Chlorella genus, such as Chlorella vulgaris. The Chlorellaceae family MEVs, including the Chlorella MEVs, have a striking capacity to pass through stringent natural barriers, such as the digestive tract, and olfactory neurons, that are not shared by other extracellular vesicles (EVs) from other sources, including mammalian EVs.

As described herein, the MEVs can be exogenously loaded (exo-loaded) with a diversity of biologically active molecules, such as siRNA, mRNA, plasmids, ASO, peptides, proteins, and/or small molecules, which allows for a variety of therapeutic, diagnostic, and other uses. The MEVs also can be loaded endogenously by the microalgae in which they are produced (see, description herein, see, also, U.S. provisional application Ser. No. 63/349,006, filed on Jun. 3, 2022). As shown herein, MEV biodistribution is determined by the route of administration. Thus, MEVs can deliver their cargo to a variety of tissues and organs, including, for example, to the lungs, to the intestine, to the GALT, to the spleen, to the liver, and to the brain, depending on whether they are administered intratracheally, orally, intravenously, or intranasally, or inhaled.

As demonstrated herein the MEVs have many uses, including therapeutic uses, including delivery of therapeutics for treatment and/or prevention (including reducing the risk or severity) of diseases, disorders, and conditions. These uses include therapeutic uses, including immunomodulation, immuno-oncology, treatment of genetic or metabolic disorders, neurologic disorders, psychiatric disorders, respiratory disorders, among others. Of particular interest herein, and described in detail in sections below, the MEVs can be loaded with cargo, such as antigens or nucleic acid encoding antigens for use as vaccines, and also for delivery of immunomodulators to organs, tissues, and cells of the immune system or that modulate the immune system.

Cargos (also referred to as “payloads”), include, but are not limited to, RNA, such as inhibitory RNAs and other RNA products, oligonucleotides, plasmids, peptides, proteins, and small molecules. Exogenously-loaded MEVs can be loaded with almost any molecule of interest; endogenously-loaded MEVs, where the microalgae cells are genetically-modified to express or encode a product produce MEVs that contain cargo, such as RNA, DNA, peptides, small peptides, polypeptides, and proteins that are produced and packaged in EVs by the microalgae.

The MEVs can deliver the cargo to organs, tissues, and cells, and can be targeted by the route of delivery, where they can be delivered. It is shown herein that the MEVs, including the Chlorella MEVs, have a striking capacity to pass through stringent natural barriers, such as the digestive tract, that is not shared by other extracellular vesicles (EVs) from other sources, including mammalian EVs. These properties are exploited herein for delivery of vaccines and immunomodulatory therapeutics. For use as vaccines and for delivery of immunomodulatory therapeutics the MEVs generally are administered orally or intramuscularly. The fate of MEVs upon oral or intramuscular (IM) administration is described in the copending applications, noted herein, with inventors and applicants in common and also described herein and in Examples below.

Provided are compositions that contain MEVs, such as exogenously cargo-loaded MEVs, particularly those produced by the order Chlorellales, in particular the Chlorellaceae family, and in particular the Chlorella genus, such as Chlorella vulgaris. The compositions include pharmaceutical compositions that can be formulated for a particular route of delivery.

Methods for loading the MEVs are described. The cargos are bioactive molecules or combinations thereof, including biomolecules and small molecules. The cargos include, for example, biomolecules, including biopolymers, such as DNA and RNA, proteins, protein complexes, protein-nucleic acid complexes, plasmids, and also include small molecules, such as small molecule drugs. The bioactive molecules include therapeutics, such as anti-cancer compounds and biomolecules, such as RNAi, oligonucleotides, and proteins, and complexes, and diagnostic molecules, such as detectable markers, molecules that are cosmetics, and molecules that act as anti-infectives for humans, animals and plants. Methods of treatment of diseases and disorders, including pathogen infections and cancers, and uses for the MEVs for treatment for the diseases and disorders are provided as are methods of diagnosis.

Target tissues for treatment and/or delivery include, for example, epithelia and mucosa cells (e.g., any kind of either external or internal mucosa: mouth, gut, uterus, trachea, bladder, and others), endothelial cells, sensory cells (e.g., visual, auditory), cancer cells, tumor cells, blood cells, blood cell precursors, neural system cells (e.g., neurons, glial cells and other CNS and peripheral nervous cells), cells of the immune system (e.g., lymphocytes, immuno-regulatory cells, effector cells), germ cells, secretory cells, gland cells, muscle cells, stem cells, including, for example, embryonic or tissue specific stem cells, liver cells, infected cells, such as cells infected with virus, bacteria, fungi, or other pathogens, native cells, and NS genetically engineered cells.

Provided are compositions that contain isolated microalgae extracellular vesicles (MEVs), where the microalgae is a species of the genus Chlorella; and the composition is formulated for administration to a subject. The Chlorella extracellular vesicles can contain a heterologous bioactive cargo molecule that has been introduced into the isolated extracellular vesicles, whereby the vesicles in the composition that contain heterologous bioactive molecule cargo contain the same bioactive molecule cargo, where: the cargo molecule is heterologous to Chlorella; and the bioactive cargo is a biomolecule or a small molecule.

For all embodiments, the Chlorella is any species of Chlorella, such as, but not limited to, Chlorella selected from among Chlorella ellipsoidea, Chlorella pyrenoidosa, Chlorella sorokiniana, Chlorella vulgaris, and Chlorella variabilis. In particular embodiments, the Chlorella is Chlorella vulgaris.

Provided are compositions that contain isolated microalgae extracellular vesicles (MEVs), where the microalgae is a species of Chlorella; the MEVs in the composition contain heterologous bioactive molecule cargo that has been introduced into the isolated MEVs, whereby the vesicles in the composition that contain the heterologous bioactive molecule cargo contain the same cargo. The cargo is heterologous, not endogenous, to Chlorella; and the cargo is a biomolecule or a small molecule drug. Each of the MEVs that contain cargo can comprise a plurality of different heterologous cargos.

Cargo includes, for example, proteins, peptides, and nucleic acids. The bioactive molecules can be synthetic, naturally-occurring, and/or modified to alter a property or activity. Included are any molecules that have been used as drugs or therapeutics or diagnostics or cosmetics or in industry. The cargo can be, but is not limited to, a therapeutic for treating or preventing a disease or disorder or condition, or treating or preventing a symptom thereof. The cargo can be a nucleic acid molecule, a polypeptide, a protein, a plasmid, an aptamer, or an antisense oligonucleotide.

The cargo in the MEVs in the compositions can comprise a biopolymer. Biopolymers include a naturally-occurring biopolymers, or synthetic biopolymers, or modified biopolymers. The biopolymer can be a nucleic acid or protein that includes modifications, where the modifications comprise insertions, deletions, replacements, and transpositions of nucleotides or amino acid residues, and/or, where the biopolymer is a protein, the modifications also can comprise post-translational modifications. Post-translational modifications include, but are not limited to, glycosylation, hyper-glycosylation, PEGylation, sialylation, albumination, other half-life extending moieties, and other modifications that improve or alter pharmacological dynamic or kinetic properties of the protein.

Nucleic acids, such as DNA and RNA, are among the molecules that can be cargo. If the cargo is RNA or protein, it can be provided as the cargo or it can be encoded by nucleic acid that then is expressed in the organism to whom it is administered. Exemplary of RNA is inhibitory RNA (RNAi) and mRNA, including modified mRNA. RNAi includes, for example, silencing RNA (siRNA) or short-hairpin RNA (shRNA), micro-RNA (miRNA), short activating RNA (saRNA), and long non-coding RNA (lncRNA). RNA products also include double stranded RNA and ribozymes. The cargo also can be an oligonucleotide, such as an anti-sense oligonucleotide or an allele-specific oligonucleotide. The cargo can comprise a gene editing system, such as a CRISPR-Cas system, and modified and improved gene editing systems, such as CRISPR-associated and CRISPR-like systems (see, e.g., published US patent application Nos. 20200332273 and 20200332274 each to Applicant Metagenomi, Inc.).

The cargo includes therapeutic or diagnostic or theranostic proteins or peptides, protein complexes, such complexes that contain two or more proteins or a protein and nucleic acid, or a protein and aptamer, or combinations of proteins, nucleic acids, and other molecules. The cargo can be or can encode a protein that is an antibody or antigen-binding fragment thereof. Antibodies can be of any form, including single chain forms, nanobodies, camelids, and other forms, such as an scFv, a bi-specific antibody, or an antigen-binding fragment thereof. Antibodies and antigen-binding fragments thereof include a checkpoint inhibitor antibody or antigen-binding fragment thereof, or a tumor antigen-specific antibody or antigen-binding fragment thereof, or an anti-oncogene specific antibody or antigen-binding fragment thereof, or a tumor-specific receptor or signaling molecule antibody or antigen-binding fragment thereof. Exemplary antibodies and antigen-binding fragments thereof specifically bind to and inhibit one or more of CTLA-4, PD-1, PD-L1, PD-L2, the PD-1/PD-L1 pathway, the PD-1/PD-L2 pathway, HER2, EGFR, TIM-3, LAG-3, BTLA-4, HHLA-2, CD28, and other checkpoints or immune suppressors, or tumor antigens.

The cargo in the MEVs in the compositions can include immune stimulating products, or antigens, and can be used as a vaccine to induce an immunoprotective response upon administration. The cargo can be a DNA, RNA, protein, or virus. The cargo can contain nucleic acid or protein or a nucleic acid encoding a protein that is a therapeutic vaccine for preventing or treating a disease, disorder, or condition, such as cancer, or an infectious disease, or another disease treated by immune modulation.

The cargo can comprise DNA. The DNA can be a plasmid, such as one that encodes a product for expression in the animal or plant to which it is administered. Exemplary products include therapeutic products and diagnostic products. These include proteins and RNA products, including the RNA products listed above. Since the MEVs are intended for administration to animals and plants, the plasmids generally encode the product under control of eukaryotic regulatory signals and sequences, including eukaryotic promoters and translation sequences, such as RNA polymerase II and III promoters. Exemplary promoters include RNA polymerase II promoters, such as from animals, plants, and plant or animal viruses. Exemplary promoters, include, but are not limited to, a cytomegalovirus promoter, a simian virus 40 promoter, a herpes simplex promoter, an Epstein Barr virus promoter, an adenovirus promoter, a synthetic promoter, an actin promoter, and synthetic chimeric promoters. Other eukaryotic transcription sequences and eukaryotic translation sequences, include, but are not limited to, one or more of an enhancer, a poly A sequence, and/or an internal ribosome entry site (IRES) sequence.

The plasmids can encode one or two or more cargo products. For expression of the cargo product the encoding nucleic acid is operably linked to regulatory sequences recognized by a eukaryotic cell.

Methods of preparing the MEVs are described. The methods include introducing the cargo into isolated MEVs. The cargo includes any molecule for whom delivery into or onto an animal or plant is desired. Generally, the cargo is or contains or provides a bioactive molecule product, including small molecules and biopolymers. The biopolymers are naturally-occurring, or synthetic, or modified, or combinations thereof. The cargo includes a protein, nucleic acid, or small molecule. The cargo can be loaded into the MEVs by any method known to those of skill in the art; these methods include, for example, one or more of electroporation, sonication, extrusion, and use of surfactants. In some embodiments the MEVs are from Chlorella, such as but not limited to a species of Chlorella selected from among Chlorella ellipsoidea, Chlorella pyrenoidosa, Chlorella sorokiniana, Chlorella vulgaris, and Chlorella variabilis. The MEVs produced by the methods and any of the MEVs provided herein, including the compositions containing the MEVs can be used as one or more of: a method of diagnosis, a vaccine, a therapy for treatment, a diagnostic of a disease, a treatment of a disease or disorder or condition, a cosmetic, an industrial application, and/or any use known to those of skill in the art.

The cargo can provide therapeutic molecules for treatment, or can induce an immune response to serve as a vaccine. The MEVs can contain a cargo that comprises an immunostimulatory protein or an antigen or encodes an immunostimulatory protein or antigen, whereby the MEVs, upon administration are immunostimulating and elicit an innate or adaptive immune response, or the MEVs and/or the cargo can elicit an immunoprotective response to prevent or treat a disease or disorder or condition.

The compositions containing the MEVs can be formulated for administration by any route of administration. Routes include, but are not limited to, local, systemic, topical, parenteral, enteral, mucosal, oral or nasal inhalation into the lung, intranasal, vaginal, rectal, aural, oral, and other routes of administration. The MEVs can be formulated in any form, including as a tablet; as a liquid, such as an emulsion; as a powder; as a cream; as a gel; or as an aerosol; the form and formulation respective to the route of administration including for oral administration, for nebulization, or for inhalation. For vaccination routes include oral and IM.

The microalgae extracellular vesicles can be loaded by any suitable method (see, methods and MEVs described in International Patent Publication No. PCT/EP2022/070371 published as International PCT publication WO2023/001894, U.S. provisional application Ser. No. 63/349,006), which include exogenous loading following production of the MEVs and also endogenous loading in vivo by microalgae genetically modified to package nucleic acids or encoded products into MEVs.

The EVs are from microalgae, which are unicellular green algae, and include those that belong to the order Chlorellales, in particular the Chlorellaceae family, and in particular those that belong to the Chlorella genus, such as Chlorella vulgaris. The MEVs are provided in compositions formulated for nasal administration. The MEVs can be loaded exogenously after isolation, or can be endogenously loaded by genetically modified microalgae that encode and package heterologous nucleic acid and/or proteins in the MEVs in vivo. An advantage of exogenously loading (exo-loading) cargo into MEVs is that the amount of cargo/MEV can be controlled, and distribution of the exogenous cargo in the MEVs is predictable, and substantially uniform, such that the average cargo molecule or amount of cargo/MEV can be known. A large variety of bioactive molecules, including biomolecules and small molecules, such as drugs and organic compounds, can be loaded into the MEVs. The MEVs also can be endogenously loaded by genetically modified microalgae to package heterologous nucleic acids and/or proteins.

The resulting MEVs, whether endo- or exo-loaded are not toxic; they can be administered into cells in vitro, or can be administered in vivo and have distribution patterns that depend upon the route of administration. The MEV is a unique vehicle for delivery of cargo to specific tissues, which delivery depends upon the route of administration. Trafficking of MEVs in vivo, as shown, can be distinct from MEVs from other sources. For example, in contrast to mammalian EVs, MEVs can be orally administered and traffic through the GALT.

The compositions provided herein and the compositions used in the methods can be formulated as a suspension or as an emulsion, such as a nanoemulsion or as a microemulsion. Those of skill in the art understand and are familiar with the properties of nanoemulsions and microemulsions and their formation. In the compositions, the MEVs contain the bioactive cargo. For example, the MEVs can be prepared so that on the average each MEV contains a pre-determined amount of bioactive molecule, such as, for example, 1 to 100 of the bioactive molecules per MEV. The selection of amount of cargo per MEV is within the level of skill in the art and depends upon factors known to the skilled artisan, such as the particular disease, disorder, or condition treated or the use of the MEVs, the subject, the particular cargo, and other such parameters and factors. Similarly, the concentration of MEVs depends upon the particular cargo and use. For example, the concentration of MEVs in the composition can be about or at 0.1 to 10 mg/mL, and lower or higher, and intermediate concentrations. The compositions can be formulated for single dosage administration (direct administration without dilution), or multiple dose administration for administration in aliquots and/or for dilution to a desired concentration. Exemplary amounts of compositions for administration are 0.1 to 100 mL, such as 1 to 10 mL, 1 to 5 mL, 0.1 to 1 mL, and any suitable amount. The compositions can be administered as a single dose or as a series of doses or other regimen. The compositions can be administrated as part of a combination therapy protocol.

The compositions can be formulated, for example, as a liquid, a powder, a troche, granules, a liquid, an oil, a suspension, or an emulsion, suitable for intranasal administration or processing, such as by dilution or dissolution for intranasal administration.

The compositions and methods include those in which the MEVs were endogenously loaded by genetically-modified microalgae that encode the bioactive molecule or a pathway for its production. The MEVs also include those in which the cargo was exogenously loaded in purified or partially purified MEVs. The MEVS can contain a plurality of different heterologous cargos.

The microalgae used to produce the MEVs for use in the methods can be microalgae from a division of microalgae selected from among Euglenophyta (Euglenoids), Chrysophyta (Golden-brown algae and Diatoms), Pyrrophyta (Fire algae), Chlorophyta (Green algae), Rhodophyta (Red algae), Phaeophyta (Brown algae), and Xanthophyta (Yellow-green algae). For example, the microalgae is a species of Chlorophyceae or Trebouxiophyceae or Chlorophyta, such as Chlorella or Chlamydomonas.

Chlorella species include, but are not limited to, Chlorella ellipsoidea, Chlorella pyrenoidosa, Chlorella sorokiniana, Chlorella vulgaris, and Chlorella variabilis, such as Chlorella vulgaris and Chlorella variabilis. In particular embodiments, the Chlorella is Chlorella vulgaris. For example, the methods and compositions include those in which the microalgae is a species of Chlorella; the MEVs in the composition contain heterologous bioactive molecule cargo that has been exogenously introduced into the isolated MEVs, whereby, on the average, the vesicles in the composition that contain the heterologous bioactive molecule cargo contain the same heterologous cargo, where: the cargo is heterologous to Chlorella; and the cargo is a biomolecule or a small molecule drug or any cargo for use as vaccines for delivery of antigens and/or immune modulators to the immune system. Also included are methods and compositions in which the MEVs are Chlorella extracellular vesicles; the Chlorella extracellular vesicles comprise a heterologous bioactive molecule cargo that is endogenously introduced into the extracellular vesicles by the microalgae, wherein the cargo molecule is heterologous to Chlorella; and the bioactive cargo is a biomolecule for treating a disease, disorder, or condition of the immune system or involving the immune system, or that can be treated by a vaccine or immunomodulator.

Provided are methods and compositions, where: the MEVs are Chlorella extracellular vesicles; the Chlorella extracellular vesicles comprise a heterologous bioactive molecule cargo that has been introduced into isolated extracellular vesicles, whereby the vesicles in the composition that contain the heterologous bioactive molecule cargo contain, on average, the same bioactive molecule cargo, where: the cargo molecule is heterologous to Chlorella; and the bioactive cargo comprises antigens and/or immunomodulators. In other embodiments, the MEVs are Chlorella extracellular vesicles; the Chlorella extracellular vesicles comprise a heterologous bioactive molecule cargo that is endogenously introduced into the extracellular vesicles by the microalgae, whereby the vesicles in the composition that contain the heterologous bioactive molecule cargo contain the same bioactive molecule cargo, where: the cargo molecule is heterologous to Chlorella; and the bioactive cargo is a biomolecule or a small molecule. In other embodiments, the MEVs in the composition contain heterologous bioactive molecule cargo that has been exogenously introduced into the isolated MEVs, whereby on the average the vesicles in the composition that contain the heterologous bioactive molecule cargo contain the same cargo, where: the cargo is heterologous to Chlorella; and the cargo is a biomolecule or a small molecule. In other embodiments, the cargo is endogenously introduced into the MEVs by modifying the microalgae to express or produce the cargo, such as a nucleic acid or protein, or biochemical pathway product. In exemplary embodiments, the Chlorella is Chlorella vulgaris.

Cargo includes, but is not limited to, a biomolecule, a biopolymer, such as a naturally-occurring biopolymer, or is a synthetic biopolymer, or is a modified biopolymer, such as, for example, a nucleic acid molecule, a polypeptide, a protein, a plasmid, an aptamer, or an antisense oligonucleotide. Cargo includes, but is not limited to, DNA or RNA, such as, for example, inhibitory RNA (RNAi), mRNA or modified mRNA, silencing RNA (siRNA), short-hairpin RNA (shRNA), micro-RNA (miRNA), self-amplifying RNA, short activating RNA (saRNA), long non-coding RNA (lncRNA), a ribozyme, or a double-stranded RNA. Cargo includes oligonucleotides, such as an anti-sense oligonucleotide or an allele-specific oligonucleotide or an anti-sense oligonucleotide (ASO), a gene editing system, such as for example a CRISPR-CAS system, a CRISPR-associated or CRISPR-like system(s). The cargo can comprise DNA, such as a plasmid, where the plasmid encodes the therapeutic and/or detectable or diagnostic product, or an RNA product, such as RNAi and the forms of RNA noted above, including an anti-sense oligonucleotide or a ribozyme or a double-stranded RNA. The plasmid can encode the cargo product under control of a eukaryotic promoter, such as an RNA polymerase II or III promoter, such as a eukaryotic virus promoter, such as, for example, a cytomegalovirus promoter, a simian virus 40 promoter, a herpes simplex promoter, an Epstein Barr virus promoter, an adenovirus promoter, a synthetic promoter, and other promoters, such as an actin promoter, or a synthetic chimeric promoter. The plasmid can also comprise other regulatory sequences for expression, such as other eukaryotic transcription sequences and eukaryotic translation sequences. The MEV cargo can comprise a small molecule for effecting treatment or detection or diagnosis or monitoring of a disease, disorder, or condition.

The MEVs can comprise two or more cargo products. Cargo can comprise a therapeutic product, or a diagnostic product, or a detectable product, or combinations thereof, for detecting, diagnosing and/or monitoring a disease, disorder, or condition involving the immune system or for modulating the immune system, including as a vaccine. The cargo can comprise one or more of bioactive small molecules, peptides (polypeptides, proteins), RNAs (mRNAs, siRNAs, miRNAs, lncRNAs), DNAs (anti-sense oligonucleotide (ASOs), plasmids, DNA fragments), and gene editing complexes. Cargo can comprise, for example, an immunomodulatory compound, and a combination of the immunomodulatory compound and an antigen, or antigen, or nucleic acid encoding the compound and/or antigen.

### MEVs for Vaccination and Immunomodulation

Provided are vaccine compositions that contain an MEV or MEVs containing cargo that is effective for vaccination and/or immune modulation. Vaccines can be used for treating or preventing, including reducing the risk of, or reducing the severity of a disease, disorder, or condition. The diseases, disorders, and conditions include, for example, infections, cancers, and other diseases, disorders, and conditions that can be treated by modulating the immune system.

The vaccine compositions include compositions comprising an MEV or MEVs, where: the MEV comprises cargo that comprises an antigen or immunogen and/or an immunomodulator, or comprises nucleic acid encoding the antigen, immunogen, and/or immunomodulator; the vaccine is for treating, preventing, or reducing the severity of a disease, disorder, or condition; the immunomodulator is an agent that acts on the immune system directly or indirectly; and the composition is formulated for administration by a route whereby the MEVs traffic to a cell, tissue, or organ of the immune system. The vaccine compositions can comprise an MEV or MEVs, where: the MEVs comprise cargo that comprises one or more of an antigen or immunogen and/or an immunomodulator, or comprises nucleic acid encoding the antigens, immunogens, and/or immunomodulators; the vaccine is for treating, preventing, or reducing the severity of a disease, disorder, or condition; the immunomodulator is an agent that acts on the immune system directly or indirectly; the composition is formulated for administration by a route whereby the MEVs traffic to a cell, tissue, or organ of the immune system. The cargo can comprise an antigen or polypeptide or portion thereof, or nucleic acid, such as mRNA or DNA, encoding the antigen or polypeptides or portion thereof. For all of these embodiments, the cargo can comprise a polypeptide or antigenic portion thereof, or an epitope or neoepitope thereof or can comprise an immunomodulator, including those described below and/or known to those of skill in the art, are a combination of a polypeptide or antigenic portion thereof, or an epitope or neoepitope thereof and an immunomodulator, or nucleic acid, such as mRNA or DNA, encoding the polypeptide or antigenic portion thereof, or an epitope or neoepitope thereof and/or the immunomodulator.

In embodiments herein, the vaccine compositions do not comprise an exogenous adjuvant or “traditional” adjuvant. As shown in the examples, the immune response to compositions comprising the MEVs and no added adjuvant, result in an immune response as robust as the immune response to adjuvant plus the same polypeptide or nucleic acid. For the compositions and the methods provided herein, the compositions do not require an exogenous adjuvant or a “traditional” adjuvant. It is shown herein that the vaccine compositions can be or are administered a plurality of times. The data show no adverse effects or immune reactions against the MEVs. Vaccine compositions provided herein result in a cellular response comprising T memory cells, T cells, and/or any other kinds of helper T cells, effector T cells, regulatory T cells or other T cells.

Exemplary cargo can comprise an antigen or polypeptide or portion thereof from a pathogen or from a tumor or cancer, or encoding nucleic acid, and/or an immunomodulator or encoding nucleic acid. Exemplary immunomodulators include, but are not limited to, a cytokine or chemokine or receptor agonist or antagonist, or a receptor, or ligand that modulate an immune response. Combinations of immunomodulators also are included. Cargo can comprise an antigen that is an immunogenic protein, polypeptide, peptide from a pathogen, or encoding nucleic acid.

Generally, the antigens from a pathogen are selected to induce a robust immune response, and can result in neutralizing antibodies. Exemplary pathogens include, but are not limited to, bacteria, such as, for example, one or more of Enterobacteriales (Shigella sp. Salmonella sp. Escherichia coli, among other species of the order), Vibrionales (Vibrio cholerae, among other species of the order), Legionellales (Legionella pneumophila, among other species of the order), Pseudomonadales (Pseudomonas aeruginosa, P syringae, Acinetobacter spp., Moraxella spp., among other species of the order), Pasteurellales (Haemophilus influenzae, Mannheimia spp., Actinobacillus spp., among other species of the order), Porphyromonas gingivalis, and or gram-positive bacteria such as Staphylococcus aureus, Staphylococcus spp., Streptococcus pneumonia, Streptococcus spp., or Bacillus spp., Listeria spp., Clostridium spp., Nocardia spp; and viruses, including, but not limited to, hepatitis viruses, herpesvirus, varicella-zoster virus (VZV), Epstein-Barr virus (EBV), human immunodeficiency virus (HIV), human T-cell leukemia virus (HTLV), respiratory syncytial virus (RSV), measles virus, influenza virus, rhinovirus, and coronaviruses, such as Severe Acute Respiratory Syndrome coronavirus (SARS-CoV), Middle East Respiratory Syndrome coronavirus (MERS-CoV), and Severe Acute Respiratory Syndrome coronavirus 2 (SARS-CoV-2). Exemplary antigens or nucleic acid encoding the antigens are selected from among the antigens and immunogenic portions thereof or epitopes thereof, see table 1, below.

TABLE 1
List of exemplary antigens for use as possible MEV cargo

| | Pathogen | |
|---|---|---|
| Antigen name | type | Species of origin |
| Heat-labile enterotoxin B subunit | Bacterial | Escherichia coli |
| Cholera toxin B (CTB) subunit | Bacterial | Vibrio cholerae |
| Extracellular capsule protein F1/ | Bacterial | Yersinia pestis |
| immune-modulator V fusion protein | | |
| Outer membrane protein receptor for | Bacterial | Shigella flexneri |
| ferrichrome | | |
| Outer membrane protein OprF | Bacterial | Pseudomonas |
| | | aeruginosa |
| N-terminal portion of the Candida | Bacterial | Staphylococcus |
| albicans agglutinin-like protein 3 | | aureus |
| (Als3p) | | |
| 27-kDa outer membrane protein | Bacterial | Salmonella enterica |
| (T2544) | | serovar Typhi |
| Hepatitis B surface antigen (HBsAg) | Viral | Hepatitis B virus |
| | | (HBV) |
| E1-E2 genome polyprotein | Viral | Hepatitis C virus |
| | | (HCV) genotype 1a |
| Inner capsid protein VP6 | Viral | Human rotavirus A |
| Outer capsid glycoprotein VP7 | Viral | Rotavirus A |
| Capsid protein | Viral | Norwalk virus (NV) |
| Spike protein S1 fragment | Viral | SARS-coronavirus |
| | | (CoV) |
| B5R antigenic ectodomain | Viral | Vaccinia virus |
| Envelope protein (E) | Viral | Japanese encephalitis |
| | | virus (JEV) |
| VP4N20 antigenic peptide | Viral | Coxsackievirus 16 |
| | | (CV-A16) |
| VP4N20 antigenic peptide | Viral | Enterovirus 71 |
| | | (EV71) |
| Minor capsid protein L | Viral | Human |
| | | papillomavirus type |
| | | 16 (HPV-16) |
| Envelope glycoprotein D | Viral | Human herpesvirus 1 |
| | | (HHV-1) |
| Envelope domain III protein | Viral | Zika virus |
| Major surface glycoprotein G | Viral | Human respiratory |
| | | syncytial virus A |
| | | (RSV strain A2) |
| Domain III fragment of dengue 2 | Viral | Dengue virus type 2 |
| envelope protein (D2EIII) | | |
| Merozoite surface protein 4 (MSP4) | Protozoan | Plasmodium |
| | | falciparum |
| Merozoite surface protein 5 (MSP5) | Protozoan | Plasmodium |
| | | falciparum |
| Trans-sialidase | Protozoan | Trypanosoma cruzi |
| A2 protein | Protozoan | Leishmania infantum |
| N-terminal portion of the Candida | Fungal | Candida albicans |
| albicans agglutinin-like protein 3 | | |
| (Als3p) | | |

These also include antigens where the sequence of the antigen is set forth in any of SEQ ID Nos:160-186 and/or is an immunogenic, antigenic, or epitope portion thereof.

The cargo in the MEV or MEVs in the vaccine compositions can comprise an immune modulator or combinations thereof or combinations thereof with an antigen or antigens. Generally, the response to the immune modulator will complement the antigen, by, for example, inducing an immune response that is enhanced or improved and/or reduces undesirable or adverse immune responses. Exemplary immune modulators include, but are not limited to, one or more of a cytokine, chemokine, co-stimulatory molecule, TNF superfamily of ligands or receptors, Toll-like receptor (TLR) agonist or antagonist, or immune checkpoint inhibitor, or a type I interferon or interferon-γ. The immune modulator can be an antibody or antigen-binding fragment thereof that specifically binds to and inhibits a receptor or ligand involved in a disease, disorder, or condition, such as one or more of CTLA-4, PD-1, PD-L1, PD-L2, the PD-1/PD-L1 pathway, the PD-1/PD-L2 pathway, HER2, EGFR, TIM-3, LAG-3, BTLA-4, HHLA-2, CD28, and other checkpoints or immune suppressors, or tumor antigens.

Exemplary vaccine compositions include cargo that comprises: an antigen or nucleic acid encoding the antigen, wherein the antigen is selected from among the antigens and immunogenic portions thereof or epitopes thereof, and an immunomodulator.

The vaccine compositions can be formulated for a route of administration, particularly routes that deliver or traffic cargo to or through cells, tissues, and/or organs of the immune system. The MEVs are unusual in that they can be administered orally where they traffic to the gut-associated lymphoid tissue (GALT), and from there to other organs of the immune system, such as the spleen, including the white spleen. They also can be administered by other routes. Provided are vaccine compositions that are formulated for oral administration, intramuscular administration, inhalation into the lungs or nose, mucosal administration, or local administration, or subcutaneous administration. Provided are vaccine compositions formulated for administration by a route that comprises the target the tissues of the spleen, and/or that targets or traverse gut-associated lymphoid tissue (GALT). The compositions can be formulated for administration by a route that comprise or target mucosal tissue. Hence provided are compositions formulated for oral administration, or formulated for administration by inhalation into the lungs or nose, or formulated for intramuscular administration. The vaccine compositions can be formulated as tablets, pills, powders, liquid solutions or suspensions (e.g., including injectable, ingestible and topical formulations, for example, eye drops, gels, pastes, creams, or ointments), aerosols (e.g., nasal sprays and inhalers), suppositories, pessaries, injectable and infusible solutions and sustained release forms.

Provided are vaccine compositions for use for oral administration for treating, preventing or reducing the severity of a disease, disorder, or condition involving a pathogen or for a disease, disorder, or condition that is cancer, or an immune system disorder. The vaccine compositions provided herein can include cargo that comprises nucleic acid encoding the antigen or portion thereof or an antigenic portion or epitope or immunomodulator, and/or cargo that comprises mRNA encoding the antigen or portion thereof or immunomodulator. The cargo can comprise DNA encoding the antigen or portion thereof or immunomodulator. The cargo can comprise a plasmid encoding the antigen or portion thereof and/or an immunomodulator. The cargo can comprise a protein antigen, or antigenic portion thereof, or an epitope.

Provided are methods of vaccination, including treatment, prevention or reduction of the severity of a disease, disorder, condition, including cancer, comprising administering any of the vaccine compositions provided herein to a subject. The methods include orally or intramuscularly administering the vaccine compositions. As discussed above and below, the compositions can be for oral administration for use for treating or preventing a disease, disorder, or condition involving a pathogen or for a disease, disorder, or condition that is cancer, or an immune system disorder. They can be for intramuscular administration for treating or preventing a disease, disorder, or condition involving a pathogen or for a disease, disorder, or condition that is cancer, or an immune system disorder. Other routes of administration also are contemplated.

It is shown herein that the vaccine compositions and methods can elicit a protective humoral response that comprises serum IgG and IgA and/or mucosal IgG and IgA, such as a protective humoral response that comprises serum IgA and/or mucosal IgA, thereby generating vaccine-induced IgA-producing memory B-cells to provide systemic and mucosal responses that protect from reinfection. These responses are observed following administration by routes that include oral and intramuscular.

The vaccine compositions and methods deliver or traffic an antigen or immunogenic portion thereof, or an epitope, or nucleic acid encoding the antigen, or immunogenic portion thereof, or epitope and an immunomodulator, to reduce or eliminate immune-tolerance to previous immunotherapies or vaccines, such as those occurring in cancer.

The MEV cargo can comprise a TLR antagonist or agonist or nucleic acid encoding the antagonist or agonist. The selection of TLR and antagonist or agonist depends upon the disease, disorder, or condition treated or prevented or at issue; for some diseases, disorders, and conditions, immunosuppression of certain responses is desirable, and for others immune stimulation or response is desirable. The TLR and agonist thereof can be, for example, one or more of

| TLR | |
|---|---|
| Member | Ligand(s)/Agonists |
| TLR1 | Triacyl lipopeptides (Pam3CSK4) |
| TLR2 | Zymosan, Porin, Modulin, Lipoproteins, Lipoteichoic acid, |
| | Diacyl lipopeptides, Atypical LPS, Peptidoglycan, Triacyl |
| | lipopeptides |
| TLR3 | dsRNA |
| TLR4 | Mannans, Taxol, LPS |
| TLR5 | bacterial flagellin, profilin, HMGB1, Small molecule agonists |
| | (CBLB502) |
| TLR6 | Zymosan, Porin, Modulin, Lipoproteins, Lipoteichoic acid, |
| | Diacyl lipopeptides (Pam2CSK4), Atypical LPS, Peptidoglycan |
| TLR7 | imidazoquinoline, loxoribine, ssRNA, bropirimine, resiquimod |
| TLR8 | ssRNA, small synthetic compounds |
| TLR9 | CpG DNA |
| TLR10 | Diacyl and Triacyl lipopeptides |
| TLR11 | Profilin-like protein, non-pathogenic bacteria. |

In other embodiments, the TLR and antagonist can be one or more of

| TLR | |
|---|---|
| Member | Ligand(s)/Antagonists |
| TLR1 | Small molecule antagonists (CU-T12-9, MMG-11) |
| TLR2 | Small Molecule Antagonists (AT1-AT8, CU-CPT22, CU-T12-9, |
| | MMG-11, NPT1220-312), Phloretin, Sulfoglycolipids |
| TLR3 | Small Molecule Antagonists (CU-CPT4a), Monoclonal |
| | antibodies (CNTO4685, CNTO5429) |
| TLR4 | Small Molecule Antagonists (Norbinaltorphimine, T4Ics, |
| | T5342126, Simvastatin |
| TLR5 | Small Molecule Antagonist (TH1020) |
| TLR6 | Simvastatin |
| TLR7 | Chloroquine, hydroxychloroquine, quinacrine |
| TLR8 | Small Molecule Antagonist (CU-CPT8m, CU-CPT9a) |
| TLR9 | Small Molecule Antagonists (NPT1220-312), chloroquine, |
| | hydroxychloroquine, quinacrine; Suppressive or inhibitory |
| | oligonucleotides |

Depending upon the disease, disorders, or conditions, and desired outcome, the cargo can comprise combinations of agonists and antagonists that activate a particular TLR and inactivate a different TLR. For example, for cancer, immunosuppression of inflammatory and other anti-bacterial type of responses can be desired; for vaccination against pathogens, bacterial and inflammatory responses can be advantageous. For autoimmune diseases, immunosuppression can be desirable.

The vaccine compositions and methods can be used to treat or prevent a disease, disorder, or condition that is an inflammatory disease, disorder or condition, or a disease, disorder, or condition in which inflammation plays a role in the etiology of the disease, disorder, or condition. The immunomodulator can be one that suppresses the inflammatory response. The disease, disorder, or condition can comprise cancer and the cargo can comprise an immunomodulator that suppresses an inflammatory response but does not suppress and anti-cancer immune response. In other embodiments, the vaccine composition can be formulated for oral administration and the disease, disorder, or condition involves the gastrointestinal tract or the immune system or the white spleen.

The MEVs for the vaccine compositions and methods provided herein can be from a microalgae, including, but not limited to, MEVS from a division of microalgae selected from among Euglenophyta (Euglenoids), Chrysophyta (Golden-brown algae and Diatoms), Pyrrophyta (Fire algae), Chlorophyta (Green algae), Rhodophyta (Red algae), Phaeophyta (Brown algae), and Xanthophyta (Yellow-green algae). For example, the MEVs can be from Chlorella, such as a species of Chlorella selected from among Chlorella ellipsoidea, Chlorella pyrenoidosa, Chlorella sorokiniana, Chlorella vulgaris, and Chlorella variabilis.

In all embodiments of the compositions and methods, the subjects include, but are not limited to, mammals, including humans and/or non-human animals. The subjects include, but are not limited to, livestock or a pet or a zoo animal or a water mammal, such as, but not limited to, a non-human animal that is a dog, a cat, a gerbil, a rabbit, or other furry animals, an ovine, a bovine, a non-human primate, a goat, an elephant, a dolphin, or a whale.

All claims as filed herein are incorporated by reference into this section.

### BRIEF DESCRIPTION OF THE DRAWINGS

FIG. 1 depicts an exemplary elution profile for higher purity of MEV preparations, where MEVs previously concentrated by TFF and purified by ultracentrifugation and formulated in PBS at concentration of 10¹¹ to 10¹³ per mL are seeded in a pre-packed column qEV1 from IZON. The MEVs are eluted using PBS solution. The elution fractions of 0.5 mL are collected. MEVs are recovered in the first fractions as shown in the figure. The most concentrated fractions (4-5) are pooled and stored at 4° C. before use. [see, Example 1]

FIG. 2 provides exemplary images of MEVs obtained using Transmission Electron Microscopy (TEM). [see, Example 2]

FIGS. 3A-3B provides exemplary images obtained with MEVs labelled with lipophilic dyes by confocal microscopy. FIG. 3A MEVs labelled with PKH26. FIG. 3B MEVs labelled with DiD. [see, Example 2]

FIGS. 4A-4C depict MEVs uptake analysis by confocal microscopy. Uptake of MEVs labelled with PKH26 into cells after 16 hs of incubation. FIG. 4A layout of cells (2D image). FIG. 4B one cell (2D image). FIG. 4C one cell (3D image). [see, Example 2]

FIG. 5 Distribution of labelled MEVs with lipophilic dyes by cytometry. [see, Example 2]

FIGS. 6A-6B depict MEVs uptake analysis by cytometry. Uptake of MEVs (labelled with lipophilic dyes) in different cell types as percentage of fluorescent cells. FIG. 6A epithelial cells. FIG. 6B monocytes. [see, Example 2]

FIG. 7 provides representative patterns of biodistribution according to the route of administration, for the Intravenous (IV), Intratracheal (IT) and Per os (PO) routes. [see, Example 6A]

FIG. 8A depicts the kinetics of accumulation in liver, lungs, and spleen (average of 6 animals) after intravenous administration, as described in Example 6A.

FIG. 8B depicts the kinetics of accumulation in lungs, spleen, and intestine (average of 6 animals) Per os administration, as described in Example 6A.

FIG. 8C depicts the kinetics of accumulation lungs and kidneys (average of 4 animals) after intranasal administration, as described in Example 6A.

FIG. 8D depicts the kinetics of accumulation in lungs, spleen, and intestine (average of 3 animals) after intratracheal administration, as described in Example 6A.

FIG. 9 shows a microscopic image of mouse intestinal epithelium 8 hours after PKH26-labeled MEV administration by Per os route. [see, Example 6B]

FIGS. 10A-B show a microscopy image of mouse GALT tissue 8 hours after PKH26-labeled MEV administration by Per os route. FIG. 10A depicts Hematoxylin and Eosin staining of intestine (G=GALT tissue). FIG. 10B depicts DAPI (nuclei) staining, and MEV-PKH26 fluorescence (for example portion labeled “ro”). [see, Example 6B]

FIGS. 11A-B show a microscopy image of mouse spleen 24 h after PKH26-labeled MEV administration by Per Os route. FIG. 11A depicts spleen pulp with DAPI (nuclei) staining, and MEV-PKH26 is indicated by fluorescence (lighter gray staining/puncta). FIG. 11B is a diagram showing the migration of MEVs from the GALT to the spleen. [see, Example 6B]

FIG. 12 shows whole-body bioluminescence imaging of a representative animal treated with MEVs loaded with luciferase mRNA. [see, Example 7]

FIG. 13 depicts whole-body bioluminescence imaging of a representative animal treated with MEVs loaded with luciferase enzyme. [see, Example 7]

FIGS. 14A-C show responses to antigen (ovalbumin (OVA)) administered orally or intramuscularly with adjuvant compared to the responses following administration of MEVs loaded with OVA by the same routes. [see, Example 9]

FIGS. 15A-B shows non-hematological toxicity response in mice after administered orally or intratracheally with MEVs. FIG. 15A depicts evaluation of MEV's toxicity by chemistry parameters: ALAT, ASAT, urea and creatine. FIG. 15B depicts evaluation of MEV's toxicity by hematology parameters: RBCs, hemoglobin, hematocrit, MCV and Eosinophils. Group 1: mice received 100 μl of PBS (White bars) by PO delivery. Group 2: mice received 100 μl of 4*10¹¹ MEVs/mouse by PO delivery (bar with black and white tiles). Group 3: mice received 100 μl of 4*10¹² MEVs/mouse by PO delivery (bars with vertical lines). Group 4: mice were administered 100 μl of 4*10¹¹ MEVs/mouse by IT delivery (squared bars). Data were obtained for 6 mice per group for each parameter. ALAT: Alanine Aminotransferase. ASAT: Aspartate Aminotransferase. MCV: Mean Corpuscular Volume. PO: per os (oral delivery). IT: Intratracheal. [see, Example 18]

FIG. 16 depicts the immune response to immunization with an adjuvant plus and antigen. MEVs plus antigen follow a similar route, except that MEVs contain the antigen (or nucleic acid encoding the antigen) as cargo inside the MEV. The antigen can be expressed on the surface of the MEV, or otherwise delivered into the host cell as a protein or nucleic acid (cDNA or mRNA or eRNA). MEVs provide delivery systems.

FIGS. 17A and 17B show MEVs and their interactions with TLRs and ligands therefor. [see, Examples 17A; 17B]

FIGS. 18-FIG. 26B present the T-cell response following intramuscular administration of MEVs and MEVs containing exemplary antigen (OVA).

FIG. 18 is a drawing of scatter density plots of results following IM administration: Gating for CD44/CD62L. [see, Example 8]

FIG. 19A is a bar graph depicting IM administration results: CD44/CD62L (spleen). [see, Example 8]

FIG. 19B is a bar graph depicting IM administration results: CD44/CD62L (spleen). [see, Example 8]

FIG. 20A is a bar graph depicting IM administration results: CD44/CD62L (lymph nodes (LN)). [see, Example 8]

FIG. 20B is a bar graph depicting IM administration results: CD44/CD62L (lymph nodes (LN)). [see, Example 8]

FIG. 21A is a drawing showing scatter density plots of results following IM administration: Gating for CD44(hi)/CD49d. [see, Example 8]

FIG. 21B is a drawing showing scatter density plots of results following IM administration: Gating for CD44(hi)/CD49d. [see, Example 8]

FIG. 22A is a bar graph depicting IM administration results: CD44(hi)/CD49d (spleen). [see, Example 8]

FIG. 22B is a bar graph depicting IM administration results: CD44(hi)/CD49d (spleen). [see, Example 8]

FIG. 23A is a bar graph depicting IM administration results: CD44(hi)/CD49d (LN). [see, Example 8]

FIG. 23B is a bar graph depicting IM administration results: CD44(hi)/CD49d (LN). [see, Example 8]

FIG. 24A is a drawing showing a scatter density plot of the results following IM administration: Gating for CD44(hi)/CD11a(hi). [see, Example 8]

FIG. 24B is a drawing showing a scatter density plot of the results following IM administration: Gating for CD44(hi)/CD11a(hi). [see, Example 8]

FIG. 25A is a bar graph depicting IM administration results: CD11a/CD49d (spleen). [see, Example 8]

FIG. 25B is a bar graph depicting IM administration results: CD11a/CD49d (spleen). [see, Example 8]

FIG. 26A is a bar graph depicting IM administration results: CD11a/CD49d (LN). [see, Example 8]

FIG. 26B is a bar graph depicting IM administration results: CD11a/CD49d (LN). [see, Example 8]

FIGS. 27-FIG. 30 present the macrophage RAW264.7 response following incubation at different timepoints of MEVs labelled with pKH26 or loaded with TLRs agonists.

FIGS. 27-27B depict MEVs internalization into RAW264.7 macrophage M0 mice cells.

FIG. 27A provides images of confocal microscopy (63× magnification) of MEV penetration into RAW264.7 cells (PKH26 fluorescence) at different time points. White arrows show, as indicated, the presence of the MEVs into the cells. FIG. 27B shows the quantification (of the total fluorescence intensity (MFI) from the PKH26) of 4 campus per treatment after confocal microscopy of MEVs penetration into RAW264.7 cells (PKH26 fluorescence) at different time points. [see, Example 17A]

FIG. 28 MEVs payload delivery into RAW264.7 cells. Images of confocal microscopy (63× magnification) of MEV penetration and payload delivery into RAW264.7 cells (FITC—fluorescence) after 16 h of incubation for all conditions. LPS, an agonist of TLR4 (line 2), and Poly (I.C), an agonist of TLR3 (line 4), are used here as a positive control. In this picture, fluorescence comes from the payload (poly IC labelled with FITC). [see, Example 17A]

FIGS. 29A-29B depict activation of NF-Kb intracellular pathway after MEV-mediated delivery of an agonist of TLR3 into RAW264.7 cells. FIG. 29A provides images of confocal microscopy (63× magnification) of activation of NF-Kb pathway on RAW264.7 cells after stimulation of TLR3. The red fluorescence corresponds to anti-pNF-Kb p65 MoAb (serine536). In this picture, fluorescence comes from the activated NF-Kbp65. FIG. 29B shows a Western Blot of total proteins from cell lysates of RAW264.7 cells after different treatments. Activation of NF-Kb pathway on murine macrophage M0 cells after stimulation of TLR3. [see, Example 17A]

FIGS. 30A-30B show differentiation of RAW264.7 cells into Macrophages and Dendritic Cells by stimulation of MEVs loaded with TLR3 agonist. FIG. 30A provides images of microscopy (20× magnification) of morphological differentiation into macrophages (M1 or M2) and dendritic cells of RAW264.7 cells (M0) after MEVs penetration and payload delivery. The payload poly-IC (HMW) is an agonist of TLR3.

FIG. 30B shows magnification by software of Image of condition 6 of A. [see, Example 17A]

FIGS. 31-FIG. 32 present the human epithelial cells response following incubation at different timepoints of MEVs labelled with pKH26 or loaded with TLRs agonists.

FIGS. 31A-31E show MEVs internalization and payload delivery into human epithelial cells in vitro. FIG. 31A and FIG. 31C provide images of confocal microscopy (63× magnification) of MEV penetration into intestinal epithelial cells (pKH26 fluorescence) at different time points. White arrows show, as indicated, the penetration of MEVs into the cells. FIG. 31B and FIG. 31D show the quantification (fluorescence intensity (MFI)) of 4 campus per treatment after confocal microscopy of MEVs penetration into intestinal epithelial cells (pKH26 fluorescence) at different time points. FIG. 31E provides images of confocal microscopy (63× magnification) of MEV penetration and payload delivery into lung epithelial cells (FITC—fluorescence) at different time points. White arrows show, as indicated, the penetration of MEVs and payload delivery into the cells. [see, Example 17B]

FIGS. 32A-32E show payload delivery and biological activity of loaded-MEV penetration into human epithelial cells in vitro. FIG. 32A provides images of confocal microscopy (63× magnification) of activation of NF-Kb pathway on BEAS-2B cells after stimulation of TLR-3. The red fluorescence corresponds to anti-pNF-Kb p65 MoAb (serine536). FIG. 32B shows a Western Blot of total proteins from cell lysates of BEAS-2B cells after different treatments. Activation of NF-Kb pathway on BEAS-2B cells after stimulation of TLR-3. FIG. 32C shows a Western Blot of total proteins from cell lysates of FHC cells after different treatments. Activation of NF-Kb pathway on FHC cells after stimulation of TLR-3. FIG. 32D shows a Western Blot quantification. Activation of NF-Kb pathway on FHC cells after stimulation of TLR-3. The quantification was normalized using the value of control cells (non-treated cells) as 1, the values of all condition are relative to the control. FIG. 32E provides images of confocal microscopy (63× magnification) of activation of IRF-3 pathway after stimulation of TLR-9 on FHC cells. The red fluorescence corresponds to anti-pIRF3 MoAb (serine396). [see, Example 17B]

### DETAILED DESCRIPTION

### Outline

A. DEFINITIONS
B. MICROALGAE AND OVERVIEW
C. EXTRACELLULAR VESICLES
   1. Types of Extracellular Vesicles (EVs)
      a. Exosomes
      b. Microvesicles
      c. Apoptotic Bodies
   2. Uptake of Evs
   3. General Methods for Isolating Evs
      a. Ultracentrifugation
      b. Size-Based Techniques
      c. Immunoaffinity Capture-Based Techniques
      d. Exosome Precipitation
      e. Microfluidic Based Isolation Techniques
   4. Microalgae and Microalgae-Derived Extracellular Vesicles (MEVs)
   5. Green algae-Chlorella species
      a. Life Cycle
      b. Genomic Analyses of Chlorella Species
      c. Commercial and Biotechnological Uses of Chlorella
      d. Chlorella MEVs
D. EXOGENOUSLY LOADED MICROALGAE EXTRACELLULAR VESICLES (MEVS), CARGO, AND TARGETS
   1. Isolation of MEVs
   2. MEV Loading and Cargos
   3. Generation of Payload-Loaded MEVs
      a. Electroporation
      b. Sonication
      c. Extrusion
      d. Surfactants
      e. Other Methods
   4. Exemplary Cargo and Exemplary Uses of the Exogenously Loaded MEVs
      a. Cargo
         1) RNA Cargo
         2) Antibody Cargo
   b. Diseases and Methods of Treatment
E. ENDOGENOUSLY LOADED (ENDO-LOADED) MICROALGAE EXTRACELLULAR VESICLES (MEVS), CARGO, AND TARGETS
   1. Choice and preparation of Cargo
   2. Genetic engineering of producer cells
   3. Cargo
      1) Protein Cargo
F. PHARMACEUTICAL COMPOSITIONS, FORMULATIONS, KITS, ARTICLES OF MANUFACTURE AND COMBINATIONS
   1. Pharmaceutical Compositions and Formulations
   2. Articles of Manufacture/Kits and Combinations
   3. Administration of Cargo=Loaded MEVs and Routes of Administration
   4. Combination Therapies
G. BIODISTRIBUTION OF MEVs FOLLOWING ADMINISTRATION VIA VARIOUS ROUTES
   1. Biodistribution of mammalian Evs
   2. Microalgae Evs Biodistribution
      a. Oral Administration
         1) Components of the Lymphatic System
         2) Targeting GALT
      3. Diseases and conditions treated by MEVs
H. THE IMMUNE SYSTEM AND MEVs FOR USE AS VACCINES AND FOR DELIVERY OF IMMUNOMODULATORS
   1. Immune system and vaccines
   2. Vaccines—oral, intramuscular, and local administration, including mucosal administration, such as inhalation to the lungs and nasal tract
      a. MEV-based oral vaccines
      b. MEV-mediated immunization upon oral delivery
      c. MEV-mediated immunization and mucosal immunity
      d. MEV-mediated immunization upon intramuscular delivery
      e. Adjuvants
      f. Isotype switching
   3. MEVs and cargo
   4. Antigens
   5. Immunomodulators
      a. MEV-mediated intracellular signaling and other receptors and ligands for preventing, reducing the risk of, or treating a disease, disorder, or condition
I. FORMULATIONS, ROUTES OF ADMINISTRATION, AND DISEASE AND DISORDERS
J. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GenBank® sequences, databases, websites, and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change, and particular information on the internet can come and go, but equivalent information can be found by searching the internet. Reference thereto evidences the availability and public dissemination of such information.

As used herein, cargo refers exogenous molecules, such as bioactive molecules, including biomolecules, and small molecules, that are loaded into the microalgae extracellular vesicles (MEVs) provided herein after the MEVs have been isolated. This includes cargo that is heterologous to the MEVs.

As used herein, in general, heterologous with respect to cargo in an MEV refers to cargo in the MEVs that does not naturally-occur in the MEVs but is loaded exogenously, as discussed above. It also refers to cargo in MEVs that have been loaded endogenously in the MEVs by genetically-modified microalgae. MEVs with heterologous cargo, comprise cargo that does not occur naturally in the MEVs.

As used herein, a bioactive molecule or bioactive agent refers to any molecule or agent that can have a biological activity, such as therapeutic activity, or as a detectable marker, or that can act in vivo on a subject. Bioactive agents and molecules include biomolecules, such as DNA, RNA, proteins, other biopolymers, and small molecules, such as small molecule drugs and pharmaceuticals, immunogens, and any molecules that would be delivered to a subject, such as a human or other animal or a plant or a microorganism (bacteria or other), in connection with a therapy, a diagnostic application, or other such uses, such as a cosmetic. The bioactive agent or molecule can function as or have an activity as, for example, a therapeutic, an immunogen, a diagnostic, a detectable marker, or a cosmetic. The bioactive molecules for use herein are any that can be loaded into a microalgae extracellular vesicle (MEV).

As used herein, a biomolecule refers to any biologically active biopolymer or molecule that occurs, or can occur, in a living organism or virus or that is a modified form of such biopolymer or molecule. Biomolecules, thus, include modified naturally-occurring biomolecules, such as, for example proteins that include a modified primary sequence, such as by deletions, insertions, and/or replacements of amino acids to alter the primary sequence, and or by modification, such as post-translational modifications of the protein.

As used herein, when it is stated that MEVs have the same or substantially the same loaded cargo or amount thereof, it is understood that this refers to an average among the population of MEVs in a composition. It is understood, that when MEVs are loaded exogenously the ratio of cargo/MEV can be selected so that each MEV has, on average, a pre-determined amount of cargo. As a simple example, to load an average of one molecule of cargo/MEV, the skilled person could calculate the amount of cargo to load into a composition of MEVs, and understands that in the composition of MEVs, some would have more than one molecule of cargo/MEV, and others would have none. On average, the MEVs would have one molecule of cargo/MEV. The skilled person understands, that, in general, the amount of cargo/MEV will be more than the one molecule/MEV, and that the amount of cargo depends upon a variety of parameters, including the cargo, the target tissues and/or cells, the disease, disorder, or condition treated, and the subject treated. Generally, more than one molecule of cargo per MEV, on the average, such as at least 10 or about 10 molecules/MEV are loaded. Substantially more cargo, 100, 500, 1000, 10⁴ molecules/MEV and more, also can be loaded. The amount loaded depends upon the target, disease, disorder, or condition, the subject, and the cargo, and the capacity of the MEV. It is within the skill in the art to select the amount.

As used herein, a subject is any organism, generally an animal or plant, into which or on which the composition containing the MEV is introduced. Subjects include, but are not limited to, humans, plants, particularly crop plants, and animals, including farm animals and pets, such as dogs and cats, and zoo animals.

As used herein, a drug delivery system refers to a composition that contains MEVs provided herein that contain cargo for delivery to tissues. As shown herein, by virtue of the formulation and route of administration of the composition containing the MEVs the trafficking route and/or ultimate destination of the MEVs, upon administration, can selected. For example, as demonstrated herein, orally administered MEVs can target gut-associated lymphoid tissue (GALT). Thus, GALT is a target (effective compartment) and/or a route through which MEVs and their therapeutic agent cargo can be used to deliver cargo. The delivery system refers to the combining of formulation for a particular route of administration to target particular tissues for treatment of diseases, disorders, and conditions of these tissues or involving these tissues.

As used herein, disease or disorder or condition refers to a pathological or undesirable or undesired condition in an organism resulting from a cause or condition including, but not limited to, infections, acquired conditions, and genetic conditions, and those characterized by identifiable symptoms or characteristics.

As used herein, treating a subject with a disease, disorder, or condition means that the subject's symptoms or manifestations of the disease or conditions are partially or totally alleviated, or remain static following treatment.

As used herein, treatment refers to any effects that ameliorate symptoms of a disease or disorder. Treatment encompasses prophylaxis, therapy and/or cure.

Treatment also encompasses any pharmaceutical use of any MEV or composition provided herein. Treatment refers to any effects that ameliorate or prevent or otherwise reduce or eliminate any symptom or manifestation of a disease or disorder.

Treatment also encompasses any pharmaceutical use of any MEV or composition provided herein.

As used herein, prophylaxis refers to prevention of a potential disease and/or a prevention of worsening of symptoms or progression of a disease. Prevention or prophylaxis, and grammatically equivalent forms thereof, refer to methods in which the risk or probability of developing a disease or condition is reduced or eliminated and products that reduce or eliminate the risk or probability of developing a disease or condition.

As used herein, a modification with reference to modification of a sequence of amino acids of a polypeptide or a sequence of nucleotides in a nucleic acid molecule refers to and includes deletions, insertions, and replacements of amino acids or nucleotides, respectively. These include modifications of the primary sequence of a polypeptide or protein. Methods of modifying a polypeptide and nucleic acid molecule are routine to those of skill in the art, such as by using recombinant DNA methodologies. Modifications, when referring to polypeptide or protein, not to a sequence, refer to post-translational or post-purification changes, such as conjugation or linkage of moieties that alter properties of polypeptide or protein, such as half-life extending moieties, glycosylation, purification tags, detectable reporters, and other such moieties.

As used herein, a modification of a genome or a plasmid or gene includes deletions, replacements, insertions, and translocations of nucleic acid. These include any changes to the native or naturally-occurring nucleic acid sequence.

As used herein, RNA interference (RNAi) is a biological process in which RNA molecules inhibit gene expression or translation, by neutralizing targeted mRNA molecules to inhibit translation and thereby expression of a targeted gene.

As used herein, RNA molecules that act via RNAi are referred to as inhibitory by virtue of their silencing of expression of a targeted gene. Silencing expression means that expression of the targeted gene is reduced or suppressed or inhibited.

As used herein, gene silencing via RNAi is said to inhibit, suppress, disrupt or silence expression of a targeted gene. A targeted gene contains sequences of nucleotides that correspond to the sequences in the inhibitory RNA, whereby the inhibitory RNA silences expression of mRNA. Small interfering RNAs (siRNAs) are small pieces of double-stranded (ds) RNA, usually about 21 nucleotides long, with 3′ overhangs (2 nucleotides) at each end that can be used to interfere with the translation of proteins by binding to and promoting the degradation of messenger RNA (mRNA) at specific sequences. In doing so, siRNAs prevent the production of specific proteins based on the nucleotide sequences of their corresponding mRNAs. The process is called RNA interference (RNAi), and also is referred to as siRNA silencing or siRNA knockdown. A short-hairpin RNA or small-hairpin RNA (shRNA) is an artificial RNA molecule with a tight hairpin turn that can be used to silence target gene expression via RNA interference (RNAi). Expression of shRNA in cells is typically accomplished by delivery of plasmids or through viral or bacterial vectors.

As used herein, non-coding RNAs are RNAs that do not encode a protein. Classes of non-coding RNA, include, but are not limited to, small interfering RNAs (siRNAs) and microRNAs (miRNAs). As used herein, inhibiting, suppressing, disrupting or silencing a targeted gene refers to processes that alter expression, such as translation, of the targeted gene, whereby activity or expression of the product encoded by the targeted gene is reduced. Reduction includes a complete knock-out or a partial knockout, whereby, with reference to the MEVs provided herein and administration herein, treatment is effected.

As used herein, an adjuvant is a substance that enhances the body's immune response to an antigen; it can be formulated with a vaccine or be part of a vaccine. For purposes herein, an adjuvant does not refer to the MEVs or compositions containing the MEVs, but refers to an additional component to enhance the immune response. It is shown herein that the vaccines comprising the MEVs do not require an adjuvant; the immune response can be as robust with the MEVs as with an adjuvant containing the cargo. For purposes herein, the MEVs are not considered an adjuvant, but are the delivery vehicle. Co-administered active agents or other agents, such as toll-like receptor agonists or antagonists, while they can enhance an immune response, they are not considered adjuvants in the compositions as provided herein. For purposes herein, the reference to a composition comprising MEVs that does not comprise an adjuvant, is a composition to which an agent has not specifically been added to enhance the immune response to the MEV antigen cargo. It is understood that MEVs can include immunomodulatory compounds as cargo. Administration of the MEVs alone does not require an adjuvant. In some embodiments, a skilled person, however, may add an adjuvant.

As used herein, an exogenous adjuvant is a separate component of a vaccine composition, containing an MEV that comprises or encodes an antigen, and/or an immunomodulatory product or agent, that enhances the immune response to cargo in the MEV.

As used herein, a vaccine treats, results in an immune response, prevents, or reduces the severity of a disease, disorder, or condition. It stimulates an immune response against an antigen, which can be part of a pathogen, cell, such as a tumor cell, whereby the immune system can interact with the target to generally inactivate it or reduce the effect thereof. The immune response is thereby immunoprotective. Prevention includes prophylaxis by reducing the risk of getting or developing a disease, disorder, or condition, or reduces the severity of a disease, disorder, or condition.

As used herein, an immune modulator refers to an agent that stimulates or suppresses the immune system. Immune system modulators, include, for example, cytokines, including, but are not limited to, interferons, interleukins, ligands, receptors, and antibodies. Immune system modulators include those that act specifically on a particular target or targets, and those that act generally on the immune system. For purposes herein, the MEVs can deliver immune modulators, and in some embodiments, immune modulators in combination with antigens, to modulate the immune response to the antigen.

As used herein, a tumor microenvironment (TME) is the cellular environment in which the tumor exists, including surrounding blood vessels, immune cells, fibroblasts, bone marrow-derived inflammatory cells, lymphocytes, signaling molecules and the extracellular matrix (ECM). Conditions that exist include, but are not limited to, increased vascularization, hypoxia, low pH, increased lactate concentration, increased pyruvate concentration, increased interstitial fluid pressure and altered metabolites or metabolism, such as higher levels of adenosine, indicative of a tumor.

As used herein, recitation that a nucleic acid or encoded RNA targets a gene means that it inhibits or suppresses or silences expression of the gene by any mechanism. Generally, such nucleic acid includes at least a portion complementary to the targeted gene, where the portion is sufficient to form a hybrid with the complementary portion.

As used herein, deletion, when referring to a nucleic acid or polypeptide sequence, refers to the deletion of one or more nucleotides or amino acids compared to a sequence, such as a target polynucleotide or polypeptide or a native or wild-type sequence.

As used herein, insertion, when referring to a nucleic acid or amino acid sequence, describes the inclusion of one or more additional nucleotides or amino acids, within a target, native, wild-type or other related sequence. Thus, a nucleic acid molecule that contains one or more insertions compared to a wild-type sequence, contains one or more additional nucleotides within the linear length of the sequence.

As used herein, additions to nucleic acid and amino acid sequences describe addition of nucleotides or amino acids onto either termini compared to another sequence.

As used herein, substitution or replacement refers to the replacing of one or more nucleotides or amino acids in a native, target, wild-type or other nucleic acid or polypeptide sequence with an alternative nucleotide or amino acid, without changing the length (as described in numbers of residues) of the molecule. Thus, one or more substitutions in a molecule does not change the number of amino acid residues or nucleotides of the molecule. Amino acid replacements compared to a particular polypeptide can be expressed in terms of the number of the amino acid residues along the length of the polypeptide sequence.

As used herein, at a position corresponding to, or a recitation that nucleotides or amino acid positions correspond to nucleotides or amino acid positions in a disclosed sequence, such as set forth in the Sequence Listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carrillo et al. (1988) SIAM J Applied Math 48:1073).

As used herein, alignment of a sequence refers to the use of homology to align two or more sequences of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence. Related or variant polypeptides or nucleic acid molecules can be aligned by any method known to those of skill in the art. Such methods typically maximize matches, and include methods, such as using manual alignments and by using the numerous alignment programs available (e.g., BLASTP) and others known to those of skill in the art. By aligning the sequences of polypeptides or nucleic acids, one skilled in the art can identify analogous portions or positions, using conserved and identical amino acid residues as guides. Further, one skilled in the art also can employ conserved amino acid or nucleotide residues as guides to find corresponding amino acid or nucleotide residues between and among human and non-human sequences. Corresponding positions also can be based on structural alignments, for example by using computer simulated alignments of protein structure. In other instances, corresponding regions can be identified. One skilled in the art also can employ conserved amino acid residues as guides to find corresponding amino acid residues between and among human and non-human sequences.

As used herein, a property of a polypeptide, such as an antibody, refers to any property exhibited by a polypeptide, including, but not limited to, binding specificity, structural configuration or conformation, protein stability, resistance to proteolysis, conformational stability, thermal tolerance, and tolerance to pH conditions. Changes in properties can alter an activity of the polypeptide. For example, a change in the binding specificity of the antibody polypeptide can alter the ability to bind an antigen, and/or various binding activities, such as affinity or avidity, or in vivo activities of the polypeptide.

As used herein, an activity or a functional activity of a polypeptide, such as an antibody, refers to any activity exhibited by the polypeptide. Such activities can be empirically determined. Exemplary activities include, but are not limited to, ability to interact with a biomolecule, for example, through antigen-binding, DNA binding, ligand binding, or dimerization, or enzymatic activity, for example, kinase activity or proteolytic activity. For an antibody (including antibody fragments), activities include, but are not limited to, the ability to specifically bind a particular antigen, affinity of antigen-binding (e.g., high or low affinity), avidity of antigen-binding (e.g., high or low avidity), on-rate, off-rate, effector functions, such as the ability to promote antigen neutralization or clearance, virus neutralization, and in vivo activities, such as the ability to prevent infection or invasion of a pathogen, or to promote clearance, or to penetrate a particular tissue or fluid or cell in the body. Activity can be assessed in vitro or in vivo using recognized assays, such as ELISA, flow cytometry, surface plasmon resonance or equivalent assays to measure on- or off-rate, immunohistochemistry and immunofluorescence histology and microscopy, cell-based assays, flow cytometry and binding assays (e.g., panning assays).

As used herein, bind, bound, and grammatical variations thereof refer to the participation of a molecule in any interaction with another molecule or among molecules, resulting in a stable association in which the molecules are in close proximity to one another. Binding includes, but is not limited to, non-covalent bonds, covalent bonds (such as reversible and irreversible covalent bonds), and includes interactions between molecules such as, but not limited to, proteins, nucleic acids, carbohydrates, lipids, and small molecules, such as chemical compounds including drugs.

As used herein, antibody refers to immunoglobulins and immunoglobulin fragments, whether natural or partially or wholly synthetically, such as recombinantly produced, including any fragment thereof containing at least a portion of the variable heavy chain and light region of the immunoglobulin molecule that is sufficient to form an antigen binding site and, when assembled, to specifically bind an antigen. Hence, an antibody includes any protein having a binding domain that is homologous or substantially homologous to an immunoglobulin antigen-binding domain (antibody combining site). For example, an antibody refers to an antibody that contains two heavy chains (which can be denoted H and H′) and two light chains (which can be denoted L and L′), where each heavy chain can be a full-length immunoglobulin heavy chain or a portion thereof sufficient to form an antigen binding site (e.g., heavy chains include, but are not limited to, VH chains, VH-CH1 chains and VH-CH1-CH2-CH3 chains), and each light chain can be a full-length light chain or a portion thereof sufficient to form an antigen binding site (e.g., light chains include, but are not limited to, VL chains and VL-CL chains). Each heavy chain (H and H′) pairs with one light chain (L and L′, respectively). Typically, antibodies minimally include all or at least a portion of the variable heavy (VH) chain and/or the variable light (VL) chain. The antibody also can include all or a portion of the constant region.

For purposes herein, the term antibody includes full-length antibodies and portions thereof including antibody fragments, such as anti-tumor antibody or anti-pathogen or gene silencing fragments. Antibody fragments, include, but are not limited to, Fab fragments, Fab′ fragments, F(ab′)₂ fragments, Fv fragments, disulfide-linked Fvs (dsFv), Fd fragments, Fd′ fragments, single-chain Fvs (scFv), single-chain Fabs (scFab), diabodies, anti-idiotypic (anti-Id) antibodies, or antigen-binding fragments of any of the above. Antibody also includes synthetic antibodies, recombinantly produced antibodies, multispecific antibodies (e.g., bispecific antibodies), human antibodies, non-human antibodies, humanized antibodies, chimeric antibodies, and intrabodies. Antibodies can include members of any immunoglobulin class (e.g., IgG, IgM, IgD, IgE, IgA and IgY), any subclass (e.g., IgG1, IgG2, IgG3, IgG4, IgAQ1 and IgA2) or sub-subclass (e.g., IgG2a and IgG2b).

As used herein, nucleic acid refers to at least two linked nucleotides or nucleotide derivatives, including a deoxyribonucleic acid (DNA) and a ribonucleic acid (RNA), joined together, typically by phosphodiester linkages. Also included in the term nucleic acid are analogs of nucleic acids such as peptide nucleic acid (PNA), phosphorothioate DNA, and other such analogs and derivatives or combinations thereof. Nucleic acids also include DNA and RNA derivatives containing, for example, a nucleotide analog or a backbone bond other than a phosphodiester bond, for example, a phosphotriester bond, a phosphoramidate bond, a phosphorothioate bond, a thioester bond, or a peptide bond (peptide nucleic acid). The term also includes, as equivalents, derivatives, variants, and analogs of either RNA or DNA made from nucleotide analogs, single (sense or antisense) and double-stranded nucleic acids. Deoxyribonucleotides include deoxyadenosine, deoxycytidine, deoxyguanosine and deoxythymidine. For RNA, the uracil base is uridine.

As used herein, an isolated nucleic acid molecule is one which is separated from other nucleic acid molecules which are present in the natural source of the nucleic acid molecule. An isolated nucleic acid molecule, such as a cDNA molecule, can be substantially free of other cellular material, or culture medium when produced by recombinant techniques, or substantially free of chemical precursors or other chemicals when chemically synthesized. Exemplary isolated nucleic acid molecules provided herein include isolated nucleic acid molecules encoding RNAi or a therapeutic protein.

As used herein, operably linked with reference to nucleic acid sequences, regions, elements or domains means that the nucleic acid regions are functionally related to each other. For example, a nucleic acid encoding a leader peptide can be operably linked to a nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to a nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, the residues of naturally occurring α-amino acids are the residues of those 20 α-amino acids found in nature which are incorporated into protein by the specific recognition of the charged tRNA molecule with its cognate mRNA codon in humans.

As used herein, polypeptide refers to two or more amino acids covalently joined. The terms polypeptide and protein are used interchangeably herein.

As used herein, a peptide refers to a polypeptide that is from 2 to about or 40 amino acids in length.

As used herein, reference to proteins, unless otherwise specified, includes all forms of peptides, polypeptides, small peptides, and proteins.

As used herein, an amino acid is an organic compound containing an amino group and a carboxylic acid group. A polypeptide contains two or more amino acids. For purposes herein, amino acids contained in the antibodies provided include the twenty naturally-occurring amino acids (see Table below), non-natural amino acids, and amino acid analogs (e.g., amino acids wherein the α-carbon has a side chain). As used herein, the amino acids, which occur in the various amino acid sequences of polypeptides appearing herein, are identified according to their well-known, three-letter or one-letter abbreviations (see Table below). The nucleotides, which occur in the various nucleic acid molecules and fragments, are designated with the standard single-letter designations used routinely in the art.

As used herein, amino acid residue refers to an amino acid formed upon chemical digestion (hydrolysis) of a polypeptide at its peptide linkages. The amino acid residues described herein are generally in the L isomeric form. Residues in the D isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3557-59 (1968) and adopted at 37 C.F.R. §§ 1.821-1.822, abbreviations for amino acid residues are shown in the following Table:

Table of Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glutamic Acid and/or Glutamine |
| W | Trp | Tryptophan |
| R | Arg | Arginine |
| D | Asp | Aspartic acid |
| N | Asn | Asparagine |
| B | Asx | Aspartic Acid and/or Asparagine |
| C | Cys | Cysteine |
| X | Xaa | Unknown or other |

All sequences of amino acid residues represented herein by a formula have a left to right orientation in the conventional direction of amino-terminus to carboxyl-terminus. The phrase “amino acid residue” is defined to include the amino acids listed in the above Table of Correspondence, modified, non-natural and unusual amino acids. A dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in the art and generally can be made without altering a biological activity of a resulting molecule. Those of skill in the art recognize that, in general, single amino acid substitutions in non-essential regions of a polypeptide do not substantially alter biological activity (see, e.g., Watson et al., Molecular Biology of the Gene, 4th Edition, 1987, The Benjamin/Cummings Pub. Co., p. 224).

Such substitutions can be made in accordance with the exemplary substitutions set forth in the following Table:

Exemplary conservative amino acid substitutions

| Original | Exemplary Conservative |
|---|---|
| residue | substitution(s) |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, naturally occurring amino acids refer to the 20 L-amino acids that occur in polypeptides.

As used herein, the term non-natural amino acid refers to an organic compound that has a structure similar to a natural amino acid but has been modified structurally to mimic the structure and reactivity of a natural amino acid. Non-naturally occurring amino acids thus include, for example, amino acids or analogs of amino acids other than the 20 naturally occurring amino acids and include, but are not limited to, the D-stereoisomers of amino acids. Exemplary non-natural amino acids are known to those of skill in the art, and include, but are not limited to, 2-Aminoadipic acid (Aad), 3-Aminoadipic acid (bAad), β-alanine/β-Amino-propionic acid (Bala), 2-Aminobutyric acid (Abu), 4-Aminobutyric acid/piperidinic acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (Ahyl), 3-Hydroxyproline (3Hyp), 4-Hydroxyproline (4Hyp), Isodesmosine (Ide), allo-Isoleucine (Aile), N-Methylglycine, sarcosine (MeGly), N-Methylisoleucine (MeIle), 6-N-Methyllysine (MeLys), N-Methylvaline (MeVal), Norvaline (Nva), Norleucine (Nle), and Ornithine (Orn).

As used herein, a DNA construct is a single- or double-stranded, linear or circular DNA molecule that contain segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated nt) or base pairs (abbreviated bp). The term “nucleotides” is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus, all nucleotides within a double-stranded polynucleotide molecule cannot be paired. Such unpaired ends will, in general, not exceed 20 nucleotides in length.

As used herein, production by recombinant methods refers to or means the use of the well-known methods of molecular biology for expressing proteins encoded by cloned DNA.

As used herein, heterologous nucleic acid is nucleic acid that encodes products (i.e., RNA and/or proteins) that are not normally produced in vivo by the cell in which it is expressed, or nucleic acid that is in a locus in which it does not normally occur, or that mediates or encodes mediators that alter expression of endogenous nucleic acid, such as DNA, by affecting transcription, translation, or other regulatable biochemical processes. Heterologous nucleic acid, such as DNA, also is referred to as foreign nucleic acid. Any nucleic acid, such as DNA, that one of skill in the art would recognize or consider as heterologous or foreign to the cell in which it is expressed, is herein encompassed by heterologous nucleic acid; heterologous nucleic acid includes exogenously added nucleic acid that is also expressed endogenously. Heterologous nucleic acid is generally not endogenous to the cell into which it is introduced, but has been obtained from another cell or prepared synthetically or is introduced into a genomic locus in which it does not occur naturally, or its expression is under the control of regulatory sequences or a sequence that differs from the natural regulatory sequence or sequences.

Examples of heterologous nucleic acid herein include, but are not limited to, a DNA molecule, an RNA molecule, a plasmid, and an antisense oligonucleotide. In the MEV, the heterologous nucleic acid can be encoded on a plasmid. Heterologous nucleic acid, such as DNA, includes nucleic acid that can, in some manner, mediate expression of DNA that encodes a therapeutic product, or it can encode a product, such as a peptide or RNA, that in some manner mediates, directly or indirectly, expression of a therapeutic product.

As used herein, cell therapy involves the delivery of MEVs to a subject to treat a disease or condition. The MEVs are exogenously loaded with cargo, so that they deliver or express products when introduced to a subject. The MEVs also can be endogenously loaded with cargo (see, e.g., copending U.S. provisional application Ser. No. 63/349,006, filed on Jun. 3, 2022, which details preparation of endogenously-loaded MEVs and producer cell lines thereof), and used as described herein. The trafficking of MEVs generally is independent of manner in which they are loaded with cargo. The microalgae can be modified to alter properties of the resulting MEVs. Endogenously-loaded MEVs can be used in the methods and compositions described herein.

As used herein, genetic therapy involves the transfer of heterologous nucleic acid, such as DNA, into certain cells, such as target cells, of a mammal, particularly a human, with a disorder or condition for which such therapy is sought. The nucleic acid, such as DNA, is introduced into the selected target cells in a manner such that the heterologous nucleic acid, such as DNA, is expressed and a therapeutic product(s) encoded thereby is produced. Genetic therapy can also be used to deliver nucleic acid encoding a gene product that replaces a defective gene or supplements a gene product produced by the mammal or the cell in which it is introduced. The introduced nucleic acid can encode a therapeutic compound, such as a growth factor or inhibitor thereof, or a tumor necrosis factor or inhibitor thereof, such as a receptor thereof, that is not normally produced in the mammalian host or that is not produced in therapeutically effective amounts or at a therapeutically useful time. The heterologous nucleic acid, such as DNA, encoding the therapeutic product, can be modified prior to introduction into the cells of the afflicted host in order to enhance or otherwise alter the product or expression thereof. Genetic therapy can also involve delivery of an inhibitor or repressor or other modulator of gene expression.

As used herein, expression refers to the process by which polypeptides are produced by transcription and translation of polynucleotides. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, a host cell is a cell that is used to receive, maintain, reproduce and/or amplify a vector. A host cell also can be used to express the polypeptide encoded by the vector. The nucleic acid contained in the vector is replicated when the host cell divides, thereby amplifying the nucleic acids.

As used herein, a vector is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction enzyme digestion and ligation. Reference to a vector also includes those vectors that contain nucleic acid encoding a polypeptide or RNA. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well-known to those of skill in the art. A vector also includes virus vectors or viral vectors. Viral vectors are engineered viruses that are operatively linked to exogenous genes to transfer (as vehicles or shuttles) the exogenous genes into cells.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well-known to those of skill in the art and include those that are replicable in eukaryotic cells and/or prokaryotic cells and those that remain episomal or those which integrate into the host cell genome.

As used herein, primary sequence refers to the sequence of amino acid residues in a polypeptide or the sequence of nucleotides in a nucleic acid molecule.

As used herein, sequence identity refers to the number of identical or similar amino acids or nucleotide bases in a comparison between a test and a reference poly-peptide or polynucleotide. Sequence identity can be determined by sequence alignment of nucleic acid or protein sequences to identify regions of similarity or identity. For purposes herein, sequence identity is generally determined by alignment to identify identical residues. The alignment can be local or global. Matches, mismatches and gaps can be identified between compared sequences. Gaps are null amino acids or nucleotides inserted between the residues of aligned sequences so that identical or similar characters are aligned. Generally, there can be internal and terminal gaps. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a global alignment is an alignment that aligns two sequences from beginning to end, aligning each letter in each sequence only once. An alignment is produced, regardless of whether or not there is similarity or identity between the sequences. For example, 50% sequence identity based on global alignment means that in an alignment of the full sequence of two compared sequences each of 100 nucleotides in length, 50% of the residues are the same. It is understood that global alignment also can be used in determining sequence identity even when the length of the aligned sequences is not the same. The differences in the terminal ends of the sequences will be taken into account in determining sequence identity, unless the no penalty for end gaps is selected. Generally, a global alignment is used on sequences that share significant similarity over most of their length. Exemplary algorithms for performing global alignment include the Needleman-Wunsch algorithm (Needleman et al. (1970) J. Mol. Biol. 48: 443). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a local alignment is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on local alignment means that in an alignment of the full sequence of two compared sequences of any length, a region of similarity or identity of 100 nucleotides in length has 50% of the residues that are the same in the region of similarity or identity.

For purposes herein, sequence identity can be determined by standard alignment algorithm programs used with default gap penalties established by each supplier. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Whether any two nucleic acid molecules have nucleotide sequences or any two polypeptides have amino acid sequences that are at least 80%, 85%, 90%, 95%, 96%, 97%, 98% or 99% identical, or other similar variations reciting a percent identity, can be determined using known computer algorithms based on local or global alignment (see e.g., wikipedia.org/wiki/Sequence_alignment_software, providing links to dozens of known and publicly available alignment databases and programs). Generally, for purposes herein sequence identity is determined using computer algorithms based on global alignment, such as the Needleman-Wunsch Global Sequence Alignment tool available from NCBI/BLAST (blast.ncbi.nlm.nih.gov/Blast.cgi?CMD=Web&Page_TYPE=BlastHome); LAlign (William Pearson implementing the Huang and Miller algorithm (Adv. Appl. Math. (1991) 12:337-357)); and program from Xiaoqui Huang available at deepc2.psi.iastate.edu/aat/align/align.html. Typically, the full-length sequence of each of the compared polypeptides or nucleotides is aligned across the full-length of each sequence in a global alignment. Local alignment also can be used when the sequences being compared are substantially the same length.

Therefore, as used herein, the term identity represents a comparison or alignment between a test and a reference polypeptide or polynucleotide. In one non-limiting example, at least 90% identical to refers to percent identities from 90 to 100% relative to the reference polypeptide or polynucleotide. Identity at a level of 90% or more is indicative of the fact that, assuming for exemplification purposes a test and reference polypeptide or polynucleotide length of 100 amino acids or nucleotides are compared, no more than 10% (i.e., 10 out of 100) of amino acids or nucleotides in the test polypeptide or polynucleotide differ from those of the reference polypeptide. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences also can be due to deletions or truncations of amino acid residues. Differences are defined as nucleic acid or amino acid substitutions, insertions or deletions. Depending on the length of the compared sequences, at the level of homologies or identities above about 85-90%, the result can be independent of the program and gap parameters set; such high levels of identity can be assessed readily, often without relying on software.

As used herein, a pharmaceutically effective agent includes any therapeutic agent or bioactive agents, including, but not limited to, for example, anesthetics, vasoconstrictors, dispersing agents, and conventional therapeutic drugs, including small molecule drugs and therapeutic proteins.

As used herein, a therapeutic effect means an effect resulting from treatment of a subject that alters, typically improves or ameliorates, the symptoms of a disease or condition or that cures a disease or condition.

As used herein, a therapeutically effective amount or a therapeutically effective dose refers to the quantity of an agent, compound, material, or composition containing a compound that is at least sufficient to produce a therapeutic effect following administration to a subject. Hence, it is the quantity necessary for preventing, curing, ameliorating, arresting or partially arresting a symptom of a disease or disorder.

As used herein, therapeutic efficacy refers to the ability of an agent, compound, material, or composition containing a compound to produce a therapeutic effect in a subject to whom the agent, compound, material, or composition containing a compound has been administered.

As used herein, a prophylactically effective amount or a prophylactically effective dose refers to the quantity of an agent, compound, material, or composition containing a compound that when administered to a subject, will have the intended prophylactic effect, e.g., preventing or delaying the onset, or reoccurrence, of disease or symptoms, reducing the likelihood of the onset, or reoccurrence, of disease or symptoms, or reducing the incidence of viral infection. The full prophylactic effect does not necessarily occur by administration of one dose, and can occur only after administration of a series of doses. Thus, a prophylactically effective amount can be administered in one or more administrations.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, an anti-cancer agent refers to any agent that is destructive or toxic to malignant cells and tissues. For example, anti-cancer agents include agents that kill cancer cells or otherwise inhibit or impair the growth of tumors or cancer cells. Exemplary anti-cancer agents are chemotherapeutic agents.

As used herein therapeutic activity refers to the in vivo activity of a therapeutic polypeptide. Generally, the therapeutic activity is the activity that is associated with treatment of a disease or condition.

As used herein, the term subject refers to an animal, including a mammal, such as a human being.

As used herein, a patient refers to a human subject.

As used herein, animal includes any animal, such as, but not limited to, primates including humans, gorillas and monkeys; rodents, such as mice and rats; fowl, such as chickens; ruminants, such as goats, cows, deer, and sheep; and pigs and other animals. Non-human animals exclude humans as the contemplated animal.

As used herein, a composition refers to any mixture. It can be a solution, suspension, liquid, powder, paste, aqueous, non-aqueous, or any combination thereof.

As used herein, a combination refers to any association between or among two or more items. The combination can be two or more separate items, such as two compositions or two collections, a mixture thereof, such as a single mixture of the two or more items, or any variation thereof. The elements of a combination are generally functionally associated or related.

As used herein, combination therapy refers to administration of two or more different therapeutics. The different therapeutic agents can be provided and administered separately, sequentially, intermittently, or can be provided in a single composition.

As used herein, a kit is a packaged combination that optionally includes other elements, such as additional reagents and instructions for use of the combination or elements thereof, for a purpose including, but not limited to, activation, administration, diagnosis, and assessment of a biological activity or property.

As used herein, a unit dose form refers to physically discrete units suitable for human and animal subjects and packaged individually as is known in the art.

As used herein, a single dosage formulation refers to a formulation for direct administration.

As used herein, a multi-dose formulation refers to a formulation that contains multiple doses of a therapeutic agent and that can be directly administered to provide several single doses of the therapeutic agent. The doses can be administered over the course of minutes, hours, weeks, days or months. Multi-dose formulations can allow dose adjustment, dose-pooling and/or dose-splitting. Because multi-dose formulations are used over time, they generally contain one or more preservatives to prevent microbial growth.

As used herein, an article of manufacture is a product that is made and sold. As used throughout this application, the term is intended to encompass any of the compositions provided herein contained in articles of packaging.

As used herein, a fluid refers to any composition that can flow. Fluids thus encompass compositions that are in the form of semi-solids, pastes, solutions, aqueous mixtures, gels, lotions, creams, and other such compositions.

As used herein, an isolated or purified polypeptide or protein (e.g., an isolated antibody or antigen-binding fragment thereof) or biologically-active portion thereof (e.g., an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

As used herein, a cellular extract or lysate refers to a preparation or fraction which is made from a lysed or disrupted cell.

As used herein, a control refers to a sample that is substantially identical to the test sample, except that it is not treated with a test parameter, or, if it is a plasma sample, it can be from a normal volunteer not affected with the condition of interest.

A control also can be an internal control.

As used herein, a tropism of an MEV refers to cells, tissues, and/or organs wherein the MEVs, upon administration, accumulate.

As used herein, natural tropism with reference to the MEVS provided herein, refers to MEVs that are not modified to provide a specific tropism or targeting property.

As used herein, the singular forms “a,” “an” and “the” include plural referents unless the context clearly dictates otherwise. Thus, for example, reference to a polypeptide, comprising an immunoglobulin domain includes polypeptides with one or a plurality of immunoglobulin domains.

As used herein, the term “or” is used to mean “and/or” unless explicitly indicated to refer to alternatives only or the alternatives are mutually exclusive.

As used herein, ranges and amounts can be expressed as about a particular value or range. “About” also includes the exact amount. Hence about 5 amino acids means about 5 amino acids and also 5 amino acids.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

For clarity of disclosure, and not by way of limitation, the detailed description is divided into the subsections that follow.

### B. MICROALGAE AND OVERVIEW

Algae are a complex, polyphyletic collection of predominantly photosynthetic organisms. These organisms include micro- and macroscopic forms. Macroalgae (seaweed) are multicellular, large-size algae, visible with the naked eye. Microalgae are microscopic single cells and include prokaryotes (e.g., cyanobacteria), and eukaryotes, such as green algae.

Compared to photosynthetic crops, microalgae have a higher growth rate and can be cultivated on non-arable land, and also in bioreactors. Many species of microalgae can be grown year-round in industrial scale photobioreactors under controlled cultivation conditions (Adamo et al. (2021) Journal of Extracellular Vesicles 10:e12081).

Algae generally are classified into eleven major phyla: Cyanophyta, Chlorophyta, Rhodophyta, Glaucophyta, Euglenophyta, Chlorarachniophyta, Charophyta, Cryptophyta, Haptophyta, Heterokontophyta, and Dinophyta (Barkia et al. (2019) Mar. Drugs 17(5):304). Different pigments occur in each algae group. Cyanobacteria (or Cyanophyta) contain chlorophyll-a, -d, and -f, in addition to the phycobiliproteins (proteins that capture light energy), phycocyanin, allophycocyanin, and phycoerythrin. Glaucophytes contain chlorophyll-a and harvest light via phycobiliproteins. Chlorophytes have chlorophyll-a and -b, as well as carotenoids, including β-carotene and various xanthophylls (e.g., astaxanthin, canthaxanthin, lutein, and zeaxanthin). The primary pigments of Rhodophyta (red algae) are phycoerythrin and phycocyanin, which can mask chlorophyll-a; red algae also produce a broad spectrum of carotenes and xanthophyll light-harvesting pigments (Barkia et al. (2019) Mar. Drugs 17(5):304).

Extracellular vesicles produced by algae, particularly unicellular green algae, such as species of Chlorella, for use for delivery of exogenously loaded cargo to animals and plants for use as vaccines and for delivery of immunomodulators are provided. The algae are unicellular eukaryotes that typically are haploid, but can have a diploid stage of the life cycle. The algae can be cultured in bioreactors and the extracellular vesicles isolated therefrom. The resulting extracellular vesicles can be loaded by methods such as electroporation, with cargo, generally a cargo of heterologous bioactive molecules to produce compositions that contain the extracellular vesicles for administration to animals and also to plants. The compositions can be formulated for any desired route of administration, including topical, local, systemic, parenteral, and oral. These routes include oral, intravenous, subcutaneous, inhalation, mucosal, rectal, vaginal, and other suitable routes. The cargo includes biomolecules, such as DNA, RNA, proteins, protein complexes, protein-nucleic acid complexes, plasmids, and also includes small molecules, such as small molecule drugs. The extracellular vesicles can be formulated as liquids, powders, including lyophilized powders, tablets, capsules, emulsions, particles, sprays, gels, ointments, creams, and other formulations. They can be used for therapeutic, diagnostic, theragnostic, cosmetic, and other uses. The extracellular vesicles can be used to treat diseases and conditions, that include cancers, inflammatory diseases and conditions in which the immune system plays a role in the etiology or symptoms, nervous system disorders, and pathogen infections, including viral and bacterial and other pathogens. They can be used to treat dermatological diseases and conditions, lung diseases and conditions, and gastric diseases and conditions. The extracellular vesicles can be targeted to specific organs or tissues or can be locally administered.

As with extracellular vesicles (EVs) from other sources, such as mammalian EVs, microalgae EVs (MEVs) have evolved to efficiently pass genetic material and other kinds of molecules from cell to cell. They orchestrate intercellular and cross-kingdom communication between cells via exchange of biologically active molecules. MEVs are natural nanoparticles. They are cell-derived, so, absent synthetic cargo, and genetic modifications, there are no synthetic components; they are safe, for example, there is no risk of endogenous viruses that are potentially dangerous to humans.

The MEVs provided herein include, but are not limited to, Chlorella MEVs, particularly Chlorella vulgaris, a freshwater microalgae. Chlorella is a unicellular haploid alga that is a natural and efficient producer of extracellular vesicles. Chlorella vulgaris has been consumed worldwide as a food supplement for decades; it is non-toxic and non-immunogenic, and can be cultured at large industrial scale at low cost.

The MEVs provided herein can be directly used to protect, convey, and deliver a broad spectrum of innovative therapeutic molecules into target cells relevant to specific diseases. Chlorella MEVs are exemplary of MEVs; their properties and results are exemplary of MEVs from other microalgae. For example, it is shown herein that the MEVs are not immunogenic; hence can be administered multiple times without adverse effects; they do alter or impair the immune response or immunomodulatory effects of the delivered cargo.

As shown and described herein, the MEVs have a number of advantageous features including, for example, biodistribution patterns by route of administration, low toxicity, good pharmacokinetic profiles in vivo. They can be administered by a variety of routes including oral administration, administration to the respiratory tract, intranasally, intravenously, among other routes. They traffic to specific organs, according to the route of administration, such as the intestine, the GALT, the spleen, the lungs, the liver, and mucosa. Based on data herein and comparison with data for other EVs and drug delivery systems, the MEVs have longer clearance rates and last longer in the targeted organs, tissues, and cells than reported for other delivery systems, including mammalian EVs.

As shown herein, the MEVs overcome natural body barriers (such as oral delivery, or specific lymphoid tissues delivery, or nose-to-brain delivery) that have not been attained with liquid nanoparticles and EVs of mammalian origin. Hence, the MEVs for use as vaccines and to deliver immunomodulatory products to spleen and other organs of the immune system can be administered orally, as well as by other routes, such as IM. The MEVs provided herein address unmet needs. These include the ability to convey and reliably deliver therapeutic molecules specifically to the site of treatment, while avoiding premature degradation or inactivation of the therapeutic agent by the immune system or by enzymes; for treatment of diseases for which a therapeutic agent already exists but cannot be properly delivered.

As shown herein, the purified or partially-purified MEVs can be loaded by physical methods (exogenous loading; exo-loading). Exo-loading is scalable and industrializable. The MEVs can be exo-loaded with a variety of molecules, varying in size, hydrophobicity, and nature, such as siRNA, mRNA, peptides, proteins, plasmids, oligonucleotides, and small molecules. The biological activity of the exo-loaded cargo is preserved, while at the same time it is protected from degradation by enzymes and other agents present in vivo. The MEVs can deliver their cargo to recipient cells of a myriad of origins, such as microalgae, bacteria, higher plant, mammal, and human. MEVs can also deliver the cargo to the proper cell compartments, ensuring the proper expression and biological activity of cargo molecules, including those having complex biological pathways such as siRNA, mRNA, receptor-binding peptides, among others. The MEVs also can be loaded endogenously by genetically modified microalgae that encode RNA, DNA, and proteins for incorporation into the MEVs (see, copending U.S. provisional application Ser. No. 63/349,006, which details endogenous loading of MEVs). Cargo includes antigens and immunomodulatory molecules, including encoding nucleic acids, nucleic acid products, such as ligands and RNAi and other double-stranded RNA, and small molecules.

### C. EXTRACELLULAR VESICLES

Extracellular vesicles (EVs) are biomolecular structures released from plant and animal cells that play a role in cell-to-cell communication. Structurally, EVs are negatively charged lipid bilayer vesicles with a density of 1.13 to 1.19 g/mL. EVs are able to cross barriers such as the plasma (or cytoplasmic) membrane and the blood/brain barrier, and provide for the horizontal transfer of their functional contents (i.e., proteins, lipids, RNA molecules, and circulating DNA) from a donor to a recipient cell (Kuruvinashetti et al. (2020) 20^{th} International Conference on Nanotechnology 354-357). EVs also are naturally stable in various biological fluids, immunologically inert, and can exhibit organ-specific targeting abilities (Picciotto et al. (2021) Biomater. Sci. doi:10.1039/dObm01696a).

EVs contain endogenous lipids, nucleic acids, and proteins. Although results differ due to variations in isolation techniques and methods of analyzing the data, EVs generally contain proteins associated with the plasma membrane, cytosol and those involved in lipid metabolism (see, e.g., Doyle and Wang (2019) Cells 8(7):727). Proteins involved in the biogenesis of EVs (e.g., components of the ESCRTs), EV formation and release (e.g., RAB27A, RAB111B, and ARF6), signal transduction, and antigen presentation, as well as tetraspinins commonly occur in EVs (Abels and Breakefield (2016) Mol. Neurobiol. 36(3):301-312). EVs are enriched for cholesterol, sphingomyelin, glycosphingolipids, and phosphatidylserine (Kuruvinashetti et al. (2020) 20^{th} International Conference on Nanotechnology 354-357). Although a small number of studies have identified genomic and mitochondrial DNA in EVs, EVs are primarily enriched with endogenous small RNAs. Studies have identified mRNAs, miRNAs, rRNAs, long and short non-coding RNA, tRNA fragments, piwi-interacting RNA, vault RNA, and Y RNA in EVs. Most of the RNA that naturally occurs in EVs is ˜200 nucleotides long (with a small portion up to 4 kb) and thus it is fragmented, although circular RNAs also have been shown to be enriched and stable in EVs. RNA in EVs is protected from RNase digestion in the extracellular environment by the lipid bilayer (Abels and Breakefield (2016) Mol. Neurobiol. 36(3):301-312). The Exocarta, Vesiclepedia, and EVpedia databases are publicly available and provide data on the protein, nucleic acid, and lipid content of EVs (generally EVs from mammalian origin, such as human origin), as well as the isolation and purification procedures used, from EV studies (Abels and Breakefield (2016) Mol. Neurobiol. 36(3):301-312).

EVs are used by cells to mediate several physiological processes or affect various pathological conditions associated with the activation of an immune response or the spread of disease or infection, and also constitute cross-species communication and are in all kingdoms of life. Sources of EVs include mammalian cells, bacteria, bovine milk and plants (Adamo et al. (2021) J Extracell. Vesicles 10:e12081). Although plants and algae possess a cell wall outside their plasma membrane, which could be a physical barrier for the release of EVs, plants and algae release EVs (Picciotto et al. (2021) Biomater. Sci. doi:10.1039/dObm01696a).

### 1. Types of Extracellular Vesicles (EVs)

a. Exosomes

There are three primary subtypes of EVs; they are classified based on their biogenesis, mode of release, size, content, and function: microvesicles (MVs), exosomes, and apoptotic bodies (Doyle and Wang (2019) Cells 8(7):727). Exosomes, or intraluminal vesicles (ILVs) generally are 30-150 nm in diameter and are released through multivesicular bodies (MVBs) in the endosomal pathway. In the endosomal pathway, early endosomes form by inward budding of the plasma membrane and can transform into late endosomes, which accumulate ILVs by inward budding of the endosomal membrane. Late endosomes which contain a number of small vesicles are called MVBs. MVBs either fuse with the lysosome and are degraded, or the plasma membrane which releases the TLVs as exosomes into the extracellular space. The endosomal sorting complexes required for transport (ESCRT) pathway regulates MVB transportation and exosome formation and is reported to be the primary driver of exosome biogenesis, although other mechanisms of exosome biogenesis exist, including those mediated by the sphingolipid ceramide, which can facilitate membrane invagination, or proteins in the tetraspanin family. The ESCRT accessory proteins Alix, TSG101, HSC70 and HSP90β are often referred to as exosomal marker proteins (Doyle and Wang (2019) Cells 8(7):727).

Exosomes are released into the extracellular space by the fusion of the MVB limiting membrane with the plasma membrane. A number of proteins are involved in the release of exosomes, including Rab GTPases, diacylglycerol kinase a, and SNARE proteins (Abels and Breakefield (2016) Cell Mol. Neurobiol. 36(3):301-312).

Exosomes are candidates for drug delivery systems: they have a long circulating half-life; exosomes are tolerated by the human body and can penetrate cell membranes and target specific cell types; and they can be loaded with genetic material, a protein, or a small molecule (Doyle and Wang (2019) Cells 8(7):727).

b. Microvesicles

Microvesicles (MVs, or ectosomes) form by outward budding, or pinching, of the cell's plasma membrane, and have a diameter of 100 nm to 1 m. The formation of MVs involves cytoskeleton components, such as actin and microtubules, molecular motors such as kinesins and myosins, and fusion machinery such as SNAREs and tethering factors. The physiological state and microenvironment of the donor cell effects the number of MVs produced, and the physiological state and microenvironment of the recipient cell effects the number of MVs consumed. MVs also have a number of marker proteins, including cytosolic and plasma membrane associated proteins, as well as cytoskeletal proteins, heat shock proteins, integrins, and proteins containing post-translational modifications, although there are no known specific markers to distinguish MVs from exosomes. Like exosomes, MVs can be loaded with cargo (such as proteins, nucleic acids, and lipids) for delivery to another cell, thereby altering the recipient cell's functions (Doyle and Wang (2019) Cells 8(7):727).

c. Apoptotic Bodies

Apoptotic bodies are released by dying cells into the extracellular space, and have a diameter from 50 nm to 5000 nm. Apoptotic bodies are formed when the cell's plasma membrane separates from the cytoskeleton due to increased hydrostatic pressure after the cell contracts. Unlike exosomes and MVs, apoptotic bodies contain intact organelles, chromatin, and small amounts of glycosylated proteins (Doyle and Wang (2019) Cells 8(7):727).

### 2. Uptake of EVs

Cells internalize EVs by fusion with the plasma membrane, or more commonly by endocytosis (Abels and Breakefield (2016) Cell Mol. Neurobiol. 36(3):301-312). Uptake via endocytosis can be through several types of endocytotic processes, and different processes have been described in different cell types: clathrin-dependent endocytosis and phagocytosis have been described in neurons, macropinocytosis in microglia, phagocytosis and receptor-mediated endocytosis in dendritic cells, caveolin-mediated endocytosis in epithelial cells, and cholesterol- and lipid raft-dependent endocytosis in tumor cells. Blocking heparin sulfate proteoglycans (HSPGs) on the plasma membrane with heparin reduces the uptake of EVs in cell culture, as does blocking the scavenger receptor type B-1 (SR-B1) with a synthetic nanoparticle mimic of HDL, which suggests a role for HSPGs and SR-B1 in EV uptake (Abels and Breakefield (2016) Cell Mol. Neurobiol. 36(3):301-312). Fusion of EVs with the plasma membrane also is a method of uptake, and requires low pH conditions; treatment of EVs with the combination of a pH-sensitive fusogenic peptide with cationic lipids resulted in increased cellular uptake of exosomes and the cytosolic release of cargo within the exosomes (Nakase and Futaki (2015) Sci. Rep. 5:10112). Low pH conditions occur in tumors (Abels and Breakefield (2016) Cell Mol. Neurobiol. 36(3):301-312), so that EVs for delivering therapeutic payloads to tumor cells can enter cells through fusion with the plasma membrane.

Like cells, EVs have extracellular receptors and ligands on the outside and cytoplasmic proteins and nucleic acid on the inside, and thus communicate with cells in different ways. EVs bind to the cell surface, undergo endocytosis, and/or fuse with the plasma membrane, and release their cargos in the extracellular space. If entering by endocytosis, the EV cargo must escape the degradative pathway; late endosomes can fuse with lysosomes or the plasma membrane, so cargo must exit before it is degraded in a lysosome or re-released through the fusion of MVBs with the plasma membrane. EVs containing cargo, including mRNAs and non-coding RNAs, can be transferred to recipient cells in culture and in vivo (Abels and Breakefield (2016) Cell Mol. Neurobiol. 36(3):301-312; Maas et al. (2017) Trends Cell Biol. 27(3):172-188).

### 3. General Methods for Isolating EVs

a. Ultracentrifugation

Ultracentrifugation methods are used to isolate exosomes; alternative methods also have been developed. Due to the complex nature of the biological fluids from which exosomes are isolated, the overlap in physiochemical and biochemical properties between exosomes and other types of EVs, and the heterogeneity among exosomes, isolation methods can result in complex mixtures of EVs and other components of the extracellular space. Differential ultracentrifugation depends on the initial sedimentation of larger and denser particles from the extracellular matrix, and results in an enrichment of exosomes, but not a complete separation of exosomes from other components in the extracellular space. Density gradient centrifugation is another ultracentrifugation method and is based on separation by size and density in the presence of a density gradient (typically made of sucrose or iodixanol) in the centrifuge tube. Density gradient centrifugation effectively separates EVs from protein aggregates and non-membranous particles but has low exosome recovery, although purity can be improved by coupling differential ultracentrifugation with types of density gradient centrifugation, such as rate-zonal centrifugation or isopycnic centrifugation (Doyle and Wang (2019) Cells 8(7):727).

b. Size-Based Techniques

There are a number of size-based techniques for isolating exosomes (Doyle and Wang (2019) Cells 8(7):727). Ultrafiltration separates particles based on the size and molecular weight cut off of the membrane, whereby particles larger than the molecular weight cut off of the membrane are retained, and particles smaller than the molecular weight cut off of the membrane are passed through into the filtrate; low isolation efficiency can occur however if the filter becomes clogged and vesicles become trapped. The ExoMir™ Kit (Bioo Scientific; Austin, TX) is a commercially available kit in which two membranes (200 nm and 20 nm) are placed into a syringe and a sample (typically pre-treated with centrifugation and proteinase K) is passed through the syringe; the larger vesicles remain above the first 200 nm filter, the smallest vesicles are passed through the syringe and discarded, and the vesicles between 20 and 200 nm remain between the two filters in the syringe. Sequential filtration also relies on a series of filtration steps to isolate exosomes (Doyle and Wang (2019) Cells 8(7):727).

Size Exclusion Chromatography (SEC), often used in parallel with ultracentrifugation methods (in which the exosome pellet obtained from ultracentrifugation is resuspended and further purified using SEC), of exosomes is similar to using SEC to separate proteins. In SEC, a column is packed with a porous stationary phase in which small particles can penetrate and thus elute after larger particles. Typically, SEC methods require several hours of run time; however, the qEV Exosome Isolation Kit (iZON Science, New Zealand) allows for rapid and precise exosome isolation by SEC within 15 minutes (Doyle and Wang (2019) Cells 8(7):727).

In Flow Field-Flow Fractionation (FFFF), a sample injected into a chamber is subjected to parabolic flow as it is pushed down the chamber, in addition to a flow perpendicular to the parabolic flow, a crossflow, to separate particles in the sample. Larger particles are more affected by the crossflow and are pushed toward the walls of the chamber, which have a slower parabolic flow, and smaller particles remain in the center. Smaller particles elute earlier, and larger particles later, in FFFF (Doyle and Wang (2019) Cells 8(7):727).

In Hydrostatic Filtration Dialysis (HFD), hydrostatic pressure forces a sample through a dialysis tube with a membrane having a molecular weight cut-off of 1000 kDa. The result is that small solutes are able to pass through the tube, but larger particles, including exosomes and EVs, remain in the tube and can then be further separated using, for example, ultracentrifugation (Doyle and Wang (2019) Cells 8(7):727).

c. Immunoaffinity Capture-Based Techniques

Immunoaffinity capture-based techniques can isolate exosomes based on expression of an antigen on the surface of the exosome, and allow for the isolation of exosomes derived from a particular source. In these methods, an antibody specific for a target antigen can be attached to a plate (e.g., in Enzyme-Linked Immunosorbent Assay, ELISA), magnetic beads (e.g., in magneto-immunoprecipitation), resins and microfluidic devices; these surfaces are then exposed to the exosome sample, resulting in the immobilization of the exosomes expressing the antigen. This assay requires that the protein/antigen for isolating the exosomes be expressed on the surface of the exosomes, and its specificity is limited by the specificity of the antibody that is used, often resulting in a lower yield but higher purity of isolated exosomes. These methods also can be used to separate exosomes within mixed populations of EVs. Immunoaffinity capture-based techniques often are used after ultracentrifugation or ultrafiltration (Doyle and Wang (2019) Cells 8(7):727).

d. Exosome Precipitation

Methods for precipitation of exosomes include precipitation by polyethylene glycol (PEG) and lectin. In PEG precipitation, the PEG polymer ties-up the water molecules, allowing the other particles, including exosomes, to precipitate out of solution. PEG precipitation is quick and is not limited to the starting volume of solution, but lacks selectivity, as other EVs, extracellular proteins, and protein aggregates are precipitated with EVs. Sample pretreatment using filtration and/or ultracentrifugation can improve exosome yield. Commercially available kits for isolating exosomes using precipitation include, for example, ExoQuick® (System Biosciences, Palo Alto, CA) and Invitrogen™ Total Exosome Isolation Kit (Thermo Fisher Scientific, Waltham, MA). Alternatively, lectin precipitation can be used, typically after ultracentrifugation, whereby lectins bind to carbohydrates on the surface of exosomes, altering their solubility and leading to their precipitation out of solution (Doyle and Wang (2019) Cells 8(7):727).

e. Microfluidic Based Isolation Techniques

Microfluidic based techniques isolate exosomes based on their physical and biochemical properties simultaneously, and are rapid, efficient, and require small starting volumes. In acoustic nanofiltration, a matrix containing EVs and other cellular components is injected into a chamber and exposed to ultrasound waves. The particles respond differently to the radiation forces exerted by the waves, depending on their size and density; large particles experience stronger forces and migrate faster toward the pressure nodes. The immuno-based microfluidic isolation technique is similar to that of an ELISA, although, unlike ELISAs, it does not require prior ultrafiltration or ultracentrifugation of exosomes (Doyle and Wang (2019) Cells 8(7):727). The ExoChip (Kanwar et al. (2014) Lab Chip. 14(11):1891-1900) and ExoSearch Chip (Zhao et al. (2016) Lab Chip. 16(3):489-496) have been developed to isolate exosomes using microfluidic technology.

### 4. Microalgae and Microalgae-Derived Extracellular Vesicles (MEVs)

Taxonomy and classification of microalgae can vary. According to some schemes there are seven (7) divisions of microalgae: Euglenophyta (Euglenoids), Chrysophyta (Golden-brown algae and Diatoms), Pyrrophyta (Fire algae), Chlorophyta (Green algae), Rhodophyta (Red algae), Phaeophyta (Brown algae), and Xanthophyta (Yellow-green algae). Of interest herein are photosynthetic microalgae, such as the species Chlorella and Chlamydomonas. The methods and uses described herein use MEVs generally from green algae. Exemplary of such algae are Chlamydomonas and Chlorella, which belong to the classes Chlorophyceae and Trebouxiophyceae, respectively.

Microalgae are bioresources for the production of EVs for use in nanomedicine and other fields. The mechanism of secretion of EVs from microalgae is known in relation to primary and motile cilia/flagella (Picciotto et al. (2021) Biomater. Sci. doi:10.1039/dObm01696a). Chlamydomonas flagella are devoid of MVBs, thus, ciliary EVs shed from Chlamydomonas are classified as ectosomes. Studies have shown the shedding of ectosomes from flagellar and ciliary tips of the chlorophyte Chlamydomonas reinhardtii. EVs also have been observed along the length of the cilium in Chlamydomonas. Membrane budding and ciliary EV formation are mediated by components of the endosomal sorting complex required for transport (ESCRT), which are found in isolated ciliary transition zones, ciliary membranes, and ciliary EVs in Chlamydomonas and can act as sensors of membrane curvature. The formation of ciliary EVs also can occur when ciliary membrane trafficking is disrupted or during ciliary resorption (Wang and Barr (2018) Essays Biochem. 62(2):205-213). Ciliary ectosomes from Chlamydomonas contain a lytic enzyme that digests the mother cell wall and is required for the release of daughter cells. Ift88-null mutants that do not have flagella were unable to be released from the mother cell, and the addition of ciliary ectosomes from wild-type cells rescued the phenotype, suggesting a role for the flagella and intraflagellar transport (IFT) machinery in EV production (Wang and Barr (2016) Cell Mol. Neurobiol. 36(3):449-457).

EVs have been extracted from algal cells using ultra-centrifugation (Kuruvinashetti et al. (2020) 20^{th} International Conference on Nanotechnology 354-357). In accord with this method, algal cells are cultured; the cultured algal cells are collected and centrifuged; the supernatant is collected (and further centrifuged); a sucrose solution is added to the supernatant; and the algal supernatant with the sucrose solution is ultra-centrifuged; because of the sucrose solution, the high-density EVs settle at the bottom of the ultra-centrifugation tube and can be collected using a pipette. Extracted algal EVs can be characterized in size and concentration using Nanoparticle Tracking Analysis (NTA). Studies using this method have isolated green algal EVs that range in size from 25-200 nm, with a concentration of 0.89E8 to 0.94E8 particles/mL (Kuruvinashetti et al. (2020) 20^{th} International Conference on Nanotechnology 354-357).

An ultra-centrifugation protocol also can be used to isolate EVs from marine microalgae grown under various conditions; NTA showed that the nano-particles have a size distribution between 100 and 200 nm, and western blotting of proteins confirmed the presence of EV markers (VES4US, Extracellular vesicles from a natural source for tailor-made nanomaterials, 2020). Subsequent studies have identified microalgal small EVs (sEVs) isolated from the marine photosynthetic microalgal chlorophyte Tetraselmis chuii, termed nanoalgosomes. The production of nanoalgosomes is an evolutionarily conserved trait within microalgal strains as similar results were obtained using sEVs isolated from batch cultures of two other microalgae species, the chlorophyte Dunaliella tertiolecta, and the dinoflagellate Amphidinium sp. The nanoalgosomes were isolated using differential ultracentrifugation (dUC) and tangential flow filtration (TFF), as well as gradient ultracentrifugation, which was used to further purify samples enriched for small EVs by TFF or dUC. The isolated nanoalgosomes were shown to share characteristics of EVs from other sources. The EV yield (measured by sEV protein content and sEV number) from dUC and TFF was consistent with reported numbers of isolated EVs, around 10⁹ EV particles/g EV proteins. Biophysical analysis of particle size using multi-angle dynamic light scattering (DLS), nanoparticle tracking analysis (NTA), fluorescence nanoparticle tracking analysis (F-NTA), and fluorescence correlation spectroscopy (FCS) yielded consistent size distributions, with the size that appeared the most frequently from DLS (DLS mode) around 70 nm. Compared to exosomes derived from mammalian cells, which have a density of 1.15-1.19 g/mol, nanoalgosomes had a slightly lower density of 1.13 g/mol. Electron microscopy revealed that the nanoalgosomes are spherical, heterogeneous in size and shape, and possess a lipid-bilayer structure. Compared to the microvesicles (or large EVs, lEVs) and lysates, the sEVs were enriched for three of the four target protein biomarkers (Alix, enolase, HSP70 and β-actin). DLS measurements indicated that the nanoalgosomes were resistant to changes in pH and stable in human blood plasma. The tumorigenic MDA-MB-231 breast cancer cell line, the non-tumorigenic 1-7 HB2 cell line, and the human hepatocarcinoma Hep G2 cell line did not show cytotoxic or genotoxic effects after nanoalgosome treatment. Furthermore, the nanoalgosomes were taken up by the MDA-MB-231 and 1-7 HB2 cell lines (Adamo et al. (2021) J. Extracell. Vesicles 10:e12081).

EVs have been isolated from at least eighteen microalgae strains (Ankistrodesmus sp., Brachiomonas sp., Chlamydomonas reinhardtii, Dunaliella tertiolecta, Tetraselmis chuii, Chloromonas sp., Rhodella violacea, Kirchneriella sp., Pediastrum sp., Nannochloropsis sp., Cyanophora paradoxa, Cryptomonas pyrenoidifera, Phaeodactylum tricornutum, Phaeothamnion sp., Diacronema sp., Isochrysis galbana, Stauroneis sp., and Amphidinium sp.) from the main microalgal lineages that have been studied, including strains with a variety of features such as saltwater and freshwater inhabitants, small and large sized cells, colonial and single cells, and species with sequenced genomes.

MEVs can be isolated using a differential ultracentrifugation protocol and characterized following the International Society for Extracellular Vesicles (ISEV) guidelines. All strains tested showed the presence of MEVs in the culture medium. EV-producing microalgae strains were established based on the EV protein content, the expression of EV protein markers (e.g., Alix, Hsp70, enolase, and β-actin), the total scattering signal (measured by dynamic light scattering, DLS) or total particle number (measured by NTA), and the sEV average size and size range. These EV-producing strains include Cyanophora paradoxa, Tetraselmis chuii, Amphidinium sp., Rhodella violacea, Diacronema sp., Dunaliella tertiolecta, Phaeodactylum tricornutum, Pediastrum sp., and Phaeothamnion sp. (Picciotto et al. (2021) Biomater. Sci. doi:10.1039/dObm01696a). The data for Cyanophora paradoxa showed ˜2×10⁹ sEV particles per mL of microalgal-conditioned media, with strong positive signals for EV markers, and a size distribution with a mode of 130±5 nm, in agreement with data from plant-derived vesicles. Cytotoxicity and genotoxicity studies showed that sEVs isolated from Cyanophora paradoxa, a freshwater Glaucophyte, did not show toxicity on the tumorigenic MDA-MB-231 breast cancer or C2C12 myoblast cell lines, neither over time nor at different concentrations, nor did MDA-MB-231 cells treated with the sEVs show morphological nuclear changes associated with apoptotic events (Picciotto et al. (2021) Biomater. Sci. doi:10.1039/dObm01696a).

EVs also have been isolated from Synechocystis sp. PCC6803 (a cyanobacterium), Chlamydomonas reinhardtii (a green microalgae), Euglena gracilis (an euglenophyte), and Haematococcus pluvialis (a chlorophyte) in work done by Zhao et al., who also performed RNomic and proteomic analyses in EVs isolated from C. reinhardtii at different stages of cell growth and under different types of abiotic stress (Zhao et al. (2020) doi:10.21203/rs.3.rs-38027/v1). EVs were isolated using differential ultracentrifugation and filtration, and the resuspension was shown to contain membrane structures with small clumps of particles 110-120 nm in diameter, in line with the reported diameter of exosomes and small MVs, although there were differences in diameters between the species of microalgae. Specifically, EVs from C. reinhardtii had diameters between 37-710 nm, with an average particle diameter of 120.1 nm. Synechocystis-derived EVs had diameters between 24-450 nm, with an average particle size of 94.68 nm. Despite the presence of a cell wall, Chlamydomonas cells were able to uptake EVs, as shown by the presence of EVs labeled with a fluorescent lipophilic dye inside microalgal cells. Thus, microalgal EVs can be absorbed by recipient cells. Non-coding RNAs were detected in microalgal EVs at different growth stages and treatment (biotic stress, nitrogen depletion, and nitrogen recovery), and proteomic analyses identified many flagellar-associated membrane proteins in microalgal EVs (Zhao et al. (2020) doi:10.21203/rs.3.rs-38027/v1).

These studies show that microalgae produce EVs that can be isolated using traditional or standard methods; microalgal-derived EVs are similar in size and concentration, and exhibit similar markers compared to EVs isolated from other species; EVs isolated from microalgae do not show cytotoxic or genotoxic effects in vitro; and that microalgal-derived EVs can be taken up by cells.

It has been shown that EVs from mammalian origin can deliver cargo to a target cell and thus have therapeutic use for delivery of a variety of cargos for use in treating a number of diseases or conditions; this has not been shown for in general for MEVs. Mammalian EVs, except for bovine milk EVs, however, cannot be administered orally because they do not survive the harsh conditions of the stomach. For example, small molecules such as hydrophobic and hydrophilic drugs can be injected into exosomes, or macromolecular proteins and nucleic acids can be embedded into the exosomes. The nucleic acids can include those encoding a gene of interest. Specific targeting ligands, imaging probes, and covalent linkage could be attached to the exosome surface and tracked using NTA, fluorescence, or by bioluminescence.

Besides a mention in a publication that microalgae EVs possibly can be used to deliver a drug of interest to a targeted cell, tissue, or organ (Kuruvinashetti et al. (2020) 20^{th} International Conference on Nanotechnology 354-357), there is no published evidence nor technical descriptions for use of MEVs for delivery for treatment of mammalian disease, disorders, or conditions. There are no publications or technical descriptions describing how knowledge for application of EV technology to microalgae-derived extracellular vesicles, nor whether it is possible to do so, nor how to do so. Prior studies have not considered Chlorella species, nor have the prior studies assessed biodistribution and related properties of the MEVs in general. Hence methods, such as methods of oral delivery, exemplified herein with Chlorella, can employ MEVs from other microalgae.

As described and shown herein, however microalgal EVs have a number of advantages over the use of existing drug delivery systems, such as, exosomes derived from mesenchymal stem cells, gold nanoparticles, liposomes and other plant- and animal-derived EVs. Mesenchymal stem cells are a commonly used source of exosomes, and exosomes derived from mesenchymal stem cells are used in drug delivery, for example, anti-cancer vaccines, because they have enhanced passive targeting (a method of preparing a drug carrier system so that it remains circulating in the blood stream). Mesenchymal stem cell derived EVs possess the ability to passively target due to their small size, indigenous nature, and their ability to cross biological barriers. Mesenchymal stem cells, however, have limited secretion of exosomes, and scaling up production of exosomes is difficult due to the need to optimize purification, increase the homogeneity of exosomes, and establish efficient transfection strategies. Nanoparticles can lead to toxicity and current techniques for synthesizing nanoparticles limit their ability to scale for manufacturing purposes. Nanoparticle and liposome-based drug delivery methods also can lead to the formation of a teratoma (a tumor comprised of several different types of tissue). Liposome-based drug delivery methods have been further shown to be less efficient for internalization into a specific cell, tissue or organ, compared to exosomes. Plant-derived EVs, such as those from curcumin, ginger, grapefruit, and lemon, have been used for drug delivery, but their extraction process and use in treatment has not yet been optimized. The production of EVs from agricultural products, such as fruits and milk, is economically impractical and need 3-4 months to grow, compared to algal EVs, which can be grown anywhere and within a few days. Algal EVs avoid phagocytosis or degradation by macrophages and circulate for prolonged times in vivo, and have low immunogenicity. Algal EVs also have a lower risk of teratoma formation. Algae, thus, provide a source from which pure, well-characterized EVs of high quality can be obtained (Kuruvinashetti et al. (2020) 20^{th} International Conference on Nanotechnology 354-357). Kuruvinashetti et al. does not describe the use of Chlorella species as a source of EVs, nor its advantages as a source. Prior art does not describe the biodistribution of MEVs per se, nor the implications thereof for administration of MEVs with drugs directed to particular organs, tissues, or systems.

It is shown and described herein that MEVs bypass stringent biological barriers, including the gastrointestinal barrier, as well as the blood-brain barrier, and choroid-retina barrier, and provide for effective delivery to the lungs and mucosal surfaces of other organs.

### 5. Green Algae—Chlorella Species

Previous studies and consideration of EVs have not focused on nor assessed Chlorella species as sources of EVs. Chlorella and the resulting EVs have advantages for growth, manipulation, and administration of drugs that other species and EVs do not provide. Green algae belong to phylum Chlorophyta, and encompass a diverse group of photosynthetic eukaryotes. Green algae include unicellular and multicellular organisms. Algae originally included in the genus Chlorella are among the most widely distributed and frequently encountered algae in freshwater. These algae exist in aqueous environments and on land. They are typically small (˜2 to 10 m in diameter), unicellular, spherical in shape, non-motile, and contain a single chloroplast, and some have a rigid cell wall (Blanc et al. (2010) Plant Cell 22(9):2943-2955).

Molecular analyses have separated Chlorella species into two classes of chlorophytes: the Trebouxiophyceae, which contains the true Chlorella; and the Chlorophyceae. For use herein, Chlorella species include any that can be or that are used as food complement or that can be consumed by humans or other animals, such as livestock. Exemplary species include, but are not limited to, the species: Chlorella ellipsoidea, Chlorella pyrenoidosa, Chlorella sorokiniana, Chlorella vulgaris, and Chlorella variabilis.

True Chlorella species are characterized by glucosamine as a major component of their rigid cell walls. Although most Chlorella species are naturally free-living, the Trebouxiophyceae include most of the known green algal endosymbionts, living in lichens, unicellular eukaryotes, plants, and animals (for example mussels and hydra). For example, Chlorella variabilis NC64A is a hereditary photosynthetic endosymbiont (or photobiont) of Paramecium bursaria, a unicellular protozoan, and NC64A also is a host for a family of large double-stranded DNA viruses that occur in freshwater (Blanc et al. (2010) Plant Cell 22(9):2943-2955).

a. Life Cycle

In unicellular organisms, such as microalgae, life cycle is the same as the cell cycle. Chlorella is a haploid organism that reproduces asexually by autosporulation. The cell cycle and proliferation of Chlorella vulgaris has been investigated using flow cytometric analysis of 5(6)carboxyfluorescein diacetate N-succinimidyl ester (CFSE)-stained algal cells by Rioboo et al. Their results indicate that, as generally described for microalgae, the growth of C. vulgaris mother cells takes place during light periods, whereas cytoplasmic division and liberation of daughter cells takes place during dark periods. C. vulgaris also shows a distinct light/dark cycle, marked by an increase in cell size, cell complexity, and autofluorescence during periods of light, measured over a 96-hour period. A monoparametric histogram of CFSE-stained C. vulgaris cells showing only one peak of daughter cells indicates that each mother cell undergoes only one division cycle in 96 hours; the cytoplasmic division was further shown to take place during periods of darkness. Thus, the strain of C. vulgaris used exhibits three life cycle phases: 1) growth of mother cells, 2) cell division, and 3) liberation of daughter cells. C. vulgaris cells grew during 2 light periods and began to divide during following dark period; cell division occurs once the mother cells are double the size of daughter cells. Furthermore, C. vulgaris cells exposed to the herbicide terbutryn need a longer growth period in order to reach a large enough cell size to divide. This suggests there is a critical threshold size needed for C. vulgaris to complete the growth phase and begin the division phase, and that this critical threshold can control the progression of the G1 phase of the C. vulgaris cell cycle. Finally, this study demonstrates that the intensity of the peak of CFSE-fluorescence of mother cells is four times greater than that of the daughter cells, indicating that 4 daughter cells are produced from each mother cell. Thus, C. vulgaris cells undergo a first mitosis followed by cytoplasmic division, and then two other simultaneous mitoses, which result in the liberation of 4 daughter cells (Rioboo et al. (2009) doi:10.1016/j.aquatox.2009.07.009).

b. Genomic Analyses of Chlorella Species

Although species of Chlorella are reported to be non-motile and lack a sexual cycle, genomic analyses of Chlorella variabilis NC64A (NC64A) and Chlorella vulgaris 211/11P (211/11P) reveal the presence of genes involved in sexual reproduction and motility (Blanc et al. (2010) Plant Cell 22(9):2943-2955; Cecchin et al. (2019) Plant J. 100(6):1289-1305). The NC64A nuclear genome (GenBank® Accession No. ADIC00000000.1) is 46.2 Mb, and composed of 12 chromosomes. The meiosis-specific proteins dosage suppressor of MCk1 DMC1, homologous-pairing proteins HOP1 and HOP2, meiotic recombination protein MER3, meiotic nuclear division protein MND1, and mutS homolog protein MSH4 are encoded in NC64A; these genes also occur in most of the other sequenced chlorophyte algal species. Nineteen homologs of the Chlamydomonas gametolysin proteins, which promote disassembly of the gametic cell walls and allow gamete fusion, also were identified in NC64A. Additionally, an ortholog of the Chlamydomonas GCS1 protein, which is essential for cell fusion, occurs in NC64A (Blanc et al. (2010) Plant Cell 22(9):2943-2955). The primary genes involved in meiosis also occur in the Chlorella vulgaris 211/11P 40 Mb genome (GenBank® Accession No. SIDB00000000), in addition to the gene encoding gametolysin (g3347), and a gene encoding a protein that contains a domain with a putative GCS1/HAP2 function (Cecchin et al. (2019) Plant J. 100(6):1289-1305). Thus, although Chlorella species have been observed only in the haploid phase, the presence of meiosis genes indicates that the life cycle of Chlorella could include a diploid phase.

Similarly, while flagella have not been observed in NC64A, orthologs of the Chlamydomonas flagellar proteins were identified in the NC64A genome, including orthologs to the intraflagellar transport (IFT) proteins IFT52, IFT57, and IFT88, kinesin-2 motor protein FLA8, the kinesin-associated protein KAP, and proteins involved in the axonemal outer dynein arm (Blanc et al. (2010) Plant Cell 22(9):2943-2955).

Sequencing of three Chlorella sorokiniana strains, strain 1228, UTEX 1230, and DOE1412, reveals the presence of sex- and flagella-related genes (Hovde et al. (2018) Algal Research 35:449-461). The genome of several other Chlorella species has been sequenced: Chlorella protothecoides sp. 0710 (Gao et al. (2014) BMC Genomics 15(1):582; GenBank® Accession No. APJO00000000); Chlorella sorokiniana UTEX 1602 (GenBank® Accession No. LHPG00000000) and Chlorella sp. strain SAG 241.80 (Micractinium conductrix; GenBank® Accession No. LHPF00000000) (Arriola et al. (2018) Plant J. 93(3):566-586); and the Chlorella vulgaris strains UTEX 395 (Guarnieri et al. (2018) Front. Bioeng. Biotechnol. 6:37; GenBank® Accession No. LDKB00000000), UMT-M1 (Teh et al. (2019) Data Brief 27:104680; GenBank® Accession No. VJNP00000000), UTEX 259 (GenBank® Accession No. VATW00000000) and NJ-7 (Wang et al. (2020) Mol. Biol. Evol. 37(3):849-863; GenBank® Accession No. VATV00000000).

c. Commercial and Biotechnological Uses of Chlorella

The commercial cultivation of microalgae for food purposes began with the production of Chlorella vulgaris in Japan and Taiwan in the 1960s. Dried biomass products from Arthrospira and Chlorella are included in dietary supplements due to reports of high protein content, nutritive value, and health benefits. For example, Chlorella extracts have been shown to lower cholesterol and have antioxidant, antibacterial, and antitumor activities. Production of high yields of Chlorella is routine, and, as detailed herein, MEVs can be isolated from the cell culture medium. For its use as a pharmaceutical, it is known that ingestion of Chlorella is non-toxic and non-immunogenic in humans.

Chlorella has been used in a variety of biotechnology applications, including biofuels, sequestering CO₂, producing molecules of high economic value, or removing heavy metals from wastewaters (Blanc et al. (2010) Plant Cell 22(9):2943-2955). Chlorella species show metabolic flexibility in response to environmental perturbations, and are capable of using nutrients, such as organic carbon and minerals, directly from wastewater for growth. Among microalgae, Chlorella species have higher photosynthetic efficiency over other photosynthetic organisms. Additionally, Chlorella vulgaris is able to grow either in autotrophic, heterotrophic or mixotrophic conditions (Zuñiga et al. (2016) Plant Physiol. 172(1):589-602).

Chlorella species also can be genetically modified by Agrobacterium-mediated transformation. A study by Cha et al. developed a method to genetically transform Chlorella vulgaris using the Agrobacterium tumefaciens strain LBA4404, and the presence of gene fragments in 30% of the transgenic lines, compared to the wild-type non-infected Chlorella, indicates the T-DNA was integrated into the Chlorella genome (Cha et al. (2012) World J. Microbiol. Biotechnol. 28:1771-1779).

d. Chlorella MEVs

As described herein, Chlorella species, such as C. vulgaris, are advantageous species for the production of EVs, referred to herein as MEVs, for use for delivery of biomolecules and small molecules for many applications, including therapeutic, diagnostic, and cosmetic uses. Of particular interest herein are MEVs produced by Chlorella species. Chlorella EVs have not been exploited as sources of MEVs for exogenous loading of biomolecular products or small molecule drugs or diagnostic agents. Chlorella, as a source of EVs for such applications, provides numerous advantages. Chlorella is a haploid organism, which means that specific and targeted variants can be produced by genetic engineering; it readily can be genetically modified or loaded to produce or contain biologically active molecules and small molecules. Stable cell lines can be produced, including stable producers of encoded products. They are defined products, and, when exogenously loaded, the resulting compositions contain EVs that contain the same cargo.

Detailed genetic maps can be obtained, and correlations between genotype and phenotype can be established. Chlorella genomes have been fully sequenced, so the structure and function of various genes can be known. Phylogenetically, Chlorella is at the very crossroads between higher plants and microalgae. As such, Chlorella shares with higher plants a significant (and useful) number of molecular biological and metabolic features, but still is a unicellular haploid microalgae. Exemplary of molecular biological features shared with eukaryotes is the intracellular machinery that involves the dicer enzyme system for processing exogenous RNA into siRNA. Chlorella is autotrophic; unlike mammalian and other animal cells, it can therefore be cultured and reproduced without the need for nutrients or factors of animal origin.

With respect to use of its EVs as therapeutics, Chlorella species are not toxic. For example, tablets made from Chlorella vulgaris biomass (i.e., compressed whole Chlorella cells) have been consumed regularly for years by the public worldwide as a dietary supplement, without constraints related to toxicity or immunogenicity. Japan is the world leader in the consumption of Chlorella biomass. It also is used, for example, in Japan, for medical treatments because it has shown to have immunomodulatory properties and purported anti-cancer activities, for use for anti-aging applications, such as for cardiovascular diseases, hypertension and cataracts; it reduces the risk of atherosclerosis and stimulates the synthesis of collagen for the skin.

Chlorella cells naturally produce extracellular vesicles (EVs) that respond to the ‘standard specifications' of better known EVs (such as mammalian EVs). EVs from plant origin bear a number of features that make them more promising/convenient than synthetic nanoparticles or semisynthetic EVs, for use as a drug delivery system in humans. These include, for example, higher stability, lower toxicity, and lower immunogenicity. Being as close to plants as it is, Chlorella provides a source of EVs with similar characteristics to plant EVs. At the same time, mass production of Chlorella in large scale is easier and cheaper than for higher plants. The glycosylation pattern of membrane proteins in Chlorella is similar/identical to the glycosylation pattern present in higher plants.

The size of the Chlorella MEVs ranges between about or between 50 nm and 200 m, with an average size of about 130 nm. The morphology resembles plant and mammalian exosomes. For use for administration, the size distribution can be rendered more uniform by separating the MEVs by size and selecting those of a size of interest, which can vary depending upon the intended use and route of administration.

### D. EXOGENOUSLY LOADED MICROALGAE EXTRACELLULAR VESICLES (MEVS), CARGO, AND TARGETS

Targets and cargo (see discussions below) include any known to those of skill in the art. Sections F and G and examples below describe the biodistribution of MEVs following administration by various routes, and the implications, uses and methods for targeting or treating particular diseases, disorders, and conditions, and for formulating and administering the MEVs.

### 1. Isolation of MEVs

Methods for isolation are discussed in the sections above and detailed in the Examples.

### 2. MEV Loading and Cargos

The MEVs can be loaded with any desired cargo (also referred to as a payload), including, but not limited to, nucleic acid molecules, including, for example RNAi, plasmids, anti-sense nucleic acids, nucleic acids encoding the RNAi or anti-sense nucleic acid, detectable marker proteins and tags, small molecule drugs, gene editing systems, and others, and combinations thereof. The MEVs can deliver therapeutic molecules, can serve as vaccines, and can be used in human and other animal health, agricultural applications, gene therapy applications, including delivery genes, modification of genes with gene editing systems, and gene silencing nucleic acids, cosmetic applications, dermatological applications, diagnostic applications, industrial uses, and others. The MEVs can deliver nutrients, or regulators of gene pathways to produce a beneficial product, and can be used to deliver gene editing systems, such as CRISPR/Cas and to effect gene editing. The MEVs can be used to deliver gene therapy vectors, such as, but not limited to, adeno-associated (AAV) virus vectors, adenovirus vectors, vaccinia virus-derived vectors, and others, including products for effecting gene therapy.

Diseases and conditions that can be treated include any known to those of skill in the art, including but not limited to, cardiovascular diseases, metabolic diseases, infections, including respiratory infections, bladder infections and other urinary tract infections, infectious diseases, including viral disease, such as hepatitis, HIV, corona viruses, including SARS-Cov-2, CNS diseases, ocular diseases, and liver diseases. As discussed, delivered cargo includes protein products, such as antibodies and antigen-binding forms thereof, RNA products, such as, but not limited to, siRNA, miRNA (micro RNA), lncRNA (long non-coding RNA), saRNA (small activating RNA), shRNA, and mRNA, nucleic acid encoding the products, such as plasmids, nucleic acid products such as DNA encoding anti-sense oligonucleotides and also the anti-sense oligonucleotides, and small molecule drugs.

The MEVs can carry cargos that include reporter genes and proteins and other detectable products, such as, for example, a fluorescent protein, such as, but not limited to an enhanced green fluorescent protein (EGFP; SEQ ID NO:10), a luciferase gene (SEQ ID NO:11), luxA (SEQ ID NO:8), luxB (SEQ ID NO:9), and the Lux operon (luxCDABE and luxABCDE; SEQ ID NO:12). Other cargos provided herein include antigens, antibodies, immunomodulators, discussed in sections throughout the disclosure.

Extracellular vesicles and exosomes also can be used to transfer therapeutic agents such as nucleic acids, such as microRNA, mRNA, tRNA, rRNA, siRNA, regulatory RNA, non-coding and encoding RNA, DNA fragments, and DNA plasmids (see, e.g., CN105821081A and CN110699382A); nucleotides or amino acids comprising a detectable moiety or a toxin or that disrupts transcription or translation, respectively; polypeptides (e.g., enzymes); lipids; carbohydrates; and small molecules (e.g., small molecule drugs and toxins) (see, U.S. Pat. No. 10,195,290). Non-limiting examples of proteins that can be encoded for by the nucleic acid cargo molecule include, but are not limited to: antibodies, intrabodies, single chain variable fragments, affibodies, enzymes, transporters, tumor suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins, structural proteins, neurotrophic factors, membrane transporters, nucleotide binding proteins, heat shock proteins, CRISPR-associated proteins, cytokines, cytokine receptors, caspases and any combination and/or derivatives thereof (see, e.g., AU2018365299).

Reporter Genes, Reporter Proteins, and/or Modulators Thereof can be Delivered in the MEVs.

### Reporter Proteins

Target sequences, in the form of siRNAs, miRNAs, anti-sense oligonucleotides (ASOs), peptides and/or tetratricopeptides, to modulate (inhibition or stimulation) each of the marker genes, such as a GFP protein, a eukaryotic luciferase, or a prokaryotic Luciferase, such as: Lux operon (luxCDABE) and lux operon (luxABCDE), can be used, for example for diagnostics and gene expression assessments (SEQ ID NOs:5-6, 7, and 62-65, respectively):

| | Type of | |
|---|---|---|
| Target gene | sequence | Sequence(s) |
| EGFP | siRNA | sense: 5′-GCAAGCUGACCCUGAAGUUCAUUU-3′ |
| | | antisense: 5′-AUGAACUUCAGGGUCAGCUUGCCG-3′ |
| firefly luciferase | shRNA | 5′-CTGACGCGGAATACTTCGA-3′ |
| luxA | siRNA | sense: 5′-CAAACAGAGGUAAUGAAAUGGUUG-3′ |
| (Lux operon) | | antisense: 3′-CAACCAUUUCAUUACCUCUGUUUG-5′ |
| luxB | siRNA | sense: 5′-AUGUUAAGUUGAAUAAGUUCUGCA-3′ |
| (Lux operon) | | antisense: 3′-UGCUCUUGAAUAAGUUGAAUUGAU-5′ |

Cargo for purposes herein includes antigens for producing or inducing an immune response against a pathogen or for treating or preventing a disease, disorder, or condition.

Cargo can include immunomodulatory agents that increase or modulate the immune response to the vaccines that increase or decrease production of one or more cytokines, up- or down-regulate self-antigen presentation, mask MHC antigens, or promote the proliferation, differentiation, migration, or activation state of one or more types of immune cells. Examples of immunomodulatory agents include but are not limited to non-steroidal anti-inflammatory drugs (NSAIDs) such as aspirin, ibuprofen, celecoxib, diclofenac, etodolac, fenoprofen, indomethacin, ketorolac, oxaprozin, nabumetone, sulindac, tolmetin, rofecoxib, naproxen, ketoprofen, and nabumetone; steroids (e.g., glucocorticoids, dexamethasone, cortisone, hydroxycortisone, methylprednisolone, prednisone, prednisolone, triamcinolone, azulfidine eicosanoids such as prostaglandins, thromboxanes, and leukotrienes; as well as topical steroids such as anthralin, calcipotriene, clobetasol, and tazarotene); cytokines such as TGFβ, IFNα, IFNβ, IFNγ, IL-2, IL-4, IL-10; cytokine, chemokine, or receptor antagonists including antibodies, soluble receptors, and receptor-Fc fusions, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D), CTLA4, eotaxin, Fas, ICAM, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, Bromocriptine, cyclophosphamide, cyclosporine A, D-penicillamine, deoxyspergualin, FK506, glutaraldehyde, gold, hydroxychloroquine, leflunomide, malononitriloamides (e.g., leflunomide), methotrexate, minocycline, mizoribine, mycophenolate mofetil, rapamycin, and sulfasalazine.

Other cargo includes cytokines which include, but are not limited to lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; Mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta, and -gamma; colony stimulating factors (CSFs) such as macrophage-CSF (M-CSF); granulocyte-macrophage-CSF (GM-CSF); and granulocyte-CSF (G-CSF); interleukins (TLs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL).

Other exemplary cargo includes cytokines and other agents that stimulate cells of the immune system and enhance desired effector function. For example, agents that stimulate NK cells include IL-2; agents that stimulate macrophages include but are not limited to C5a, formyl peptides such as N-formyl-methionyl-leucyl-phenylalanine. Cargo include agents that stimulate neutrophils, such as, for example, G-CSF and GM-CSF. Additional agents include, but are not limited to, interferon gamma, IL-3 and IL-7.

### 3. Generation of Payload-Loaded MEVs

As shown herein, the isolated Chlorella can be loaded with cargo for delivery to humans by any suitable route, including but not limited to intravenous, oral, topical, mucosal, inhalation, and any other routes known to those of skill in the art for delivery of vehicles, such as lipid nanoparticles, vectors, therapeutic bacteria, and therapeutic viruses. Upon administration, the MEVs are taken up by cells. Any cargo presently delivered in vectors, bacteria, exosomes, nanoparticles, and other such delivery vehicles can be loaded into the MEVs provided herein. The loaded cargo can be selected so that it only is expressed or produced in targeted cells, such as in instances in which the cargo is a plasmid encoding a therapeutic product. Transcription regulatory signals can be selected so that the encoded product is expressed in targeted cells. For example, for expression in the liver, the encoded product can be expressed under control of a liver-specific promoter, or the product can be targeted to a receptor or target expressed in targeted cells, such as in tumors or in the tumor microenvironment. Loading methods, described above, and in the Examples below, include, but are not limited to:
a. Electroporation
b. Sonication
c. Extrusion
d. Surfactants
e. Other Methods known to those of skill in the art for introducing exosomes into cells.

### 4. Exemplary Cargo and Exemplary Uses of the Exogenously Loaded MEVs

a. Cargo

As described above, the MEVs are loaded with cargo that can be used for any purpose of interest, including any for which other delivery vehicles are used. These uses include delivery of mRNA, such as mRNA encoding corona virus spike proteins and modified spike proteins to improve the immune response to the viruses, RNAi, such as siRNA, and anti-sense RNA, or anti-sense DNA (ASO), to silence genes, such as bacterial and viral pathogen virulence genes, antibiotic resistance genes, antimicrobial resistance genes, genes that suppress the immune system, tumor genes, such as oncogenes, and host factors for viral infection, such as targeting angiotensin-converting enzyme-2 (ACE2), transmembrane protein serine 2 (TMPRSS2), and other such genes. The cargo also can include any therapeutic antibodies. Therapeutic antibodies, include, but are not limited to, anti-cancer antibodies, antibodies to treat autoimmune or inflammatory disease, antibodies to treat transplant rejection, antibodies to treat graft-versus-host-disease (GVHD), and antibodies to treat infectious diseases.

### 1) RNA Cargo

The mechanism of RNA interference or RNAi was originally described as a process of sequence-specific silencing of gene expression in the nematode Caenorhabditis elegans (Fire et al. (1998) Nature 391(6669):806-11; Fire and Mello, 2006 Nobel Prize in Medicine awarded to Andrew Fire and Craig Mello). The process of small RNAs targeting (and silencing) messenger RNAs involves a particular RNAi machinery (including silencing factors, such as DICER and ARGONAUTE).

In the plant kingdom, RNAi is involved in antiviral defense mechanisms, and in defense mechanisms against phytopathogenic fungi and oomycetes. Small regulatory RNAs can be active in silencing genes inside bacterial cells, which lack the said RNAi machinery. The silencing activity of siRNA has been demonstrated to be inter-kingdom (see, e.g., PCT/EP2019/072169, published as International PCT Publication No: WO2020/035619; PCT/EP2019/072170, published as WO2020/035620; Singla et al. (2019c) bioRxiv, doi: doi.org/10.1101/863902).

RNAi-mediated regulation of gene expression has been exploited for several years in the field of biotechnology to confer resistance to viruses (Baulcombe (2015) Current Opinion in Plant Biology 26:141-146). The inter-kingdom RNAi has been used to characterize the function of genes of eukaryotic pathogens/parasites as well as to induce protection against these organisms.

In Drosophila and Caenorhabditis, RNAi plays a crucial role in antiviral defense by directly targeting viral RNAs via the small RNAs produced by the host in response to viruses. Recent work has shown that plant EVs naturally loaded (loaded by the plant cells producing the EVs) with small RNAs, from human edible plants, can modify the composition of the human gut microbiota and oral microbiota by silencing the expression of specific genes in certain commensal bacteria (Teng et al. (2018) Cell Host & Microbes 24:637-652; Sundaram et al. (2019) iScience 21:308-327).

Small interfering RNAs (siRNAs) and microRNAs (miRNAs) are noncoding RNAs with important roles in gene regulation. They have recently been investigated as novel classes of therapeutic agents for the treatment of a wide range of disorders including cancers and infections. Clinical trials of siRNA- and miRNA-based drugs have already been initiated. siRNAs and miRNAs share many similarities, both are short duplex RNA molecules that exert gene silencing effects at the post-transcriptional level by targeting messenger RNA (mRNA), yet their mechanisms of action and clinical applications are distinct. The major difference between siRNAs and miRNAs is that the former are highly specific with only one mRNA target, whereas the latter have multiple targets. The siRNAs and miRNAs have a role in gene regulation, and serve as targets for drug discovery and development. Compared with conventional small therapeutic molecules, siRNAs and miRNAs offer the potential to be highly potent and able to act on “non-druggable” targets (for example, proteins which lack an enzymatic function); moreover, RNAi can be designed to target and/or affect expression of any gene of interest.

### 2) Antibody Cargo

Examples of anti-cancer antibodies and other antibodies, include, but are not limited to, anti-17-1A cell surface antigen antibodies such as the antibody sold or provided under the trademark Panorex® (edrecolomab); anti-4-1BB antibodies; anti-4Dc antibodies; anti-A33 antibodies such as A33 and CDP-833; anti-α1 integrin antibodies such as natalizumab; anti-α4β7 integrin antibodies such as LDP-02; anti-αVβ1 integrin antibodies such as F-200, M-200, and SJ-749; anti-aV03 integrin antibodies such as abciximab, CNTO-95, Mab-17E6, and Vitaxin®; anti-complement factor 5 (C5) antibodies such as 5G1.1; anti-CA125 antibodies such as sold or provided under the trademark OvaRex® (oregovomab); anti-CD3 antibodies such as those sold or provided under the trademark Nuvion® (visilizumab) and Rexomab™; anti-CD4 antibodies such as IDEC-151, MDX-CD4, OKT4A; anti-CD6 antibodies such as Oncolysin B and Oncolysin CD6; anti-CD7 antibodies such as HB2; anti-CD19 antibodies such as B43, MT-103, and Oncolysin B; anti-CD20 antibodies such as 2H7, 2H7.v16, 2H7.v114, 2H7.v115, the product sold or provided under the trademark Bexxar® (tositumomab), the antibody sold or provided under the trademark Rituxan® (rituximab), and the antibody sold or provided under the trademark Zevalin® (Ibritumomab tiuxetan); anti-CD22 antibodies such as the those sold or provided under the following generic names, tradenames, or trademarks: Lymphocide® (epratuzumab); anti-CD23 antibodies such as IDEC-152; anti-CD25 antibodies such as basiliximab and Zenapax® (daclizumab); anti-CD30 antibodies such as AC10, MDX-060, and SGN-30; anti-CD33 antibodies such as gemtuzumab ozogamicin (sold under the trademark Mylotarg®), Oncolysin M, and Smart M195; anti-CD38 antibodies; anti-CD40 antibodies such as SGN-40 and toralizumab; anti-CD40L antibodies such as 5c8, Antova®, and IDEC-131; anti-CD44 antibodies such as bivatuzumab; anti-CD46 antibodies; anti-CD52 antibodies such as alemtuzumab (sold under the trademark Campath®); anti-CD55 antibodies such as SC-1; anti-CD56 antibodies such as huN901-DM1; anti-CD64 antibodies such as MDX-33; anti-CD66e antibodies such as XR-303; anti-CD74 antibodies such as IMMU-110; anti-CD80 antibodies such as galiximab and IDEC-114; anti-CD89 antibodies such as MDX-214; anti-CD123 antibodies; anti-CD138 antibodies such as B-B4-DM1; anti-CD146 antibodies such as AA-98; anti-CD148 antibodies; anti-CEA antibodies such as cT84.66, labetuzumab, and Pentacea®; anti-CTLA-4 antibodies such as MDX-101; anti-CXCR4 antibodies; anti-EGFR antibodies such as ABX-EGF, cetuximab (such as the product sold under the trademark Erbitux®), IMC-C225, and Merck Mab 425; anti-EpCAM antibodies such as Crucell's anti-EpCAM, ING-1, and KS-IL-2; anti-ephrin B2/EphB4 antibodies; anti-Her2 antibodies such as tratuzumab (trademark Herceptin®), MDX-210; anti-FAP (fibroblast activation protein) antibodies such as sibrotuzumab; anti-ferritin antibodies such as NXT-211; anti-FGF-1 antibodies; anti-FGF-3 antibodies; anti-FGF-8 antibodies; anti-FGFR antibodies, anti-fibrin antibodies; anti-G250 antibodies such as WX-G250 and Girentuximab (sold under the trademark Rencarex®); anti-GD2 ganglioside antibodies such as EMD-273063 and TriGem®; anti-GD3 ganglioside antibodies such as BEC2, KW-2871, and mitumomab; anti-gpIIb/IIIa antibodies such as ReoPro®; anti-heparinase antibodies; anti-Her2/ErbB2 antibodies such as trastuzumab, MDX-210, and pertuzumab; anti-HLA antibodies (such as the product sold under the trademark Oncolym®), Smart 1D10; anti-HM1.24 antibodies; anti-ICAM antibodies such as ICM3; anti-IgA receptor antibodies; anti-IGF-1 antibodies such as CP-751871 and EM-164; anti-IGF-1R antibodies such as IMC-A12; anti-IL-6 antibodies such as CNTO-328 and elsilimomab; anti-IL-15 antibodies (such as the product sold under the trademark HuMax®-IL15); anti-KDR antibodies; anti-laminin 5 antibodies; anti-Lewis Y antigen antibodies such as Hu3S193 and IGN-311; anti-MCAM antibodies; anti-Muc antibodies such as BravaRex and TriAb; anti-NCAM antibodies such as ERIC-1 and ICRT; anti-PEM antigen antibodies such as Theragyn and Therex; anti-PSA antibodies; anti-PSCA antibodies such as IG8; anti-Ptk antibodies; anti-PTN antibodies; anti-RANKL antibodies such as AMG-162; anti-RLIP76 antibodies; anti-SK-1 antigen antibodies such as Monopharm C; anti-STEAP antibodies; anti-TAG72 antibodies such as CC49-SCA and MDX-220; anti-TGF-0 antibodies such as CAT-152; anti-TNF-α antibodies such as CDP571, CDP870, D2E7, adalimumab (such as the product sold under the trademark Humira®), and infliximab (such as the product sold under the trademark Remicade®); anti-TRAIL-R1 and TRAIL-R2 antibodies; anti-VE-cadherin-2 antibodies; and anti-VLA-4 antibodies (such as the product sold under the trademark Antegren®). Furthermore, anti-idiotype antibodies including but not limited to the GD3 epitope antibody BEC2 and the gp72 epitope antibody 105AD7, can be used. In addition, bispecific antibodies including but not limited to the anti-CD3/CD20 antibody Bi20 can be used.

Additional exemplary cargo, uses and treatments that can be effected with cargo-loaded MEVs are described, by way of example, as follows.

b. Diseases and Methods of Treatment

As described above, the MEVs can be loaded with any desired cargo, including, but not limited to, nucleic acid molecules, detectable marker proteins and tags, small molecule drugs, gene editing systems, and others, and combinations thereof for delivering therapeutic molecules, serving as vaccines, and for use in human and other animal health, agricultural, cosmetic, dermatological and diagnostic applications, industrial uses, and other uses. The MEVS provided herein are for use as vaccines and immunomodulators, and for treating or preventing (reducing the risk of) a disease, disorder, or condition involving a pathogen or a cancer or a disease, disorder, or condition in which treatment or prevention involves immune modulation. In particular, the MEVs provided herein can elicit an immune response that includes IgG antibodies and/or IgA antibodies. The MEVs can contain cargo for treatment or prevention of a disease, disorder, or condition involving or caused by a pathogen, such as a bacterium, virus, fungi, or parasite, or an inflammatory a disease, disorder, or condition, such as an allergy, asthma, autoimmune disease, cancer, or any such disease, disorder, or condition.

MEVs can be used to deliver DNA or mRNA molecules that encode therapeutically useful polypeptides, and/or to deliver polypeptides, peptides, and proteins.

### E. ENDOGENOUSLY LOADED (ENDO-LOADED) MICROALGAE EXTRACELLULAR VESICLES (MEVS), CARGO, AND TARGETS

For use as vaccines and/or for delivery of any nucleic acid or polypeptides cargo, the MEVs can be endogenously loaded (endo-loaded). The uses and selection of cargo, with the understanding that the cargo has to be produced by the microalgae and/or the host cell, administration is the same as for exo-loaded. For endo-loaded MEVs, DNA encoding a product of interest, such as a protein, mRNA, synthetic pathway, or other product, is introduced into the microalgae cell by any suitable method. Methods for introducing DNA into a microalgae cell are known in the art (for a review see, e.g., Gutierrez et al. (2021) Biology 10:265).

Heterologous DNA can be introduced into microalgae by a variety of methods, including but not limited to, mechanical agitation, surfactant permeabilization, electroporation, particle bombardment, bacterial DNA transfer, nanoparticles, liposomes, and cell penetrating peptides or cell penetrating polymers to mediate penetration into the cell, and other methods known to those of skill in the art for introducing DNA into plant cells, particularly microalgae cells. For example, microalgae cells can be transformed by Agrobacterium tumefaciens transformation using the Ti plasmid of the agrobacterium. This process is well-known to those of skill in the art. The Ti plasmid, into which DNA of interest can be cloned, introduces DNA into the microalgae genome. The DNA of interest integrates into the microalgae genome. To prepare endo-loaded MEVs, DNA that encodes the heterologous product to be endo-loaded in the MEVs, is introduced into the microalgae and the microalgae produces the heterologous product, such as a protein, or mRNA.

Targets and cargo (see discussions below) include any known to those of skill in the art. For endo-loading, the heterologous product must be one that is produced by or loaded into the microalgae cell, and from the cell into the cell-produced MEVs.

### 1. Choice and Preparation of Cargo

The MEVs can be endogenously loaded with any suitable heterologous cargo, including, but not limited to, nucleic acid molecules, including, for example RNAi, such as siRNA, miRNA, lncRNA, and mRNA, including modified mRNA, encoding coding any protein, polypeptide and peptide, detectable marker proteins and tags or any therapeutic or prophylactic or vaccine polypeptide or peptide, gene editing systems, and others, and combinations thereof. The MEVs can deliver therapeutic molecules, can serve as vaccines, and can be used in human health, gene therapy applications, including delivery genes, modification of genes with gene editing systems, and gene silencing nucleic acids, cosmetic applications, dermatological applications, diagnostic applications, industrial uses, and others. The MEVs can deliver regulators of gene pathways to produce a beneficial product, and can be used to deliver gene editing systems, such as CRISPR/cas (see e.g., SEQ ID NOs: 73 and 74 for exemplary CRISPR/cas protein and encoding nucleic sequences, respectively) to effect gene editing.

Diseases and conditions that can be treated include any known to those of skill in the art, including but not limited to, cardiovascular diseases, metabolic diseases, infections, including respiratory infections, bladder infections and other urinary tract infections, infectious diseases, including viral disease, such as hepatitis, HIV, corona viruses, including SARS-CoV-2, CNS diseases, ocular diseases, and liver diseases. As discussed, delivered cargo includes protein products, such as, but not limited to, enzymes, regulatory factors, signaling proteins, antigens, antibodies and antigen-binding forms thereof, RNA products, such as, but not limited to, siRNA, miRNA (micro RNA), lncRNA (long non-coding RNA), saRNA (small activating RNA), shRNA, and mRNA, including modified mRNA, such as modified mRNA to increase stability for delivery.

An advantage of MEVs for delivery, is that the RNA is a labile molecule, and so, mRNAs delivered by other kinds of nanoparticles, like lipid nanoparticles (LNPs) have been modified to increase RNA stability. For delivery in MEVs, the mRNA does not necessarily have to be modified. For endo-loading, the mRNA, in general, the mRNA will be unmodified. In all instances, the cargo for endo-loaded MEVs includes peptides, small peptides, polypeptides and proteins, nucleic acid encoding the proteins, including various forms of RNA, such as mRNA. The nucleic acids can be operably linked to regulatory elements that are recognized in the particular subject, such as a mammal, in which they are to be delivered.

| Target | | |
|---|---|---|
| organ/ | | Exemplary route of |
| tissue | Exemplary indication | administration |
| Lung | Cystic Fibrosis | Inhalation |
| Lung | Cystic Fibrosis | Inhalation |
| Lung | Idiopathic Pulmonary Fibrosis | Inhalation |
| Lung | Primary Ciliary Dyskinesia | Inhalation |
| Lung | Pulmonary Arterial | Inhalation |
| | Hypertension | |
| Liver | Inborn error of metabolism | Intravenous or direct |
| | | injection into the liver |
| Lung | Covid-19 (preventive or | Intranasal or Inhalation |
| | therapeutic) | |
| Lymphatic | Covid-19 (vaccine) | Intravenous or intramuscular |
| Lung | Influenza (preventive or | Intranasal or Inhalation |
| | therapeutic) | |
| Lymphatic | Influenza (vaccine) | Intravenous or intramuscular |
| Lymphatic | Viral pathogen | Intravenous or intramuscular |
| Lymphatic | Bacterial pathogen | Intravenous or intramuscular |

### 2. Genetic Engineering of Producer Cells

The endogenously loaded MEVs from Chlorella can be used for delivery to humans by any suitable route, including but not limited to intravenous, oral, topical, mucosal, intratracheal, inhalation, intranasal, and any other routes known to those of skill in the art for delivery of vehicles, such as lipid nanoparticles, vectors, therapeutic bacteria, and therapeutic viruses. Upon administration, the MEVs are taken up by cells. Any heterologous cargo suitable to be obtained in the producer cells can be loaded into the MEVs provided herein. The loaded heterologous cargo can be selected so that it only is expressed or produced in targeted cells. Transcription regulatory signals can be selected so that the encoded product is expressed in targeted cells. For example, for expression in the liver, the encoded product can be expressed under control of a liver-specific promoter, or the product can be targeted to a receptor or target expressed in targeted cells, such as in tumors or in the tumor microenvironment. MEVs, once produced, are isolated by methods used for isolating MEVs for subsequent exogenous loading.

### 3. Cargo

Cargo includes any of the antigens and immunomodulators as described herein for vaccines and polypeptides and nucleic acids as described above for exogenously-loaded cargo. As described above, the MEVs are loaded with cargo that can be used for any purpose of interest, including any for which other delivery vehicles are used. These uses include delivery of mRNA, such as mRNA encoding an antigen, therapeutic antibodies, and other such cargo.

Microalgae MEVs, for example, can be endogenously loaded with mRNA, For delivery of mRNA, however, the microalgae can be transformed, such as with a plasmid encoding the mRNA, where the encoded mRNA contains regulatory signals, or generally lacks one or more regulatory signals or sites for binding ribosomal proteins, so that the mRNA is produced, but is not translated by the microalgae cells or other eukaryotic cells. The nucleic acid, such as plasmid encoding the mRNA can be designed so that the mRNA is produced in abundance. This can be accomplished by operatively linking the mRNA sequences of interest to a strong eukaryotic promoter, including plant promoters, algae and microalgae promoters, or virus promoters, and optionally other regulatory sequences, such enhancers, in a plasmid that is introduced into the microalgae cells, such as by methods exemplified herein or any other methods known to those of skill in the art. The mRNA then is expressed at high levels, but is not translated, and becomes packaged in the MEVs produced by the microalgae cells. Exemplary promoters include plant promoters. Sequences of plant promoters are well known (see, e.g., Shahmuradov et al. (2003) PlantProm: a database of plant promoter sequences Nucleic Acids Res. 31: 114-117, softberry.com/plantprom2016/).

Regulatory signals and binding sites for controlling translation are well known. The following is an overview and description of signals and sites in mRNA that can be modified or deleted so that the mRNA is not translated (for a review, see, Fatima Gebauer et al. (2004) Nature Reviews Molecular Cell Biology 5:827-835). Structural features and regulatory sequences within the mRNA include: the canonical end modifications of mRNA molecules—the cap structure and the poly(a) tail—which are required for translation initiation; internal ribosome-entry sequences (IRESs), which mediate cap-independent translation initiation; upstream open reading frames (uORFs and sORFs), which normally reduce translation from the main ORF; secondary or tertiary RNA structures, such as hairpins and pseudoknots, which generally block initiation, but can also be part of IRES elements and therefore promote cap-independent translation; and, specific binding sites for regulatory complexes. Most of the regulatory mechanisms that are inhibitory; absent any change, mRNAs are translated. For endogenously packaging mRNA in MEVs, the mRNA that is encoded by DNA introduced into the microalgae cells can be modified, such as by deletion of the IRES, or modifying or interfering with ribosome binding proteins, or other such methods known to those of skill in the art.

In some embodiments, the microalgae cells are transformed with a plasmid that is then integrated into the genome, and mRNA is transcribed (produced). Alternatively, a plasmid that remains episomal can be introduced. The mRNA can be translated by the microalgae ribosomes. In other embodiments, the mRNA can contain modifications so that it is optimized or designed for translation in an animal, such as a human, subject. For example, the mRNA can contain optimized codons for expression in a subject, such as human, for translation so that it is not efficiently or not translated by the microalgae ribosomes, but is translated by higher order species, such as animal, such as a human. The mRNA can be “optimized for codons” (“codon optimization”) that translate well in the cell type where the mRNA is intended to be translated. The encoding plasmid sequence can be “optimized for codons” such that the mRNA will not be translated, or translated inefficiently by microalgae ribosomes, but is translated by mammalian ribosomes, or will in such a way that the mRNA transcribed out of that plasmid will not translate (or will do it very inefficiently) in the microalgae cell but will efficiently translate in the cells of those to whom the MEVs are administered. For example, the IRES and/or Kozak sequences encoded in the mRNA can be optimized or designed for expression or efficient or high expression in a mammal, not microalgae. Other regulatory elements can be optimized or designed for translation in cells of the target host, such as mammalian host, or a particular cell type.

mRNA generally includes the m7GpppN cap structure at the 5′ end of the mRNA, and the poly(A) tail at the 3′ end, which are motifs that promote translation initiation. Secondary structures, such as hairpins, block translation. Internal ribosome entry sequences (IRESs) mediate cap-independent translation. Upstream open reading frames (uORFs) normally function as negative regulators by reducing translation from the main ORF. Also included are binding sites for proteins and/or RNA regulators, which usually inhibit, but also promote, translation. These sequences can be optimized for translation in the intended host, such as mammalian cell, and/or selected so that they are not or not efficiently translated by the microalgae ribosomes, but are translated by mammalian, such as human, ribosomes. For example, the mRNA can include a Kozak sequence that is optimized for mammalian translation.

### 1) Protein Cargo

Protein cargo includes therapeutic proteins. These can be encoded by DNA introduced into the microalgae cell by any method known to the skilled person, such as those discussed above. The DNA can include regulatory sequences, such as strong promoters, to ensure production of a relatively large amount of the protein, that is then packaged in the MEVs. The protein cargo is encoded by DNA constructs that include regulatory sequences, as well as codon optimization, for an efficient transcription and, subsequently, translation in the microalgae. The nucleic acid will include appropriate sequences for translation into proteins. In general, the constructs will include strong promoters, such as strong plant promoters, and eukaryotic viral promoters, as well as enhancers to ensure that high levels of proteins are produced in the microalgae cells and packaged in the MEVs.

### F. PHARMACEUTICAL COMPOSITIONS, FORMULATIONS, KITS, ARTICLES OF MANUFACTURE AND COMBINATIONS

### 1. Pharmaceutical Compositions and Formulations

The compositions containing the MEVs and loaded MEVs provided herein can be formulated as pharmaceutical compositions provided for administration by a desired route, such as oral and intramuscular (IM) delivery of the cargo to the immune system. Pharmaceutically acceptable compositions are prepared in view of approvals for a regulatory agency or other agency prepared in accordance with generally recognized pharmacopeia for use in animals and in humans, and also, for agricultural applications, for plants. Typically, compounds are formulated into pharmaceutical compositions using techniques and procedures well-known in the art (see e.g., Ansel Introduction to Pharmaceutical Dosage Forms, Fourth Edition, 1985, 126).

The pharmaceutical compositions provided herein are for use for therapeutic and prophylactic applications. The MEVs and cargo-loaded MEVs provided herein can be formulated with a pharmaceutically acceptable carrier or diluent. Generally, such pharmaceutical compositions include components that do not significantly impair the biological properties or other properties of the cargo. Each component is pharmaceutically and physiologically acceptable so that it is compatible with the other ingredients and not injurious to the subject to whom it is to be administered. The formulations can be provided in unit dosage form and can be prepared by methods well-known in the art of pharmacy, including but not limited to, tablets, pills, powders, liquid solutions or suspensions (e.g., including injectable, ingestible and topical formulations, for example, eye drops, gels, pastes, creams, or ointments), aerosols (e.g., nasal sprays and inhalers), liposomes, suppositories, pessaries, injectable and infusible solutions and sustained release forms. See, e.g., Gilman, et al. (eds. 1990) Goodman and Gilman's: The Pharmacological Bases of Therapeutics, 8^{th} Ed., Pergamon Press; and Remington's Pharmaceutical Sciences, 17^{th} ed. (1990), Mack Publishing Co., Easton, Pa.; Avis, et al. (eds. 1993) Pharmaceutical Dosage Forms: Parenteral Medications Dekker, NY; Lieberman, et al. (eds. 1990) Pharmaceutical Dosage Forms: Tablets Dekker, NY; and Lieberman, et al. (eds. 1990) Pharmaceutical Dosage Forms: Disperse Systems Dekker, NY. When administered systemically, the therapeutic composition is sterile, pyrogen-free, generally free of particulate matter, and in a parenterally acceptable solution having due regard for pH, isotonicity, and stability. These conditions are known to those skilled in the art. Methods for preparing parenterally administrable compositions are well-known or will be apparent to those skilled in the art and are described in more detail in, e.g., “Remington: The Science and Practice of Pharmacy (Formerly Remington's Pharmaceutical Sciences)”, 19^{th} ed., Mack Publishing Company, Easton, Pa. (1995).

Pharmaceutical compositions provided herein can be in various forms, e.g., in solid, semi-solid, liquid, powder, aqueous, and lyophilized form. Examples of suitable pharmaceutical carriers are known in the art and include but are not limited to water, buffering agents, saline solutions, phosphate buffered saline solutions, various types of wetting agents, sterile solutions, alcohols, gum arabic, vegetable oils, benzyl alcohols, gelatin, glycerin, carbohydrates such as lactose, sucrose, amylose or starch, magnesium stearate, talc, silicic acid, viscous paraffin, perfume oil, fatty acid monoglycerides and diglycerides, pentaerythritol fatty acid esters, hydroxy methylcellulose, and powders, among others. Pharmaceutical compositions provided herein can contain other additives including, for example, antioxidants, preservatives, antimicrobial agents, analgesic agents, binders, disintegrants, coloring, diluents, excipients, extenders, glidants, solubilizers, stabilizers, tonicity agents, vehicles, viscosity agents, flavoring agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science and Practice of Pharmacy, 20^{th} Edition. Baltimore, MD: Lippincott Williams & Wilkins). Such carriers and/or additives can be formulated by conventional methods and can be administered to the subject at a suitable dose. Stabilizing agents such as lipids, nuclease inhibitors, polymers, and chelating agents can preserve the compositions from degradation within the body.

The route of administration is in accord with known methods, e.g., injection or infusion by intravenous, intraperitoneal, intracerebral, intramuscular, subcutaneous, intraocular, intraarterial, intrathecal, inhalation or intralesional routes, topical, rectal, mucosal, and by sustained release systems. The MEVs or cargo-loaded MEVs can be administered continuously by infusion or by bolus injection. One can administer the MEVs or cargo-loaded MEVs in a local or systemic manner.

The MEVs or cargo-loaded MEVs can be prepared in a mixture with a pharmaceutically acceptable carrier. Techniques for formulation and administration of the compounds are known to one of skill in the art (see e.g., “Remington's Pharmaceutical Sciences,” Mack Publishing Co., Easton, Pa.). This therapeutic composition can be administered intravenously or through the nose or lung, such as a liquid or powder aerosol (lyophilized). The composition also can be administered parenterally or subcutaneously as desired. When administered systematically, the therapeutic composition should be sterile, pyrogen-free and in a parenterally acceptable solution having due regard for pH, isotonicity, and stability. These conditions are known to those skilled in the art.

Pharmaceutical compositions suitable for use include compositions wherein the MEVs or cargo-loaded MEVs are contained in an amount effective to achieve their intended purpose. Determination of a therapeutically effective amount is well within the capability of those skilled in the art. Therapeutically effective dosages can be determined by using in vitro and in vivo methods, and/or by a skilled person.

Therapeutic formulations can be administered in many conventional dosage formulations. Dosage formulations of MEVs and cargo-loaded MEVs provided herein are prepared for storage or administration by mixing the compound having the desired degree of purity with physiologically acceptable carriers, excipients, or stabilizers. Such materials are non-toxic to the recipients at the dosages and concentrations employed, and can include buffers such as Tris HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium, and/or nonionic surfactants such as polysorbates (TWEEN), pluronics, polyethylene glycol, and others.

In particular examples herein, provided are pharmaceutical compositions that contain a stabilizing agent. The stabilizing agent can be an amino acid, amino acid derivative, amine, sugar, polyol, salt or surfactant. In some examples, the stable co-formulations contain a single stabilizing agent. In other examples, the stable co-formulations contain 2, 3, 4, 5 or 6 different stabilizing agents. For example, the stabilizing agent can be a sugar or polyol, such as a glycerol, sorbitol, mannitol, inositol, sucrose or trehalose. In particular examples, the stabilizing agent is sucrose. In other examples, the stabilizing agent is trehalose. The concentration of the sugar or polyol is from or from about 100 mM to 500 mM, 100 mM to 400 mM, 100 mM to 300 mM, 100 mM to 200 mM, 200 mM to 500 mM, 200 mM to 400 mM, 200 mM to 300 mM, 250 mM to 500 mM, 250 mM to 400 mM, 250 mM to 300 mM, 300 mM to 500 mM, 300 mM to 400 mM, or 400 mM to 500 mM, each inclusive.

In examples, the stabilizing agent can be a surfactant that is a polypropylene glycol, polyethylene glycol, glycerin, sorbitol, poloxamer and polysorbate. For example, the surfactant can be a polypropylene glycol, polyethylene glycol, glycerin, sorbitol, poloxamer and polysorbate, such as a poloxamer 188, polysorbate 20 and polysorbate 80. In particular examples, the stabilizing agent is polysorbate 80. The concentration of surfactant, as a % of mass concentration (w/v) in the formulation, is between or about between 0.005% to 1.0%, 0.01% to 0.5%, 0.01% to 0.1%, 0.01% to 0.05%, or 0.01% to 0.02%, each inclusive.

When used for in vivo administration, the formulation should be sterile and can be formulated according to conventional pharmaceutical practice. This is readily accomplished by filtration through sterile filtration membranes, prior to or following lyophilization and reconstitution. The MEVs or cargo-loaded MEVs can be stored in lyophilized form or in solution; they can be frozen or refrigerated. Other vehicles such as naturally occurring vegetable oil like sesame, peanut, or cottonseed oil or a synthetic fatty vehicle like ethyl oleate can be included. Buffers, preservatives, and antioxidants can be incorporated according to accepted pharmaceutical practice.

The MEVs or cargo-loaded MEVs provided herein, can be provided at a concentration in the composition of from or from about 0.1 to 10 mg/mL or higher or lower amounts, depending upon the application and the subject, such as, for example a concentration that is at least or at least about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10 mg/mL or more. The volume of the solution can be at or about 1 to 100 mL, such as, for example, at least or about at least or 0.5, 1, 2, 3, 4, 5, 10, 15, 20, 25, 30, 35, 40, 45, 50, 55, 60, 65, 70, 75, 80, 85, 90, 95, 100 mL or more. In some examples, the MEVs or cargo-loaded MEVs are supplied in phosphate buffered saline.

The MEVs or cargo-loaded MEVs provided herein can be provided as a controlled release or sustained release composition. Polymeric materials are known in the art for the formulation of pills and capsules which can achieve controlled or sustained release of the MEVs and cargo-loaded MEVs provided herein (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Langer and Peppas (1983) J Macromol. Sci. 23:61; see also Levy et al. (1985) Science 228:190; During et al. (1989) Ann. Neurol. 25:351; Howard et al. (1989) J. Neurosurg. 71:105; U.S. Pat. Nos. 5,679,377, 5,916,597, 5,912,015, 5,989,463, 5,128,326; and PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. Generally, the polymer used in a sustained release formulation is inert, free of leachable impurities, stable on storage, sterile, and biodegradable. Any technique known in the art for the production of sustained release formulation can be used to produce a sustained release formulation containing the MEVs or cargo-loaded MEVs provided herein.

In some examples, the pharmaceutical composition contains the MEVs or cargo-loaded MEVs provided herein and one or more additional agents, such as an antibody or other therapeutic, for combination therapy.

### 2. Articles of Manufacture/Kits and Combinations

Pharmaceutical compositions of the MEVs or cargo-loaded MEVs can be packaged as articles of manufacture containing packaging material, a pharmaceutical composition which is effective for treating a disease or condition that can be treated by administration of the particular MEVs or cargo-loaded MEVs, such as the diseases and conditions described herein or known in the art, and a label that indicates that the cargo, such as an antibody or nucleic acid molecule, is to be used for treating the infection, disease or disorder. The pharmaceutical compositions can be packaged in unit dosage forms containing an amount of the pharmaceutical composition for a single dose or multiple doses. The packaged compositions can contain a lyophilized powder of the pharmaceutical compositions containing the cargo-loaded MEVs which can be reconstituted (e.g., with water or saline) prior to administration.

The articles of manufacture provided herein contain packaging materials. Packaging materials for use in packaging pharmaceutical products are well-known to those of skill in the art (see, e.g., U.S. Pat. Nos. 5,323,907, 5,052,558 and 5,033,252). Examples of pharmaceutical packaging materials include, but are not limited to, blister packs, bottles, tubes, inhalers (e.g., pressurized metered dose inhalers (MDI), dry powder inhalers (DPI), nebulizers (e.g., jet or ultrasonic nebulizers) and other single breath liquid systems), pumps, bags, vials, containers, syringes, bottles, and any packaging material suitable for a selected formulation and intended mode of administration and treatment.

The MEVs or cargo-loaded MEVs can be provided as combinations and as kits. Kits optionally can include one or more components such as instructions for use, devices and additional reagents (e.g., sterilized water or saline solutions for dilution of the compositions and/or reconstitution of lyophilized protein), and components, such as tubes, containers and syringes for practice of the methods. Exemplary kits can include the MEVs or cargo-loaded MEVs provided herein, and can optionally include instructions for use, a device for administering the MEVs or cargo-loaded MEVs to a subject, a device for detecting MEVs or cargo-loaded MEVs in samples obtained from a subject, and a device for administering an additional therapeutic agent to a subject.

The kit can, optionally, include instructions. Instructions typically include a tangible expression describing the MEVs or cargo-loaded MEVs, and, optionally, other components included in the kit, and methods for administration, including methods for determining the proper state of the subject, the proper dosage amount, dosing regimens, and the proper administration method for administering the MEVs or cargo-loaded MEVs. Instructions also can include guidance for monitoring the subject over the duration of the treatment time.

Kits also can include a pharmaceutical composition described herein and an item for diagnosis. For example, such kits can include an item for measuring the concentration, amount or activity of the MEVs and cargo-loaded MEVs, in a subject.

In some examples, the MEVs or cargo-loaded MEVs are provided in a diagnostic kit for the detection of the MEVs or cargo-loaded MEVs or cargo in an isolated biological sample (e.g., tumor cells, such as circulating tumor cells obtained from a subject or tumor cells excised from a subject).

Kits provided herein also can include a device for administering the MEVs to a subject. Any of a variety of devices known in the art for administering medications to a subject can be included in the kits provided herein. Exemplary devices include, but are not limited to, a hypodermic needle, an intravenous needle, a catheter, a nebulizer, and an inhaler. Typically, the device for administering the compositions is compatible with the desired method of administration of the composition.

### 3. Administration of Cargo=Loaded MEVs and Routes of Administration

The cargo-loaded MEVs provided herein can be administered to a subject by any method known in the art for the administration of polypeptides, including for example systemic or local administration. For vaccination purposes, the MEVs can be administered orally or intramuscularly, or any other route whereby the MEVs traffic to the immune system.

In general, the cargo-loaded MEVs can be administered by routes, such as parenteral (e.g., routes, such as intradermal, intramuscular, intraperitoneal, intravenous, subcutaneous, and intracavity), topical, epidural, or mucosal (e.g., routes, such as topical, intranasal, oral, vaginally, vulvovaginal, esophageal, or esophageal, bronchial, rectal, and pulmonary). The cargo-loaded MEVs can be administered externally to a subject, at the site of the disease for exertion of local or transdermal action. Compositions containing the cargo-loaded MEVs can be administered, for example by infusion, inhalation, by bolus injection, or by absorption through epithelial or mucocutaneous linings (e.g., topical, oral, vaginal, rectal and intestinal mucosa). Compositions containing the cargo-loaded MEVs can be administered together with or sequentially with other biologically active agents. For example, the cargo-loaded MEVs are administered by infusion delivery, such as by infusion pump or syringe pump, and can be administered in combination with another therapeutic agent or as a monotherapy.

The method and/or route of administration can be altered to alleviate adverse side effects associated with administration provided herein. For example, if a patient experiences a mild or moderate (i.e., Grade 1 or 2) infusion reaction, the infusion rate can be reduced (e.g., reduced by 10%, 20%, 30%, 40%, 50%, 60%, 70%, 80%, 90% or more). If the patient experiences severe (i.e., Grade 3 or 4) infusion reactions, the infusion can be temporarily or permanently discontinued.

In some examples, if the subject experiences an adverse side effect, such as severe skin toxicity, for example severe acneiform rash, treatment adjustments can be made. For example, after the occurrence of an adverse side effect, administration can be delayed, such as for 1 to 2 weeks or until the adverse side effect improves. In some examples, after additional occurrences of an adverse side effect, the dosage can be reduced. A particular regimen and treatment protocol can be established by the skilled physician or other practitioner.

Appropriate methods for delivery, can be selected by one of skill in the art based on the properties of the dosage amount of the cargo-loaded MEVs or the pharmaceutical composition containing the cargo-loaded MEVs. Such properties include, but are not limited to, solubility, hygroscopicity, crystallization properties, melting point, density, viscosity, flow, stability and degradation profile.

### 4. Combination Therapies

The cargo-loaded MEVs provided herein can be administered before, after, or concomitantly with one or more other therapeutic regimens or agents. The skilled medical practitioner can determine empirically, or by considering the pharmacokinetics and modes of action of the agents, the appropriate dose or doses of each therapeutic regimen or agent, as well as the appropriate timings and methods of administration. The additional therapeutic regimens or agents can improve the efficacy or safety or other properties of the cargo-loaded MEVs. In some examples, the additional therapeutic regimens or agents can treat the same disease or a comorbidity. In some examples, the additional therapeutic regimens or agents can ameliorate, reduce or eliminate one or more side effects known in the art or described herein that are associated with administration of the cargo-loaded MEVs or the cargo.

For example, the cargo-loaded MEVs described herein can be administered with other immunomodulatory agents or treatments. The cargo-loaded MEVs can be administered with other anti-pathogen therapeutics and treatments. The cargo-loaded MEVs can be administered in combination with one or more other prophylactic or therapeutic agents, including but not limited to antibodies, cytotoxic agents, chemotherapeutic agents, cytokines, growth inhibitory agents, anti-hormonal agents, kinase inhibitors, anti-angiogenic agents, cardio-protectants, immunostimulatory agents, immunosuppressive agents, agents that promote proliferation of hematological cells, angiogenesis inhibitors, protein tyrosine kinase (PTK) inhibitors, FcTRIIb or other Fc receptor inhibitors, or other therapeutic agents.

The one or more additional agents can be administered simultaneously, sequentially or intermittently with the cargo-loaded MEVs. The agents can be co-administered, for example, as part of the same pharmaceutical composition or same method of delivery. In some examples, the agents can be co-administered at the same time as the cargo-loaded MEVs, but by a different means of delivery. The agents also can be administered at a different time than administration of the cargo-loaded MEVs, but close enough in time to have a combined prophylactic or therapeutic effect. In some examples, the one or more additional agents are administered subsequent to or prior to the administration of the cargo-loaded MEVs separated by a selected time period. In some examples, the time period is 1 day, 2 days, 3 days, 4 days, 5 days, 6 days, 1 week, 2 weeks, 3 weeks, 1 month, 2 months, or 3 months. In some examples, the one or more additional agents are administered multiple times and/or the cargo-loaded MEVs provided herein are administered multiple times.

### G. BIODISTRIBUTION OF MEVs FOLLOWING ADMINISTRATION VIA VARIOUS ROUTES

### 1. Biodistribution of Mammalian EVs

Pharmacokinetics and biodistribution in organs and tissues of mammalian EVs have been extensively studied for their pharmacokinetics and distribution in organs and tissues (Vader et al. (2016) Advanced drug delivery reviews 106(PtA):148-156, doi.org/10.1016/j.addr.2016.02.006; Morishita et al. (2017) Journal of pharmaceutical sciences 106(9):2265-2269, hdoi.org/10.1016/j.xphs.2017.02.030). Treatments with mammalian cell-derived EVs are generally based on intravenous or intraperitoneal routes of administration. Primary target organs upon systemic administration of mammalian EVs are the liver, spleen and lungs. A comprehensive study (see, Wiklander et al. (2015) J. Extracellular Vesicles 4:26316) of the tissue distribution of fluorescently-labelled mammalian Evs from various cell sources demonstrated that 24 hours after intravenous (i.v.) injection in mice, the highest fluorescence signal was in the liver, followed by spleen, gastrointestinal tract and lungs. Furthermore, cell source, EV dose, and route of administration was shown to affect EV distribution; for example, injection of higher EV doses resulted in relatively lower liver accumulation compared to lower doses, possibly caused by saturation of the mononuclear phagocyte system (MPS). Comparison between intraperitoneal (i.p.), subcutaneous (s.c.) and i.v. administrations showed that intraperitoneal and subcutaneous doses resulted in reduced EV accumulation in liver and spleen and enhanced pancreas and gastrointestinal tract accumulation compared to i.v. injections. Systemically administered Evs are reported to be rapidly taken up by the mononuclear phagocyte system (MPS), particularly in the liver and spleen. The mechanism of clearance resembles that described for synthetic nanoparticles, such as liposomes (Van der Meel et al. (2014) J. Control. Release 195:72-85). The majority of splenic accumulation is caused by EV storage in the spleen rather than uptake by the spleen (Lai C.P. et al. (2014) ACS Nano 8:483-494). Biodistribution of mammalian EVs following other routes of administration also has been investigated. For targeting of the central nervous system, intranasal administration of curcumin-loaded mammalian EVs resulted in EV localization in the brain. Drug levels peaked at 1 hour after administration, and a significant amount detected after 12 hours with no toxic effects observed (Zhuang et al. (2011) Mol. Ther. 19:1769-1779).

In general, mammalian EVs are not employed for oral delivery because of their low stability at various pH and temperatures, rapid degradation of biomolecules in the digestive tract, and the limitations of industrial scale production for oral dosing (Cheng et al. (2019) Protein Cell 10:295-299). The only exception so far are bovine milk-derived EVs, which upon oral delivery to mice have shown a pattern of distribution that, analyzed with whole-body in vivo imaging system (IVIS), included rapid accumulation in the intestine, where the EVs were detectable after 2 and 6 hours, followed by fluorescence signal observed in liver, spleen, lungs, kidney, heart, and the gastrointestinal tract at 24 hours. After 48 hours, the fluorescence signal subsided within most of the organs indicating the clearance of nanovesicles from the system (Samuel et al. (2021) Nat Commun 12:3950, doi.org/10.1038/s41467-021-24273-8). Thus, mammalian EVs (derived from sources other than milk) cannot be absorbed by the intestinal tract and from the intestines to become bioavailable in target organs (Zhong et al. (2021) Biomaterials. 277:121126. Doi: 10.1016/j.biomaterials.2021.121126).

Treatments with mammalian cell-derived EVs generally employ intravenous or intraperitoneal routes of administration for systemic administration where the target organs are the liver, spleen and lungs. As noted, most mammalian EVs have not been employed for oral delivery due to their low stability at various pH and temperatures, rapid degradation of biomolecules in the digestive tract, and the limitations of industrial scale production for oral dosing (Cheng et al. (2019) Protein Cell 10(4):295-299). The only exception are bovine milk-derived EVs, which upon oral delivery to mice have shown a pattern of distribution that, analyzed with whole-body in vivo imaging system (IVIS), include rapid accumulation in the intestine, where the EVs were detectable after 2 and 6 hours, followed by fluorescence signal observed in liver, spleen, lungs, kidney, heart, and the gastrointestinal tract at 24-hour time point. After 48 hours, the fluorescence signal subsided within most of the organs indicating the clearance of nanovesicles from the system.

As described herein, and in copending commonly owned applications (see, PCT/EP2023/051650), MEVs have different properties from mammalian EVs. For example, they are stable in the harsh environment of the gastrointestinal tract compared to mammalian cell-derived EVs. Thus, the microalgae EVs, as described herein, are particularly suitable for oral administration and drug delivery, as well as other routes of delivery as described herein.

### 2. Microalgae EVs Biodistribution

MEVs, including those provided herein from Chlorella, have properties that are distinct from mammalian EVs, including bovine milk EVs (see, commonly owned PCT/EP2023/051650). For example, a striking difference, as described below, is that the MEVs can be administered orally, and that the primary target is the spleen, particularly the white pulp of the spleen (white spleen). This renders the MEVs of use as vaccines and for delivery of immunomodulatory cargo.

The MEVs provided herein can deliver a variety of bioactive molecules, such as RNAs, such as mRNA, siRNA, and miRNA; proteins; peptides; and small molecules, which can be exogenously or endogenously loaded. These include products such as tissue-specific products and/or disease specific products. Each route can be used to target particular organs and treat particular diseases. The MEVs can be formulated for administration by each route. Thus, provided are compositions containing MEVs that are for treating particular disease and for particular routes of administration. For immune modulation and vaccination, the MEVs are delivered by routes, such as oral and IM and mucosally, including by inhalation into the lungs, nose, and intestinal mucosa, that result in delivery of the MEV cargo to organs and tissue of the immune system.

The route of administration determines the fate of the MEVs, and that the ultimate location of the MEVs is a function of the route of administration. Targets and endpoints of the MEVs include, but are not limited to, the liver, spleen, lungs, the intestines, and brain. Routes of administration include, but are not limited to, respiratory (nose, lungs), oral (digestive), intravenous, central nervous system (CNS), and topical. The selection of route depends upon the ultimate target and the payload. It is shown herein that intranasal administration goes to the lungs, intratracheal via a spray goes to the lung(s), intravenous accumulates in the spleen and liver, oral (per Os) goes to the digestive tract and spleen. In contrast, mammalian EVs cannot be taken orally. As described herein, routes for delivery of cargo to the immune system include oral and IM.

MEVs are readily internalized by human cells. For example, in vitro, when administered to cells in culture, such as A549 cells, at a ratio of MEV/cell of 1000/1, 93% of the cells internalized the MEVs, and this occurred within 24 to 48 hours after contacting the cells with the MEVs.

DIR-labeled MEVs were administered to mice via four routes: intranasal (IN), intratracheal (IT), intravenous (IV), and oral, and, by full-body imaging as a function of time, the fate of the MEVs was visualized for 3 days, followed by sacrificing the mice to harvest organs for study. As shown in the examples, intravenous administration targets the liver at about 4-12 hours following administration, and the spleen, appearing to be in the red pulp of the spleen (red spleen), at 10-30 hours. Oral administration targets the intestine and spleen. It is shown herein that the MEVs are orally available; they resist passage through the stomach, and reach the intestine at 0.5 hour to 4 hours, and then the spleen at 0.5 hour to 10 hours. Of interest is the route to the spleen; there are two possible routes to the spleen, via the blood (to red spleen), and via lymphocytes (to white spleen), which has implications for targeting and delivering cargo to the immune system, accumulating from 4 hours to 28 hours. This can be effected by internalization by lymphocytes that are activated and end up in the spleen where they multiply, and/or by lymphocytes that phagocytose the MEVs, which are not activated, and go to the white pulp of the spleen (white spleen) from where they are disseminated through the immune system.

a. Oral Administration

Orally ingested MEVs go into the intestine, then end up in the spleen, likely the white spleen. The spleen is responsible for initiating immune reactions to blood-borne antigens, and for filtering foreign material and old or damaged red blood cells from the blood. These functions are performed by two different compartments in the spleen: the white spleen, and red spleen. The two compartments are vastly different in structure, vascular organization, and cellular composition (see, e.g., Cesta (2006) Toxicologic Pathology 34:455-465 for a review of the structure, function and histology of the spleen).

White blood cells, which are plentiful in the intestine, migrate to the white spleen. When ingested orally the MEVs can be internalized by intestinal cells and, as discussed below, including by intestinal lymphocytes, which carry the MEVs to the spleen. This is in contrast to mammalian vesicles, which cannot be administered orally. Thus, MEVs provide a delivery vehicle for agents for which the immune system is a target, such as for example, immune-modulating cargo. As discussed above, the pathway to the white spleen can occur, for example, via activated lymphocytes and/or phagocytic lymphocytes. Lymphocytes can phagocytose the MEVs, and are homed to the spleen. The MEVs, unlike mammalian EVs, provide a way to orally deliver small molecule drugs and proteins and other therapeutics, such as nucleic acid therapeutics, that cannot be administered orally. In particular, orally administered MEVs provide a route for treatment of diseases, such as cancers and inflammatory diseases, in which the immune system is involved or in which the treatment can be effected by targeting the immune system. Such diseases include, but are not limited to, infectious disease, autoimmune diseases, cancers, and prevention of organ transplant rejection. These diseases are treated by suppressing or augmenting the activity of immune cells.

### 1) Components of the Lymphatic System

The lymphatic system includes lymph, lymphatic vessels and lymphatic organs (see, discussion in Zgair et al., (2016) Targeting Immunomodulatory Agents to the Gut-Associated Lymphoid Tissue. In: Constantinescu C., Arsenescu R., Arsenescu V. (eds) Neuro-Immuno-Gastroenterology. Springer, Chain. (doi.org/10.1007/978-3-319-28609-9_14) and summarized below).

### Lymph

Lymph is a generally clear and colorless fluid that drains from the interstitium, and contains recovered fluids and plasma proteins, and also can contain lipids, immune cells, hormones, bacteria, viruses, cellular debris, and cancer cells.

### Lymphatic Vessels

The lymphatic system is the body's second circulatory system. The lymphatic system is a unidirectional, blind-ended and thin-walled system of capillary vessels where lymph is driven. Lymphatic capillaries drain in the afferent collecting vessels, which then pass through one or more gatherings of lymph nodes. Lymph fluid then passes through the efferent collecting vessels, larger trunks and then the lymphatic duct, which drain lymph to the systemic circulation. Primary lymphatic organs include the thymus gland and bone marrow, which produce mature lymphocytes, which identify and respond to antigens; secondary lymphatic organs include lymph nodes, spleen and mucosa-associated lymph tissues (MALTs). Within the secondary lymphatic organs, lymphocytes initiate immune responses. MALTs are distributed throughout mucous membranes and provide a defensive mechanism against a wide variety of inhaled or ingested antigens. MALTs are categorized according to their anatomical location as: bronchus-associated lymphoid tissue (BALT), nasal-associated lymphoid tissue (NALT), salivary gland duct-associated lymphoid tissue (DALT), conjunctiva-associated lymphoid tissue (CALT), lacrimal duct-associated lymphoid tissue (LDALT) and gut-associated lymphoid tissue (GALT).

### Gut-Associated Lymphoid Tissue (GALT)

GALT is composed of effector and immune induction sites. Effector sites include lymphocytes distributed throughout the lamina propria (LP) and intestinal epithelium; induction sites involve tissues, such as such as mesenteric lymph nodes (MLN), PP and smaller isolated lymphoid follicles (ILF). Mesenteric lymph nodes (MLN), which occur in the base of the mesentery, are the largest gatherings of lymph nodes in the body. The structure of MLN is divided into two regions: the medulla and cortex. The cortex primarily is composed of T-cell areas and B-cell follicles. Within the T-cell area, circulating lymphocytes enter the lymph node, and dendritic cells (DC) present antigens to T-cells. Lymph (containing cells, antigens and chylomicrons) is collected from the intestinal mucosa and reaches the MLN via the afferent lymphatics. Lymph fluid subsequently leaves the MLN through efferent lymphatics to reach the thoracic duct that drains to the blood.

Peyer's patches (PP) are a collection of lymphoid nodules distributed in the mucosa and submucosa of the intestine. They contain a sub-epithelial dome area and B-cell follicles dispersed in a T-cell area. A single layer of epithelial cells, called follicle-associated epithelium (FAE), separates lymphoid areas of PP from the intestinal lumen. FAE is permeated by specialized enterocytes called microfold (M) cells. These cells are a gate for the transport of luminal antigens to PP.

Isolated lymphoid follicles (ILF) are a combination of lymphoid cells in the intestinal LP. ILF are composed of germinal centers covered by FAE containing M-cells. ILF is a complementary system to PP for the induction of intestinal immunity.

GALT is the largest lymphatic organ in the human body and contains more than half of the body's lymphocytes. GALT is exposed to more antigens in the form of commensal bacteria and alimentary antigens, in addition to those from invasive pathogens, than any other part of the body. Intestinal lymphatic transport avoids hepatic first-pass metabolic loss by diverting the absorption of lipophilic drugs towards intestinal lymphatics rather than the portal vein. The intestinal immune system must distinguish antigens that require a protective immune response and develop a state of immune hypo-responsiveness (oral tolerance) for harmless antigens. This is effected by sampling of luminal antigens in the intestinal epithelium by DC. Antigens can cross the epithelium through M-cells, which are specialized epithelial cells of the follicle-associated epithelium of the GI tract. The antigens interact with DC in the underlying sub-epithelial dome region. Antigens are presented to local T-cells in PP by DC.

DC also migrate to the draining MLN where they present antigens to local lymphocytes. Alternative pathways for antigen transport across the intestinal epithelial cells involve receptor-mediated transport, and direct sampling from the lumen by DC projections. Antigen-loaded DC then migrate to the MLN through afferent lymphatics where they present antigens to T-cells. Subsequently, differentiated lymphocytes migrate from MLN through the thoracic duct and blood stream and eventually accumulate in the mucosa for an appropriate immune response.

### 2) Targeting GALT

Orally administered MEVs can target gut-associated lymphoid tissue (GALT).

Upon oral administration the MEVs pass through the epithelial layer of the lumen into the GALT, where they are internalized by macrophages and dendritic cells. Antigen presenting cells (APCs), carrying the MEVs enter the bloodstream, where they are protected from hepatic first-pass metabolic loss since they are effectively invisible inside the APCs. In the APCs, the MEVs traffic from the liver to the red and white pulp of the spleen. By reaching the spleen, the MEVs can deliver vaccines and immunomodulatory therapeutics. This can be effected by oral administration. (Also as shown herein, administration to cells of the immune system can be effected by inhalation into the lungs and via intramuscular administration.)

Thus, GALT is a target (effective compartment) and/or a route through which MEVs and their therapeutic agent cargo can be used to deliver cargo to organs, tissues, and/or systemic circulation; the MEVs can be used to deliver vaccines and immunomodulatory cargo via entry through the GALT, into APCs, and into the spleen. GALT is an advantageous target for various pharmacological agents such as, for example, immunomodulators, chemotherapeutic agents, and anti-infective agents. The lymphatic system is a main pathway for intestinal and other tumor metastases; therefore, targeting cytotoxic drugs to the intestinal lymphatics can be used to treat tumor metastases. GALT is a delivery target for antiviral agents, as some viruses, such as, for example, human immunodeficiency virus (HIV), morbillivirus, canine distemper virus, severe acute respiratory syndrome (SARS)-associated coronaviruses, hepatitis B and hepatitis C, spread and develop within the lymphatic system. The MEVs, including the Chlorella MEVs exemplified herein, can be used to target immune cells upon oral delivery. As described above, the microalgae MEVs show a distinct pattern of biodistribution when administered orally. This pattern includes initial intestine accumulation followed by targeting the spleen; whereas, as described and shown herein, they are detectable up to 24 hours.

Since the microalgae MEVs are delivered to the spleen, the mechanism of this delivery can be based on cells of the immune system. Immune cells are abundant in the single-cell layer of intestinal epithelium and underlying lamina propria of the gut-associated lymphoid tissue (GALT). The immune cells include, T cells, plasma cells, mast cells, dendritic cells, and macrophages (Luongo et al. (2009) Current perspectives. International Reviews of Immunology 28(6):446-464, doi.org/10.3109/08830180903236486). Macrophages, dendritic cells, neutrophils, and also B cells perform phagocytosis. The immune cells in the gut, thus, can phagocytose the MEVs to deliver them to the spleen. After phagocytosis, the fate of the MEV cargo can depend upon the type of cargo. For example, macrophage and dendritic cells participate in antigen presentation, and present proteins delivered in the MEVS, or the products in the MEVs can be secreted, or the products, such RNA, can be translated.

Immune cells present in the intestinal epithelium and lamina propria of the intestine migrate to the spleen and back to the intestine. This homing to the spleen can be involved in MEV transfer from the gut to secondary lymphatic organs, especially to the spleen. T cells exhibit a specific lymphocyte recirculation pathway (Mackay et al. (1990) J Exp Med 171:801-17) that can be part of MEV trafficking to the spleen upon oral delivery. Therefore, cells of the immune system are targeted by orally-administered MEVs, and this phenomenon contributes to MEV localization in the spleen within hours post-administration.

Upon oral administration, the MEVs go to the intestine and then migrate to the spleen. The route to the spleen can be via absorption into the blood and/or by internalization by immune cells in the intestine. The blood route is an unlikely route, because the MEVs then would appear in the liver as shown for intravenous administration. When MEVs are administered intravenously they primarily reach the liver (massively) and to a much lesser extent the spleen. It is shown herein that clearance of the MEVs from the spleen follows different kinetics depending upon their origin (oral or IV). The migration to the spleen following oral administration therefore uses a different a pathway from the MEVs administered intravenously.

When MEVs are administered by mouth, they reach the spleen after having passed through the intestine. These results indicate that the MEVs are located in “different compartments” inside the spleen, depending on the route of arrival: either from the intestine or from the blood. As discussed, upon oral administration, the likely route is that the MEVs in the intestine are internalized by lymphocytes present in the GALT, and that the subsequent migration of the MEVs from the intestine/GALT to the spleen occurs because the MEVs are transported by the lymphocytes. Coming from the intestine/GALT, the MEVs end up in the white spleen compartment. Thus, the MEVs provide a way to deliver cargo to different organs from mammalian EVs, which cannot be administered orally.

### 3. Diseases and Conditions Treated by MEVs

Based upon the targeted organs, a variety of diseases and disorders can be treated by MEVs. The MEVs can be loaded or produced to contain therapeutic agents for treating these diseases and conditions. The appropriate route of administration for the targeted organ and disease is selected. For example, for targeting the spleen and intestines, oral administration is selected; and for targeting the lungs, inhalation or nasal administration is selected. Based on the biodistribution and pharmacokinetic data the following organs can be targeted to treat diseases exemplified as follows.
liver: cancer, cancer metastases, metabolic syndrome, genetic disorders (delivery of gene therapy), alpha-anti-trypsin (AAT) deficiency and other inborn errors of metabolism, hemophilia, hypercholesterolemia, liver inflammation, steatohepatitis, and other diseases and disorders that can be treated by delivery of a therapeutic to the liver;
spleen: diseases treated by immune modulation, including cancers, and immune cell disorders, and cancer, and other diseases that can be treated by administration to the spleen, particularly by immune cells that occur in or traffic to the white spleen;
intestine: diseases and disorders treated or prevented by vaccines, intestinal infections, microbiota modulation, Crohn's disease, cancer, ulcers, diseases treated by orally administered drugs, such as small molecules and proteins, and other such diseases, disorders, and conditions; and
lungs: infectious diseases, particularly respiratory diseases, chronic obstructive pulmonary disease (COPD), pulmonary hypertension, asthma, other inflammatory lung diseases, cystic fibrosis, ATT-deficiency, lung disease, cancer, cancer metastases, and other such diseases and disorders.

### H. THE IMMUNE SYSTEM AND MEVs FOR USE AS VACCINES AND FOR DELIVERY OF IMMUNOMODULATORS

The MEVS can be used as vaccines where the cargo comprises an antigen, as a protein, or nucleic acid, for inducing an immune response. The vaccines can be administered by any route that delivers cargo to the immune system or cells involved in a disease, disorder, or condition that can be targeted for immunization or treatment. Routes of administration of vaccines include, but are not limited to, oral, intramuscular, and local administration, including mucosal administration, such as inhalation to the lungs and nasal tract.

### 1. Immune System and Vaccines

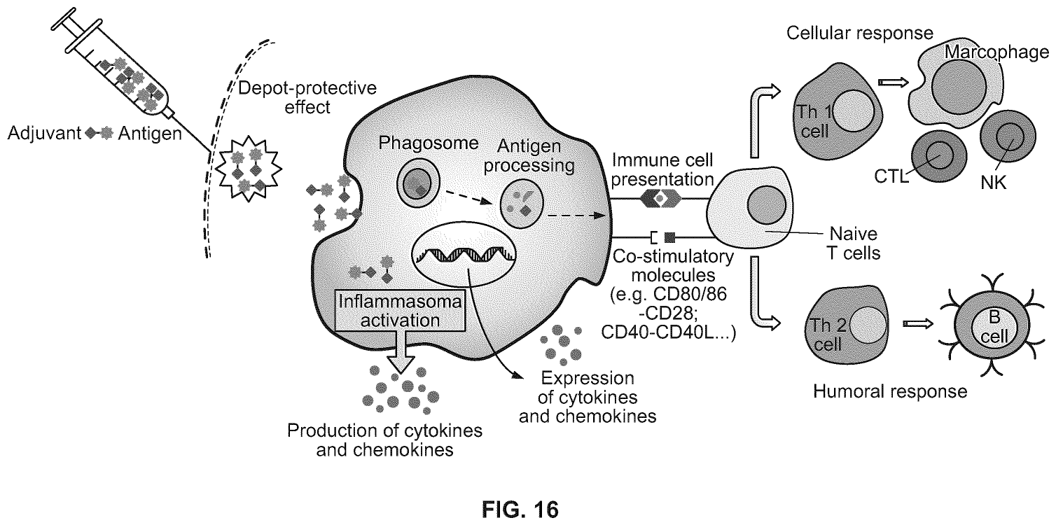

The immune systems of mammals have evolved to defend and eliminate pathogens and other foreign invaders or cells in the body. Those of skill in the art are familiar with the immune system and components thereof. The mammalian immune system has two major branches, innate and adaptive immunity (see, e.g., FIG. 16, which includes components of each branch). The innate immune system recognizes pathogen-associated molecular patterns through a limited number of germ-line encoded pathogen recognition receptors. The innate immune response is relatively nonspecific in recognizing pathogens and does not induce immune memory. The adaptive immune system employs a large repertoire of rearranged receptors, adaptive immunity plays a major role in eliminating pathogens in the later phase of infection as well as in generating immunological memory.

Acquired immunity develops by clonal selection from a vast repertoire of lymphocytes bearing antigen-specific receptors that are pre-generated via a mechanism generally known as gene rearrangement during an early developmental stage. This fundamental characteristic of the host immune system to generate immunological memory provides a rationale for vaccination as the most effective measure in preventing infectious diseases or reducing the risk of acquiring an infectious disease or the severity of such disease. Induction of long-term protective immunity is a goal of developing successful and safe vaccines.

### 2. Vaccines—Oral, Intramuscular, and Local Administration, Including Mucosal Administration, Such as Inhalation to the Lungs and Nasal Tract

The MEVs, which deliver cargo to particular organs, cells and tissues, can be used as vaccines to deliver proteins, nucleic acid, or other therapeutics for the treatment and previous immunotherapies or vaccines of diseases, disorders, and conditions. For use as vaccines routes of administration include oral administration, and intramuscular administration (IM) for trafficking via routes discussed above. Vaccines can be administered locally and can be administered mucosally. Routes also include inhalation into the lungs and/or nose.

a. MEV-Based Oral Vaccines

The oral route of vaccine administration provides the advantage of stimulating mucosal immunity (as discussed herein). While mucosal epithelium covers the largest surface area in the body, it also constitutes the first line of defense against external pathogens. Physicochemical and biological barriers are present on these mucosal surfaces, both to regulate nutrient uptake and provide defensive responses. Vaccine-mediated stimulation of mucosal immunity can improve protective efficacy and enhance disease prevention. Oral immunization can also improve vaccine efficacy by increasing accessibility and coverage. In fact, vaccine distribution represents one of the main limiting factors, particularly in developing countries. One of the key advantages of oral vaccines compared to traditional injections it their capacity to facilitate distribution with easy administration, including self-administration of oral formulations. Self-administration is an ideal method to achieve widespread and rapid distribution of vaccines as it minimizes the need for trained healthcare personnel and visits to healthcare facilities. This enhances the use of vaccines, reduces the cost of vaccination programs, and eliminates occupational hazard of needle injuries for health-care workers. Oral vaccines provide regulatory benefits and more cost-effective production as there are different purity requirements between oral and injected formulations. Traditional injectable vaccines generate considerable amount of biohazardous waste and the cost of its disposal.

Oral delivery is the most desirable and patient-accepted route of administration, with more than 60% of commercialized small molecule drug products using the oral route. Despite this, only a small fraction of vaccines approved so far are orally-available due to the inherent obstacles presented by the gastrointestinal system. The induction of a robust protective immune response by oral immunization requires: (i) successful delivery of the intact and active antigen to the intestine, (ii) transport across the mucosal barrier, and (iii) subsequent activation of antigen-presenting cells. Each of those steps can be hindered by multiple physicochemical and biological barriers in the gastrointestinal tract. Labile antigens undergo degradation in the harsh environment of the stomach and by the digestive enzymes. Intestinal epithelium and its mucus-secreting layers provide biological barriers that protect the organism from pathogen invasion. Furthermore, the time window for vaccine absorption is narrow, as the residence time in the small intestine, where the majority of absorption occurs, is about 3-4 hours. The oral delivery route requires administering adequate doses of the vaccine to generate immunity instead of tolerance. Since the gastrointestinal tract is constantly exposed to a variety of pathogens, the vaccine formulation must trigger appropriate danger signals to sufficiently stimulate the immune system and shift the immune balance of the gut from immune tolerance to protective immune response.

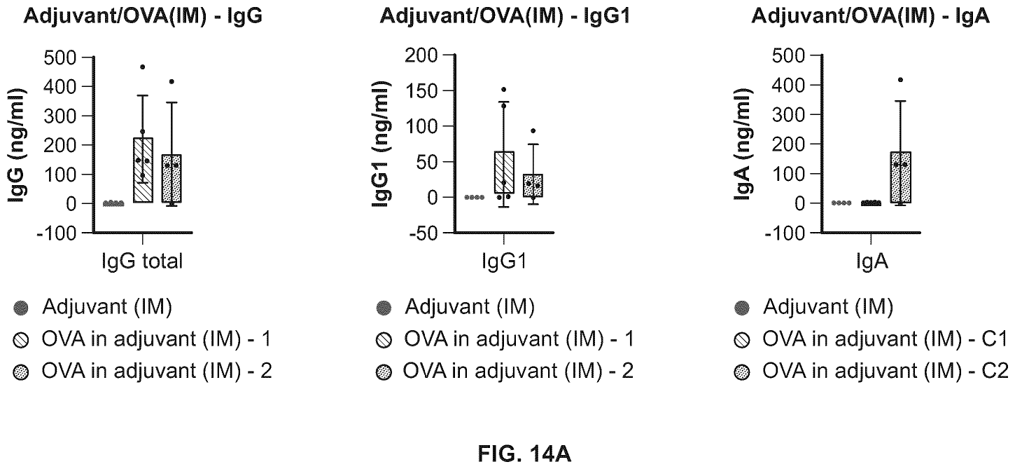

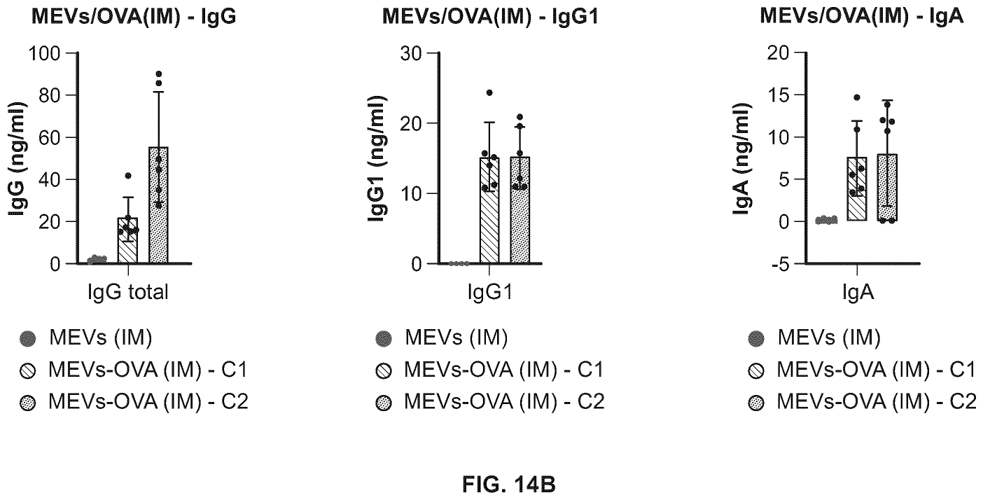

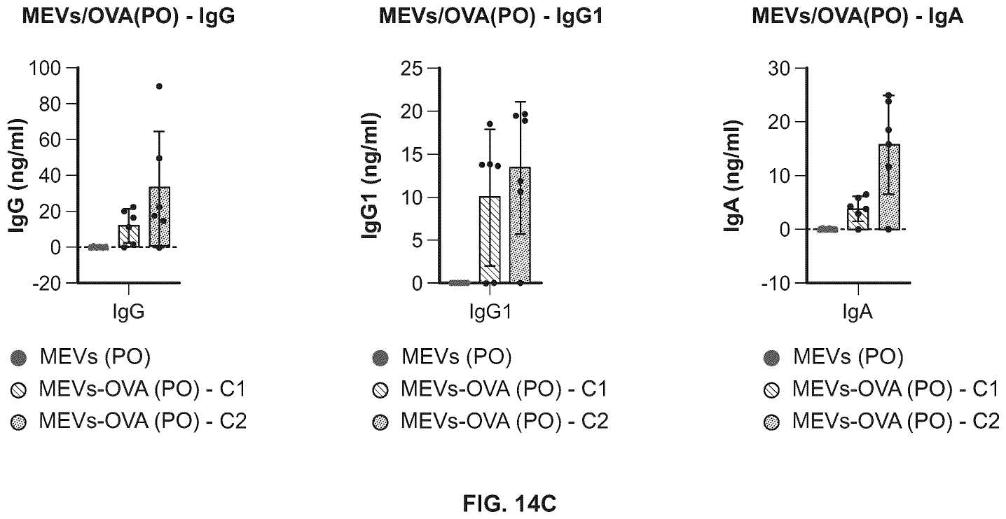

It is shown herein that MEVs provide a vehicle for oral delivery of vaccines and other cargo to the immune system via oral delivery. Results herein evidence that adjuvants are not necessarily required to result in a robust immune response when delivery (oral or via other routes) is effected via MEVs. Data herein also show that the MEVs are not immunogenic (see, e.g., FIGS. 14A-14C), and thus, can be used a plurality of times for immunization.

b. MEV-Mediated Immunization Upon Oral Delivery

As described and exemplified herein, the MEVs show a particular pattern (intestine-GALT-mesenteric lymphoid nodes-spleen) of biodistribution when administered orally, which includes initial intestine accumulation followed by targeting the spleen, where they are detectable up to 24 hours (see Example 5 below). The MEVs specifically are delivered to the spleen by immune cell-mediated transport of the MEVs. In the course of gastrointestinal tract migration, the MEVs cross the barrier of intestinal epithelium and become available to meet with immune cells of the gut-associated lymphoid tissue (GALT) and mesenteric lymphoid nodes—the largest mass of lymphoid tissue in the body. This tissue includes rich cellular populations of T cells, plasma cells, mast cells, dendritic cells (DC) and macrophages. The immune cells present in the GALT are also known to constantly circulate between the intestine and the spleen. The MEVs are internalized by the GALT cells and can be transferred from the gut to other secondary lymphoid organs (SLO), including mesenteric lymph nodes and the spleen.

During an immune response against a foreign antigen, a number of immune cells are competent to interact with the antigen. This includes naïve B cells and other cells within the lymphoid tissue microenvironment (such as CD4⁺ T cells, macrophages and dendritic cells). Antigen-specific naïve B cells then undergo one of two fates: some of them rapidly differentiate into short-lived plasma cells, which provide a first wave of defense against the invading pathogen, while others migrate to the B-cell follicles of SLO, where transient structures called germinal centers (GCs) are formed. This process is triggered by follicular helper T cells (T_{FH}) interacting with their cognate follicular B cells. Upon contact with the T_{FH} cells, the B cells obtain T cell help required for B cell activation and proliferation. Within the formed GC, B cells undergo somatic hypermutation, class switch recombination and selection of high-affinity variants. Ultimately, they differentiate either into long-lived plasma cells capable of producing high-affinity antibodies against foreign antigen, or germinal center-dependent memory B cells capable of quick immune re-activation in the future if ever the same antigen is re-encountered. Memory B cells can be produced not only during the T cell regulated immune response, but also from conventional marginal B2 lymphocytes, in the marginal zone of spleen white pulp.

Cell-mediated immunity, involving CD4⁺ and CD8⁺ T cells, plays a major role in defense against intracellular and extracellular bacteria, as well as immunity against tumors. Naïve T cells leave the thymus, enter the circulation, and then traffic preferentially through SLO, where they screen antigen-presenting cells in search of their cognate peptide-MHC complex. The antigens are presented by migratory dendritic cells, which migrate from the periphery into lymph nodes and the spleen. Once the specific encounter occurs, naïve T cells are primed by the dendritic cells and differentiate into activated effector/memory T cells. Although T cell activation occurs mainly in the lymph nodes, some antigen-specific T cells are primed in the spleen; those T cells are transcriptionally distinct and have enhanced ability to differentiate into long-lived memory cells compared with lymph node-primed counterparts.

The MEV vaccines as described and provided herein, elicit potent immune and serological memory. Long-term cell-mediated immunity depends on spleen-primed T cells, which differentiate into long-lived memory cells, thereby contributing to vaccine efficacy. On the other hand, generation of sustained serological memory is dependent on the GC reactions occurring in the secondary lymphoid organs. This results from MEV-mediated targeting of secondary lymphoid organs upon oral vaccine administration. This primarily includes the GALT (Peyer's patches), followed by transport (by macrophages and dendritic cells (DC)) of MEVs into lymph nodes and the spleen. MEV-assisted vaccine delivery results in enhanced T cell priming in the spleen, T_{FH} cell activation, GC formation, affinity-matured B cell generation and improved production of memory T cells, long-lived plasma cells and durable memory B cells. As described herein, and understood by those skilled in the art, the vaccine delivery system can be further engineered with protein and non-protein immunomodulatory agents either loaded into the MEV lumen or associated with the MEV membrane, and/or for combination therapy as separate compositions or co-formulations.

c. MEV-Mediated Immunization and Mucosal Immunity

Traditionally, intramuscularly or intradermally administered vaccines generate strong IgM and IgG predominant responses. In particular, the intramuscular route induces an immune response in the axillary draining lymph node that is biased towards class switch to IgG rather than to IgA. As shown herein, however, the response to IM administration includes an IgA response. In evaluation of the immune response to some infections, e.g., influenza, IgA together with IgG are more important in protection against secondary infection; whereas IgG and IgM predominate in the primary immune response. IgA is the predominant immunoglobulin expressed in the respiratory tract, cornea, and gastrointestinal tract mucosal surfaces, and IgA responses with neutralizing capability are described for several viral pathogens. Neutralizing antibody titers are the best correlates of protection in most vaccines, and memory responses are responsible for protection from re-infection and are essential for effective vaccination.

The results described in the Examples herein include analysis of the antigen-specific antibody response after intramuscular or oral administration of MEV-based vaccine formulations. As can be seen in FIGS. 14A-14C, the antigen-specific antibodies produced include immunoglobulin A (IgA)-class antibodies that are specific to the MEV cargo. This indicates that MEV-based formulations elicit protective humoral responses that comprise serum and mucosal IgA, thereby generating vaccine-induced IgA-producing memory B-cells to provide systemic and mucosal responses that protect from reinfection.

As described herein, the MEVs can be delivered to the mucosa, which include by oral administration, by inhalation into the lungs and/or nose, ocularly, and via urogenital tissues, and other routes by which the MEVs contact mucosal tissues.

The mucosal immune system is comprised of anatomically remote and physiologically independent compartments that protect ocular, nasopharyngeal, respiratory, oral, gastrointestinal, and genitourinary mucosae. Collectively, the mucosal surface exceeds 300 m². In many infections, the mucosa, such as the nasal cavity for respiratory pathogens, is a primary checkpoint for the systemic invasion. Numerous pathogens, including, for example, S. aureus, S. pneumoniae, and viruses, such as flu viruses, and corona viruses, including SARS-CoV-2, can adhere/colonize the mucosal lining to trigger an infection. Secretory IgA (sIgA), discussed further below, serves as the first line of immune defense against foreign pathogens. IgA is the predominant immunoglobulin expressed in the respiratory tract, cornea, and gastrointestinal tract mucosal surfaces, and IgA responses with neutralizing capability are described for several viral pathogens. sIgA facilitates clearance of pathogenic microbes by intercepting their access to epithelial receptors and mucus entrapment through immune exclusion. The significance of IgA-mediated mucosal immunity provides a mechanism to mount protection against a number of pathogens within the respiratory, gastrointestinal and genitourinary tracts.

The challenge, however, remains that when a systemic immune response is induced, it is not necessarily communicated to the mucosal IgA system. Traditionally, intramuscularly or intradermally administered vaccines generate strong systemic IgM and IgG predominant responses. The intramuscular route induces an immune response in the axillary draining lymph node that is biased towards class switch to IgG rather than IgA. In contrast, IgA-based antibody response in body fluids (e.g., the serum, saliva, and bronchoalveolar lavage fluid) is required for protective immunity against many pathogens, and it is associated with expansion of IgA plasmablasts with mucosal homing characteristics.

Results described in the Examples herein include analysis of the antigen-specific antibody response after intramuscular or oral administration of MEV-based vaccine formulations. As can be seen in FIGS. 14A-14C, the antigen-specific antibodies produced were shown to include immunoglobulin A (IgA)-class antibodies that are specific to the MEV cargo. MEV-based formulations, thus, can elicit a protective humoral response that comprises serum and mucosal IgA, and also generates vaccine-induced IgA-producing memory B-cells that provide systemic and mucosal responses needed to protect from reinfection. As recognized by those skilled in the art, neutralizing antibody titers are correlated with vaccine protection, while immune memory is essential for effective vaccination.

d. MEV-Mediated Immunization Upon Intramuscular Delivery

Efficient or effective immunization relies on concurrent delivery of two signals to the immune system: a molecular pattern (antigen) to be specifically recognized and a danger signal (immunostimulation) to activate the response. The immune system identifies threats to initiate an immune response based on the presence of non-self-molecular patterns (including pathogens) and/or alarm signals from cells under stress. Therefore, classical approaches to vaccination are based on adjuvants used to accelerate, prolong, or enhance antigen-specific immune responses when used in combination with specific vaccine antigens. The results described in the Examples herein include analysis of the immune response after intramuscular administration of classical and MEV-based vaccine formulations. Direct comparison between adjuvant-assisted and MEV-mediated intramuscular immunization demonstrates a similar profile of the immune response. The results indicate that MEVs deliver the effective immunostimulatory signal equivalent to a classically used adjuvant.

e. Adjuvants

Adjuvants help antigen to elicit an early, high and long-lasting immune response with less antigen, thus saving on vaccine production costs. In recent years, adjuvants received much attention because of the development of purified, subunit and synthetic vaccines which are poor immunogens and require adjuvants to evoke the immune response. With the use of adjuvants immune response can be selectively modulated to major histocompatibility complex (MHC) class I or MHC class II and Th1 or Th2 type, which is very important for protection against diseases caused by intracellular pathogens such as viruses, parasites and bacteria (Mycobacterium). A number of problems are encountered in the development and use of adjuvants for human vaccines. An issue with the use of adjuvants for human vaccines, particularly routine childhood vaccines, is the toxicity and adverse side-effects of most of the adjuvant formulations. Adjuvants for human vaccination often balance a requirement for adjuvanticity and an acceptable low level of side-effects. Other problems with the development of adjuvants include restricted adjuvanticity of certain formulations to a few antigens, use of aluminum adjuvants as reference adjuvant preparations under suboptimal conditions, non-availability of reliable animal models, use of non-standard assays and biological differences between animal models and humans leading to the failure of promising formulations to show adjuvanticity in clinical trials. The most common adjuvants for human use today are still aluminum hydroxide and aluminum phosphate, although calcium phosphate and oil emulsions also have some use in human vaccinations.

Adjuvants can be classified based on their mechanisms of action, dividing them into two main categories: delivery systems (particulate) and immune potentiators. A further class of adjuvants comprises mucosal adjuvants. In delivery system adjuvants, antigens are associated with an adjuvant that works especially as an antigen carrier. They induce a local proinflammatory response by activating the innate immune system, leading to the recruitment of immune cells to the site of injection. The antigen-adjuvant complex activates pattern-recognition receptor (PRR) pathways by acting as pathogen-associated molecular patterns (PAMPs). This causes the activation of innate immune cells with the production of cytokines and chemokines. The same pathway is directly activated by immune potentiators. FIG. 16 depicts the immune response to immunization with an adjuvant plus and antigen. MEVs plus antigen follow a similar route, except that MEVs contain the antigen (or nucleic acid encoding the antigen) as cargo inside the MEV. The antigen can be expressed in the MEV; it can be expressed on the surface of the MEV, or other delivered into the host cell as a protein. MEVs provide delivery systems. TLRs can recognize exogenous ligands (PAMPS) and endogenous ligands (DAMPS).

f. Isotype Switching

Immunoglobulin class switching, also known as isotype switching, isotypic commutation or class-switch recombination (CSR), is a biological mechanism that changes a B cell's production of immunoglobulin from one type to another, such as from the isotype IgM to the isotype IgG. During this process, the constant-region portion of the antibody heavy chain is changed, but the variable region of the heavy chain stays the same. Since the variable region does not change, class switching does not affect antigen specificity. Instead, the antibody retains affinity for the same antigens, but can interact with different effector molecules. Isotype switching confers functional diversity to the immune response, and the generation of memory B cells providing long-lasting immunity to reinfections.

IgA class switching is the process whereby B cells acquire the expression of IgA, the most abundant antibody isotype in mucosal secretions. In general, for example, intramuscularly or intradermally administered vaccines generate strong IgM and IgG predominant responses, which provide strong protection against lower respiratory tract disease. In influenza, for example, IgA together with IgG is more important in protection against secondary infection; IgG and IgM predominate in the primary immune response. IgA antibodies primarily are involved in the immune response at the level of the mucosa. IgA is the primary immunoglobulin found in mucous secretions, including tears, saliva, sweat, colostrum and secretions from the genitourinary tract, gastrointestinal tract, prostate, and respiratory epithelium.

As shown herein, the responses, following IM and oral administration show isotype switching; IgG and IgA antibodies are observed (see, FIGS. 14A-14C). The results also showed that there was an absence of ‘neutralization’ against the MEVs, or the appearance of a ‘neutralizing’ immune response against the MEVs themselves, upon repeat administration. This allows for redosing (repeat administration of the MEVs). Eight (8) repeat administrations of the MEV over 60 days were done, no antibody response against the MEVs was observed. A cellular immune response involving T memory cells following IM and oral administration was generated. The T-cell response (involving memory cells) was antigen specific.

Results shown herein demonstrate generation of an ‘antigen-specific’ immune response following oral administration of the antigen-loaded MEVs. The results show the phenomenon known as ‘isotype switching’ Particularly, with the antigen-loaded MEVs, the isotype switching primarily was from IgG to IgA. For vaccines the humoral ‘antigen-specific’ immune response involving or including IgA antibodies, following IM (intramuscular) administration; and, even more, the generation of a humoral ‘antigen-specific’ immune response involving mainly IgA antibodies, following oral administration is advantageous for vaccines, particularly for mucosally administered vaccines.

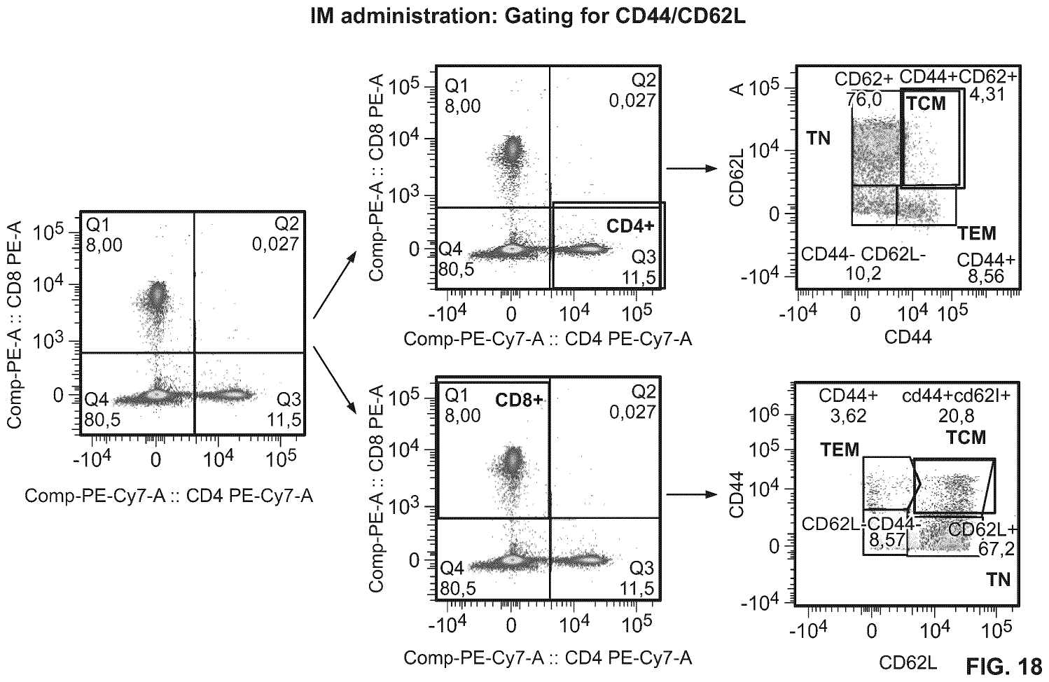

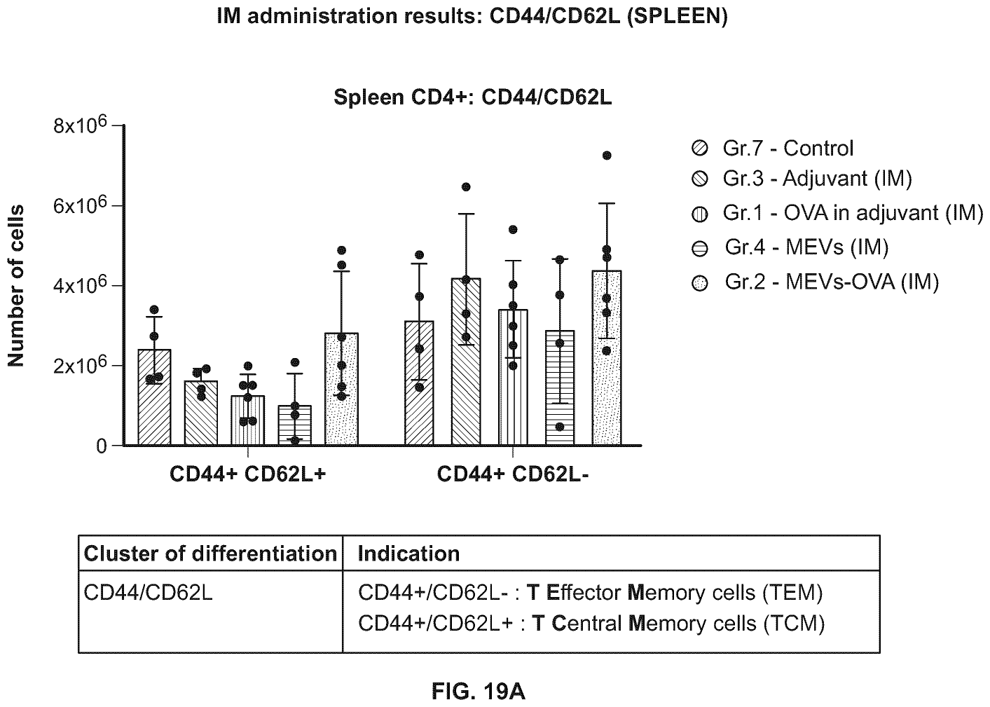

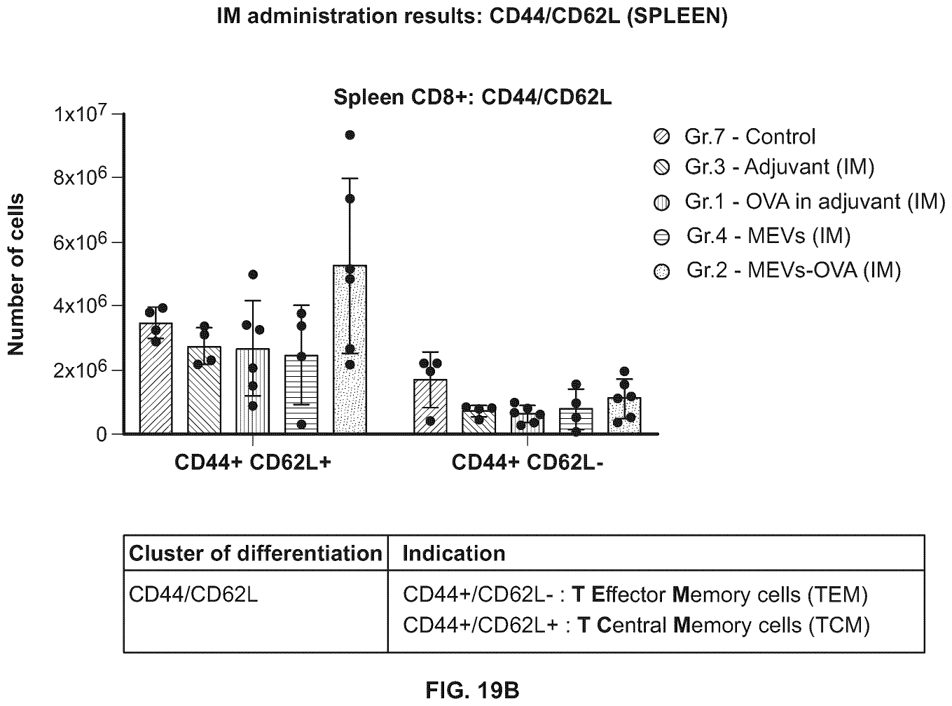

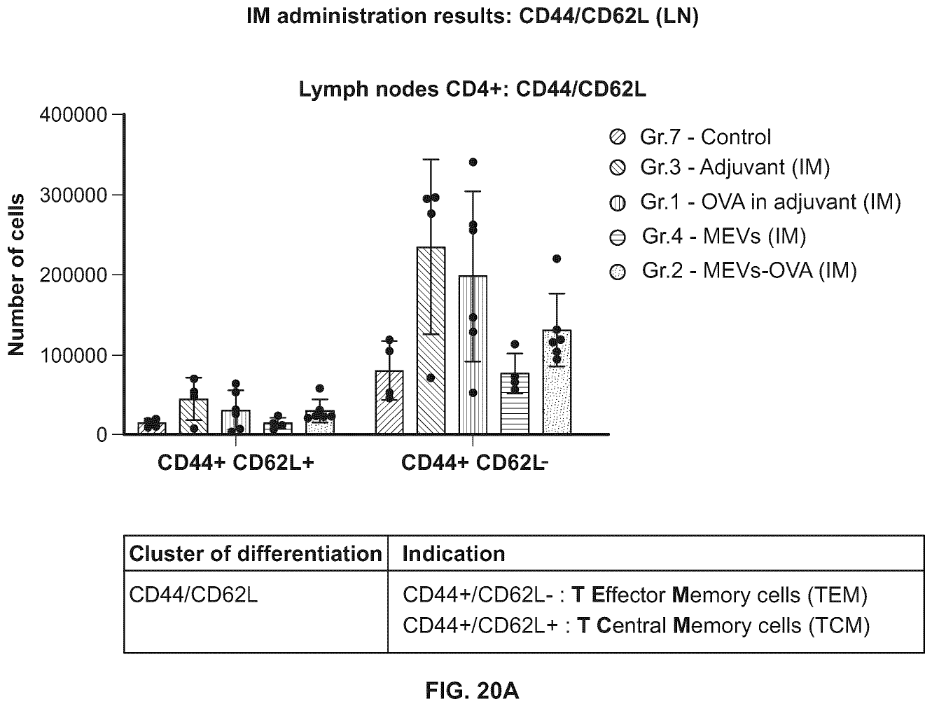

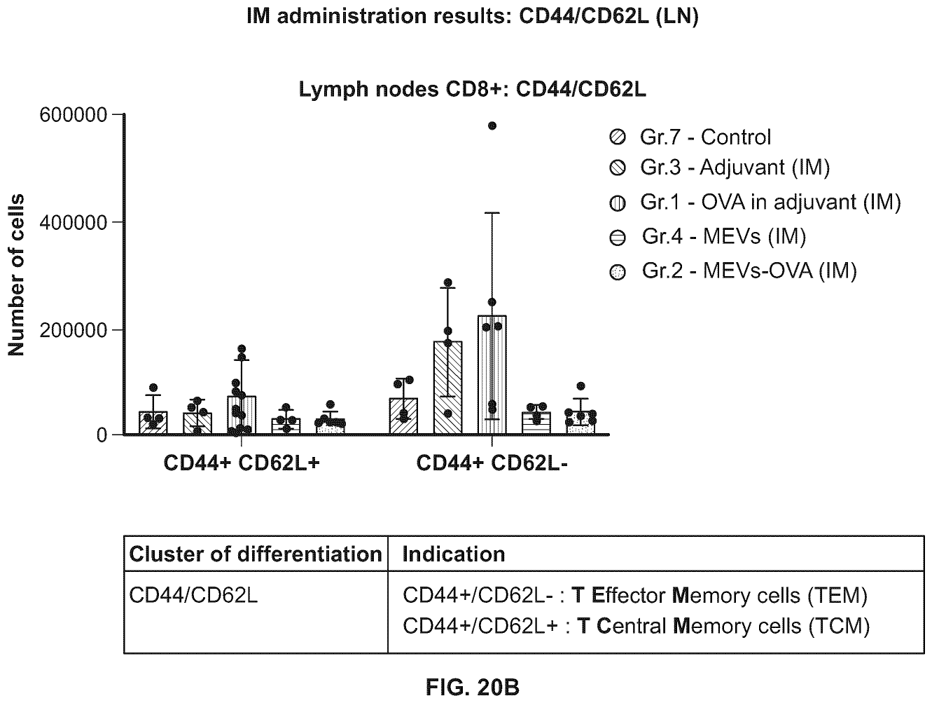

Results of T-cell responses following IM administration are shown in FIGS. 18-26. FIGS. 18-20 show CD44/CD62L analysis. FIG. 18 shows how gating of cell subsets was effected: these include CD4+ and CD4−, CD8+ and CD8−, CD44+ and CD44− and CD62L+ and CD62L−. CD44+/CD62L− cells are indicative of T Effector Memory Cells (TEM) and CD44+/CD62L+ cells and indicative of T Central Memory Cells (TCM). The results show that in CD4+ and CD8+ cells recovered from the spleen (FIGS. 19A and 19B), there is a marked increase in CD44+/CD62L+ cells (or TCM cells) upon IM administration with MEVs-OVA (Gr.2 MEVs-OVA (IM)), as compared to the IM administration of non-loaded MEV (Gr.4—MEVs (IM)), or to the IM administration of OVA in adjuvant (Gr. 1—OVA in adjuvant (IM)). Gr.7-control and Gr.3—Adjuvant (IM) bars are negative and positive controls, respectively. The results indicate that IM administration of MEV-OVA generates a T cell reaction that involves T memory cells. T memory cells are cells that secure the ‘memory’ of the response in the eventuality of a new challenge with the same antigen. The generation of T memory cells is a prerequisite for a good vaccine. These changes above, are observed in the spleen (FIGS. 19A and 19B), but are not clearly observed or are absent in the lymph nodes (FIGS. 20A and 20B).

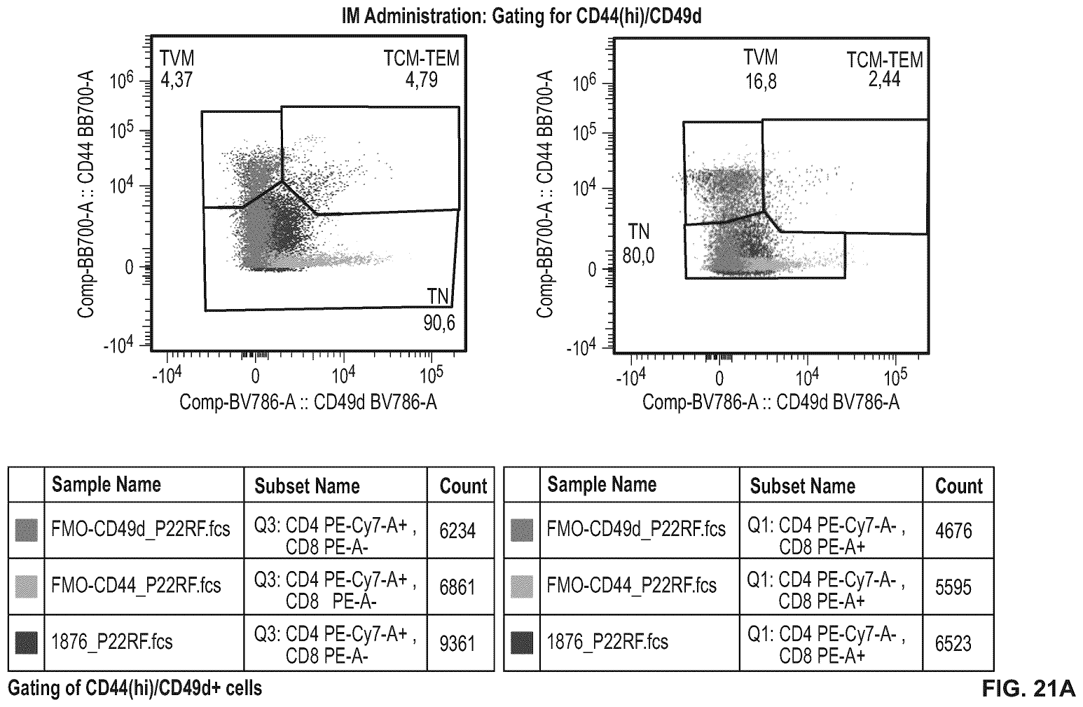

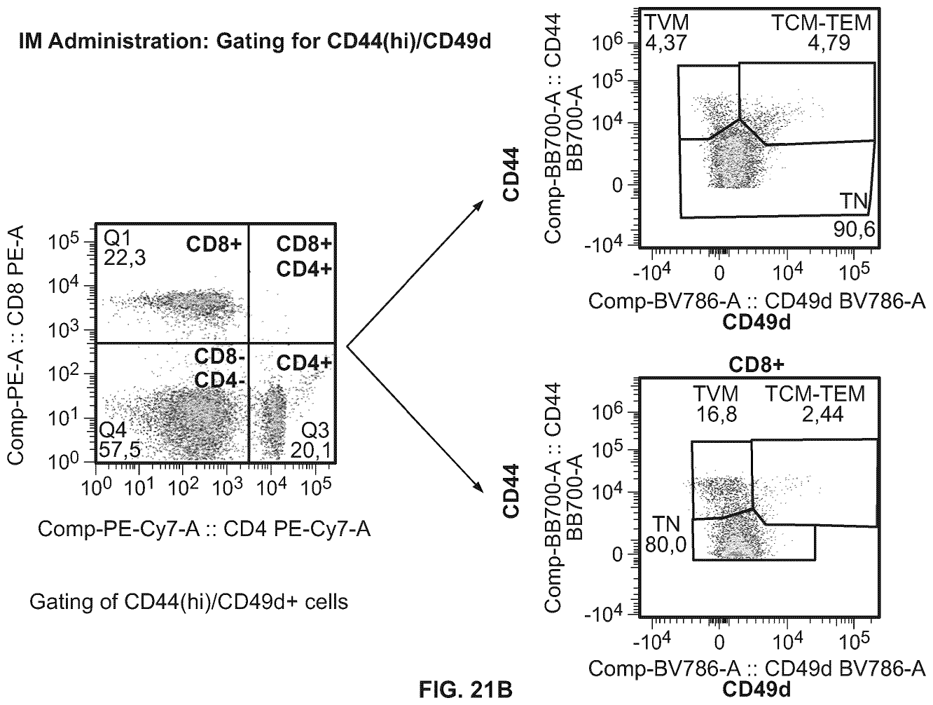

FIGS. 21-23 show a CD44(high)/CD49 analysis. FIGS. 21A-21B shows how the gating of the different cell subsets was made: CD4+ and CD4−, CD8+ and CD8−, CD44(hi)+ and CD44(hi)− and CD49+ and CD49−. CD44(hi)+/CD49− cells are indicative of T Virtual Memory Cells (TVM) and CD44(hi)+/CD49+ cells and indicative of T Central Memory Cells (TCM).

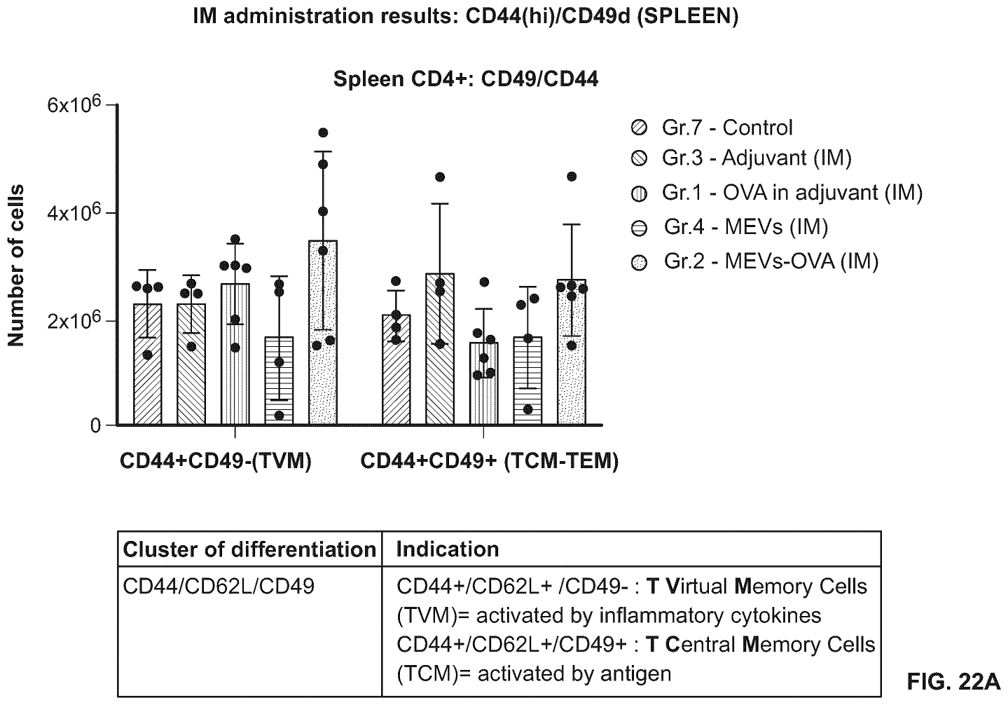

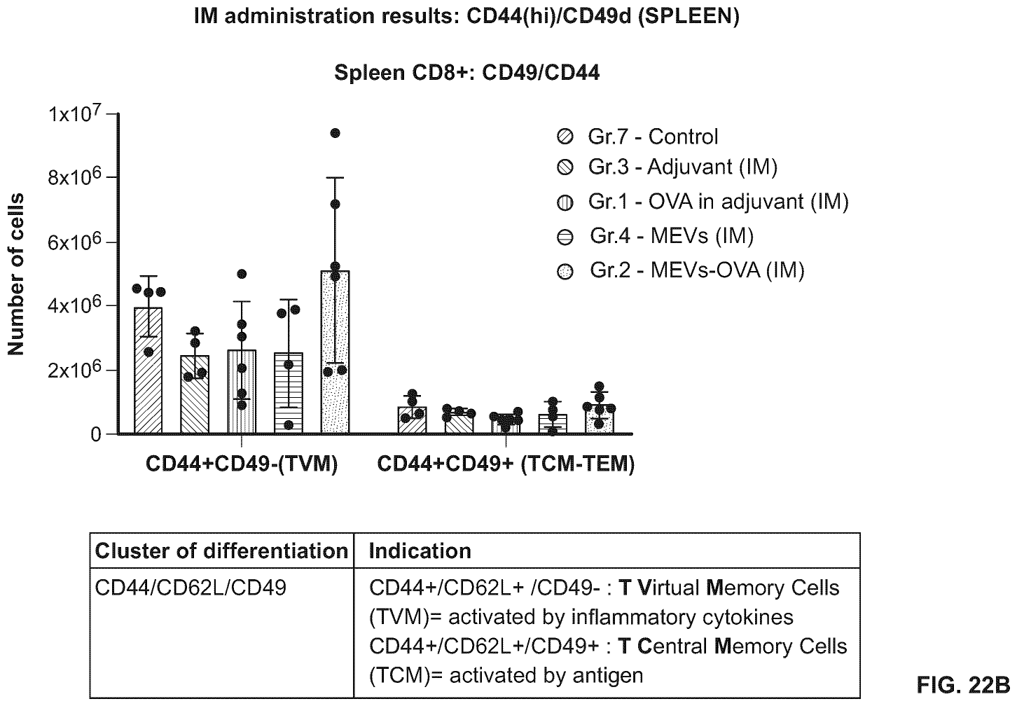

The results show that in CD4+ and CD8+ cells recovered from the spleen (FIGS. 22A and 22B), there is a marked increase in CD44(hi)+/CD49+ cells (or TCM cells) upon IM administration with MEVs-OVA (Gr.2—MEVs-OVA (IM)), as compared to the IM administration of non-loaded MEV (Gr.4—MEVs (IM)), or to the IM administration of OVA in adjuvant (blue bar). Gr.7—control and Gr. 3—Adjuvant (IM) bars are negative and positive controls, respectively. These results are consistent with the results discussed above (FIGS. 18-20B) for CD44+/CD62L+ (FIGS. 18-20) and lead to the same conclusions.

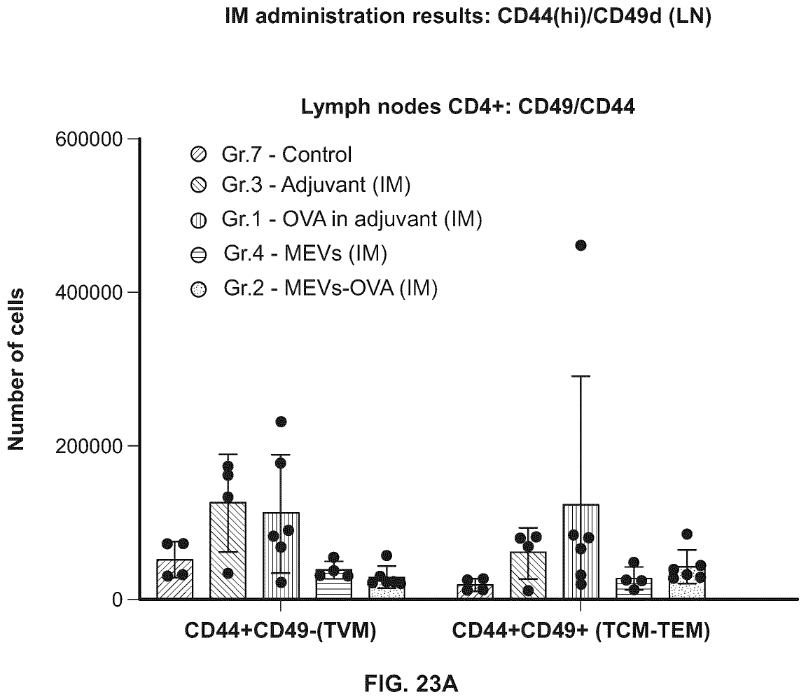

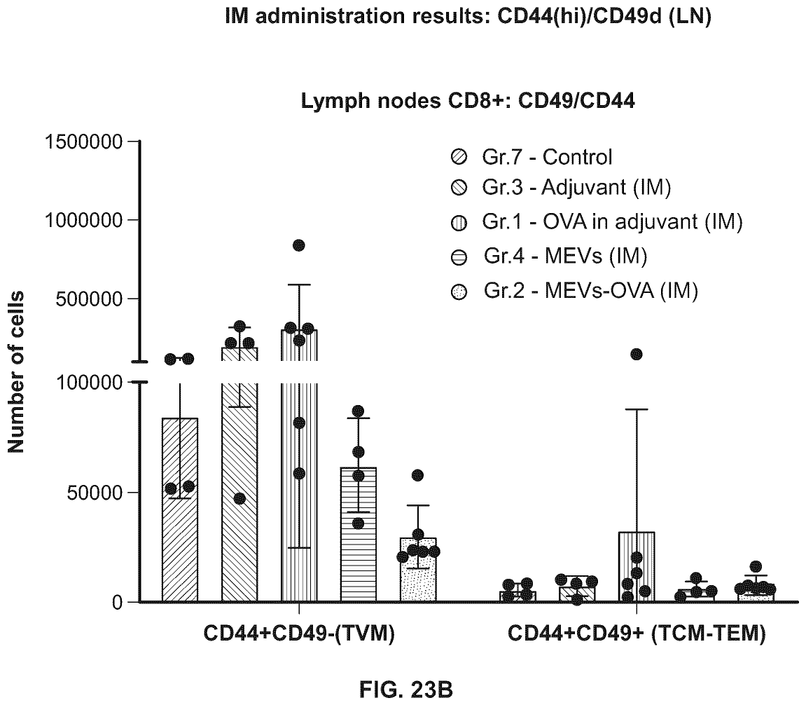

The results indicate that IM administration of MEV-OVA generates a T cell reaction that involves T memory cells. Again, T memory cells are cells that secure the ‘memory’ of the response in the eventuality of a new challenge with the same antigen. The generation of T memory cells is a prerequisite for a good vaccine. Similarly, the changes above, observed in the spleen (FIGS. 22A-B) are not clearly observed or are absent in the lymph nodes (FIGS. 23A and 23B).

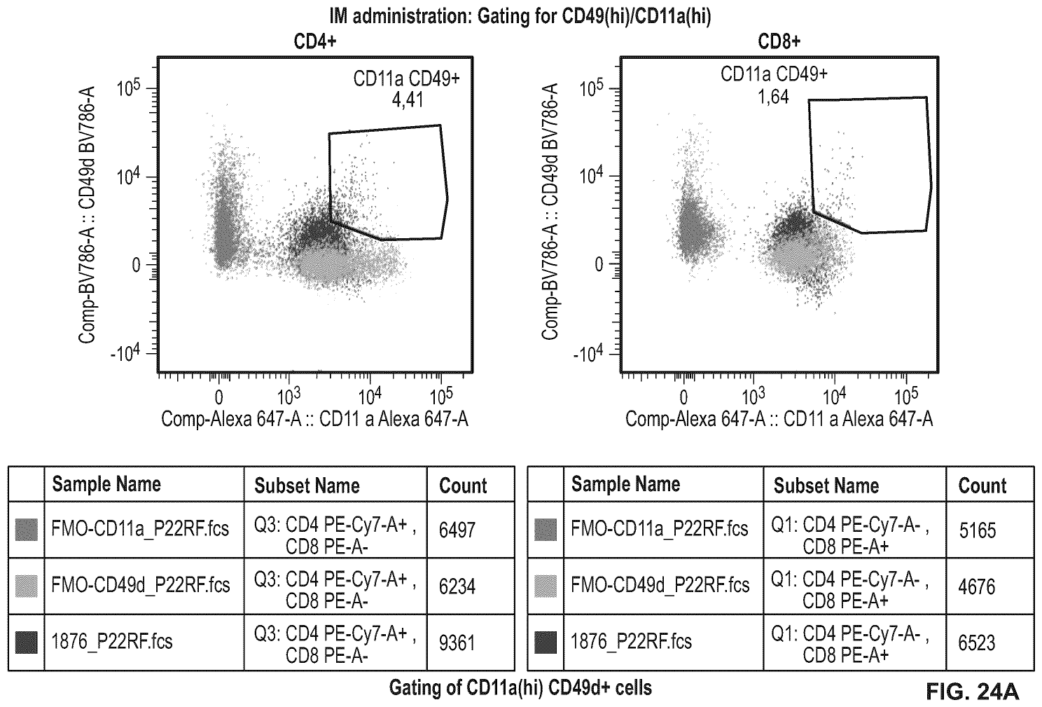

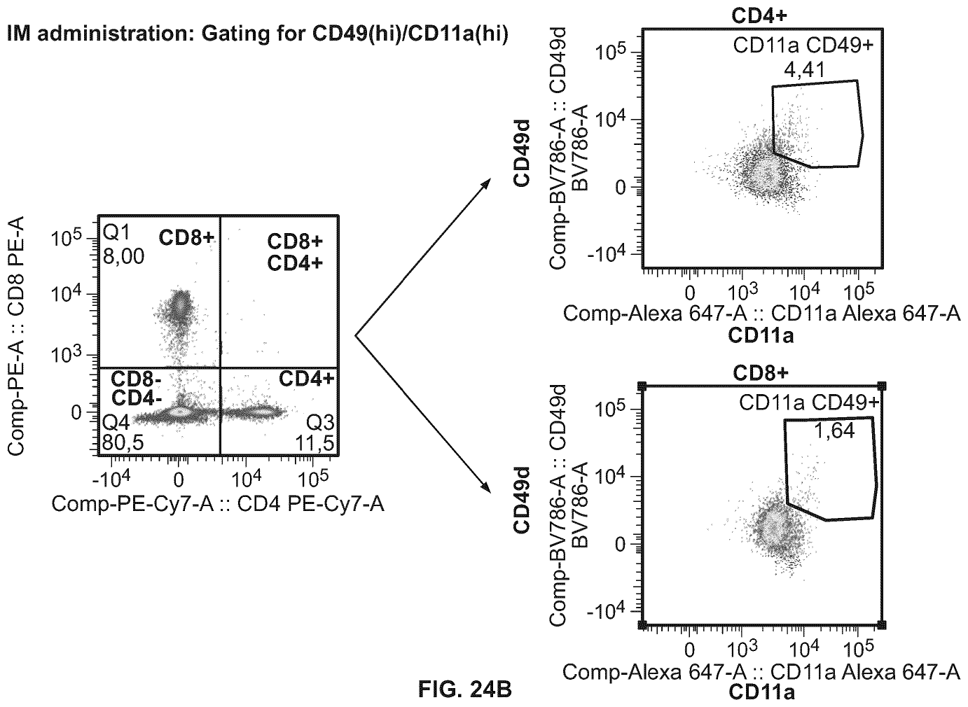

FIGS. 24A-26B show a CD49/CD11a analysis. FIGS. 24A-24B show how the gating of the different cell subsets was made: CD4+ and CD4−, CD8+ and CD8−, CD49+ and CD49−, and CD11a+ and CD11a−. CD49+/CD11a+ cells are indicative of antigen specific T cells following immunization.

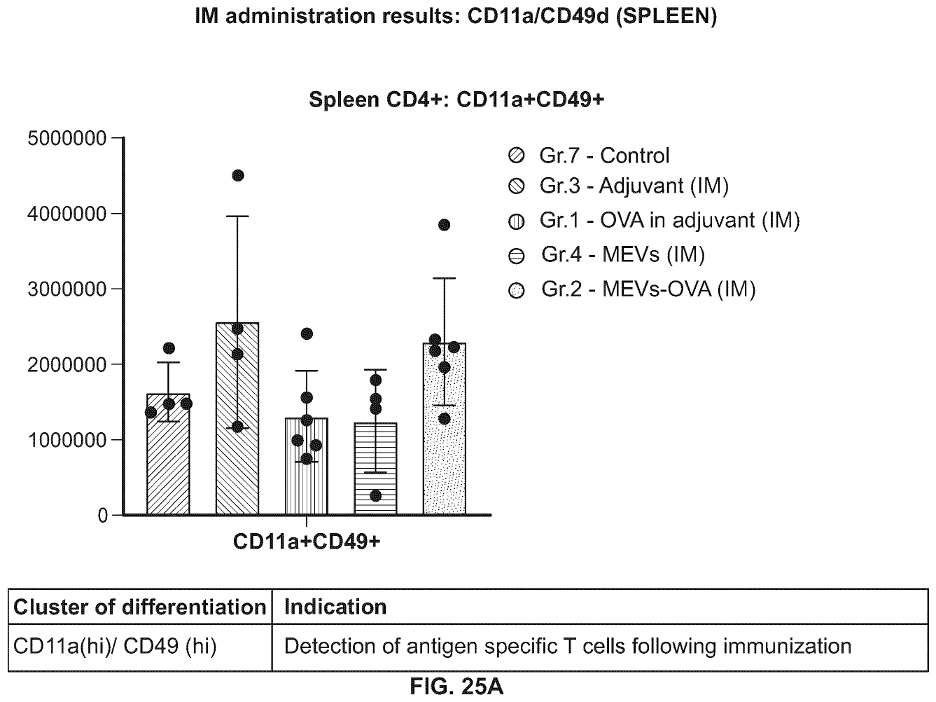

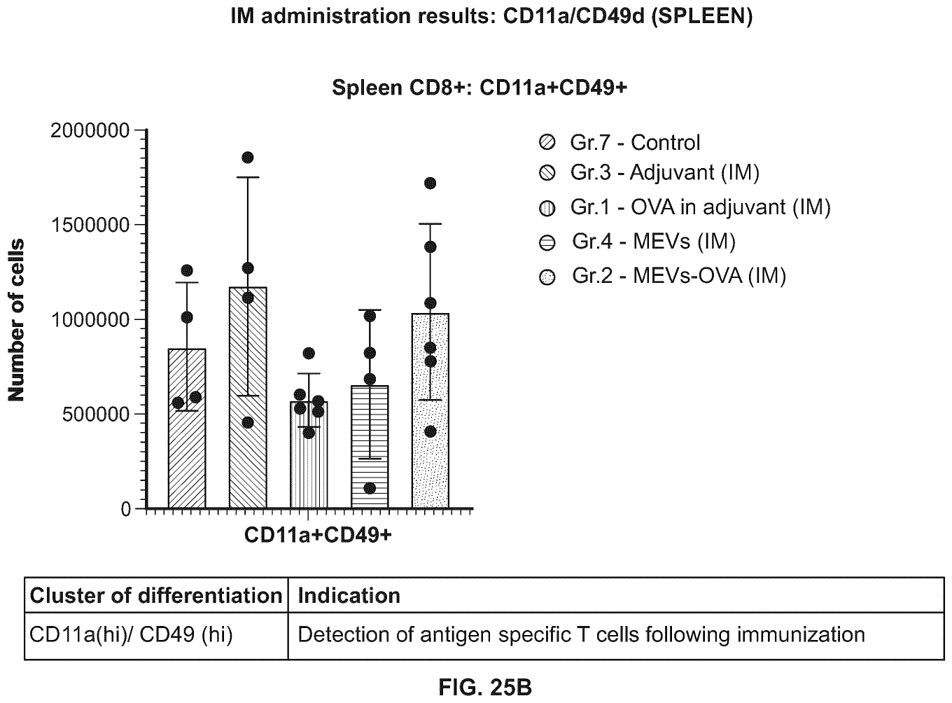

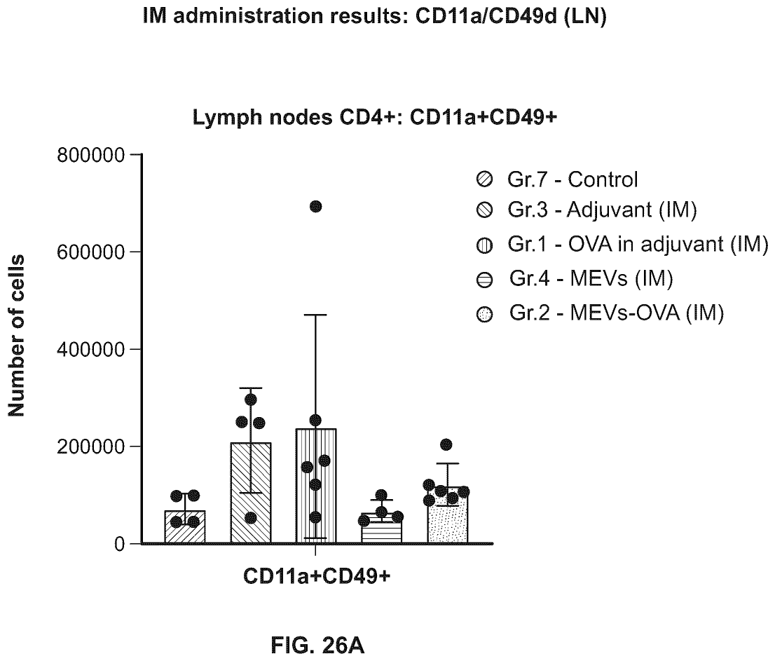

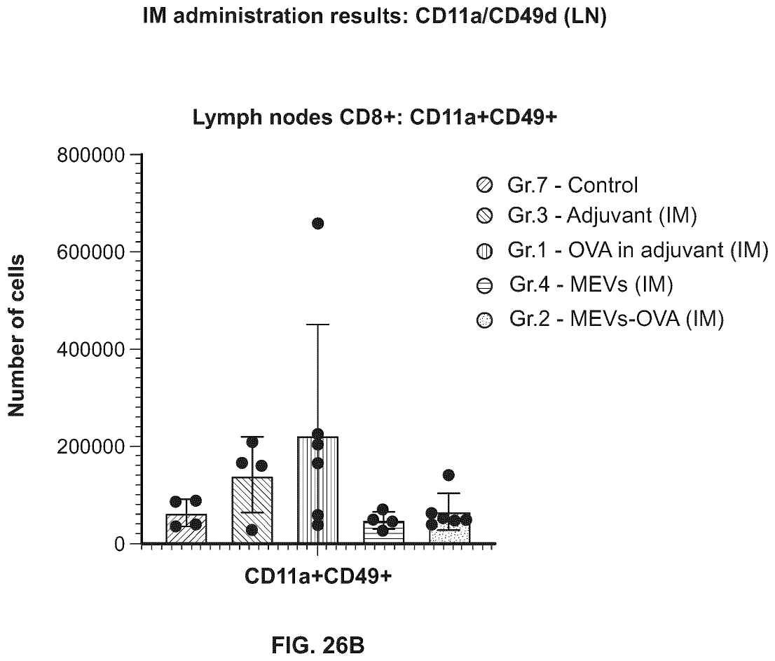

The results show that in CD4+ and CD8+ cells recovered from the spleen (FIGS. 25A and 25B), there is a marked increase in CD49+/CD11a+ cells (or antigen specific T cells) upon IM administration with MEVs-OVA (Gr.2—MEVs-OVA(IM)), as compared to the IM administration of non-loaded MEV (Gr.4—MEVs (IM)), or to the IM administration of OVA in adjuvant (Gr.1—OVA in adjuvant (IM)). Gr.7—control and Gr. 3—Adjuvant (IM) bars are negative and positive controls, respectively. The results indicate that IM administration of MEV-OVA generates an antigen specific T cell reaction. Similar to the changes above, the changes observed in the spleen (FIGS. 22A and 22B) are not clearly observed or are absent in the lymph nodes (FIGS. 26A and 26B).

### 3. MEVs and Cargo

MEVs provide numerous advantages for delivery of bioactive molecules, including therapeutic and diagnostic or detectable molecules, compared to other vehicles, including EVs from other sources, including plant sources (see discussion in the section below, and throughout the disclosure). For vaccines and immunomodulation, the MEVs can deliver antigens, including polypeptides, proteins, and antigenic epitopes thereof. The MEVs also can deliver immunomodulators, including, but not limited to, cytokines, chemokines, receptors and/or ligands involved in a disease, disorder, or condition, checkpoint inhibitors, and other such agents. They can be delivered as proteins or portions thereof loaded in the MEVs, and/or as nucleic acids in the MEVs for delivery to targeted cells, tissues, and organs of the immune system or involved in the immune system for expression, if the product is a polypeptide, protein, or portion thereof, or as the therapeutic agent, such as inhibitory RNA. Also provided are MEVs that deliver combinations of products for modulating the immune system and/or serving as vaccines. For example, antigens can be provided with immune modulators that stimulate a complementary immune response. Those of skill in immunology and the vaccine arts can select antigens, immune modulators and combinations thereof to achieve a desired response for immunoprotection, treatments, and other uses and combinations thereof. As discussed, vaccines can be designed to protect against infection by pathogens, to treat infections, to prevent or treat cancers, and other uses known to those of skill in the art.

### 4. Antigens

Antigens can include full-length proteins and polypeptides, or portions thereof that include or are an epitope. Antigens include antigenic proteins from pathogens, including bacteria, viruses, fungi, and parasites, that stimulate an immunoprotective response or an immune response against the pathogen. The antigens are provided as cargo in the MEVs. They can be provided as polypeptides and peptides, or a nucleic acid, DNA or RNA, encoding the antigen, as cargo in the MEVs. Antigens include those associated with pathogens or with conditions, such as cancer and include tumor-associated antigens expressed by tumor cells, receptors and ligands associated with cancers, oncofetal, and oncoviral antigens. Antigens include any antigen, epitope, neoepitope that results in an immunoprotective response that can be used to treat, or reduce the risk of developing, or reduce the severity, of a disease, disorder, or condition.

The MEVs can encode as DNA or RNA or deliver a protein, peptide, or portion thereof of an anti-viral or anti-bacterial therapeutic or anti-fungal or anti-parasitic, such as an inhibitor of a viral or bacterial product, or an inhibitor of the expression of a viral or bacterial product, or a viral or bacterial antigen or other immunizing antigen. The MEVs can deliver combinations of immune modulators and the anti-pathogen therapeutic, such as an antigen for mounting an immunoprotective immune response. The combination of the immune response to the antigen and/or the effects of the immune modulator provides a vaccine. The MEVs can be vaccines against and/or for treating or reducing the severity of infectious diseases, including, for example, diseases associated with viral infections, such as chronic viral infections and latent viral infections, such as infections by hepatitis viruses, herpesviruses, varicella zoster virus (VZV), Epstein-Barr virus (EBV), human immunodeficiency virus (HV), human T-cell leukemia virus (HTLV), Respiratory Syncytial Virus (RSV), measles virus, and other such viruses that chronically infect subjects, and/or also acute infections as well, such as initial infections with chronic influenza, P. gingivalis, and coronaviruses, such as Severe Acute Respiratory Syndrome coronavirus (SARS-CoV), Middle East Respiratory Syndrome coronavirus (MERS-CoV), and Severe Acute Respiratory Syndrome coronavirus 2 (SARS-CoV-2, which causes COVID-19). They also can be used as vaccines against respiratory viruses, for example by delivery to the lungs or nasal passages, orally for treating or preventing diseases caused by Enterobacteriaceae, such as E. coli, Klebsiella, Salmonella, and Shigella. The following table provides the sequences of exemplary antigens or antigenic polypeptides (see SEQ ID NOs.160-186). The MEVs can deliver the full-length polypeptides, or portions thereof that comprise an epitope; the MEVs also can deliver nucleic acid, such as encoded in a DNA plasmid, or RNA, such as mRNA.

The table below lists exemplary antigens for immunization or treatment of exemplary pathogens. As noted, vaccines can be used for treatment of a disease, disorder, or condition, or can be used prophylactically to prevent (including reducing the risk of a disease, disorder, or condition), or reducing the severity of a disease, disorder, or condition. Vaccines, not only can be used for pathogens, but also for treatment/prevention of cancers and other conditions. It is understood that the skilled person can select antigens for particular diseases, disorders, and condition for treatment, prevention, and reducing the risk or severity of a disease, disorder, or condition. The MEVs provide the vehicle for delivery. As described throughout the disclosure herein, MEVs can be conveniently administered via various routes, including orally and intramuscularly, or via inhalation for direct contact with mucosal tissues.

TABLE 2
Examples of vaccine antigens to be used for MEV-mediated immunization and
immunomodulation

| | Pathogen | Species of | |
|---|---|---|---|
| Antigen name | type | origin | Sequence (SEQ ID NOs: 160-186, respectively |
| Heat-labile | Bacterial | Escherichia | >tr\|B8XN37\|B8XN37_ECOLX Heat-labile |
| enterotoxin B | | coli | enterotoxin B subunit (Fragment) |
| subunit | | | OS = Escherichia coli OX = 562 GN = ltb |
| | | | PE = 4 SV = 1 |
| | | | MAPQSITELCSEYRNTQIYTINDKILSYTES |
| | | | MAGKREMVIITFKSGATFQVEVPGSQHIDS |
| | | | QKKAIERMKDTLRITYLTETKIDKLCVWN |
| | | | NKTPNSIAAISMEN |
| Cholera toxin B | Bacterial | Vibrio | >tr\|Q7X2D2\|Q7X2D2_VIBCL Toxin B subunit |
| (CTB) subunit | | cholerae | (Fragment) OS = Vibrio cholerae OX = 666 |
| | | | GN = CTB PE = 2 SV = 1 |
| | | | MTPQNITDLCAEYHNTQIYTLNDKIFSYTE |
| | | | SLAGKREMAIITFKNGAIFQVEVPGSQHIDS |
| | | | QKKAIERMKDTLRIAYLTEAKVEKLCVWN |
| | | | NKTPHAIAAISMANGT |
| extracellular | Bacterial | Yersinia | ncbi.nlm.nih.gov/pmc/articles/PMC1326254/¹ |
| capsule protein | | pestis | MADLTASTTATATLVEPARITL TYKEGAPI |
| F1/immune- | | | TIMDNGNIDTELLVGTLTLGGYKTGTTSTS |
| modulator V fusion | | | VNFTDAAGDPMYLTFTSQDGNNHQFTTKV |
| protein | | | IGKDSRDFDISPKVNGENLVGDDVVLATGS |
| | | | QDFFVRSIGSKGGKLAAGKYTDAVTVTVS |
| | | | NQEFMIRAYEQNPQHFIEDLEKVRVEQLTG |
| | | | HGSSVLEELVQLVKDKNIDISIKYDPRKDS |
| | | | EVFANRVITDDIELLKKILAYFLPEDAILKG |
| | | | GHYDNQLQNGIKRVKEFLESSPNTQWELR |
| | | | AFMAVMHFSLTADRIDDDILKVIVDSMNH |
| | | | HGDARSKLREELAELTAELKIYSVIQAEIN |
| | | | KHLSSSGTINIHDKSINLMDKNLYGYTDEEI |
| | | | FKASAEYKILEKMPQTTIQVDGSEKKIVSIK |
| | | | DFLGSENKRTGALGNLKNSYSYNKDNNEL |
| | | | SHFATTCSDKSRPLNDLVSQKTTQLSDITSR |
| | | | FNSAIEALNRFIQKYDSVMQRLLDDTSGK |
| Outer membrane | Bacterial | Shigella | >tr\|A0A0H2VSY6\|A0A0H2VSY6_SHIFL |
| protein receptor | | flexneri | Outer membrane protein receptor for |
| for ferrichrome | | | ferrichrome, colicin M, and phages T1, T5, |
| | | | and phi80 OS = Shigella flexneri OX = 623 |
| | | | GN = fhuA PE = 3 SV = 1 |
| | | | MARSKTAQPKHSLRKIAVVVATAVSGMSV |
| | | | YAAAVEPKEDTITVTAAPAPQESAWGPAA |
| | | | TIAARQSATGTKTDTPIQKVPQSISVVTAEE |
| | | | MALHQPKSVKEALSYTPGVSVGTRGASNT |
| | | | YDHLIIRGFAAEGQSQNNYLNGLKLQGNF |
| | | | YNDAVIDPYMLERAEIMRGPVSVLYGKSS |
| | | | PGGLLNMVSKRPTTEPLKEVQFKAGTDSLF |
| | | | QTGFDFSDALDDDGVYSYRLTGLARSANA |
| | | | QQKGSEEQRYAIAPAFTWRPDDKTNFTFLS |
| | | | YFQNEPETGYYGWLPKEGTVEPLPNGKRL |
| | | | PTDFNEGAKNNTYSRNEKMVGYSFDHEFN |
| | | | DTFTVRQNLRFAENKTSQNSVYGYGVCSD |
| | | | PANAYSKQCAALAPADKGHYLARKYVVD |
| | | | DEKLQNFSVDTQLQSKFATGDIDHTLLTGV |
| | | | DFMRMRNDINAWFGYDDSVPLFNLYNPV |
| | | | NPDFDFNAKDPANSGPYRILNKQKQTGVY |
| | | | VQDQAQWDKVLVTLGGRYDWADQESLN |
| | | | RVAGTTDKRDDKQFTWRGGVNYLFDNGV |
| | | | TPYFSYSESFEPSSQVGKDGNIFAPSKGKQ |
| | | | YEVGVKYVPEDRPIVVTGAVYNLTKTNNL |
| | | | MADPEGSFFSVEGGEIRARGVEIEAKAALS |
| | | | ASVNVVGSYTYTDAEYTTDTTYKGNTPAQ |
| | | | VPKHMASLWADYTFFDGPLSGLTLGTGGR |
| | | | YTGSSYGDPANSFKVGSYTVVDALVRYDL |
| | | | ARVGMAGSNVALHVNNLFDREYVASCFN |
| | | | TYGCFWGAERQVVATATFRF |
| Outer membrane | Bacterial | Pseudomonas | >tr\|A6V748\|A6V748_PSEA7 Outer membrane |
| protein OprF | | aeruginosa | protein OprF OS = Pseudomonas aeruginosa |
| | | | (strain PA7) OX = 381754 GN = oprF PE = 4 |
| | | | SV = 1 |
| | | | MKLKNTLGVVIGSLVAASAMNAFAQGQN |
| | | | SVEIEAFGKRYFTDSVRNMKNADLYGGSI |
| | | | GYFLTDDVELALSYGEYHDVRGTYETGNK |
| | | | KVHGNLTSLDAIYHFGTPGVGLRPYVSAG |
| | | | LAHQNITNINSDSQGRQQMTMANIGAGLK |
| | | | YYFTENFFAKASLDGQYGLEKRDNGHQGE |
| | | | WMAGLGVGFNFGGSKAAPAPEPVADVCS |
| | | | DSDNDGVCDNVDKCPDTPANVTVDANGC |
| | | | PAVAEVVRVQLDVKFDFDKSKVKENSYA |
| | | | DIKNLADFMKQYPSTSTTVEGHTDSVGTD |
| | | | AYNQKLSERRANAVRDVLVNEYGVEGGR |
| | | | VNAVGYGESRPVADNATAEGRAINRRVEA |
| | | | EVEAEAK |
| N-terminal portion | Bacterial | Staphylococcus | ncbi.nlm.nih.gov/pmc/articles/PMC8876328/² |
| of the Candida | | aureus | AKTITGVFNSFNSLTWSNAATYNYKGPGT |
| albicans | | | PTWNAVLGWSLDGTSASPGDTFTLNMPCV |
| agglutinin- | | | FKFTTSQTSVDLTAHGVKYATCQFQAGEE |
| like protein 3 | | | FMTFSTLTCTVSNTLTPSIKALGTVTLPLAF |
| (Als3p) | | | NVGGTGSSVDLEDSKCFTAGTNTVTFNDG |
| | | | GKKISINVDFERSNVDPKGYLTDSR VIPSLN |
| | | | KVSTLFVAPQCANGYTSGTMGFANTYGD |
| | | | VQIDCSNIHVGITKGLNDWNYPVSSESFSY |
| | | | TKTCSSNGIFITYKNVPAGYRPFVDAYISAT |
| | | | DVNSYTLSYANEYTCAGGYWQRAPFTLR |
| | | | WTGYRNSDAGSNGIVIVATTRTVTDSTTA |
| | | | VTTLPFDPNRDKTKTIEILKPIPTTTITTSYV |
| | | | GVTTSYSTKTAPIGETATVIVDIPYHTTTTV |
| | | | TSKWTGTITSTTTHTNPTDSIDTVIVQVP |
| 27-kDa outer | Bacterial | Salmonella | pubmed.ncbi.nlm.nih.gov/21300870/³ |
| membrane protein | | enterica | MKNFFAVCIIPLVVTWSATASAKEGIYITG |
| (T2544) | | serovar Typhi | KAGTSVVNVYGINSTFSQEEIVNGHATLPD |
| | | | RTKGVFGGGVAIGYDFYDPFQLPVRLELD |
| | | | TTFRGETDAKGGQDIIAFGQPVHINVKNQV |
| | | | RMTTYIVNGYYDFHNSTAFTPYISAGVGLA |
| | | | HVKLSNNTIPVGFGINETLSASKNNFAWGA |
| | | | GIGAKYAVTDNIMIDASYKYINAGKVSISK |
| | | | NHYAGDEHTAYDADTKAASNDFMLGITY |
| | | | AF |
| Hepatitis B | Viral | Hepatitis B | >tr\|Q81158\|Q81158_HBV Surface antigen |
| surface antigen | | virus (HBV) | (HBsAg) OS = Hepatitis B virus OX = 10407 |
| (HBsAg) | | | PE = 2 SV = 1 |
| | | | MENTTSGFLGPLLVLQAGFFLLTRILTIPQS |
| | | | LDSWWTSLNFQGGAPTCPGQNSQSPTSNH |
| | | | SPTSCPPICPGYRWMCLRRFIIFLFILLLCLIF |
| | | | LLVLLDYQGMLPVCPLLPGTSTTGTGPCRT |
| | | | CTIPAQGTSMFPSCCCTKPSDGNCTCIPIPSS |
| | | | WAFARFLWEWASVRFSWLSLLVPFVQWF |
| | | | AGLSPTVWLSVIWMMWYRGPSLYNTLSPF |
| | | | LPLLPISFCLWVYI |
| E1-E2 genome | Viral | Hepatitis C | ncbi.nlm.nih.gov/pmc/articles/PMC43144/⁴ |
| polyprotein | | virus (HCV) | >tr\|B5DA15\|B5DA15_9HEPC Genome |
| | | genotype | polyprotein (Fragment) OS = hepatitis C |
| | | 1a | virus genotype 1a OX = 2847144 GN = E1-E2 |
| | | | PE = 3 SV = 1 |
| | | | PTVALVTAQLLRIPQAILDMIAGAHWGVL |
| | | | AGIAYFSMVGNWAKVLVVLLLFAGVDAE |
| | | | TYTTGGNAGRVTARLTGLFSPGAKQKIQL |
| | | | VNTNGSWHINSTALNCDDSLNTGWVAGLF |
| | | | YHHKFDSSGCPERLASCRPLADFAQGWGPI |
| | | | SHASGSGPDQRPYCWHYPPKPCGIVPAKS |
| | | | VCG |
| Inner capsid | Viral | Human rotavirus | >tr\|B6CSK9\|B6CSK9_9VIRU Inner capsid |
| protein VP6 | | A | protein VP6 (Fragment) OS = Human rotavirus |
| | | | A OX = 10941 PE = 4 SV = 1 |
| | | | FNPIILRPNNVEVEFLLNGQIINTYQARFGTI |
| | | | VARNFDTIRLSFQLMRPPNMTPAVNALFPQ |
| | | | AQPFQHHATVGLTLRIESAVCESVLADANE |
| | | | TLLANVTAVRQEYAIPVGPVF |
| Outer capsid | Viral | Rotavirus A | >tr\|Q6SKR8\|Q6SKR8_9VIRU Outer capsid |
| glycoprotein VP7 | | | glycoprotein VP7 OS = Rotavirus A OX = 28875 |
| | | | GN = VP7 PE = 1 SV = 1 |
| | | | MYGIEYTTVLTFLISFILLNYILKSLTRMMD |
| | | | FVIYRFLFVIVVLSPLLKAQNYGINLPITGS |
| | | | MDTAYANSTQEETFLTSTLCLYYPTEAATE |
| | | | INDNSWKDTLSQLFLTKGWPTGSIYFREYT |
| | | | DIVSFSVDPQLYCDYNVVLMKYDAALQLD |
| | | | MSELADLILNEWLCNPMDITLYYYQQTDE |
| | | | ANKWISMGSSCTIKVCPLNTQTLGIGCLTT |
| | | | DTATFEEVATAEKLVITDVVDGVNHKLDV |
| | | | TTATCTIRNCKKLGPRENVAVIQVGGSDVL |
| | | | DITADPTTAPQTERMMRINWKKWWQVFY |
| | | | TVVDYVNQIIQLMSKRSRSLNSAAFYYRV |
| Capsid protein | Viral | Norwalk virus | >tr\|Q8QWP1\|Q8QWP1_9CALI Capsid protein |
| | | (NV) | (Fragment) OS = Norwalk virus OX = 11983 |
| | | | PE = 4 SV = 1 |
| | | | MKMASNDAAPSNDGAAGLVPEINNEAMA |
| | | | LEPVAGAIAAPLTGQQNIIDPWIMNNFVQA |
| | | | PGGEFTVSPRNSPGEVLLNLELGPEINPYLA |
| | | | HLA |
| spike protein S1 | Viral | SARS- | ncbi.nlm.nih.gov/pmc/articles/PMC1157057/⁵ |
| fragment | | coronavirus | SDLDRCTTFDDVQAPNYTQHTSSMRGVYY |
| | | (CoV) | PDEIFRSDTLYLTQDLFLPFYSNVTGFHTIN |
| | | | HTFGNPVIPFKDGIYFAATEKSNVVRGWVF |
| | | | GSTMNNKSQSVIIINNSTNVVIRACNFELCD |
| | | | NPFFAVSKPMGTQTHTMIFDNAFNCTFEYI |
| | | | SDAFSLDVSEKSGNFKHLREFVFKNKDGFL |
| | | | YVYKGYQPIDVVRDLPSGFNTLKPIFKLPL |
| | | | GINITNFRAILTAFSPAQDIWGTSAAAYFVG |
| | | | YLKPTTFMLKYDENGTITDAVDCSQNPLA |
| | | | ELKCSVKSFEIDKGIYQTSNFRVVPSGDVV |
| | | | RFPNITNLCPFGEVFNATKFPSVYAWERKK |
| | | | ISNCVADYSVLYNSTFFSTFKCYGVSATKL |
| | | | NDLCFSNVYADSFVVKGDDVRQIAPGQTG |
| | | | VIADYNYKLPDDFMGCVLAWNTRNIDATS |
| | | | TGNYNYKYRYLRHGKLRPFERDISNVPFSP |
| | | | DGKPCTPPALNCYWPLNDYGFYTTTGIGY |
| | | | QPYRVVVLSFELLNAPATVCGPKLSTDLIK |
| | | | NQCVNFNFNGLTGTGVLTPSSKRFQPFQQF |
| | | | GRDVSDFTDSVRDPKTSEILDISPCSFGGVS |
| | | | VITPGTNASSEVAVLYQDVNCTDVSTAIHA |
| | | | DQLTPAWRIYSTGNNVFQTQAGCLIGAEH |
| | | | VDTSYECDIPIGAGICASYHTVSLLRSTSQK |
| | | | SIVAYTMSLGADSSIAYSNNTIAIPTNFSISIT |
| | | | TEVMPVSM |
| B5R antigenic | Viral | Vaccinia virus | ncbi.nlm.nih.gov/pmc/articles/PMC1871876/⁶ |
| ectodomain | | | TCTVPTMNNAKLTSTETSFNDKQKVTFTC |
| | | | DQGYHSSDPNAVCETDKWKYENPCKKMC |
| | | | TVSDYISELYNKPLYEVNSTMTLSCNGETK |
| | | | YFRCEEKNGNTSWNDTVTCPNAECQPLQL |
| | | | EHGSCQPVKEKYSFGEYMTINCDVGYEVI |
| | | | GASYISCTANSWNVIPSCQQKCDMPSLSNG |
| | | | LISGSTFSIGGVIHLSCKSGFTLTGSPSSTCID |
| | | | GKWNPVLPICVRTNEEFDPVDDGPDDETD |
| | | | LSKLSKDVVQYEQEIESL |
| envelope protein | Viral | Japanese | uniprot.org/uniprotkb/P27395/entry#Envelope_ |
| (E) | | encephalitis | protein E |
| | | virus (JEV) | FNCLGMGNRDFIEGASGATWVDLVLEGDS |
| | | | CLTIMADKPTLDVRMINIEASQLAEVRSYC |
| | | | YHASVTDISTVARCPTTGEAHNEKRADSSY |
| | | | VCKQGFTDRGWGNGCGLFGKGSIDTCAKF |
| | | | SCTSKAIGRTIQPENIKYEVGIFVHGTTTSE |
| | | | NHGNYSAQVGASQAAKFTVTPNAPSITLK |
| | | | LGDYGEVTLDCEPRSGLNTEAFYVMTVGS |
| | | | KSFLVHREWFHDLALPWTSPSSTAWRNRE |
| | | | LLMEFEGAHATKQSVVALGSQEGGLHQAL |
| | | | AGAIVVEYSSSVKLTSGHLKCRLKMDKLA |
| | | | LKGTTYGMCTEKFSFAKNPVDTGHGTVVI |
| | | | ELSYSGSDGPCKIPIVSVASLNDMTPVGRL |
| | | | VTVNPFVATSSANSKVLVEMEPPFGDSYIV |
| | | | VGRGDKQINHHWHKAGSTLGKAFSTTLK |
| | | | GAQRLAALGDTAWDFGSIGGVFNSIGRAV |
| | | | HQVFGGAFRTLFGGMSWITQGLMGALLL |
| | | | WMGVNARDRSIALAFLATGGVLVFLATN |
| | | | VHA |
| VP4N20 antigenic | Viral | Coxsackievirus | pubmed.ncbi.nlm.nih.gov/26073737/⁷ |
| peptide | | 16 (CV-A16) | GSQVSTQRSGSHENSNSASE |
| VP4N20 antigenic | Viral | Enterovirus 71 | pubmed.ncbi.nlm.nih.gov/26073737/⁷ |
| peptide | | (EV71) | GSQVSTQRSGSHENSNSASE |
| Minor capsid | Viral | Human | >sp\|P03107\|VL2_HPV16 Minor capsid protein |
| protein L | | papillomavirus | L2 OS = Human papillomavirus type 16 |
| | | type 16 | OX = 333760 GN = L2 PE = 1 SV = 1 |
| | | (HPV-16) | MRHKRSAKRTKRASATQLYKTCKQAGTC |
| | | | PPDIIPKEGKTIAEQILQYGSMGVFFGGLGI |
| | | | GTGSGTGGRTGYIPLGTRPPTATDTLAPVR |
| | | | PPLTVDPVGPSDPSIVSLVEETSFIDAGAPTS |
| | | | VPSIPPDVSGFSITTSTDTTPAILDINNTVTT |
| | | | VTTHNNPTFTDPSVLQPPTPAETGGHFTLSS |
| | | | STISTHNYEEIPMDTFIVSTNPNTVTSSTPIP |
| | | | GSRPVARLGLYSRTTQQVKVVDPAFVTTP |
| | | | TKLITYDNPAYEGIDVDNTLYFSSNDNSINI |
| | | | APDPDFLDIVALHRPALTSRRTGIRYSRIGN |
| | | | KQTLRTRSGKSIGAKVHYYYDLSTIDPAEE |
| | | | IELQTITPSTYTTTSHAASPTSINNGLYDIYA |
| | | | DDFITDTSTTPVPSVPSTSLSGYIPANTTIPF |
| | | | GGAYNIPLVSGPDIPINITDQAPSLIPIVPGSP |
| | | | QYTIIADAGDFYLHPSYYMLRKRRKRLPYF |
| | | | FSDVSLAA |
| Envelope | Viral | Human | >sp\|Q69091\|GD_HHV11 Envelope glycoprotein |
| glycoprotein D | | herpesvirus 1 | D OS = Human herpesvirus 1 (strain 17) |
| | | (HHV-1) | OX = 10299 GN = gD PE = 1 SV = 1 |
| | | | MGGAAARLGAVILFVVIVGLHGVRSKYAL |
| | | | VDASLKMADPNRFRGKDLPVLDQLTDPPG |
| | | | VRRVYHIQAGLPDPFQPPSLPITVYYAVLE |
| | | | RACRSVLLNAPSEAPQIVRGASEDVRKQPY |
| | | | NLTIAWFRMGGNCAIPITVMEYTECSYNKS |
| | | | LGACPIRTQPRWNYYDSFSAVSEDNLGFL |
| | | | MHAPAFETAGTYLRLVKINDWTEITQFILE |
| | | | HRAKGSCKYALPLRIPPSACLSPQAYQQGV |
| | | | TVDSIGMLPRFIPENQRTVAVYSLKIAGWH |
| | | | GPKAPYTSTLLPPELSETPNATQPELAPEDP |
| | | | EDSALLEDPVGTVAPQIPPNWHIPSIQDAAT |
| | | | PYHPPATPNNMGLIAGAVGGSLLAALVICG |
| | | | IVYWMRRHTQKAPKRIRLPHIREDDQPSSH |
| | | | QPLFY |
| envelope domain | Viral | Zika virus | ncbi.nlm.nih.gov/pmc/articles/PMC9721077/⁸ |
| III protein | | | GVSYSLCTAAFTFTKIPAETLHGTVTVEVQ |
| | | | YAGTDGPCKVPAQMAVDMQTLTPVGRLIT |
| | | | ANPVITESTENSKMMLELDPPFGDSYIVIGV |
| | | | GEKKITHHWHRSGSTIGKAFEATVRGAKR |
| | | | MAVLGDTAWDFGSVGGALNSLGKGIHQIF |
| | | | GAAFKS |
| Major surface | Viral | Human | >sp\|P03423\|GLYC_HRSVA Major surface |
| glycoprotein G | | respiratory | glycoprotein G OS = Human respiratory |
| | | syncytial | syncytial virus A (strain A2) OX = 11259 |
| | | virus A | GN = G PE = 1 SV = 1 |
| | | (RSV strain A2) | MSKNKDQRTAKTLERTWDTLNHLLFISSC |
| | | | LYKLNLKSVAQITLSILAMIISTSLIIAAIIFIA |
| | | | SANHKVTPTTAIIQDATSQIKNTTPTYLTQN |
| | | | PQLGISPSNPSEITSQITTILASTTPGVKSTLQ |
| | | | STTVKTKNTTTTQTQPSKPTTKQRQNKPPS |
| | | | KPNNDFHFEVFNFVPCSICSNNPTCWAICK |
| | | | RIPNKKPGKKTTTKPTKKPTLKTTKKDPKP |
| | | | QTTKSKEVPTTKPTEEPTINTTKTNIITTLLT |
| | | | SNTTGNPELTSQMETFHSTSSEGNPSPSQVS |
| | | | TTSEYPSQPSSPPNTPRQ |
| domain III | Viral | Dengue virus | pubmed.ncbi.nlm.nih.gov/17659815/⁹ |
| fragment of dengue | | type 2 | YSMCTGKFKVVKEIAETQHGTIVIRVQYEG |
| 2 envelope protein | | | DGSPCKIPFEIMDLEKRHVLGRLITVNPIVT |
| (D2EIII) | | | EKDSPVNIEAEPPFGDSYIIIGVEPGQLKLS |
| | | | WFKKGSSIGQMFETTMRGAKRMAILGDTA |
| | | | W |
| Merozoite surface | Protozoan | Plasmodium | >tr\|076244\|076244 PLAFA Merozoite surface |
| protein 4 (MSP4) | | falciparum | protein 4 OS = Plasmodium falciparum |
| | | | OX = 5833 GN = MSP4 PE= 4 SV = 1 |
| | | | MWIVKFLIVVHFFIICTINFDKLYISYSYNIV |
| | | | PENGRMLNMRILGEEKPNVDGVSTSNTPG |
| | | | GNESSSASPNLSDAAEKKDEKEASEQGEES |
| | | | HKKENSQESANGKDDVKEEKKTNEKKDD |
| | | | GKTDKVQEKVLEKSPKESQMVDDKKKTE |
| | | | AIPKKVVQPSSSNSGGHVGEEEDHNEGEGE |
| | | | HEEEEEHEEDDDDEDDDTYNKDDLEDEDL |
| | | | CKHNNGGCGDDKLCEYVGNRRVKCKCKE |
| | | | GYKLEGIECVELLSLASSSLNLIFNSFITIFV |
| | | | VILLIN |
| Merozoite surface | Protozoan | Plasmodium | >tr\|Q9U8B4\|Q9U8B4_PLAFA Merozoite |
| protein 5 (MSP5) | | falciparum | surface protein 5 OS = Plasmodium falciparum |
| | | | OX = 5833 GN = msp5 PE = 4 SV = 1 |
| | | | MNILCILSYIYFLVIFYSLNLNNKNENFLVV |
| | | | RRLMNDEKGEGGFTSKNKENGNNNRNNE |
| | | | NELKEEGSLPTKMNEKNSNSSDKQPNDISH |
| | | | DESKSNSNNSQNIQKEPEEKENSNPNLDSS |
| | | | ENSSESATRSVDISEHNSNNPETKEENGEEP |
| | | | LDLEINENAEIGQEPPNRLHFDNVDDEVPH |
| | | | YSALRYNKVEKNVTDEMLLYNMMSDQNR |
| | | | KSCAINNGGCSDDQICININNIGVKCICKDG |
| | | | YLLGTKCIILNSYSCHPFFSILIYITLFLLLFV |
| Trans-sialidase | Protozoan | Trypanosoma | >tr\|Q26966\|Q26966_TRYCR Trans-sialidase |
| | | cruzi | OS = Trypanosoma cruzi OX = 5693 PE = 1 |
| | | | SV = 1 |
| | | | MLAPGSSRVELFKRQSSKVPFEKDGKVTE |
| | | | RVVHSFRLPALVNVDGVMVAIADARYETS |
| | | | NDNSLIDTVAKYSVDDGETWETQIAIKNSR |
| | | | ASSVSRVVDPTVIVKGNKLYVLVGSYNSS |
| | | | GKITASIKWGSPVSLKEFFPAEMEGMHTNQ |
| | | | FLGGAGVAIVASNGNLVYPVQVTNKKKQ |
| | | | VFSKIFYSEDEGKTWKFGKGRSAFGCSEPV |
| | | | ALEWEGKLIINTRVDYRRRLVYESSDMGN |
| | | | SWLEAVGTLSRVWGPSPKSNQPGSQSSFT |
| | | | AVTIEGMRVMLFTHPLNFKGRWLRDRLNL |
| | | | WLTDNQRIYNVGQVSIGDENSAYSSVLYK |
| | | | DDKLYCLHEINSNEVYSLVFARLVGELRII |
| | | | KSVLQSWKNWDSHLSSICTPADPAASSSER |
| | | | GCGPAVTTVGLVGFLSHSATKTEWEDAYR |
| | | | CVNASTANAERVPNGLKFAGVGGGALWP |
| | | | VSQQGQNQRYRFANHAFTVVASVTIHEVP |
| | | | SVASPLLGASLDSSGGKKLLGLSYDERHQ |
| | | | WQPIYGSTPVTPTGSWEMGKRYHVVLTM |
| | | | ANKIGSEYIDGEPLEGSGQTVVPDERTPDIS |
| | | | HFYVGGYKRSDMPTISHVTVNNVLLYNRQ |
| | | | LNAEEIRTLFLSQDLIGTEAHMDSSSDTSA |
| A2 protein | Protozoan | Leishmania | >tr\|A4HZU7\|A4HZU7_LEIIN A2 protein |
| | | infantum | OS = Leishmania infantum OX = 5671 |
| | | | GN = LINJ_22_0670 PE = 4 SV = 1 |
| | | | MKIRSVRPL VVLL VCVAA VLAL SASAEPH |
| | | | KAAVDVGPLSVDVGPLSVGPQSVGPLSVG |
| | | | PQSVGPLSVDVGPLSVGPQSVGPLSVDVGP |
| | | | LSVGPQSVGPLSVGPQAVGPLSVGPQSVGP |
| | | | LSVDVGPQAVGPQSVGPLSVGPQSVGPLS |
| | | | VGPQSVGPLSVGPLSVGPQSVGSLSVGPQS |
| | | | VGPLSVGPLSVDVGPQAVGPLSVGPQAVG |
| | | | PLSVGPQSVGPLSVGPQSVGPLSVGPQSVG |
| | | | PLSVGPQSVGPLSVGPQSVGPLSVDVGPQS |
| | | | VGPLSVGPQSVGPLSVGPQSVGPLSVGPQS |
| | | | VGPLSVGPQSVGPLSVGPQSVGPLSVGPQA |
| | | | VGPLSVDVGPQSVGPLSVGPQAVGPLSVG |
| | | | PQSVGPLSVGPQSVGPLSVDVGQQSVGPLS |
| | | | VGPQSVGPLSVGPQSVGPLSVGPQAVGPLS |
| | | | VGPQAVGPLSVGPQAVGPLSVGPQSVGPL |
| | | | SVGPQAVGPLSVGPQAVGPLSVGPQSVGP |
| | | | LSVGLQAVDVSPVS |
| N-terminal portion | Fungal | Candida | pubmed.ncbi.nlm.nih.gov/16779733/₁₀ |
| of the Candida | | albicans | AKTITGVFNSFNSLTWSNAATYNYKGPGT |
| albicans | | | PTWNAVLGWSLDGTSASPGDTFTLNMPCV |
| agglutinin- | | | FKFTTSQTSVDLTAHGVKYATCQFQAGEE |
| like protein 3 | | | FMTFSTLTCTVSNTLTPSIKALGTVTLPLAF |
| (Als3p) | | | NVGGTGSSVDLEDSKCFTAGTNTVTFNDG |
| | | | GKKISINVDFERSNVDPKGYLTDSRVIPSLN |
| | | | KVSTLFVAPQCANGYTSGTMGFANTYGD |
| | | | VQIDCSNIHVGITKGLNDWNYPVSSESFSY |
| | | | TKTCSSNGIFITYKNVPAGYRPFVDAYISAT |
| | | | DVNSYTLSYANEYTCAGGYWQRAPFTLR |
| | | | WTGYRNSDAGSNGIVIVATTRTVTDSTTA |
| | | | VTTLPFDPNRDKTKTIEILKPIPTTTITTSYV |
| | | | GVTTSYSTKTAPIGETATVIVDIPYHTTTTV |
| | | | TSKWTGTITSTTTHTNPTDSIDTVIVQVP |

¹Santi et al. (2006). Protection conferred by recombinant Yersinia pestis antigens produced by a rapid and highly scalable plant expression system. Proceedings of the National Academy of Sciences of the United States of America, 103(4), 861-866. doi.org/10.1073/pnas.0510014103
²Jahantigh et al. (2022). The Candidate Antigens to Achieving an Effective Vaccine against Staphylococcus aureus. Vaccines, 10(2), 199, doi.org/10.3390/vaccines10020199
³Ghosh et al. (2011). An adhesion protein of Salmonella enterica serovar Typhi is required for pathogenesis and potential target for vaccine development. Proceedings of the National Academy of Sciences of the United States of America
⁴Choo et al., (1994). Vaccination of chimpanzees against infection by the hepatitis C virus. Proceedings of the National Academy of Sciences of the United States of America, 91(4), 1294-1298. doi.org/10.1073/pnas.91.4.1294
⁵Pogrebnyak et al. (2005). Severe acute respiratory syndrome (SARS) S protein production in plants: development of recombinant vaccine. Proceedings of the National Academy of Sciences of the United States of America, 102(25), 9062-9067, doi.org/10.1073/pnas.0503760102
⁶Golovkin et al. (2007). Smallpox subunit vaccine produced in Planta confers protection in mice. Proceedings of the National Academy of Sciences of the United States of America, 104(16), 6864-6869, doi.org/10.1073/pnas.0701451104
⁷Zhang et al. (2016). Phage Display-Derived Cross-Reactive Neutralizing Antibody against Enterovirus 71 and Coxsackievirus A16. Japanese journal of infectious diseases, 69(1), 66-74, doi.org/10.7883/yoken.JJID.2015.060
⁸Shin et al. (2022). Vaccination with a Zika virus envelope domain III protein induces neutralizing antibodies and partial protection against Asian genotype in immunocompetent mice. Tropical medicine and health, 50(1), 91, doi.org/10.1186/s41182-022-00485-6
⁹Saejung, et al. (2007). Production of dengue 2 envelope domain III in plant using TMV-based vector system. Vaccine, 25(36), 6646-6654, doi.org/10.1016/j.vaccine.2007.06.029
¹⁰Spellberg et al. (2006). Efficacy of the anti-Candida rAls3p-N or rAlslp-N vaccines against disseminated and mucosal candidiasis. The Journal of infectious diseases, 194(2), 256-260, doi.org/10.1086/504691

### 5. Immunomodulators

a. MEV-Mediated Intracellular Signaling and Other Receptors and Ligands for Preventing, Reducing the Risk of, or Treating a Disease, Disorder, or Condition

### MEV-Mediated Immunomodulation Via Toll-Like Receptor (TLR) Signaling

Toll-like receptors (TLRs) are a family of evolutionary conserved pattern recognition receptors, by which the immune system senses microbes via recognition of a wide range of microbial components. In mammals, 11 different TLRs have been described and most of them are widely expressed by different cell types in the immune system including dendritic cells (DCs), macrophages, NK cells, mast cells, neutrophils, B cells, T cells and by non-immune cells such as fibroblasts, epithelial cells and keratinocytes. Most TLRs (TLR 1, 2, 4, 5, 6, 10 and 11) are expressed on the cell surface, whereas other TLRs (TLR 3, 7, 8, 9) are present within the endosomal compartments. For example, on the cell surface TLR2 recognizes several bacterial and fungal cell wall components, TLR4 recognizes lipopolysaccharides (LPS) from most bacteria, and TLR5 recognizes flagellin. Intracellular receptors detect bacterial and viral nucleic acids. For example, TLR3 senses viral dsRNA, whereas TLR9 recognizes CpG motifs present in bacterial and viral DNA. MEVs are internalized into cells, into endosomes, which fuse with those bearing internalized TLRs; the MEVs release their cargo, such as TLR agonists or antagonists to interact with the TLRs, thereby modulating TLR-mediated responses to affect immune responses, including, but not limited to pro- and anti-inflammatory response, including immunostimulatory, such as antibacterial responses and anti-cancer responses, immunosuppressive responses, such as for treating autoimmune diseases, allergies, and other diseases, disorders, and conditions that involve the immune system.

Upon activation, TLRs propagate the pattern-induced signal transduction pathway leading to innate immune response, including cytokine production, cell proliferation (or apoptosis) and stimulation of phagocytosis. These initiate inflammatory responses and activate phagocytes such as neutrophils and macrophages. TLRs also represent an important link between innate and adaptive immunity through their presence in DCs, which play a central role in coordinated activation of innate and adaptive immune mechanisms triggered by pathogen-derived signals. Multiple ligands of TLRs were proved effective in augmenting adaptive immune responses. For example, combined activation of TLR3 and TLR7 signaling resulted in rapid DC activation, secretion of proinflammatory cytokines and expression of costimulatory molecules that were able to increase cytotoxic T-cells (CTL) effector functions. TLR ligands, like monophosphoryl lipid A (TLR4 agonist) or CpG motifs (TLR9 agonists), have been found to enhance immunogenicity of vaccine candidates. Moreover, the innate immune system (including TLR signaling) modulates the quantity and quality of long-term T and B cell memory and protective immune responses to pathogens. Activation of TLR4 receptors on B cells, and concomitant antigen stimulation of cognate B-cell receptors, induced antigen-specific IgG responses totally independent of T-cell help. TLR-regulated innate immunity can be effectively used to modulate both the cellular and humoral arms of adaptive immune response.

It is shown herein that administration of MEVs containing agonists or antagonists of TLRs can be administered, such as orally, and the effect of the agonist or antagonist will be manifested biologically demonstrating that MEVs can be delivered to target cells and can modulate activities of TLRs in those cells to manifest a response mediated by the cargo delivered by the MEV.

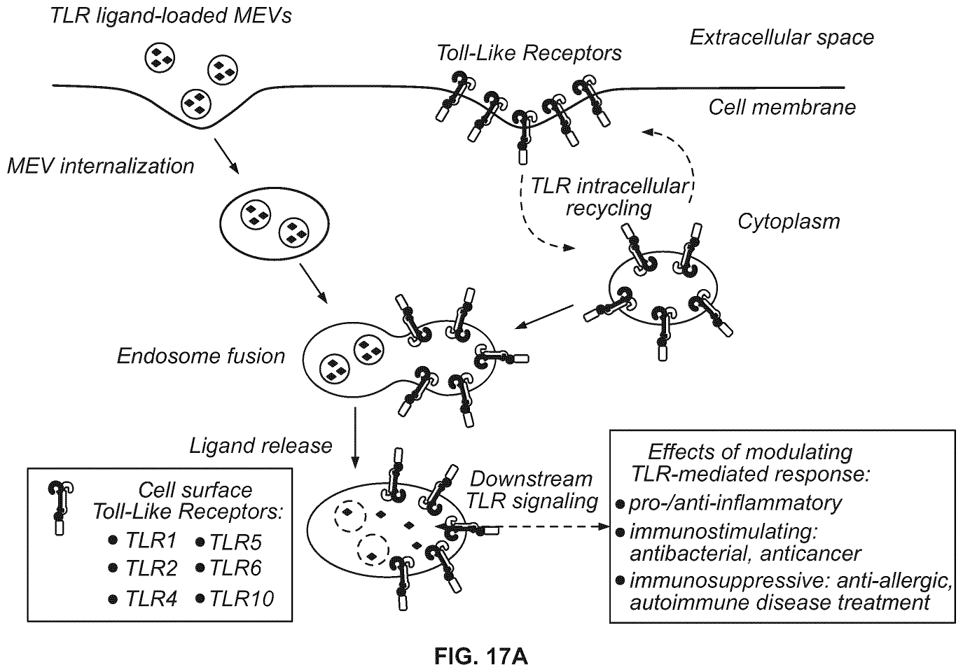

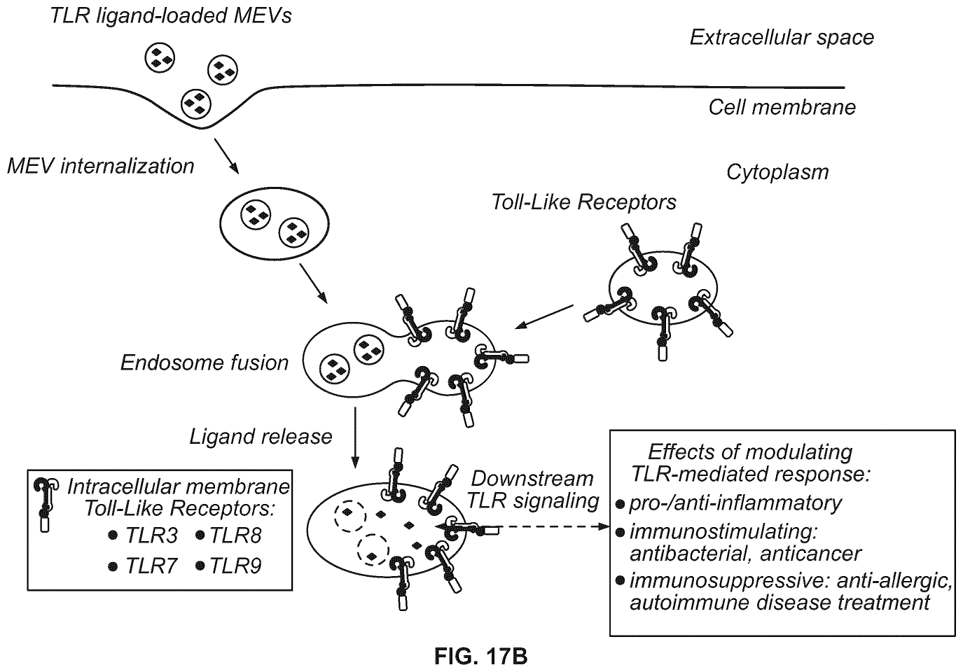

Uncontrolled regulation of TLR-mediated signaling can lead to excessive or persistent inflammation and severe immune pathologies. Several diseases including septic shock, autoimmunity, atherosclerosis, metabolic syndrome and gastric cancer have been linked to chronic or acute inflammatory response. Thus, TLR signaling pathway suppression also is a therapeutic approach for inhibiting disease-associated inflammation in disorders such as rheumatoid arthritis, sepsis, allergies, Alzheimer's disease, Parkinson's disease, inflammatory bowel diseases, ulcerative colitis, or Crohn's disease. MEVs loaded with TLR ligand(s), either agonists or antagonists, can be used for MEV-mediated immunomodulation via TLR signaling, immunostimulatory, such as vaccines, and immunosuppressive, such as anti-inflammatory, applications. See FIGS. 17A and 17B that depict MEVs and their interactions with TLRs and ligands.

In vivo therapeutic targets, in general, thus, include receptors that are involved in a disease, disorder, or condition. These include cell surface receptors, and internalized receptors, in which the MEVs deliver agonists, antagonists, ligands, or other modulators of activity. Targets of interest include, for example, modulation of toll-like receptors (TLRs) and other receptors, including receptors, such as the TLRs, that are internalized. Such receptors are internalized into vesicles and can interact with ligands delivered by MEVs. This is exemplified in the Examples in which the ligand flagellin is delivered into cells via MEVs; flagellin activates TLR-5, which in turn activates inflammatory cytokines.

Flagellin is a conserved protein, a component of the bacterial flagellum in mobile bacteria. The immune systems in plants and in animals that assure the defense against bacterial infections are set to recognize flagellin, or subrogate peptides thereof, and to react against the invading bacteria. Thus, the presence of bacteria, their flagella, or fragments thereof triggers a signaling pathway, that leads to a reaction of the host to the putative infection agent. In plants, flagellin is recognized by the FLS2 receptor (Flagellin Sensing 2 receptor). For instance, the presence of flagellin, or of surrogate peptides of it, is detected by the leaf epithelium that surrounds the stomata (the respiratory pores on the surface of the leaves). Open stomata are entry doors into the leaf parenchyma for infective agents such as flagellin-bearing bacteria. When the presence of flagellin is detected in the leaf epithelium, the plant triggers an immune response, which includes the immediate closing of the stomata in order to physically prevent the entry of bacteria thereby. The signaling pathway, that starts by the detection of flagellin (or of subrogate peptides) and ends with the closing of the stomata, is triggered by the binding of flagellin (or of subrogate peptides) to the FLS2 receptor. The transmembrane protein receptor, FLS2, is the very first component in the signaling pathway. In Arabidopsis thaliana, a species commonly used as a plant model, the FLS2 receptor is found in the plasma membrane and in the membranes of endosomal vesicles inside the plant cell (see, Beck et al., (2012) The Plant cell 24(10):4205-4219 and Otegui et al. (2008) Traffic (Copenhagen, Denmark) 9(10):1589-1598). The flagellin binding domains are oriented either to the “extracellular space” (for the FLS2 molecules located in the plasma membrane); or to the “intra-endosomal space” (for the FLS2 molecules located in intracellular endosomal vesicles). The FLS2 domains in charge of the triggering of the signaling pathway are in both cases oriented towards the cytoplasmic side of the membranes. A signaling pathway and the subsequent biological immune response triggered by the binding of flagellin (or of a surrogate peptide) to FLS2 are indistinguishable whether the triggering FLS2 is located in the plasma membrane and detects flagellin in the cell surface or located in an endosomal vesicle and detects flagellin from within the same endosomal vesicle. Although, it is straightforward for FLS2 located in the plasma membrane to identify and bind to flagellin (or to subrogate or surrogate peptides), it is less likely that FLS2 will find flagellin (or a subrogate or surrogate peptide) inside the endosomal vesicle where the intracellular FLS2 form is located.

Experiments shown in the working Examples herein, demonstrate that MEVs that have been exo-loaded with flp22, a subrogate or surrogate peptide of flagellin, can trigger the expected biological immune reaction, i.e., the immediate closing of the stomata, presumably by the delivery of the bioactive peptide straight into the endosomal vesicles where FLS2 is located. As flp22-loaded MEVs are treated with proteases to destroy any trace of external flp22 that remain from the exo-loading reaction; the immune reaction cannot be explained by free flp22 binding to the FLS2 in the plasma membrane. There is no evidence that MEVs (or other EVs) spontaneously release their cargo in the extracellular or culture media which might trigger a signaling pathway from the FLS2 in the plant's membrane. Thus, the observed data are difficult to explain unless the flp22-loaded MEVs are endocytosed by the epithelial plant cells, and that once inside the cells the endocytic vesicles carrying the loaded-MEVs can fuse with endosomal vesicles that carry the FLS2 protein.

Experiments described in the Examples show delivery of flg22 into A. thaliana cells via intracellular signaling that is mediated by MEVs. The flg22 peptide, is a 22-amino acid synthetic peptide, which mimics a conserved N-terminal region of bacterial flagellin. It has been shown that flp22 binds to plasma membrane FLS2 triggering a defense response of the cell against bacteria. But it had not been shown that free flp22 can be delivered directly to endosomal vesicles or that from there it can trigger the same biological response that it triggers from the cell surface. Effective delivery of the peptide inside the endosomal vesicles, mediated by peptide loaded-MEVs, allows such phenomenon to take place and to be observed.

In plants, this 22-amino acid synthetic peptide, mimicking a conserved N-terminal region of the bacterial flagellin, is bound by the surface receptor Flagellin Sensing 2 (FLS2) triggering a defense response. The signal upon ligand binding is transduced from the membrane receptor to intracellular FLS2 kinase and results in altering gene expression profile. Our findings indicate that flg22-loaded Chlorella MEVs are able to induce stomata closure. This response is triggered by the bioactive peptide present in the intact MEVs, while any remaining non-loaded flg22 is degraded with proteinase K treatment. Therefore, the FLS2 ligand is delivered into the host cells and is able to bind to the receptor inside the cell. This interaction can result from internalization of the surface receptor.

Multiple clathrin-independent mechanisms of endocytosis have been described and characterized; some of which play a role in membrane bulk flow and cell membrane turnover. It is also known that FLS2 can be internalized into vesicles, not only upon ligand binding, but also in a ligand-independent manner for constitutive recycling (see, Beck et al., (2012) The Plant cell 24(10):4205-4219). FLS2 signaling also occurs from endosomes after internalization (see, Otegui et al. (2008) Traffic (Copenhagen, Denmark) 9(10):1589-1598). Plant endosomes are highly dynamic organelles; hence a rendezvous of ligand-carrying MEVs and receptor-carrying vesicles is possible inside the cell. This intracellular interaction results in fusion of MEVs and endosomes, providing ligand-mediated activation of FLS2 and production of the effector signaling. Similar mechanisms of endosome turnover are present in yeast and mammals, where endosomes are also known to recycle vacuolar cargo receptors back to the trans Golgi network and sort membrane proteins for degradation in the vacuole/lysosome. FLS2 can be internalized into vesicles not only upon ligand binding, but also in a ligand-independent manner for constitutive recycling (Beck et al. (2012). Spatio-temporal cellular dynamics of the Arabidopsis flagellin receptor reveal activation status-dependent endosomal sorting. The Plant Cell, 24(10), 4205-4219). Moreover, FLS2 signaling can also occur from endosomes after internalization (Otegui et al. (2008). Endosomal functions in plants, Traffic (Copenhagen, Denmark), 9(10), 1589-1598). Plant endosomes are highly dynamic organelles, hence a rendezvous between MEVs and receptor-carrying vesicles can occur inside the cell. This intracellular interaction results in fusion of MEVs and endosomes, providing ligand-mediated activation of FLS2 and production of the effector signaling. Similar mechanisms of endosome turnover are present in yeast and mammals, where endosomes are also known to recycle vacuolar cargo receptors back to the trans Golgi network and sort membrane proteins for degradation in the vacuole/lysosome.

Cellular defense against invading pathogens relies on innate immune responses that are initiated by activation of pattern recognition receptors, which include Toll-like receptors (TLRs). These receptors also can be activated inadvertently by nucleic acid-based therapeutics, thereby hampering drug development. TLRs7/8/9 is a subfamily of structurally related endosomal receptors that respond to specific single-stranded nucleic acid molecules. TLR7 and TLR8 respond to RNA, and TLR9 responds to DNA.

Bacterial DNA is a potent immunostimulant and the abundance of CpG dinucleotides (CpGs) in genomes of prokaryotes is a factor contributing to its immunostimulatory properties. Toll-like receptor 9 (TLR9) is a major player in the innate immune response to bacterial DNA and synthetic oligodeoxynucleotides (ODNs) that contain unmethylated CpG motifs (see, Hemmi et al. A Toll-like receptor recognizes bacterial DNA. Nature. 2000, 408 (6813): 740-745).

The members of the TLR family are well-conserved type I transmembrane proteins characterized by a leucine-rich domain involved in ligand recognition and a toll/interleukin-1 receptor (TTR) intracellular signaling domain (see, Takeda et al. Toll-like receptors in innate immunity. Int Immunol. 2005, 17 (1): 1-14). Acting as innate immune receptors, members of this family mediate the response to different bacteria- and virus-derived molecules, including lipopolysaccharide, bacterial lipopeptides, double-stranded RNA and CpGs.

In mammals, the TLR9 occurs in the endoplasmic reticulum in resting immune cells. Upon exposure of the cells to CpG DNA, TLR9 translocates to the endosomal compartments (see, Latz et al., TLR9 signals after translocating from the ER to CpG DNA in the lysosome. Nat Immunol. 2004, 5 (2): 190-198), where it can interact with its ligand. Once activated, the receptor signals through Myeloid differentiation primary response gene 88 (MyD88) to activate transcription factors including nuclear factor kappa-B (NF-κB) and interferon-regulatory factor 3 (IRF3), and interferon-regulatory factor 7 (IRF7), each involved in the upregulation of proinflammatory genes and type I interferon (IFN), respectively (Kumagai et al. TLR9 as a key receptor for the recognition of DNA. Adv Drug Deliv Rev. 2008, 60 (7): 795-804).

TLR signaling is divided into two types of pathways: one of which is MyD88-dependent and the other MyD88-independent but Toll-interleukin-1 receptor (TIR)-domain-containing adaptor-inducing IFN-β (TRIF)-dependent. Downstream of the TLR signaling pathways, activated NF-κB and IRF (interferon (IFN) regulatory factor) control their target genes to produce an abundance of inflammatory cytokines and IFNs, which improve resistance to and clearance of pathogens from the body, and can also promote inflammation (see, e.g., Lu et al. Toll-like Receptors and Inflammatory Bowel Disease. Front Immunol. 9:72, 2018). Other functions of the latter factors are to upregulate the expression of related genes responsible for phagocytosis and the ability to enhance phagocytic function and to kill microbes. From these functions, TLR signaling recruits activated natural killer cells (NK cells) and DCs. DCs are prompted by TLRs to present antigens to T cells and initiate T cell responses, thus providing a bridge between innate immunity and adaptive immunity. TLRs and the signaling pathways also exist in T cells. Follicular helper T cells are produced abundantly in germinal centers and interact with B cells, a process that is also mediated by T cell intrinsic TLR signaling. TLRs additionally regulate B-cell responses for the purpose of producing monospecific IgM, IgG, and IgA antibodies, which are involved in adaptive immunity that can mediate mucosal homeostasis. TLRs and TLR-activated signaling pathways are involved not only in the pathogenesis but also in the efficacy of treatment of IBD and other inflammatory diseases.

TLRs have a role in the detection of microbial infection in mammals and insects, and also in pathways to effect treatment of diseases, disorders, and conditions. These roles can be exploited herein through delivery of ligands by the MEVs to modulate TLR activities.

In mammals, the TLRs recognize conserved products unique to microbial metabolism. This specificity allows the Toll proteins to detect the presence of infection and to induce activation of inflammatory and antimicrobial innate immune responses. Recognition of microbial products by Toll-like receptors expressed on dendritic cells triggers functional maturation of dendritic cells and leads to initiation of antigen-specific adaptive immune responses. It is shown herein that MEVs can deliver, such as via oral administration or inhalation or other routes to immune cells, agonists and/or antagonists of TLRs (or encoding nucleic acid), such as TLR9 and TLR3, and result in a biological response indicative of activation of the TLR-induced pathway or response or of inhibition of the TLR-induced pathway or response. Thus, MEVs are an ideal delivery vehicle for modulating the immune system via TLRs and other intracellular receptors.

There are similarities between how plants and animals perceive pathogens. Plants have highly sensitive perception systems that stimulate defense responses, but innate immunity in animals is based on the recognition of similar pathogen-associated molecular patterns. FLS2 in Arabidopsis, which is essential for flagellin perception, shares homology with the Toll-like receptor (TLR) family, which is a first line of defense against infectious diseases in animals (Gõmez-Gómez et al. (2002). Flagellin perception: a paradigm for innate immunity. Trends in Plant Science, 7(6), 251-256). TLR5 is responsible for flagellin perception in mammals, but other receptors from this family, including TLR1, TLR2, TLR3, TLR4 and TLR6, can also be considered as targets for intracellular MEV-mediated ligand delivery. This phenomenon indicates that there are number of therapeutic indications for TLR agonists. For example, immunomodulatory of TLR agonists can be used in infections (e.g., sepsis) and inflammation, (e.g., inflammatory bowel diseases, including ulcerative colitis and Crohn's disease, and rheumatoid arthritis), as part of or with a vaccine (including cancer vaccines) and for allergy treatment to enhance an immune response or modulate an immune response from the vaccine.

These results indicate that when a MEV loaded with a ligand (agonist or antagonist) for either an internalized receptor, or for an endosomal intracellular receptor, such as the FLS2 receptor, the TLRs or other such receptors, are internalized, as shown in the Examples, they reach and deliver their payload inside the vesicles where the target receptors are located and thus elicit a biological response from “within the cell”. Thus, MEVs can deliver agonists/antagonists or ligands to internalized or to intracellular receptors. No other alternative technologies have shown to be able of such an endeavor. See FIGS. 17A and 17B, discussed in the Examples.

Plant FLS2 protein is a member of the large family of the so-called TLRs (or Toll-like Receptors), which play a pivotal role in innate immunity and in the modulation and triggering of immune responses in humans, mammals, and other animals. There are similarities between how plants and animals perceive pathogens. Plants have highly sensitive perception systems that stimulate defense responses, and innate immunity in animals is based on the recognition of similar pathogen-associated molecular patterns. FLS2 in Arabidopsis, which is essential for flagellin perception, shares homology with the Toll-like receptor (TLR) family, which is a first line of defense against infectious diseases in animals (see, Gómez-Gómez et al. (2002) Trends in Plant Science 7(6):251-256).

TLR5 is responsible for flagellin perception in mammals, but other receptors from this family, including TLR3, TLR7, TLR8, TLR9, TLR13, (naturally located in cellular endocytic vesicles) also can be targets for intracellular MEV-mediated ligand delivery. This phenomenon paves the way for treatment of a number of therapeutic indications using specific ligand-loaded MEVs to trigger the immunomodulatory signaling pathways related to the different TLR family members; such as for infections (like sepsis and other), for inflammation (like rheumatoid arthritis and other), to enhance or modulate the immune response to a vaccine (including cancer vaccines) as well as for allergy treatment. The following tables summarize TLR members, their ligands and agonists/antagonists, and downstream effects:

| TLR | | |
|---|---|---|
| Member | Ligand(s)/Agonists | Actions/Downstream effects |
| TLR1 | Triacyl lipopeptides | Myeloid differentiation primary response |
| | (Pam3CSK4) | (MyD88)* and MAL/TIRAP**-dependent |
| | | activation of inflammatory cytokines, NF-KB and |
| | | AP-1; Induces phagocytosis of ligand, or bound |
| | | molecules |
| TLR2 | Zymosan, Porin, Modulin, | MyD88 and MAL/TIRAP-dependent activation of |
| | Lipoproteins, Lipotechoic acid, | inflammatory cytokines, NF-KB and AP-1; |
| | Diacyl lipopeptides, Atypical | Induces phagocytosis of ligand, or bound |
| | LPS, Peptidoglycan, Triacyl | molecules |
| | lipopeptides | |
| TLR3 | dsRNA | Trif dependent induction of Inflammatory |
| | | cytokines and Type I IFN; Initiates adaptive |
| | | immunity; Activates local cytokine burst and local |
| | | inflammatory response; Production of TNF-alpha, |
| | | IL-12, MCP1. |
| TLR4 | Mannans, Taxol, LPS | Trif and TRAM dependent induction of |
| | | inflammatory cytokines and Type -I IFN, TIRAP |
| | | and MyD88 induction of inflammatory cytokines; |
| | | Promotes addiction; Neuroinflammatory response |
| TLR5 | bacterial flagellin, profilin, | MyD88-dependent induction of NF-kB and |
| | HMGB1, Small molecule | inflammatory cytokines, activation of innate and |
| | agonists (CBLB502) | adaptive immunity |
| TLR6 | Zymosan, Porin, Modulin, | MyD88 and MAL/TIRAP-dependent activation of |
| | Lipoproteins, Lipoteichoic acid, | inflammatory cytokines, NF-KB and AP-1; |
| | Diacyl lipopeptides | Induces phagocytosis of ligand, or bound |
| | (Pam2CSK4), Atypical LPS, | molecules |
| | Peptidoglycan | |
| TLR7 | imidazoquinolinone, loxoribine, | MyD88 dependent induction of inflammatory |
| | ssRNA, bropirimine, resiquimod | cytokines and Type I IFN |
| TLR8 | ssRNA, small synthetic | MyD88 dependent induction of inflammatory |
| | compounds | cytokines and Type I IFN |
| TLR9 | CpG DNA | MyD88 dependent induction of inflammatory |
| | | cytokines and Type I IFN |
| TLR10 | Diacyl and Triacyl lipopeptides | TRIF-dependent activation of IFN-B; TRADD |
| | | dependent and/or MyD88 and TIRAP-dependent |
| | | activation of pro-inflammatory cytokines; |
| | | PI3K/Akt dependent activation of IL-1Ra; Direct |
| | | inhibition of MyD88 or MAPK--Anti- |
| | | inflammatory; |
| TLR11 | Profilin-like protein, non- | MyD88 dependent induction of inflammatory |
| | pathogenic bacteria | cytokines |

*TIR-containing adaptor protein (Tirap), also called MyD88 adaptor-like protein (MAL)
**toll/Interleukin-1 receptor domain containing adaptor protein

| TLR | | |
|---|---|---|
| Member | Ligand(s)/Antagonists | Action/Downstream Effects |
| TLR1 | Small molecule antagonists (CU- | Competitive inhibition of signaling cascade; |
| | T12-9, MMG-11) | Inhibition heterodimerization of TLR1/2; |
| | | Induce M1 macrophages to M2 phenotype; |
| | | Inhibition of immune response; |
| | | Inhibition of inflammatory response |
| TLR2 | Small Molecule Antagonists | Competitive inhibition of signaling cascade; |
| | (AT1-AT8, CU-CPT22, CU- | Inhibition of heterodimerization of TLR1/2; |
| | T12-9, MMG-11, NPT1220- | Induction of M1 macrophages to M2 phenotype; |
| | 312), Phloretin, Sulfoglycolipids | Reduction of immune response; |
| | | Reduction of inflammatory Response; |
| | | Reduction of TNF-alpha production, reduced nitric |
| | | oxide (NO) formation; Reduced TLR2-induced |
| | | NF-KB transcriptional activity; |
| | | Reduction of NLRP3 including IL-1B, and 1L-18 |
| TLR3 | Small Molecule Antagonists | Decreased production of IL-6, IL-8, MIP1-alpha, |
| | (CU-CPT4a), Monoclonal | and TNF-alpha |
| | antibodies (CNTO4685, | |
| | CNTO5429) | |
| TLR4 | Small Molecule Antagonists | Decreased NF-kB activation and cytokine |
| | (Norbinaltorphimine, T4ICs, | production, reduction of nitric oxide (NO) |
| | T5342126, Simvastatin | formation; Decreased addiction |
| TLR5 | Small Molecule Antagonist | Decreased NF-kB, AP-1 signaling and decreased |
| | (TH1020) | induction of proinflammatory cytokines; Altered |
| | | gut microbiome, tumor growth, metabolic |
| | | syndrome, liver fibrosis |
| TLR6 | Simvastatin | Reduction in cytokine production; Prevention of |
| | | macrophage activation |
| TLR7 | Chloroquine, | Reduction of IFN-1 and cytokine signaling, |
| | hydroxychloroquine, quinacrine | reduction of inflammation |
| TLR8 | Small Molecule Antagonist (CU- | Reduced induction of TNF, IL-6, and IL-10 |
| | CPT8m, CU-CPT9a) | |
| TLR9 | Small Molecule Antagonists | Blocks release of inflammatory cytokines in |
| | (NPT1220-312), chloroquine, | monocyte/macrophage cells; decreased |
| | hydroxychloroquine, quinacrine; | immunostimulatory response |
| | Suppressive or inhibitory | |
| | oligonucleotides | |
| TLR10 | — | — |

### Cytokines and Chemokines, Co-Stimulatory Molecules, and Others

Immune modulators can be delivered as cargo in the MEVs, and/or combined with antigens. Immunomodulators include cytokines and chemokines, which modulate immune responses. Some consider chemokines to be a subset of cytokines. Chemokines, include interleukins, and interferons. The immune modulators include the cytokines or chemokines, or molecules that upregulate or downregulate expression of the cytokines and/or chemokines in a treated subject.

### Cytokines

Interleukin-2 (IL-2) is implicated in the activation of the immune system by several mechanisms, including the activation and promotion of cytotoxic T lymphocyte (CTL) growth, the generation of lymphokine-activated killer (LAK) cells, the promotion of regulatory T-cell (Treg cell) growth and proliferation, the stimulation of tumor-infiltrating lymphocytes (TILs), and the promotion of T-cell, B cell, and NK cell proliferation and differentiation. IL-7, which is a member of the IL-2 superfamily, is implicated in the survival, proliferation, and homeostasis of T-cells. IL-7 is a homeostatic cytokine that provides continuous signals to resting naïve and memory T-cells, and which accumulates during conditions of lymphopenia, leading to an increase in both T-cell proliferation and T-cell repertoire diversity. In comparison to IL-2, IL-7 is selective for expanding CD8⁺ T-cells over CD4⁺FOXP3⁺ regulatory T-cells. Recombinant IL-7 can augment antigen-specific T-cell responses following vaccination and adoptive cell therapy in mice. IL-7 has antitumor effects in tumors, such as gliomas, melanomas, lymphomas, leukemia, prostate cancer, and glioblastomas, and the in vivo administration of IL-7 in murine models resulted in decreased cancer cell growth, and can enhance the antitumor effects of IFN-γ. IL-12, and forms thereof, is secreted by antigen-presenting cells, promotes the secretion of IFN-7 from NK and T-cells, inhibits tumor angiogenesis, results in the activation and proliferation of NK cells, CD8⁺ T-cells, and CD4⁺ T-cells, enhances the differentiation of naïve CD4⁺ T-cells into Th1 cells, and promotes antibody-dependent cell-mediated cytotoxicity (ADCC) against tumor cells. IL-15 and forms thereof, provides stimulation for the proliferation and activation of T-cells, IL-15 blocks IL-2 induced apoptosis, which is a process that leads to the elimination of stimulated T-cells and the induction of T-cell tolerance, limiting memory T-cell responses, and potentially limiting the therapeutic efficacy of IL-2 alone. IL-18 induces the secretion of IFN-7 by NK and CD8⁺ T-cells, enhancing their toxicity. IL-18 also activates macrophages and stimulates the development of Th1 helper CD4⁺ T-cells.

Cytokines for delivery include, but are not limited to, any used therapeutically, such as for example, IL-15, IL-15/IL-15R alpha chain complex, IL-12, and others, type I interferon (IFN), including IFN-alpha, and IFN-beta, interferon-gamma, and others.

### Chemokines

Chemokines are a family of small cytokines that mediate leukocyte migration to areas of injury or inflammation, and that are involved in mediating immune and inflammatory responses. Chemokines are classified into four subfamilies, based on the position of cysteine residues in their sequences, namely XC-, CC-, CXC-, and CX3C-chemokine ligands, or XCL, CCL, CXCL, and CX3CL. The chemokine ligands bind to their cognate receptors and regulate the circulation, homing, and retention of immune cells, with each chemokine ligand-receptor pair selectively regulating a certain type of immune cell. Different chemokines attract different leukocyte populations, and form a concentration gradient in vivo, with attracted immune cells moving through the gradient towards the higher concentration of chemokine (see, e.g., Argyle D. and Kitamura, T. (2018) Front. Immunol. 9:2629; and Dubinett et al. (2010) Cancer J. 16(4):325-335). Chemokines can improve the anti-tumor immune response by increasing the infiltration of immune cells into the tumor, and facilitating the movement of antigen-presenting cells (APCs) to tumor-draining lymph nodes, which primes naïve T-cells and B cells (see, e.g., Lechner et al. (2011) Immunotherapy 3(11):1317-1340). The immunostimulatory bacteria provided herein can be engineered to encode chemokines or deliver cytokines or active forms thereof. Chemokines, include, but are not limited to CCL3, CCL4, CCL5, CXCL9, CXCL10, and CXCL 11. CCL3, CCL4, and CCL5 share a high degree of homology, and bind to CCR5 (CCL3, CCL4, and CCL5) and CCR1 (CCL3 and CCL5) on several cell types, including immature DCs and T-cells. The induction of the T helper cell type 1 (Th1) response releases CCL3. CCL3 and CCL4 play a role in directing CD8⁺ T-cell infiltration into primary tumor sites in melanoma and colon cancers. The binding of CCL3 or CCL5 to their receptors (CCR1 and CCR5), moves immature DCs, monocytes, and memory and T effector cells from the circulation into sites of inflammation or infection. For example, CCL5 expression in colorectal tumors contributes to T lymphocyte chemoattraction and survival. CXCL9 (MIG), CXCL10 (IP10), and CXCL11 (ITAC) are induced by the production of IFN-7. These chemokines bind CXCR3, preferentially expressed on activated T-cells, and function angiostatically, and in the recruitment and activation of leukocytes. In vivo, CXCL9 functions as a chemoattractant for tumor-infiltrating lymphocytes (TILs), activated peripheral blood lymphocytes, natural killer (NK) cells, and Th1 lymphocytes. CXCL10, produced by activated monocytes, fibroblasts, endothelial cells and keratinocytes, is chemotactic for activated T-cells, and can act as an inhibitor of angiogenesis in vivo. CXCL10/11 and CXCR3 expression has been established in human keratinocytes derived from basal cell carcinomas (BCCs). CXCL11 also is capable of promoting immunosuppressive indoleamine 2,3-dioxygenase (IDO) expression in human basal cell carcinoma, as well as enhancing keratinocyte proliferation, which could reduce the anti-tumor activity of any infiltrating CXCR3⁺ effector T-cells (see, e.g., Kuo et al. (2018) Front. Med. (Lausanne) 5:271).

### Co-Stimulatory Molecules

Co-stimulatory molecules enhance the immune response against tumor cells, and co-stimulatory pathways are inhibited by tumor cells to promote tumorigenesis. Exemplary of co-stimulatory molecules are CD40, CD40L, 4-1BB, 4-1BBL, 4-1BBL, CD80, CD86, CD27L, B7RP1, OX40L, and CD28. CD28 is a co-stimulatory molecule expressed on the surface of T-cells that acts as a receptor for B7-1 (CD80) and B7-2 (CD86), which are co-stimulatory molecules expressed on antigen-presenting cells. CD28-B7 signaling is required for T-cell activation and survival, and for the prevention of T-cell anergy, and results in the production of interleukins, such as IL-6.

### TNF Receptor Superfamily

The TNF superfamily of ligands (TNFSF) and their receptors (TNFRSF) are involved in the proliferation, differentiation, activation and survival of tumor and immune effector cells. Members of this family include CD30, Fas-L, TRAIL-R, and TNF-R, which induce apoptosis, and CD27, OX40L, CD40L, GITR-L, and 4-1BBL, which regulate B and T-cell immune responses. Other members include herpesvirus entry mediator (HVEM). CD40, which is a member of the TNF receptor superfamily, is expressed by APCs and B cells, while its ligand, CD40L (CD154), is expressed by activated T-cells. Interaction between CD40 and CD40L stimulates B cells to produce cytokines, resulting in T-cell activation and tumor cell death. Studies have shown that anti-tumor immune responses are impaired with reduced expression of CD40L on T-cells or CD40 on dendritic cells.

Therapeutic targets for MEV-mediated delivery and expression, include receptors involved in diseases, disorders, or conditions, such that said receptors are cell surface receptors, and internalized receptors or internal (intracellular endosomal) receptors, such TLRs and others. MEVs may deliver agonists, antagonists, ligands, or other modulators of activity of such receptors. Targets of interest include, for example, modulation of toll-like receptors (TLRs). Such receptors are internalized into endosomal vesicles and can interact with ligands delivered by MEVs. This is exemplified in the Examples in which flagellin, surrogates thereof, and other known ligands for TLRs are delivered into cells via MEVs; activation of TLRs in turn activates inflammatory cytokines.

These can be provided in MEVs in combination with vaccines, such as antigens or epitopes, that elicit an immune response to protect against, or treat a disease, disorder, or condition. These receptors and other immune modulators can be introduced into the same MEVs, or can be provided in other MEVs, which can be administered with the vaccine MEVs together, in the same composition, or in separate compositions, or intermittently, depending upon a particular vaccination or treatment regimen. MEVs carrying different cargo can be co-formulated, or provided separately.

### Other Immunomodulators

Those of skill in the art are familiar with immunomodulators, which include, but are not limited to, checkpoint inhibitors, such as inhibitors of PD-1, PD-L1, and CTLA4. The cargo can comprise checkpoint inhibitor antibodies, generally single chain antibodies and antigen-binding fragments thereof, and other immunomodulatory antibodies and fragments thereof.

### I. FORMULATIONS, ROUTES OF ADMINISTRATION, AND DISEASE AND DISORDERS

Provided are compositions containing the MEVs in an amount suitable for effecting treatment for a particular disease or disorder. The amount can depend upon the therapeutic cargo, the disease, or disorder, and the subject treated. It is within the level of skill in the art to ascertain a particular dosage of MEVs. Formulations include any known to those of skill and include, for example:
injectables for intravenous administration, to reach the liver and the spleen;
oral, such as, for example tablets, capsules, films, and troches;
drops for per os administration, to reach the intestine, such as a vaccine, the immune system (immune cells), and the spleen;
compositions, such as emulsions (microemulsions and nanoemulsions) for inhalation, such for intratracheal, intrapulmonary administration; to reach the lungs;
drops for intranasal administration; and
formulations, such as creams, oils, gels, lotions, ointments for the skin and the mucosa.

Provided are pharmaceutical compositions containing, in a pharmaceutically acceptable vehicle microalgae extracellular vesicles (MEVs). The MEVs can contain an agent, generally a therapeutic or biologically active agent, such as nucleic acid, particularly an RNA, a protein, a small molecule, and other such agents. The compositions contain an amount of the MEV that can be diluted to deliver a therapeutically effective amount of the agent, or are formulated for direct administration without dilution. The particular concentration of MEVs depends upon a variety of parameters within the skill of a skilled artisan, including, for example, the treated indication; the active agent; the route of administration; the disease, disorder, or condition to be treated; and the regimen. Routes of administration include systemic and local routes, oral, rectal, intravenous, intramuscular, subcutaneous, mucosal, inhalation, nasal, eye, peritoneal, intratracheal, intravitreal, vaginal, and any suitable route known to the skilled person.

Pharmaceutical carriers or vehicles suitable for administration of the compounds provided herein include any such carriers known to those skilled in the art to be suitable for the particular mode of administration.

### Exemplary Formulations

Pharmaceutical compositions containing the MEVs can be formulated in any conventional manner, by mixing a selected amount of the active compound with one or more physiologically acceptable carriers or excipients. Selection of the carrier or excipient is within the skill of the administering professional, and can depend upon a number of parameters. These include, for example, the mode of administration (i.e., systemic, oral, nasal, pulmonary, local, topical, or any other mode), and the disorder treated. The formulations also can be co-formulations with other active agents for combination therapy.

A selected amount of MEVs are formulated in a suitable vehicle for administration by a selected route. The pharmaceutical compositions can be formulated in any conventional manner, by mixing a selected amount of MEVs with one or more physiologically acceptable carriers or excipients or vehicles. The pharmaceutical composition can be used for therapeutic, prophylactic, cosmetic and/or diagnostic applications. The concentration of the MEVs in a composition, depends on a variety of factors, including those noted above, as well as the absorption, inactivation, and excretion rates of the active agent cargo, the release of the cargo, the mechanism of release, the dosage schedule, and the amount administered, the age and size of the subject, as well as other factors known to those of skill in the art, and related to the properties of the MEVs.

The pharmaceutical compositions provided herein can be in various forms, such as, but not limited to, in solid, semi-solid, liquid, emulsions, powder, aqueous, and lyophilized forms. The pharmaceutical compositions provided herein can be formulated for single dosage (direct) administration, or for dilution, or other regimen. The concentrations of the compounds in the formulations are effective, either following dilution or mixing with another composition, or for direct administration, for delivery of an amount, upon administration, that is effective for the intended treatment. The compositions can be formulated in an amount for single or multiple dosage direct administration. The form of composition depends a variety of factors, including the intended mode of administration. The resulting mixtures are solutions, suspensions, emulsions and other such mixtures, and can be formulated as creams, gels, ointments, emulsions, solutions, lotions, suspensions, tinctures, pastes, foams, aerosols, irrigations, and sprays. For oral administration, the MEVs can be formulated as tablets, capsules, lozenges, liquids, and others.

For local internal administration, such as intramuscular, parenteral or intra-articular administration, the MEVs can be formulated in isotonically buffered saline. The effective concentration of the MEVs is sufficient to provide a sufficient amount of the cargo agent for the intended purpose, and can be empirically determined.

Generally, pharmaceutically acceptable compositions are prepared in view of approvals for a regulatory agency, or other agency, and/or are prepared in accordance with generally recognized pharmacopeia for use in animals and in humans. Pharmaceutical compositions can include a carrier, such as a diluent, adjuvant, excipient, or vehicle, with which a polypeptide is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain, along with an active ingredient, a diluent, such as lactose, sucrose, dicalcium phosphate, and carboxymethylcellulose; a lubricant, such as magnesium stearate, calcium stearate and talc; and a binder, such as starch, natural gums, such as gum acacia, gelatin, glucose, molasses, polyvinylpyrrolidone, celluloses and derivatives thereof, povidone, crospovidone, and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, and ethanol. A composition, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents. These compositions can take the form of solutions, suspensions, emulsions, tablets, pills, capsules, powders, granules, and sustained release formulations. Capsules and cartridges of, e.g., gelatin, for use in an inhaler or insufflator, can be formulated containing a powder mix of a therapeutic compound and a suitable powder base, such as lactose or starch. A composition can be formulated as a suppository, with traditional binders and carriers, such as triglycerides. Oral formulations can include standard carriers, such as pharmaceutical grades of mannitol, lactose, starch, magnesium stearate, sodium saccharine, cellulose, magnesium carbonate, and other such agents. Preparations for oral administration also can be suitably formulated with protease inhibitors, such as a Bowman-Birk inhibitor, a conjugated Bowman-Birk inhibitor, aprotinin and camostat. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier, so as to provide the compound in a form for proper administration to a subject or patient.

The pharmaceutical compositions provided herein can contain other additives, including, for example, antioxidants, preservatives, antimicrobial agents, analgesic agents, binders, disintegrants, colorings, diluents, excipients, extenders, glidants, solubilizers, stabilizers, tonicity agents, vehicles, viscosity agents, flavoring agents, emulsions, such as oil-in-water or water-in-oil emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol-9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as crystalline cellulose, microcrystalline cellulose, citric acid, dextrin, dextrose, liquid glucose, lactic acid, lactose, magnesium chloride, potassium metaphosphate, and starch, among others (see, generally, Alfonso R. Gennaro (2000) Remington: The Science andPractice of Pharmacy, 20th Edition. Baltimore, MD: Lippincott Williams & Wilkins). Such carriers and/or additives can be formulated by conventional methods and can be administered to the subject at a suitable dose. Stabilizing agents, such as lipids, nuclease inhibitors, polymers, and chelating agents, can preserve the compositions from degradation within the body.

The formulation should suit the mode of administration. For example, the MEVs can be formulated for parenteral administration by injection (e.g., by bolus injection, or continuous infusion). The injectable compositions can take such forms as suspensions, solutions or emulsions in oily or aqueous vehicles. The sterile injectable preparation also can be a sterile injectable solution, or a suspension in a non-toxic parenterally-acceptable diluent or solvent, for example, as a solution in 1,4-butanediol. Sterile, fixed oils are conventionally employed as a solvent or suspending medium. For this purpose, any bland fixed oil can be employed, including, but not limited to, synthetic mono- or diglycerides, fatty acids (including oleic acid), naturally occurring vegetable oils, such as sesame oil, coconut oil, peanut oil, cottonseed oil, and other oils, or synthetic fatty vehicles like ethyl oleate. Buffers, preservatives, antioxidants, and the suitable ingredients, can be incorporated as required, or, alternatively, can comprise the formulation. The MEVs provided herein, can be formulated as a pharmaceutically active ingredient in the composition, or can be combined with other active ingredients. Suspension of the MEVs can be suitable for administration. These can be prepared according to methods known to those skilled in the art.

Suitable compositions for inhalation, including into the nose, administration include but are not limited to, powders, sprays, liquids, suspensions, emulsions, and any other form that can be administered directly to the nose and that can contain the MEVs. The concentration of MEVs can be empirically determined, and depends upon the cargo, the indication treated, or intended use.

The therapeutically effective concentration of the MEVs can be determined empirically by testing the compounds in known in vitro and in vivo systems Determination of a therapeutically effective amount is well within the capability of those skilled in the art.

### J. EXAMPLES

The following examples are included for illustrative purposes only and are not intended to limit the scope of the invention(s).

### Example 1

### Production of Chlorella Cells and Isolation of MEVs

### A. Batch Production of the Inoculum

Chlorella vulgaris of any strain can be used to produce MEVs. Exemplary strains include, but are not limited to, UTEX 265 strain, UTEX 395 strain, UTEX 26 strain, 15 UTEX 30 strain, UTEX 259 strain, UTEX 2219 strain, UTEX 2714 strain UTEX B 1811 (available from the UTEX Culture Collection), the strain designated CCAP 211/19, GEPEA, University of Nantes, France, and any other suitable strain, either transformed or not, can be used to produce the algal cell material. For exemplary purposes, the UTEX 265 strain was used.

Chlorella was stored on nutrient agar slopes until flask/photobioreactor (PBR) inoculation. For different experiments, different scales of production, between 400 mL (flasks) to 170 L (several PBRs with different total volume) cultures, were used. This description relates to the highest volume of PBR used (170 L, HECTOR PBR “Hector” photobioreactor designed by the Laboratory of Process Engineering—Environment—Agri-food (GEPEA)/CNRS for the culture of microalgae. Reference: 20160067_0017. Year of production: 2016. Maximum size: 56.43×37.66 cm/170 L/300 dpi]).

A 5-Liter PBR was filled with 4 L of sterile BG11 medium (see, e.g., utex.org/products/bg-11-medium for a description of its preparation) and inoculated directly from the stock algal slope on nutrient agar. Then, the Chlorella strain was grown as a batch culture in a bubble column using the following culture parameters: temperature of 23° C.; medium pH 7.5-8.0; light intensity: 100 μmol·m⁻²·s⁻¹; light cycle: continuous. Biomass concentration, specific growth rate and biomass productivity of Chlorella were estimated daily. Typically, after 6 days of continuous growth the cultures reached biomass concentrations of approx. 1.2 g/L. The total crop volume of 20 L was collected for subsequent production scale-up to the HECTOR PBR.

### B. Production Scale-Up in a Semi-Industrial Photobioreactor (PBR)

The Chlorella cells were cultured further in a 170-Liter photobioreactor system (HECTOR). The inoculum was added to sterile BG11 medium to the total volume of 150 L and the cells were grown autotrophically as a semi-batch culture with bubble column mixing. The following culture parameters were used: temperature of 18±4° C.; medium pH 8.0±0.05; light intensity: 150-300 μmol m⁻²·s⁻¹; between 150 μmol·m⁻²·s⁻¹ the three first days of each batch, 250 μmol·m⁻²·s⁻¹ days four and five and 300 μmol·m⁻²·s⁻¹ days six and seven before the harvesting as light cycle: continuous, with gradual increase in light intensity.

Biomass concentration, growth rate and biomass productivity of Chlorella were estimated daily. On the 6^{th} day of the cultivation, at the biomass concentration of approx. 1.5 g/L Chlorella harvesting was performed.

### C. Production of 3 Consecutive Batches of Chlorella

The Chlorella production was performed in 3 semi-batches of 130 L, from which about 80% of the culture volume was aseptically removed for downstream treatment and supplemented with sterile BG11 medium. Following the harvest, the light intensity was lowered to 140 μmol m⁻²·s⁻¹ to avoid excessive photon intake. A seeding line was set up to go from 100 mL of culture to 150 L of culture. Three consecutive batches lasting 6-7 days were carried out with the aim of extracting a vesicle concentrate devoid of microalgae.

### Culture Parameters Monitoring

### 1. Determination of the Protein Content

The protein content of cultures was determined by elemental analysis, resorting to Vario el III (Vario EL, Elementar Analyser systeme, GmbH, Hanau, Germany), according to the procedure provided by the manufacturer. The final protein content was calculated by multiplying the percentage of nitrogen given by the elemental analysis by 6.25.

### 2. Estimation of Chlorophyll Content

Culture samples were centrifuged at 2547 g for 15 min using a Hermle centrifuge (HERMLE Labortechnik GmbH, Wehingen, Germany). Pigments were extracted from the resulting pellet by bead milling in acetone. The full absorbance spectrum of the extract was obtained with a Genesys™ 10S UV-VIS spectrophotometer (Thermo Fisher Scientific, Massachusetts, USA) and iteratively decomposed to the standard pigment spectra to obtain the total chlorophyll content.

### 3. Growth Estimation

Dry weight was obtained by filtration of culture samples using pre-weighed 0.7 μm GF/C 698 filters (VWR, Pennsylvania, USA) and dried at 120° C. until constant mass was obtained using a DBS 60-30 electronic moisture analyser (KERN & SOHN GmbH, Balingen, Germany). All dry weight samples were washed with demineralized water to remove growth medium salts.

### D. Isolation of Microalgae Extracellular Vesicles: Production of Concentrated MEV Preparation (Down-Stream Processing: Clarification and Concentration Step)

The culture harvested from the photobioreactor was centrifuged at 2,700 g for 5 minutes at room temperature for cell removal. The supernatant was transferred into fresh bottles and centrifuged again at 2,700 g for 5 minutes at room temperature. The clear MEV-containing solution was then subjected to membrane filtering using a 1.2 μm cut-off cartridge filter. The filtrate was concentrated with the use of a 100 kDa MWCO tangential filtration system. At each isolation step the material was analyzed spectrophotometrically for chlorophyll and particulate matter. Dry weight of the final product was <0.01 g/L and the concentration factor relative to the initial volume of the processed culture was approx. 20. The suspension of MEV thus obtained was stored at −50° C. in 1-1.2 L pockets for further purification.

### E. Purification of Chlorella Microalgae Extracellular Vesicles

1. Thaw in a cold room at 4° C., overnight, the preparation of MEVs previously clarified, concentrated, and stored (1.0-1.2 L) as described in section D.
2. When the preparation is thawed, harvest the biomass by centrifugation (set the temperature to 4° C.): 2×10000 g for 10 minutes at 4° C.
3. Collect the supernatant (MEVs) and filter by vacuum filter onto 0.65 μm filters to get rid of the remaining cells.
4. The MEVs are concentrated and purified by tangential flow filtration (TFF) using Sartorius VivaFlow® filtration systems.
   a. The membrane is washed by running water at ˜100 ml/minute, as described by the manufacturer. After that, the circuit is washed with cell-free medium (BG-11 medium) at ˜200 ml/minute (pressure reading at 2/2,5 bars).
   b. The MEV preparation (supernatant) is run in the circuit at ˜200 ml/minute (pressure reading at 2/2,5 bars).
   When the residual volume in the circuit plus the reservoir is about 200 mL, the TFF is used to diafiltrate and change the medium from BG-11 to PBS using 1L of PBS.
   c. When the residual volume in the circuit plus the reservoir is about 200 ml in PBS, slow the flow to ˜100 ml/minute (20 minutes, 1 bar).
   d. From 30-60 ml of residual volume, slow the flow to a speed lower than 50 ml/minutes and allow the MEVs in PBS to recirculate for 30 minutes to recover the particles trapped on the membrane surface.
   e. MEVs are then filtered using 0.45 μm filters and purified by ultracentrifugation.
   f. The filtered MEVs are loaded on the ultracentrifuge tubes and centrifuged for 1 h at 4° C., at 100000 g (27400 rpm) (acceleration and deceleration at 10 max), for example in a SorVall™ WX ultra 80 TST 28.38. Pellets containing the MEVs are resuspended in 1-2 ml of PBS buffer and sterilized by filtration using a 0.2 μm filter and analysed the particles by nanoparticle tracking analysis (NTA; dilute up to 1:1000 before the NTA analysis).

### F. Purification of Chlorella Microalgae Extracellular Vesicles by Size Exclusion Chromatography (SEC)

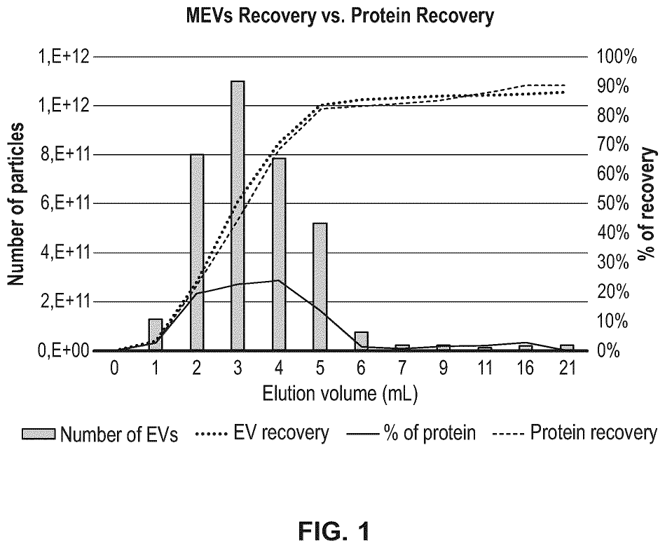

When higher purity of MEVs is needed, a last step of purification is added. The MEVs previously concentrated by TFF and purified by ultracentrifugation and formulated in PBS at concentration of 10Exp11 to 10Exp13 per mL are seeded in a pre-packed column qEV1 from IZON. The MEVs are eluted with PBS solution. The elution fractions of 0.5 mL are collected. MEVs are recovered in the first fractions as shown in FIG. 1.

MEV concentrations in the initial sample and in the fractions collected throughout the elution are evaluated with the ZetaView® engine (Nanoparticle Tracking Analyzer from Particle Metrix) as the quantity of proteins by Bradford assay. The most concentrated fractions (4-5) are pooled and stored at 4° C. before use. FIG. 1 shows purification of MEVs by Size Exclusion Chromatography.

### Example 2

### Characterization of the MEVs

### A. Nanoparticle Tracking Analysis (NTA)

MEVs were analyzed for size and dispersity (size distribution) using a NanoSight NS500 system (Malvern Panalytical Instruments). The instrument was equipped with a 488 nm laser, a high sensitivity sCMOS camera and a syringe pump. The MEV samples were diluted in particle-free PBS (0.02 μm filtered) to obtain a concentration within the recommended measurement range (1-10×10⁸ particles/mL), corresponding to dilutions of from 1/1000 to 1/10000 depending on the initial sample concentration.

For each sample, 5 experiment videos of 60 seconds duration were analyzed using NTA 3.4 Build 3.4.003 (camera level 15-16) with syringe pump speed 30. A total of 1500 frames were examined per sample, which were captured and analyzed by applying instrument-optimized settings using a suitable detection threshold so that the observed particles are marked with a red cross and that no more than 5 blue crosses are seen. Further settings were set to “automatic” and viscosity to “water.”

For the characterization of the MEVs, MEV samples are produced and purified as described in Example 1 as follows: MEV are clarified and concentrated by tangential flow filtration (TFF), diafiltration and ultracentrifugation, purified by SEC and sterilized by 0.22 μm filtration. After SEC purification and filtration, MEVs are diluted between 1,000 to 10,000 times in PBS (1×) and measured in the ZetaView® analyzer (Particle Metrix GmbH, Ammersee, Germany).

### B. Z Potential Measurement

The zeta potential of MEVs was measured three times at 25° C. under the following settings: sensitivity of 85, a shutter value of 70, and a frame rate of 30 frames per second, while ZetaView® software was used to collect and analyze the data. Table 1, below, shows the results of 3 independent measurements.

TABLE 1
Results of concentration, size, and Zeta potential measurements
for 3 representative samples of SEC-purified MEVs.

| Samples (MEVs | Concentration | MEVs Median | MEVs Zeta |
|---|---|---|---|
| After SEC) | (MEVs/mL) | size (nm) | potential (mV) |
| #1 | 2.9Exp12 | 159.9 | −20 |
| #2 | 2.9Exp12 | 163.6 | −22.58 |
| #3 | 2.9Exp12 | 161.7 | −22.3 |
| MEAN | 2.9Exp12 | 161.6 | −21.63 |
| SD | 5.7Exp8 | 2.6 | −2.3 |

### C. Transmission Electron Microscopy (TEM)

To verify the presence of intact MEVs, the preparations were analyzed using transmission electron microscopy (TEM). Fixed (4% formaldehyde, 0.2% glutaraldehyde) MEV samples were allowed to attach to Formvar/carbon-coated grids for 15-20 min, washed again with PBS followed by distilled water and finally stained with 0.4% uranyl acetate/1.8% methyl cellulose and then dried. The preparations were observed using a JEOL-JEM 1230 (JEOL Ltd., Tokyo, Japan) at 80 kV and images were acquired using a Morada digital camera and iTEM software (Olympus, Munster, Germany).

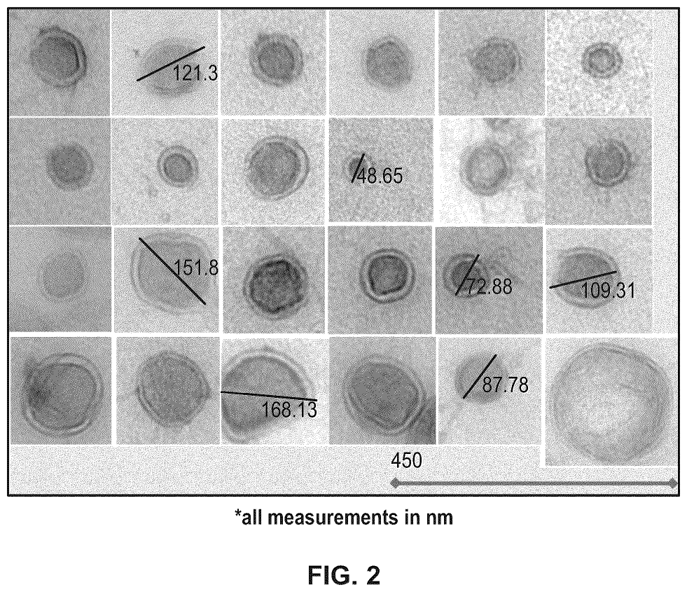

The TEM imaging, presented in FIG. 2, demonstrates that the MEVs are round shaped vesicles sized ˜50-250 nm in diameter. MEVs are enveloped in a single lipid bilayer membrane, their lumen had slightly higher electron density, and the thickness of the membrane was estimated as ˜5-10 nm, which matches the thickness of a plasma membrane. FIG. 2 shows exemplary images of MEVs obtained using Transmission Electron Microscopy (TEM).

### D. DiR Fluorescent Labelling

For uptake and internalization studies, and well as for further characterization in vivo, MEVs were labelled with DiR, a lipophilic carbocyanine derivative (1,1′-Dioctadecyl-3,3,3′,3′-Tetramethylindotricarbocyanine Iodide; Thermo Fisher Scientific) that has low fluorescence in water, but becomes highly fluorescent upon membrane incorporation, and diffuses laterally within the plasma membrane. Fresh samples of the P40 fraction (prepared as above) were re-suspended in 1 ml of BG11 culture medium. 5 μl of 1 mg/ml DiR solution was added to the samples, following incubation at 37° C. for 1 hour. Then, the samples were ultra-centrifuged at 100,000 g for 30 min using a Kontron TST 55.5 rotor at 28,100 rpm. The supernatant was removed, while the pellets were washed twice with 1 ml of PBS and centrifugation at 100,000 g for 30 min. Finally, the pellet was re-suspended in 1 ml of PBS. The DiR-labelled MEVs were stored at 4° C. and used promptly to ensure highest possible fluorescent intensity. DiR fluorescence of the labelled MEVs was measured using a SpectraMax® fluorescence microplate reader (Molecular Devices, USA) with excitation at 750 nm and emission at 780 nm.

### E. PKH26 Fluorescent Labelling

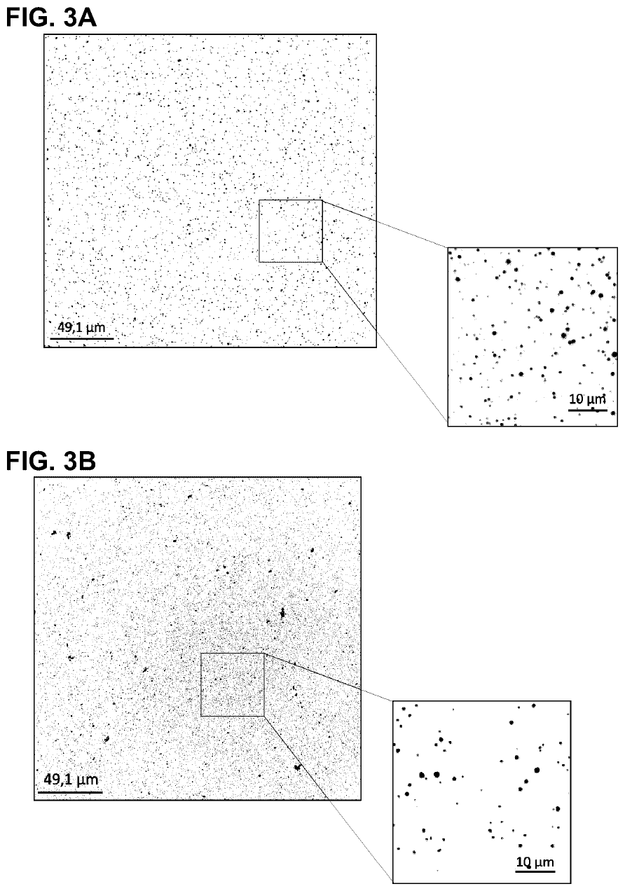

For uptake and internalization studies, and well as for further characterization in vivo, MEVs alternatively are labelled with PKH26 (Merck-Sigma), a fluorochrome in the red spectrum with peak excitation (551 nm) and emission (567 nm) that also can be excited by a 488 nm laser. MEVs fresh samples (prepared as described above) are re-suspended in 1 ml of Diluent C from the PKH26 kit. 6 μl of PKH26 dye is added to the samples, followed by continuous mixing for 30 seconds by gentle pipetting. After 5-minute incubation at room temperature, the samples are quenched by adding 2 ml of 10% BSA in 1×PBS. The volume is brought up to 8.5 ml in media and 1.5 ml of 0.971 M sucrose solution is added by pipetting slowly and carefully into the bottom of the tube, making sure not to create turbulence. The PKH26-labelled MEVs remain on top of a sucrose cushion. Then, the samples are ultra-centrifuged at 190,000 g for 2 hours at 2-8° C. using a Kontron TST 55.5 rotor. The supernatant is removed, while the pellets are washed with 1×PBS by gentle pipetting and centrifuged again at 100,000 g for 30 min, the pellet is re-suspended in 1 ml of 1×PBS. Alternatively labelled MEVs were purified from free dye by size exclusion chromatography using a qVE1/70 nm column (IC1-70—IZON Science) and washed with PBS. The MEVs were recovered in the first two fractions. The PKH26-labelled MEVs are stored at 4° C. and filtered with 0.45 μm filter before adding to cells. FIG. 3A shows the labelled MEVs with PKH26 by confocal microscopy.

### F. DiD Fluorescent Labelling

For uptake and internalization studies, MEVs alternatively are labelled with DiD (ThermoFisher Scientific), a fluorochrome in the red spectrum with peak excitation (650 nm) and emission (670 nm). MEVs fresh samples (prepared as described above) are re-suspended in 1 ml and incubated 2 μl of DiD dye added to the samples, followed by continuous mixing for 30 seconds by gentle pipetting. After 5-minute incubation at room temperature, the samples are quenched by adding 2 ml of 10% BSA in 1×PBS. The labelled MEVs were purified from free dye by size exclusion chromatography using a qVE1/70 nm column (Product Code: IC1-70, IZON Science) and washed with PBS. The MEVs were recovered in the first two fractions. The PKH26-labelled MEVs are stored at 4° C. and filtered with 0.45 μm filter before adding to cells. FIG. 3B shows the labelled MEVs with DiD by confocal microscopy.

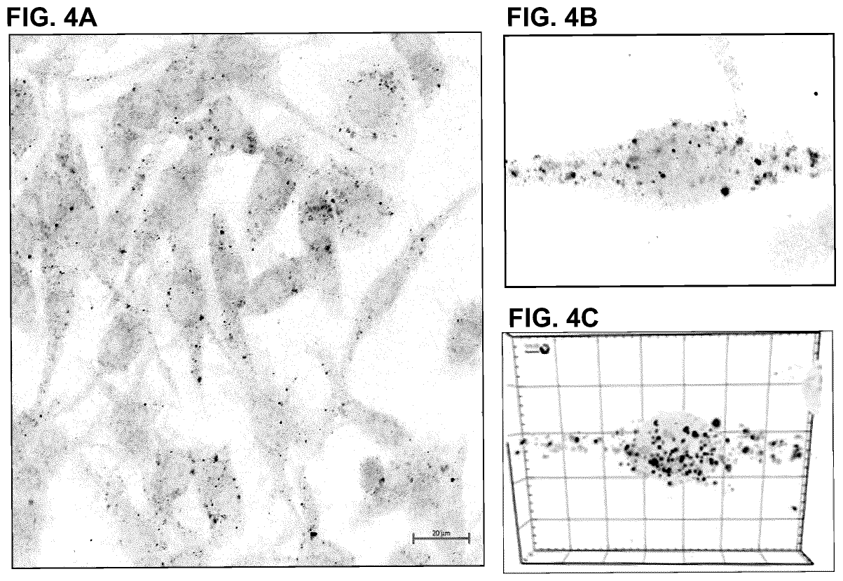

MEVs were labelled with PKH-26 or DiD and visualized using LEICA SP5 confocal microscope using 63× objective. FIG. 3A shows MEVs labelled with PKH26 (excitation 546 nm). FIG. 3B: MEVs labelled with DiD (excitation 647 nm). FIGS. 4A-C show the uptake of PKH26-labelled MEVs into cells after 16 hours of incubation. The visualization using Zeiss® LSM8 confocal microscope using 63× objective. Hoechst (Nucleus): PKH26 (MEVs) FIG. 4A: cells uptake of MEVs (2D image). FIG. 4B: individual cell image (2D). FIG. 4C: individual cell image (3D).

### G. Flow Cytometry

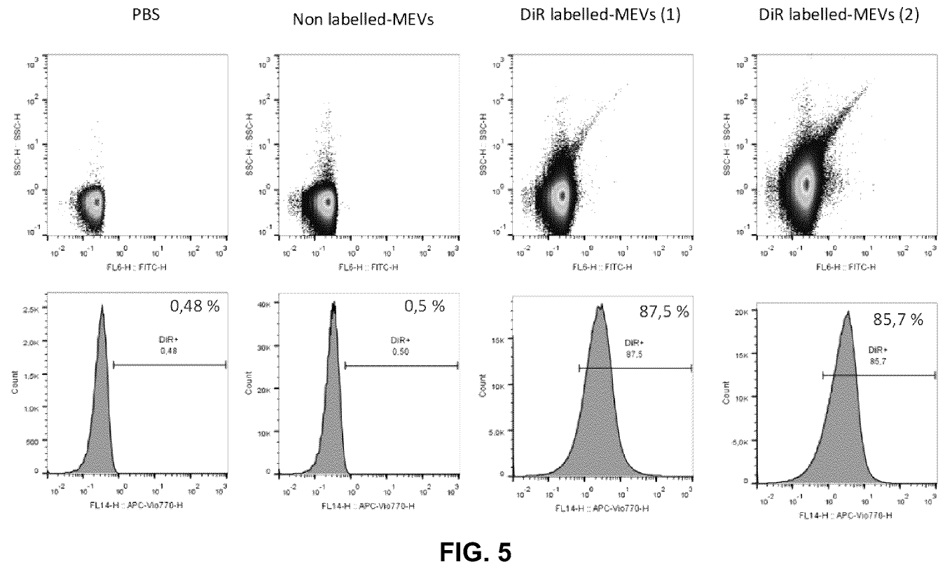

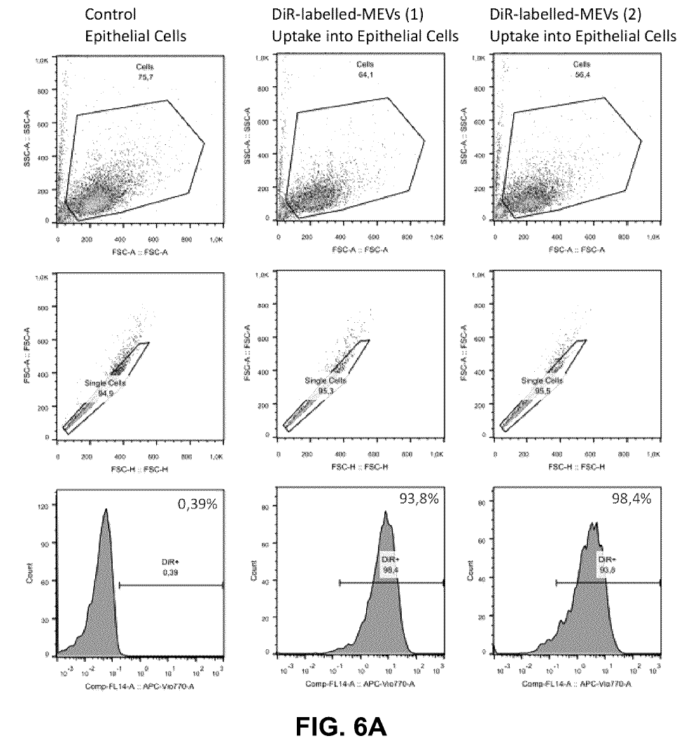

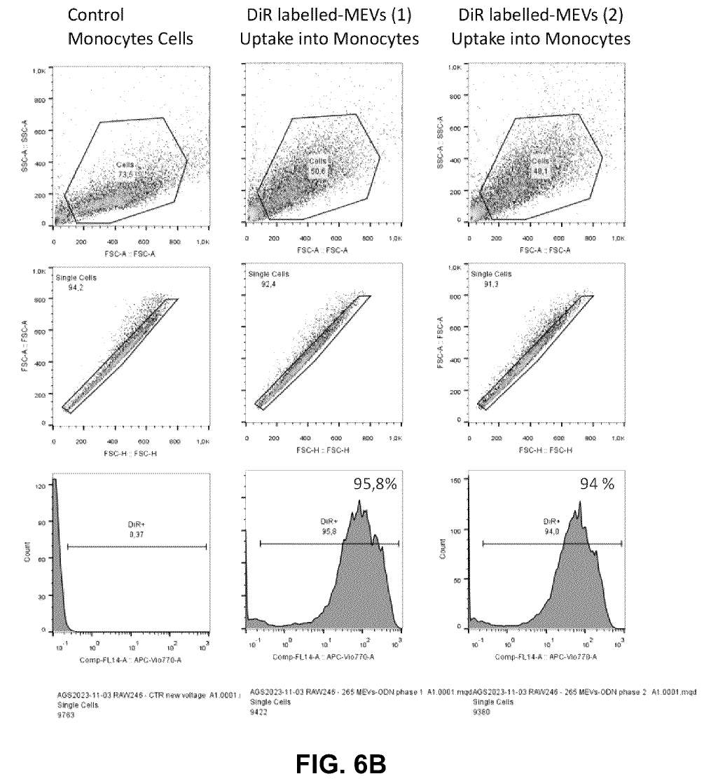

Flow cytometry analyses were conducted using LSRII flow cytometer with CellQuest™ Pro software (BD Biosciences). Latex beads of 0.3 and 1.1 m diameters were prepared and used according to the manufacturer's recommendation to define the MEV gate. Since latex beads typically have higher refractive index and thus lower limits of size detection by flow cytometry than MEVs, the thresholds for forward and side scatter were adjusted to avoid background noise during acquisition. The predefined MEV gate was applied to all samples during analysis. FIG. 5 shows the cytometry analysis of labelled MEVs. FIGS. 6A-6B show the uptake of MEVs by different types of cells by cytometry.

FIG. 5 shows the distribution of MEVs labelled with lipophilic dyes by cytometry. The figure shows the DiR dye incorporated into MEV's membrane and the percentage of labelled-MEVs. FIGS. 6A and 6B show the high uptake of MEVs in different type of cells as percentage of fluorescence cells after the treatment with DiR-labelled-MEVs by cytometry.

### Example 3

Exogenous Loading of MEVs/Loading of Biomolecules into the MEVs
A. Passive and Surfactant-Assisted Loading of Biomolecules (e.g., Proteins, Peptides, siRNA, mRNA, Antisense Oligonucleotides (ASOs), Plasmids, Complexes) Using Plasmid DNA (pDNA) and GFP (Protein) as Exemplary Biomolecules

Purified MEVs, as described in Example 1, were diluted in PBS to a specific concentration (10\hat{ }8, 10\hat{ }9 or 10\hat{ }10). Next, different concentrations of cargos (0.2, 1 or 2 μg/ml for pDNA; 2 or 20 μg/ml for GFP) were added to the MEV suspension (total volume of 500-1000 μl) and incubated for 1 or 24 hours for pDNA and 0, 1 or 6 hours for GFP (passive loading). For surfactant-assisted loading, the mixture of MEVs and pDNA was supplemented with 0.2% or 0.5% of saponin and incubated at room temperature for 5 or 30 min, with or without an agitation at 700 rpm.

B. Freeze-Thaw Cycles Loading of Biomolecules (e.g., Proteins, Peptides, siRNA, mRNA, ASOs, Plasmids, Complexes) Using Plasmid DNA (pDNA), GFP (Protein) and mRNA (Encoding eGFP) as Exemplary Biomolecules

Purified MEVs, as described in Example 1, were diluted in PBS to a specific concentration (10\hat{ }8, 10\hat{ }9, 10\hat{ }10, 10\hat{ }11 and 10\hat{ }12). Next, various concentrations of cargos (0.2, 1, 2 or 40 μg/ml for pDNA; 0.2 and 2 μg/ml for mRNA) were added to the MEV suspension (total volume of 500 μl). The mixture of MEVs and the cargo was frozen at −80° C. or immersed into liquid nitrogen (−196° C.). Then the sample was thawed at 37° C. in a water bath. This freeze-thaw cycle was repeated 2 or 4 times for each sample.

C. Sonication Loading of Biomolecules (Proteins, Peptides, siRNA, mRNA, ASOs, Plasmids, Complexes) Using Plasmid DNA (pDNA), mRNA (Encoding eGFP) and GFP (Protein) as Exemplary Biomolecules

Purified MEVs, as described in Example 1, were diluted in PBS to a pre-determined concentration ((10\hat{ }8, 10\hat{ }9, 10\hat{ }10, 10\hat{ }11 and 10\hat{ }12 MEVS/mL). Next, various concentrations of cargos (0.2, 1, 2 or 20 μg/ml for pDNA; 2 and 20 μg/ml for mRNA; 2 or 20 μg/ml for GFP) were added to the MEV suspension (total volume of 500-1000 μl). The mixture of MEVs and the cargo was sonicated using Fisherbrand Model 50 Sonic Dismembrator (frequency: 20 kHz, wattage: 50 W). Before sonication, the signal amplitude was set to 20%, 40%, 50% or 60%. Each sample underwent a sonication for 30 seconds followed by a pause during 30 seconds on ice, or 4 seconds followed by a rest period of 120 seconds on ice. This cycle was repeated 2, 6 or 8 times for each sample.

D. Extrusion Loading of Biomolecules (e.g., Proteins, Peptides, siRNA, mRNA, ASOs, Plasmids, Complexes) Using Plasmid DNA (pDNA), mRNA and GFP as Exemplary Biomolecules

Purified MEVs, as described in Example 1, were diluted in PBS to a specific concentration (10\hat{ }10). Next, different concentrations of cargos (2 or 20 μg/ml for pDNA; 0.1, 1, 2 and 10 μg/ml for mRNA; 2 or 20 μg/ml for GFP) were added to the MEV suspension (total volume of 500-1000 μl). The mixture of MEVs and the cargo was extruded through a syringe-based hand-held mini-extruder (SKU: 610023-1 EA, Avanti® Polar Lipids). The sample was extruded from 10 to 15 times across a membrane filter with 100 nm diameter pores, using two facing syringes. In some experiments, the sample was extruded sequentially through a 200 nm, a 100 nm and 50 nm diameter pore membranes. The extrusion was performed at room temperature or at 65° C.

E. Sonication and Extrusion-Assisted Active Loading (SEAL) of Biomolecules (e.g., Proteins, Peptides, siRNA, mRNA, ASOs, Plasmids, Complexes) Loading mRNA and GFP as Exemplary Biomolecules

Purified MEVs, as described in Example 1, were diluted in PBS to a specific concentration (10\hat{ }11). Next, different concentrations of cargos (2 or 20 μg/ml for mRNA; 2 or 20 μg/ml for GFP) were added to the MEV suspension (total volume of 500-600 μl). The mixture of MEVs and the cargo was sonicated for 8 min (30 sec on/30 sec off, on ice) at 20% amplitude by a Fisherbrand™ Model 50 Sonic Dismembrator (frequency: 20 kHz, wattage: 50 W). Then the sonicated MEVs were further extruded with the cargo through 100 nm polycarbonate (PC) membranes for ten cycles at room temperature.

F. Electroporation of Biomolecules (e.g., Proteins, Peptides, siRNA, mRNA, ASOs, Plasmids, Complexes) Using Plasmid DNA (pDNA) and mRNA as Exemplary Biomolecules

Purified MEVs, as described in Example 1, were diluted in PBS to a specific concentration (10\hat{ }8, 10\hat{ }9 or 10\hat{ }10). Next, different concentrations of cargos (1, 2, 5, or 20 μg/ml for pDNA; 2 and 20 μg/ml for mRNA) were added to the MEV suspension (total volume of 110-150 μl). The mixture of MEVs and the cargo was transferred into a 100 μl electroporation cuvette and placed into a Super Electroporator NEPA 21 (NEPAGENE) device. The following parameters were set for each electroporation: the voltage (50 V, 100 V, or 200 V), the pulse length (5 ms, ms, or 15 ms), the pulse interval (set to 50 ms), the decay rate (set to 10%), the polarity (set to positive), the number of pulse (1, 9 or 15), and the presence or absence of a transfer pulse (5 pulses of 20 V during 50 ms with 50 ms interval and 10% decay rate). To assess aggregation, 20 mM of EDTA or 10 mM of citrate have been added before electroporation for each condition.

### G. DNase Treatment:

After each loading method, a part of the samples underwent a treatment with a Deoxyribonuclease I (DNase) (10104159001, Roche) to eliminate all free nucleic acids (non-encapsulated by the loading). The DNase and a DNase buffer (Tris-HCl 10 mM, MgCl₂ 2.5 mM, CaCl₂ 0.5 mM, pH 7.6) were mixed with the sample and incubated for 40 minutes in an incubator at 37° C. Finally, Ethylenediaminetetraacetic acid (EDTA) (20 mM) was added to inhibit DNase action. Samples then were stored at 4° C.

### H. RNase Treatment:

After each loading method, a portion of the samples were treated with Ribonuclease I (RNase) (EN0601, Thermo Scientific) to remove any free (non-encapsulated by the loading) ribonucleic acids. The RNase was mixed with the sample and incubated for 15 minutes in a 37° C. incubator. Finally, SUPERase-In (AM2694, Invitrogen), an RNAase inhibitor, was added to inhibit the action of RNase. The samples were then incubated for 4 hours in a 37° C. incubator. Finally, the samples were stored at 4° C.

### I. Proteinases Treatment

Where proteins or nucleic acid were the payload, and after each loading method, the samples were treated with benzonase (Product No. E1014, Merck-Sigma) or micrococcal nuclease (Product No. MO247S, New England Biolabs), for 30 minutes following supplier's conditions. Samples were purified by SEC using qVE1/70 nm (Product code: IC1-70, IZON Science). The samples then were incubated for 4 hours in a 37° C. incubator. The samples were stored at 4° C.

### J. Exo-Loading Efficiency

The efficiency of exo-loading of MEVs obtained, purified, characterized, loaded, and internalization of payload determined, as described herein, is shown in the following Table (Table 2). The efficiency of exo-loading using the different loading methods for different payloads is calculated using the parameters: (i) loading-efficacy, and/or (ii) loading capacity, or (iii) the percentage of loaded MEV.

TABLE 2
Results of MEV exo-loading studies with loading
efficiency and loading capacity evaluated for
specific payloads and different loading methods.

| | | Tested | Loading | Loading |
|---|---|---|---|---|
| Payload | Method | Conditions | Efficiency | Capacity |
| DNA | Incubation | 9 | (0) | (0) |
| (plasmids/ | Saponification | 12 | (+) | (++) |
| ASOs) | Freeze thaw cycle | 28 | (++) | (+++) |
| | Electroporation | 46 | (+++) | (+++) |
| | Sonication | 23 | (++) | (++) |
| | Extrusion | 25 | (+) | (+) |
| | Serial Extrusion | 12 | (+) | (+) |
| | SEAL | (−) | ND | ND |
| RNA | Incubation | (−) | ND | ND |
| (mRNA/ | Saponification | (−) | ND | ND |
| siRNA/ | Freeze thaw cycle | 6 | (++) | (++) |
| miRNA) | Electroporation | 12 | (++) | (++) |
| | Sonication | 6 | (++) | (++) |
| | Extrusion | 24 | (++) | (++) |
| | Serial Extrusion | 21 | (+++) | (+++) |
| | SEAL | 10 | (0) | (0) |
| Proteins | Incubation | 9 | (+) | (+) |
| (peptides/ | Saponification | 12 | (++) | (++) |
| GFP/other | Freeze thaw cycle | 12 | (++) | (++) |
| proteins) | Electroporation | 12 | (++) | (++) |
| | Sonication | 40 | (+++) | (+++) |
| | Extrusion | 14 | (++) | (++) |
| | Serial Extrusion | 14 | (++) | (++) |
| | SEAL | 15 | (++) | (++) |

Key: Loading (0) very low; (+) low; (++) acceptable; (+++) best results; ND not determined, where the parameters are as follows:

Internalized payload is the total amount of payload molecules (DNA, RNA, protein, or other molecules) measure into the MEVs after the loading reaction. The internalized payload is determined by a specific quantification method (such as qPCR, RT-qpCR, determination of proteins, and other), after the elimination of the remaining payload molecules in the loading reaction medium at the end of reaction by specific enzymatic, treatment, (such as DNAse, RNAse, protease, other).

Copy number of payloads: Number of copies of payload molecule (DNA or RNA) in the either initial amount or the internalized payload amount. The number of copies is obtained using the following calculation:

$$Number of copies = ( ng \times [ 6.022 Exp 23 ] ) / ( length \times [ 1 Exp 9 ] \times 650 )$$

ng is the number of nucleic acids (plasmid, RNA.)
6.022Exp23 Avogadro's number.
Length is the length of the DNA fragment in base pairs multiply by 1000 (kb)
Multiply by I Exp9 to convert to nanograms, Loading efficiency is the percentage of the initial amount of payload molecule, in the loading reaction medium at the initial timepoint, internalized into extracellular vesicles from microalgae (MEVs).

Loading capacity is the copy number of payload internalized per MEV. The loading capacity calculated as the ratio between the internalized payload copies and the number of MEVs in the reaction.

As an example: if in a loading reaction are initially 3.6Exp10 copy of DNA and 1Exp9 MEVs, and by qPCR is determined that 7.8Exp9 copies of DNA are internalized into the MEVs. For this loading reaction the loading parameters are the following.

$$\begin{matrix}loading efficiency = ( 1.8 Exp 9 DNA copies / 3.6 Exp 10 DNA copies ) * 100 = 5 \% \\ loading capacity = 1.8 Exp 9 DNA copies / 1 Exp 9 MEV = 1.8 DNA copies / MEV .\end{matrix}$$

Percentage of loading MEVs: is the number of fluorescent MEV per total number of MEVs per 100. The fluorescence is determined using ZetaView® device at f90 and f95 are sensitivity values, Sensitivity values are expressed in arbitrary units ranges from 0 to 100. It represents the sensitivity of the camera sensor in the ZetaView© device. This sensitivity setting is comparable to the sensitivity setting of a camera used in digital photography. The number of detected particles in the field is associated to the sensitivity camera: increase of sensitivity->increase of the number of particles detected in each sample. Sensitivity is calibrated before the measurement based on the negative control chose the highest sensitivity for which no event is detectable,

### Example 4

### Endogenous-Loading of MEVs

Chlorella vulgaris engineering to generate producer cell lines carrying exogenous coding regions and endogenous loading of biomolecule cargo into the Microalgae Extracellular Vesicles (MEVs) is described in this Example.

A. Transformation Using a Tumor Inducing (Ti) Plasmid, and Agrobacterium tumefaciens to Generate Stable Chlorella Producer Cell Lines for Endogenous Loading of Biomolecules, Such as Proteins, Peptides, siRNA, mRNA, Complexes) into the MEVs

### 1. Chlorella Culture Conditions

Chlorella cells (Chlorella vulgaris UTEX 265, UTEX 395, and CCAP 211/19 from GEPEA, University of Nantes, France) are maintained in BG11, 1% agar plates and grown in BG11 liquid medium pH 7, in autotrophic conditions in growth chamber under the following conditions: i) temperature: 25° C.; ii) photoperiod:14 h/10 h; iii) light intensity:100 μmol m⁻²·s⁻¹.

### 2. Agrobacterium Strain and Vectors

The plasmid vectors used for Agrobacterium transformation are generated using the green gate assembly strategy (see, Lamproulos et al. (2013) J. PLoS One 8:e83043; PMID24376629). The gene specific or chimeric constructs are cloned in modules “B” or “D” and/or “B and D” according to the cloning strategy (coding or non-coding RNA) and assembled in expression plasmid constructs under the control of Cauliflower Mosaic Virus (CaMV) 35S promoter or other promoters known to those of skill in the art for expression in microalgae, including any described herein, such as those listed in the table in the detailed description, and a specific construct encoding a product of interest, and a resistance cassette (Hygromycin resistance gene or NPTII gene (neomycin phosphotransferase II). All chimeric constructs are obtained by simultaneous ligations of the different fragments into the “B” or “D” and/or “B and D” module. All plasmids are verified by restriction analysis, and Sanger sequencing.

The binary vector pCAMBIA1304 (cambia.org) encoding a GFP:gusA fusion reporter and a selectable marker for hygromycin B resistance driven by the CaMV 35S promoter is used for some transformations.

The Agrobacterium tumefaciens used for Chlorella transformations is a disarmed strain C58C1. Plasmids are introduced into A. tumefaciens by electroporation.

3. Transformation of Chlorella Cells with Agrobacterium tumefaciens

Chlorella cells (10⁸ cells) from an exponentially growing culture are plated on BG11 agar plates and kept under normal light for 5 days. For genetic transformation, A. tumefaciens carrying the appropriate plasmid vector is pre-inoculated the day before the transformation. The day of the transformation 5 mL of A. tumefaciens pre-inoculum is seeded in 50 mL LB medium and grown up to OD₆₀₀=around 1. At the defined optical density A. tumefaciens pre-inoculum is washed and resuspended in 200 μL induction medium (BG1 Imedium at pH 5.6 plus acetosyringone 100 μM). Chlorella cells are gently harvested from the plates and resuspended in the 200 μL of induction medium plus the A. tumefaciens and co-cultivated for 2 days in induction medium in dark. After the co-cultivation, the cells are harvested and put in BG11 medium pH 7 supplemented with cefotaxime and kept in dark for 2 days at 25° C. Finally, cells are harvested and plated onto BG11 agar plates supplemented with the relevant antibiotic according to the plasmid vector used for the transformation. Plates are kept in dark for 2 more days and then exposed to light. Around 2-3 weeks colonies are replicated on fresh BG11 agar plate containing relevant antibiotics. Resistant colonies are propagated on non-selective media and used for PCR analysis. Detection of contaminating Agrobacterium is performed by growing cells on LB agar plates for at least 7 days at 25° C. in the dark. The expression of the gusA reporter gene is confirmed by GUS histochemical assay, while visualization of GFP expression is performed using a fluorescent microscope (Leica DM Ire2, Wetzlar, Germany).

B. Transformation by Electroporation to Generate Stable Producer Cell Lines that Endogenously Load Biomolecules (e.g., Proteins, Peptides, RNAi, mRNA, Complexes) into the MEVs; GFP or RNAi Against Luciferase Coding Regions are Exemplary Biomolecules.

### 1. Chlorella Culture Conditions

Chlorella cells (Chlorella vulgaris UTEX 265, UTEX 395 and CCAP 211/19 from GEPEA, University of Nantes, France) are maintained in BG11, 1% agar plates and grown in BG11 liquid medium pH 7, in autotrophic conditions in growth chamber under the following conditions: i) temperature: 25° C.; ii) photoperiod: 14 h/10 h; iii) light intensity: 100 μmol m⁻²·s⁻¹.

### 2. Enzyme Digestion for Chlorella Cells to Prepare Protoplast

To prepare protoplasts, Chlorella cells are treated with an enzyme mixture containing 0.6 M sorbitol, 0.1% MES, 50 mM CaCl₂·2H₂O, 1.0 mg/mL lysozyme, 0.25 mg/mL chitinase, and 1.0 mg/mL sulfatase in 10 mL of sterile water. A total of 1×10⁷ cells 100 mL at early exponential growth phase are used for preparing protoplasts in 10 mL of the mixture solution. Cells are incubated at room temperature in the dark up to 24 h with gentle rotation at 25 rpm. Cells are harvested by centrifugation at 1350×g for 10 min. The viability of protoplasts after enzymatic treatment is about 7%.

### 3. Electroporation Conditions

Chlorella cells and Chlorella protoplasts, at a concentration of 10⁶ in 100 mL, are transformed at different conditions between 600V to 1500V pulse voltage with 3 to 5 ms pulse width and using 60 ng plasmid using a Bio-Rad Gene Pulser® X cell electroporation system. Electroporation is slightly modified from previously described methods (Bai et al. (2013) PLoS one 8:e54966, doi:10.1371/journal.pone.0054966; Run et al. (2016) Algal Res 17:196-201, doi:10.10.106/j.algal 2016.05.002; and Kumar et al. (2018) Journal of Applied Phycology 30:1735-1745, doi.org/10.1007/s10811-018-1396-3) as follows: 45 μL of pre-cooled osmotic buffer (0.2 M mannitol and 0.2 M sorbitol) is added for the suspension of the harvested cells (1350×g for 10 min) and then incubated for 40 min on ice. Again, 45 μL of pre-cooled electroporation buffer (0.2 M mannitol, 0.2 M sorbitol, 0.08 M KCl, 0.005 M CaCl₂, and 0.01 M Hepes; pH 7.2) is added in the suspension solution. 60 ng of plasmids of pCAMBIA1302 or pIT69 (in 10 mL) is added to the suspension cells immediately along with 1.5 g of sonicated salmon sperm DNA.

a. Screening

After electroporation, cells are kept on ice for 60 min, transferred to a 12-well plate containing 1.5 mL of BG11 medium, and cultured in the dark at 25° C. for 24 h. The cultured cells are harvested by centrifugation, suspended in 200 μL of BG11 medium, plated onto BG11 agar plates containing 20 g/mL hygromycin, and incubated in continuous fluorescent light with 60 μmol photons m⁻¹ s⁻¹ at 25° C.

Two weeks after transformation and plating, clones are picked and cultured in 96-well plates for 1 week with BG11 medium and cultured in the dark at 25° C. and 20 μg/mL hygromycin. Chlorella cells are then divided into two groups: 1) for PCR analysis and 2) to test ability to grow concentrations of hygromycin between 0 and 100 g/mL. Hygromycin resistance test of selected clones indicates that clones are obtained after transformation of Chlorella protoplasts under all of the electroporation conditions tested, strains and clones description: 265 (A1) is Chlorella vulgaris UTEX 265 strain, Ath1 to Ath7 are transformed clones obtained from Chlorella vulgaris UTEX 265 strain by electroporation, 395(K1) is Chlorella vulgaris UTEX 395 strain, Kth1 and Kth2 are transformed clones obtained from Chlorella vulgaris UTEX 395 strain by electroporation and Hr5 is a clone transformed from Chlorella vulgaris UTEX 265 strain obtained by Agrobacterium tumefaciens.

PCR analysis, of each of 20 clones obtained by electroporation from Chlorella vulgaris UTEX 265 strain, and 10 clones obtained by electroporation from Chlorella vulgaris UTEX 395 strain were tested and confirmed the integration of hygromycin phosphotransferase gene (hpt) at the molecular level. The efficiency of transformation by electroporation of both strains of Chlorella vulgaris tested was between 30-50%.

b. Clone Selection

PCR analysis, of each of 20 clones obtained by electroporation from Chlorella vulgaris UTEX 265 strain, and 10 clones obtained by electroporation from Chlorella vulgaris UTEX 395 strain were tested and confirmed the integration of hygromycin phosphotransferase gene (hpt) at the molecular level. The efficiency of transformation by electroporation of both strains of Chlorella vulgaris tested was between 30-50%.

C. Particle Gun Transformation to Generate Stable Producer Cell Lines for Endogenous Loading of Biomolecules (e.g., Proteins, Peptides, RNAi, mRNA, Complexes) into the MEVs Using Gfp or RNAi Against Luciferase Coding Regions as Exemplary Biomolecules.
Transformation of Chlorella vulgaris Cells Using Gun Microparticles Method

Chlorella vulgaris cells (1×10⁸) are collected from exponentially growing liquid cultures in BG-11 medium and spread on 10 cm 1% BG-11 agar plates containing. Two hours later, transformations are carried out using the microparticle bombardment method (Biolistic PDS-1000/Particle Delivery System (BioRad)) adapted from Apt and collaborators (see reference) with minor modifications. In brief, gold particles (particle diameter of 0.6 μm, BioRad) are coated with DNA using 1.25 M CaCl₂ and 20 mM spermidine. Agar plates Chlorella cells to be transformed are positioned at 7.5 cm from the stopping screen within the bombardment chamber. A burst pressure of 1,550 psi and a vacuum of 25 Hg are used. For the experiments, 5 μg of each plasmid encoding hygromycin resistance gene (pIT069) or not (p¹6604) are used as negative control. Five bombardments are performed using each DNA.

### D. Cloning, Selection, and Screening of Transformed Clones (Producer Cell Lines).

### 1. Cloning and Selection of Transformed Clones

Two days post-transformation, bombarded cells are spread with 50 μg/ml or 100 μg/ml of hygromycin on the agar plates and placed in the incubator under a 12 h light:12 h dark cycle for at least 3 weeks. Chlorella colonies appear about 2 weeks after transformation by bombardment. Chlorella colonies are cultured either in 50 μg/ml (A) or 100 μg/ml (B) of hygromycin. After 2 weeks, colonies are visible on the plate with cells that contain and express the hygromycin resistance gene, and not visible on the plates containing the negative control. Colonies are observed on plates on which the cells that express the hygromycin resistance gene (pIT0609) are cultured. Negative control (p16604) clones do not grow in the presence of hygromycin.

For subcloning, colonies from transformations are re-suspended in BG-11 culture medium and plated at a low density (600 cells on a 10 cm agar plate containing hygromycin antibiotic), providing for the isolation of subclones 2-3 weeks later.

### 2. Chlorella Genomic DNA Extraction

Genomic DNA is extracted from exponentially growing cultures using a NucleoSpin® DNA protocol. Genomic DNA (gDNA) concentration is measured using a QuBit fluorometer.

### 3. PCR Analysis of Transformants

Direct PCR colony analysis on gDNA is performed by collecting a little bit of the colony to be analyzed (use a pipette tip or an inoculation tool or a toothpick) and resuspending it in 20 μl of HS5 buffer (125 mM NaOH, 1 mM EDTA, 0.1% Tween 20). After 20 s of vortexing at max speed, the samples are incubated for 10-15 minutes at RT and boiled at 95° C. for 10-15 s. Next, 100 μl of H₂O is added, mixed well and briefly centrifuged to spin down the debris. 1-5 μl are used as a template for PCR reaction. The plasmid used for the transformation is the positive control; wild-type gDNA served as the negative control.

PCR analysis is performed in a 25 μL reaction containing 150 ng DNA, 1.25 mM dNTP, 2 mM MgCl₂, 1.25 μM of each primer and 1 U OneTaq® DNA polymerase. The primers used to amplify a 650 bp fragment of the hygromycin resistance gene are Hygro 1: 5′-AGCGTCTCCGACCTGATG-3′ (SEQ ID NO:259) and Hygro 2: 5′-CGACGGACGACTGACGG-3′; (SEQ ID NO:260). Amplification is carried out in a thermal cycler (Eppendorf). The amplification of hygromycin resistance gene shows the expected 650 bp fragment in all positive clones (i.e., growing in BG-11 plus 50 mg/mL hygromycin).

### E. Storage of Chlorella Modified Producer Cell Lines.

Cryopreservation of Chlorella vulgaris Cells

Two conditions of cryopreservation and storage of isolated cells from Chlorella vulgaris in exponential growing state and in stationary state are evaluated. The first condition corresponds to the standard method recommended by UTEX collection using 10% methanol as a cryoprotective agent in BG-11 medium and the second condition using 25% glycerol in BG-11 medium as routinely used for animal cell lines.

Both cryo-storage conditions are evaluated in exponential growing phase (4-5×10⁶ cells) or stationary phase (3-5×10⁷ cells). After congelation, the cells are thawed at 37° C. Cell viability is determined in BG-11 medium for 5 days (liquid medium) or BG-11 solid medium (stria) for 15 days culture. Viability is determined after one or two cycles of freezing/thawing at 3 days and 30 days (one cycle) or 30, 60, 180 days (two cycles).

A higher viability and a better reproducibility of the results is obtained with the 25% of glycerol in BG-11 medium (freezing medium), with Chlorella cells in exponential growing phase and a dilution of 100 mL of cryomedium in 5 mL BG-11 medium liquid culture as thawing culture conditions.

F. Determination of the Genetic Stability of Chlorella Producer Cell Lines. PCR Detection of Chlorella Transformants

Genomic DNA from transformed and wild-type (WT) Chlorella strains is isolated from exponentially growing cultures using NucleoSpin® DNA protocol. Genomic DNA concentration is measured using a QuBit fluorometer. PCR analysis is performed to detect the presence of the hygromycin resistance gene using specific primers as described above or firefly luciferase primers: f-LUC1—5′-CCAGGGATTTCAGTCGATGT-3′(SEQ ID NO: 261) and f-LUC2—5′-AATCTCACGCAGGCAGTTCT-3′ (SEQ ID NO: 262); or GFP (SEQ ID NO:263 and 264) or eGFP (SEQ ID NOs:265 and 266) or mCherry (SEQ ID NOs:267 and 268) or GUS (SEQ ID NOs: 269 and 270) as described below.

G. Transformation by Multisequence Pulses Electroporation to Generate Stable Producer Cell Lines that Endogenously Load Biomolecules (e.g., Proteins, Peptides, RNAi, mRNA, Complexes) into the MEVs; eGFP (Modified Codon or not) or fLUC (Modified Codon or not) or RNAi Against Luciferase Coding Regions are Exemplary Biomolecules.

A highly efficient multisequence pulses method was optimized to transform Chlorella vulgaris cells; with the objective of generating stable producer cell lines and to obtain MEVs endo-loaded (with tailored mRNA, proteins, or siRNAs) by the microalgae. The multisequence pulses method generates, first, pores in the wall and membranes and, second, pulses the offered DNA into the cells.

### 1. Generation of Plasmid Vectors

For the generation of Chlorella vulgaris producer cell lines a series of plasmids (SEQ NOs.:271-318) constructs were obtained using the Green-Gate system combining: 12 different promoters-enhancers, and 2 marker proteins eGFP and fly fire luciferase, the cDNAs for each of the two proteins was cloned in plasmid vectors using mammalian codons or Chlorella codons (different GC %), and 2 antibiotic resistance genes in order to obtain Chlorella producer cell lines and RNAi against fLUC. The constructs are verified by sequencing the complete region of interest, from the promoter of the transgene to the poly-A of the antibiotic resistance gene.

Table 3 shows a compilation of the constructs tested by multiple sequence pulses electroporation aiming at the transformation of Chlorella vulgaris to generate cells lines producers of MEVs endo-loaded with tailored mRNAs, proteins, or RNAi.

TABLE 3

| plasmid | | promoter | | | Resistance | Seq |
|---|---|---|---|---|---|---|
| name # | promoter | type | CDS | Terminator | Gene | ID # |
| pAGS - | EF 2 | constitutive | FLUC | RBCS | Hygromycin | 271 |
| 0001 | (Phaeodactylum | | modified | | | |
| | tricornutum) | | codon to | | | |
| | | | Chlorella GC | | | |
| | | | content | | | |
| pAGS - | Histone 4 | constitutive | FLUC | RBCS | Hygromycin | 272 |
| 0002 | (Penicillium | | modified | | | |
| | funiculosum) | | codon to | | | |
| | | | Chlorella GC | | | |
| | | | content | | | |
| pAGS - | Lhcf.1 | inducible | FLUC | RBCS | Hygromycin | 273 |
| 0003 | (Phaeodactylum) | (light) | modified | | | |
| | | | codon to | | | |
| | | | Chlorella GC | | | |
| | | | content | | | |
| pAGS - | Ubi.U4 | constitutive | FLUC | RBCS | Hygromycin | 274 |
| 0004 | (Nicotiana | | modified | | | |
| | tabacum) | | codon to | | | |
| | | | Chlorella GC | | | |
| | | | content | | | |
| pAGS - | CAM 35S | constitutive | FLUC | RBCS | Hygromycin | 275 |
| 0005 | (Cauliflower | | modified | | | |
| | mosaic virus) | | codon to | | | |
| | | | Chlorella GC | | | |
| | | | content | | | |
| pAGS - | MAS | constitutive | FLUC | RBCS | Hygromycin | 276 |
| 0006 | (Agrobacterium | | modified | | | |
| | tumefaciens) | | codon to | | | |
| | | | Chlorella GC | | | |
| | | | content | | | |
| pAGS - | PAR | inducible | FLUC | RBCS | Hygromycin | 277 |
| 0007 | (Arabidopsis | (light) | modified | | | |
| | thaliana) | | codon to | | | |
| | | | Chlorella GC | | | |
| | | | content | | | |
| pAGS - | CV PAS D | inducible | FLUC | RBCS | Hygromycin | 278 |
| 0008 | (Chlorella | (light and | modified | | | |
| | vulgaris) | stress) | codon to | | | |
| | | | Chlorella GC | | | |
| | | | content | | | |
| pAGS - | CMV | constitutive | FLUC | RBCS | Hygromycin | 279 |
| 0009 | | | modified | | | |
| | | | codon to | | | |
| | | | Chlorella GC | | | |
| | | | content | | | |
| pAGS - | CMV + | constitutive | FLUC | RBCS | Hygromycin | 280 |
| 0010 | enhancer | | modified | | | |
| | | | codon to | | | |
| | | | Chlorella GC | | | |
| | | | content | | | |
| pAGS - | SV40 | constitutive | FLUC | RBCS | Hygromycin | 281 |
| 0011 | | | modified | | | |
| | | | codon to | | | |
| | | | Chlorella GC | | | |
| | | | content | | | |
| pAGS - | SV40 + | constitutive | FLUC | RBCS | Hygromycin | 282 |
| 0012 | enhancer | | modified | | | |
| | | | codon to | | | |
| | | | Chlorella GC | | | |
| | | | content | | | |
| pAGS - | EF 2 | constitutive | eGFP | RBCS | Hygromycin | 283 |
| 0013 | (Phaeodactylum | | modified | | | |
| | tricornutum) | | codon to | | | |
| | | | Chlorella GC | | | |
| | | | content | | | |
| pAGS - | Histone 4 | constitutive | eGFP | RBCS | Hygromycin | 284 |
| 0014 | (Penicillium | | modified | | | |
| | funiculosum) | | codon to | | | |
| | | | Chlorella GC | | | |
| | | | content | | | |
| pAGS - | Lhcf. 1 | inducible | eGFP | RBCS | Hygromycin | 285 |
| 0015 | (Phaeodactylum) | (light) | modified | | | |
| | | | codon to | | | |
| | | | Chlorella GC | | | |
| | | | content | | | |
| pAGS - | Ubi.U4 | constitutive | eGFP | RBCS | Hygromycin | 286 |
| 0016 | (Nicotiana | | modified | | | |
| | tabacum) | | codon to | | | |
| | | | Chlorella GC | | | |
| | | | content | | | |
| pAGS - | CAM 35S | constitutive | eGFP | RBCS | Hygromycin | 287 |
| 0017 | (Cauliflower | | modified | | | |
| | mosaic virus) | | codon to | | | |
| | | | Chlorella GC | | | |
| | | | content | | | |
| pAGS - | MAS | constitutive | eGFP | RBCS | Hygromycin | 288 |
| 0018 | (Agrobacterium | | modified | | | |
| | tumefaciens) | | codon to | | | |
| | | | Chlorella GC | | | |
| | | | content | | | |
| pAGS - | PAR | inducible | eGFP | RBCS | Hygromycin | 289 |
| 0019 | (Arabidopsis | (light) | modified | | | |
| | thaliana) | | codon to | | | |
| | | | Chlorella GC | | | |
| | | | content | | | |
| pAGS - | CV PASD | inducible | eGFP | RBCS | Hygromycin | 290 |
| 0020 | (Chlorella | (light and | modified | | | |
| | vulgaris) | stress) | codon to | | | |
| | | | Chlorella GC | | | |
| | | | content | | | |
| pAGS - | CMV | constitutive | eGFP | RBCS | Hygromycin | 291 |
| 0021 | | | modified | | | |
| | | | codon to | | | |
| | | | Chlorella GC | | | |
| | | | content | | | |
| pAGS - | CMV + | constitutive | eGFP | RBCS | Hygromycin | 292 |
| 0022 | enhancer | | modified | | | |
| | | | codon to | | | |
| | | | Chlorella GC | | | |
| | | | content | | | |
| pAGS - | SV40 | constitutive | eGFP | RBCS | Hygromycin | 293 |
| 0023 | | | modified | | | |
| | | | codon to | | | |
| | | | Chlorella GC | | | |
| | | | content | | | |
| pAGS - | SV40 + | constitutive | eGFP | RBCS | Hygromycin | 294 |
| 0024 | enhancer | | modified | | | |
| | | | codon to | | | |
| | | | Chlorella GC | | | |
| | | | content | | | |
| pAGS - | EF 2 | constitutive | eGFP non | RBCS | Hygromycin | 295 |
| 0025 | (Phaeodactylum | | modified | | | |
| | tricornutum) | | sequence | | | |
| pAGS - | Histone 4 | constitutive | eGFP non | RBCS | Hygromycin | 296 |
| 0026 | (Penicillium | | modified | | | |
| | funiculosum) | | sequence | | | |
| pAGS - | Lhcf. 1 | inducible | eGFP non | RBCS | Hygromycin | 297 |
| 0027 | (Phaeodactylum) | (light) | modified | | | |
| | | | sequence | | | |
| pAGS - | Ubi.u4 | constitutive | eGFP non | RBCS | Hygromycin | 298 |
| 0028 | (Nicotiana | | modified | | | |
| | tabacum) | | sequence | | | |
| pAGS - | CAM 35S | constitutive | eGFP non | RBCS | Hygromycin | 299 |
| 0029 | (Cauliflower | | modified | | | |
| | mosaic virus) | | sequence | | | |
| pAGS - | MAS | constitutive | eGFP non | RBCS | Hygromycin | 300 |
| 0030 | (Agrobacterium | | modified | | | |
| | tumefaciens) | | sequence | | | |
| pAGS - | PAR | inducible | eGFP non | RBCS | Hygromycin | 301 |
| 0031 | (Arabidopsis | (light) | modified | | | |
| | thaliana) | | sequence | | | |
| pAGS - | CV PAS D | inducible | eGFP non | RBCS | Hygromycin | 302 |
| 0032 | (Chlorella | (light and | modified | | | |
| | vulgaris) | stress) | sequence | | | |
| pAGS - | CMV | constitutive | eGFP non | RBCS | Hygromycin | 303 |
| 0033 | | | modified | | | |
| | | | sequence | | | |
| pAGS - | CMV + | constitutive | eGFP non | RBCS | Hygromycin | 304 |
| 0034 | enhancer | | modified | | | |
| | | | sequence | | | |
| pAGS - | SV40 | constitutive | eGFP non | RBCS | Hygromycin | 305 |
| 0035 | | | modified | | | |
| | | | sequence | | | |
| pAGS - | SV40 + | constitutive | eGFP non | RBCS | Hygromycin | 306 |
| 0036 | enhancer | | modified | | | |
| | | | sequence | | | |
| pAGS - | EF 2 | constitutive | FLUC non | RBCS | Hygromycin | 307 |
| 0037 | (Phaeodactylum | | modified | | | |
| | tricornutum) | | sequence | | | |
| pAGS - | Histone 4 | constitutive | FLUC non | RBCS | Hygromycin | 308 |
| 0038 | (Penicillium | | modified | | | |
| | funiculosum) | | sequence | | | |
| pAGS - | Lhcf.1 | inducible | FLUC non | RBCS | Hygromycin | 309 |
| 0039 | (Phaeodactylum) | (light) | modified | | | |
| | | | sequence | | | |
| pAGS - | Ubi.U4 | constitutive | FLUC non | RBCS | Hygromycin | 310 |
| 0040 | (Nicotiana | | modified | | | |
| | tabacum) | | sequence | | | |
| pAGS | CAM 35S | constitutive | FLUC non | RBCS | Hygromycin | 311 |
| 0041 | (Cauliflower | | modified | | | |
| | mosaic virus) | | sequence | | | |
| pAGS - | MAS | constitutive | FLUC non | RBCS | Hygromycin | 312 |
| 0042 | (Agrobacterium | | modified | | | |
| | tumefaciens) | | sequence | | | |
| pAGS- | PAR | inducible | FLUC non | RBCS | Hygromycin | 313 |
| 0043 | (Arabidopsis | (light) | modified | | | |
| | thaliana) | | sequence | | | |
| pAGS - | CV PAS D | inducible | FLUC non | RBCS | Hygromycin | 314 |
| 0044 | (Chlorella | (light and | modified | | | |
| | vulgaris) | stress) | sequence | | | |
| pAGS - | CMV | constitutive | FLUC non | RBCS | Hygromycin | 315 |
| 0045 | | | modified | | | |
| | | | sequence | | | |
| pAGS - | CMV + | constitutive | FLUC non | RBCS | Hygromycin | 316 |
| 0046 | enhancer | | modified | | | |
| | | | sequence | | | |
| pAGS - | SV40 | constitutive | FLUC non | RBCS | Hygromycin | 317 |
| 0047 | | | modified | | | |
| | | | sequence | | | |
| pAGS - | SV40 + | constitutive | FLUC non | RBCS | Hygromycin | 318 |
| 0048 | enhancer | | modified | | | |
| | | | sequence | | | |

### 2. Transformation by Multisequence Pulses

Cells of Chlorella vulgaris in the phase of exponential growth are electroporated using Nepa21 Type II Electroporator (Nepagene), as follows: after determination of the number of cells/mL, an aliquot of ten million cells per aliquot are placed in separate 1.5 mL Eppendorf® tubes (one tube per transformation condition) in 100 μL of BG-11 media plus 0.7 M of mannitol; 6 μg (in 20 μL at maximum) of linearized plasmids are added to the cell suspensions. The preparations are homogenized by tapping the tubes, then placed into electroporation chambers of 2 mm of diameter (Product No. EC002S, Sonidel) and electroporated. The conditions of electroporation are described in Table 4.

TABLE 4A
Conditions for poring Chlorella cells
First Sequence: Poring Pulses

| | Pulse Time | Interval between | Pulse | Decay | |
|---|---|---|---|---|---|
| Voltage | (ms) | Pulses (ms) | Number | Rate % | Polarity |
| 300 | 5 | 50 | 7 | 10 | non |

TABLE 4B
Conditions for transferring DNA into Chlorella cells
Second Sequence: Transferring Pulses

| | Pulse Time | Interval between | Pulse | Decay | |
|---|---|---|---|---|---|
| Voltage | (ms) | Pulses (ms) | Number | Rate % | Polarity |
| 8 | 50 | 50 | 5 | 40 | non |

After transformation, Chlorella cells are diluted in 4 mL BG-11 media+0.7M mannitol in T25 flasks. Cells are incubated in a refrigerated incubator, at 25° C. overnight, protected from light. The next day, they are resuspended using a sterile pipette, and transferred into 15 mL Falcon tubes. Cells are centrifuged 5 min at 700 g, the supernatant removed and resuspended in 200 μL of BG-11 media. The total volume (200 μL) of each cell suspension is plated on Agar/BG-11 agar plates (+70 μg/mL hygromycin or kanamycin according to the plasmid vector used for transformation) and incubated in the refrigerated incubator at 25° C., for 12 hours light/12 hours dark (12L/12D). Clones were visible at day 10 and are harvested for PCR screening at day 15.

### 3. Clone Screening

For each transformation, 5 to 10 clones are picked and cultured in BG-11 medium containing an antibiotic (either 40-70 mg/mL of Hygromycin or 70-100 mg/mL of Kanamycin). After 10 days of culture clones are tested to identify positive clones, and in parallel frozen for banking. Positive clones are identified by PCR using the corresponding primers in the following list, that match the coding regions of either the resistance gene or the transgene, or the promoter region of the vector. Screening is performed by either multiplex or simplex PCR analysis. The number of positive clones obtained after the multiple sequence pulses is an average of 4 out of 10 clones picked.

After screening, 24 positive clones of Chlorella producer cell lines are produced MEVs are collected and semi-purified as described in the Example above. Semi purified MEVs are tested to verify the presence of a payload via the endo-loading, as described below in Example 5.

### Example 5

### MEV Characterization Following Loading a Biomolecule Cargo

### A. DNA Extraction:

QIAprep® Spin Miniprep Kit (acquired from QIAGEN) was used to extract nucleic acid encapsulated by the MEVs according to the manufacturer's recommendations. At the end of the protocol, nucleic acids were eluted in 50 μL of nuclease-free water.

### B. RNA Extraction:

The RNeasy® Micro Kit (50) (acquired from QIAGEN) was used to extract the ribonucleic acid encapsulated by the MEVs according to the manufacturer's recommendations. At the end of the protocol, the nucleic acids were eluted in 14 μL of nuclease-free water.

### C. Reverse Transcription:

RNA was isolated from loading methods of MEVs with CleanCap® mRNA coding for eGFP (Tebu-bio) (to which 50 ng of an RNA (mCherry (Tebu-bio)) has been added) by the single-step purification method with RNeasy® Micro Kit (ref No. 74004, Qiagen GmbH, Germany) described by the manufacturer's protocol (Qiagen-RNeasy Micro Handbook). At the end of the protocol, the nucleic acids were eluted in 14 μL of nuclease-free water.

Reverse transcription and real-time PCR: Quantitative RT-PCR (RT-Q-PCR) was performed as follows: 4 μl of extracted total RNA was reverse transcribed using SuperScript™ II Reverse Transcriptase (Thermo Fisher Scientific cat. No. 18064-071, manufacturer's protocol: Part no. 18064.pps, MAN0001342) in a 16 μL reaction volume (constituted with hexamer random primer, dNTP Mix, Buffer, DTT, pure RNA, SuperScript™ II RT and RNaseOUT™). The reaction was mixed by very gentle pipetting to ensure that all reagents were thoroughly mixed. After mixing Hexamer Random primer, dNTP Mix, RNA and water, samples were spun down and incubated for 5 minutes at 65° C., put on ice and finally incubated 12 minutes at 25° C. where buffer, DTT, RNaseOUT™ and SuperScript™ II RT were added. Next the reaction was incubated at 42° C. for 50 min. For the last step of the mRNA reverse transcription protocol, the Reverse Transcriptase was heat-inactivated for 15 minutes at 70° C. The obtained volume of cDNA after RT was 16 μl.

After the RT reaction, 56 μl of H₂O were added to the sample to obtain in total a 72 μl cDNA starting solution per sample. Before Real-time PCR measurements, the needed dilution factor of the 72 μl cDNA starting solution was determined via prescreening experiments as X1000. Defining the right dilution factor ensures that the measured Cq output values will have values between 20 and 30. This Cq 20-30 range assures better linearity and avoids the signal's noise region which is expected to start at Cqs higher than 30.

D. qPCR:

To quantify the plasmid extracted from the MEVs, analysis by quantitative Polymerase Chain Reaction (qPCR) have been performed. DNA concentration was measured in the samples using a NanoDrop™ 2000 analyzer (acquired from ThermoScientific) and the samples were diluted if necessary to be correctly amplified during the qPCR (volumes for each condition are listed in annexes). A plasmid range was realized from 0.4×10⁻⁶ ng to 5.1×10⁻⁶ ng of pDNA. A master mix was prepared with 5 μL of PowerUp™ SYBR™ Green per well (Cat. No. A25776, from Fisher Scientific™ Applied Biosystems™), 0.5 μL of forward primer per well (concentration=10 μM), and 0.5 μL of reverse primer per well (concentration=10 μM). Primers complementary of the resistance gene sequence of the pDNA have been previously designed to amplify only the plasmid of interest. A MicroAmp™ Optical 384-Well reaction plate (Cat. No. 10411785, from Fisher Scientific™, Applied Biosystems™) was used, 6 μL of master mix were put in each well, followed by 4 μL of the sample. Two negative controls were made: nuclease-free water with SYBR™ green dye, and nuclease-free water with SYBR™ green dye and primers. All conditions were done in duplicates. The plate was sealed and briefly centrifuged. The thermocycler was a CFX384 Touch™ Real Time PCR detection system (from Bio-Rad). The three-step cycling, followed by the melt curve protocols are summarized in the tables 5 and 6 below.

TABLE 5
Three-step cycling protocol for qPCR

| | Temperature | | Number of |
|---|---|---|---|
| Step | (° C.) | Time | cycles |
| Dual lock DNA polymerase | 95 | 10 minutes | 1 |
| Denaturation | 95 | 15 seconds | 40 |
| Annealing | 60 | 1 minute | |

TABLE 6
Melt curve protocol for qPCR

| Step | Ramp rate | Temperature (° C.) | Time |
|---|---|---|---|
| 1 | 1.6° C./second | 95 | 15 seconds |
| 2 | 1.6° C./second | 60 | 1 minute |
| 3 | 0.15° C./second | 95 | 15 seconds |

For RNA detection after real-time (RT) qPCR was performed in technical duplicates in qPCR plates with a rapid thermal cycler system (LightCycler® 480 II, 384 (well)). The protocol was the following: 3 μl cDNA (with defined dilution factor of X1000 were mixed with one forward and one reverse primer (added at optimized concentrations usually between 200 nM and 1.0 μM), mix Takyon® No ROX SYBR 2× MasterMix blue dTTP (Eurogentec, UF-NSMT-B0701) with dNTPs, MgCl₂, Taq DNA polymerase and buffer, constituting a volume of 10 μl in total. Then, this mix was placed in LightCycler® plates.

The amplification protocol started with an initial incubation at 95° C. for 10 min (serving for activation of Taq DNA polymerase), followed by 45 amplification cycles composed of two steps: step I) a 95° C. denaturation for 10 s, step II) 60° C. primer annealing and extension for 40 seconds, (the detection of the fluorescent product was performed at the end of this 60° C. extension period with a single acquisition mode). The amplification protocol is followed by the step to perform the melting curve with one cycle of 95° C. denaturation for 5 seconds, 60° C. annealing for seconds, and a 95° C. end point (achieved by a temperature raise with a ramp rate of 0.04° C./s from 60° C. to 95° C. and accompanied by continuous detection of the fluorescent product). The amplification protocol ended by cooling at 37° C. for 10 seconds.

For plasmid quantification (dilution by 1000), the same protocol (mix and cycles) of the LightCycler® instrument is used as with cDNA quantification. Only the primers are modified.

The qPCR's primers and their concentrations are listed below:

RNA Primers (SEQ ID NOs: 265 and 266):
eGfP(1-1)For3
AGCAAAGACCCCAACGAGA
eGfP(1-1)Rev3
TCGTCCATGCCGAGAGTG
eGfP(1-1) For3Rev3:
400 nM
RNA reference Primers (SEQ ID NOs: 267 and 268):
mCherry(1-1)F1:
GACCACCTACAAGGCCAAGA
mCherry(1-1)R1:
CCGCTCGTACTGCTCCAC
mCherry(1-1) FR1:
500 nM
Plasmid (pDNA) pcDNA3.1+ Primers (SEQ ID NOs: 319 and 320):
Left primer PCDNA3.1 + 1:
GACCACCAAGCGAAACATGG
Right primer PCDNA3.1 + 1:
CCATGGGTCACGACGAGATC
PCDNA3.1 LR1(Left primer PCDNA3.1 + 1, Right primer PCDNA3.1 + 1):
700 nM

### Controls for RT-qPCR Assays

To confirm the correct functioning of the RT-qPCR analysis, two different controls were included in the experiments: a negative control and an efficacy and quantification control. All two controls were applied on each qPCR plate.

One negative control, which was an RNA-free sample (H₂O only), was added to each qPCR plate and mixed with the according primers on the qPCR plate. This negative H₂O control allowed the estimation of the Cq background levels for each amplicon, that hybridizing primers create. Efficacy and quantification controls were employed on each qPCR plate to control the primers' annealing efficacies at different cDNA concentrations and quantified.

For the “RNA efficacy and quantification control” sample, a mix of CleanCap® mRNA coding for eGFP was prepared. Subsequently, 9 different concentrations (to which 50 ng of an RNA (mCherry (Tebu-bio)) has been added) were prepared with RNAse/DNase free water (1000 ng, 500 ng, 100 ng, 50 ng, 10 ng, ng, 1 ng, 0.5 ng, 0.1 ng), extracted, reverse transcribed, and then diluted (identically as the samples, dilution factor of X1000) and mixed with the according primers used on a qPCR plate. Efficacy and quantification controls were employed on each qPCR plate to control the primers' annealing efficacies at different DNA concentrations and quantified.

For the “DNA efficacy and quantification control” sample, a mix of Plasmid (pDNA) pcDNA3.1⁺ was prepared. Subsequently, 9 different concentrations were prepared with RNAse/DNase-free water (1000 ng, 500 ng, 100 ng, 50 ng, 10 ng, 5 ng, 1 ng, 0.5 ng, 0.1 ng), extracted and diluted (identically as the samples, dilution factor of X1000) and mixed with the according primers used on a qPCR plate.

### E. Fluorescence Readout by ZetaView® Nanoparticle Tracking Analysis (NTA) Instrument.

ZetaView® NTA instrument (engine) from Particle Metrix is a Nanoparticle Tracking Analysis engine for measuring hydrodynamic particle size, zeta potential, concentration, and fluorescence. It was calibrated before the experiment according to the manufacturer's recommendations with polystyrene beads. The PBS used for the day's experiments was evaluated with the ZetaView® engine (normal average number of particles on screen in PBS: 0-5). Samples were diluted with PBS to be measured by ZetaView® within the manufacturer's recommended reading range (50-200 particles per frame). Dilutions were made in PBS, then the sample was vortexed and placed in a 1 mL syringe for the analysis.

The samples were analyzed with ZetaView® engine in scatter mode (laser 488 nm) to determine the number and size distribution of the particles. The samples were then analyzed with the ZetaView® engine in fluorescence using a laser at 488 nm and a fluorescence filter at 500 nm at different percentages of sensitivity (95% or 90%). The analog view of the ZetaView® engine was activated during the fluorescence analysis to visualize the background noise.

F. Quantification of the siRNA Cargo

The cargo-loaded MEVs were lysed as above. Next, the siRNA cargo was detected using Quant-iT® PicoGreen® Assay kit (from Life Technologies), which is able to quantify nucleic acids with picogram sensitivity. Briefly, 100 μL of dye reagent working solution was added to 100 μL of each sample to make final volume of 200 μL. A control sample prepared in the same buffer contained 10 μmol of siRNA alone. Samples were transferred into black-walled clear bottom non-treated polystyrene 96-well plates and incubated in the dark for 10 min at room temperature. The sample fluorescence was measured using a SpectraMax® fluorescence microplate reader (from Molecular Devices, USA) with excitation at 480 nm and emission at 520 nm.

### G. Quantification of Total Proteins

Total proteins were quantified by BCA or Bradford test.

### H. Statistical Analysis:

Screening and optimized experimental designs were generated with NemrodW software. The results have been analyzed using R Studio (1.4.1717 version) and Prism (8.3 version). Significance of the variables compared to the controls was evaluated with unpaired parametric t tests. “ns” stands for “not significant” meaning a p-value superior to 0.05, “*” stands for a p-value inferior to 0.05, “**” stands for a p-value inferior to 0.01, and “***” stands for a p-value inferior to 0.001. Loading efficiency were calculated based on the pDNA quantity obtained by qPCR compared to the initial pDNA quantity for each condition. Linear regression modeling was realized to evaluate the effect of tested variables on plasmid encapsulation.

### Example 6A

### Biodistribution Studies

### In Vivo Biodistribution of MEVs

Experiments were performed to determine the pathway and fate of MEVs when administered via various routes.

### 1. MEV Labelling Using DiR Fluorescent Lipophilic Tracer

As described in example 2, the fluorescent, lipophilic carbocyanine DiR (1,1-dioctadecyl-3,3,3,3-tetramethylindotricarbocyanine iodide) is weakly fluorescent in water but highly fluorescent and photostable when incorporated into membranes and can be tracked in vivo (see, Example 2 above; ThermoFisher Scientific). The DiR labelled (MEVs) are incubated with human cells. Their uptake by human cells is measured by fluorimetry analysis (fluorescence spectroscopy) on microplate readers. DiR has an excitation of 750 nm and an emission of 780 nm.

The methodology for the biodistribution studies is summarized as follows: Chlorella cells were grown in the photobioreactor as described above (120L per batch), were isolated and labeled with DiR, and labelling efficiency was assessed as described in Examples 1 and 2.

### 2. Biodistribution of MEVs in Mice

The labeled MEVs were administered to the mouse model animals via one of four routes, via: intranasal, intratracheal, oral, and intravenous administration, to determine the pathways and fate of the MEVs by each route. Treated mice were subjected to full body imaging as a function of time, and at day 3 mice were sacrificed and the organs were imaged ex vivo, and by histology in several mice organs after different routes of application.

### Animal Model

MEVs' pharmacokinetics and biodistribution were studied in Specific Pathogen Free (SPF), 7-week-old male Balb/cByJ mice (Charles River Laboratories). The animals were labelled with unique ear ID tags and acclimatized for at least 2 days. Background (control) mice were housed individually, while MEV-treated mice were housed collectively in disposable standard cages in ventilated racks at 21±3° C. (temperature recorded and controlled) with a 12 hr-12 hr light/dark cycle. Filtered water and low fluorescence laboratory food for rodents were provided ad libitum. All mice were euthanized at the end of the in vivo experiments.

Prior to the imaging, the fur of each animal was clipped using an electric clipper in the following areas: abdomen, thorax, head, and whole back. Care was taken in order to clip the fur as homogeneously as possible. Next, DiR-labelled MEV preparations were re-suspended by vortexing before filling the syringes for injection.

### Routes of Administration

The following routes were used to administer the MEVs to the animals: Intravenous (IV): 50 μL of MEV suspension containing 0.6×10¹² MEV/mL were injected in a tail vein by disposable plastic syringe and appropriate needle. Dosage of MEV administered: 3×10¹⁰ MEV/mouse.

Intranasal (IN): Animals were induced and maintained under anesthesia during IN administration by a mixture of isoflurane and oxygen as a carrier gas. A volume of 100 μL of MEV suspension containing 0.3×10¹² MEVs/mL was administered into the nostrils of the mouse using a thin pipette cone. Dosage of MEV administered: 3×10¹⁰ MEV/mouse.

Per os (PO): 100 μL of MEV suspension containing 0.3×10¹² MEVs/mL was administered orally using a disposable plastic syringe and an appropriate feeding probe. Dosage of MEV administered: 3×10¹⁰ MEV/mouse.

Intratracheal (IT): Animals were induced and maintained under anesthesia during IT administration by a mixture of isoflurane and oxygen as a carrier gas. Once adequate anesthesia was observed, animals were placed on a mouse intubation platform, suspended from the front incisors in the supine position, to maximize view of the trachea. Then, a cold light was placed on the skin near the trachea localization in order to backlight the trachea. If needed, a laryngoscope was inserted to guide the syringe of Microsprayer® sprayer into the trachea. An injection of 50 L of MEV suspension containing 0.6×10¹² MEVs/mL was instilled into the trachea. Anesthesia was maintained throughout the procedure. After injection, the mice were maintained in the same position on the intubating platform for at least 30 seconds, before being replaced in their cage. Dosage of MEVs administered: 3×10¹⁰ MEV/mouse.

### In Vivo Imaging for Biodistribution Studies

Fluorescence acquisitions were performed with the IVIS® Spectrum optical imaging system (acquired from PerkinElmer). Bidimensional (2D) fluorescence imaging was performed by sensitive detection of light emitted by DiR-labelled MEVs. Mice were anesthetized and imaged 1 hour before MEV administration. Next, fluorescence of the material to be administered was confirmed with an in vitro acquisition performed on at least 1 drop of a minimum of 20 μL of MEV suspensions, deposited in 2 different Petri dishes. The fluorescence emission was measured and detected with the selected parameters (see below). Finally, in vivo fluorescence acquisitions were performed on mice administered MEVs. The animals were induced and maintained under anesthesia with a mixture of isoflurane and oxygen. Mice were positioned in dorsal recumbency to obtain ventral images and in ventral recumbency to obtain dorsal images. At least 2 acquisitions/mouse/timepoint were taken and mice were imaged in groups of maximum 3 mice/acquisition. Images were analyzed and fluorescence quantified on 6 organs (liver, spleen, lung, kidney, intestine, and brain).

The following timepoints were used (T0=MEV administration):
Day 0-6 time points: T0-1 h±10 min; T0+30 min 5 min; T0+2 h±15 min; T0+4 h±30 min; T0+8 h±30 min; T0+10 h±30 min
Day 1—1 timepoint: T0±24 h 3 h
Day 2—1 timepoint: T0±48 h 3 h
Day 3—1 timepoint: T0±72 h±3 h

The data were analyzed with the IVIS software (Living Image Software for IVIS).

The following parameters were used for the in vivo and ex-vivo fluorescence acquisitions:
Field of View (FOV) 14×14 cm (FOV C)
Image number: In vivo: For each mouse at each applicable timepoint, at least 2 acquisitions (ventral and dorsal) were performed; 3 mice (at maximum) were imaged in one acquisition.
Ex-vivo: the organs of at least one mouse were imaged in one acquisition
Image format—TIFF format
Fluorescent probe—DiR
Excitation wavelength—745 nm
Emission wavelength—800 nm
Exposition time—Automatic, depending on the fluorescence signal detected
Minimum counts—20,000
Binning—Between 16 and 1 (depending on the fluorescence signal detected)
F/STOP—2 (if fluorescence signal reached saturation, it was automatically increased to 4, or if signal was too weak, it was decreased to 1)
Subject height—In vivo: 1.5 cm
Ex-vivo: 0.5 cm

### Ex Vivo Imaging for Biodistribution Studies

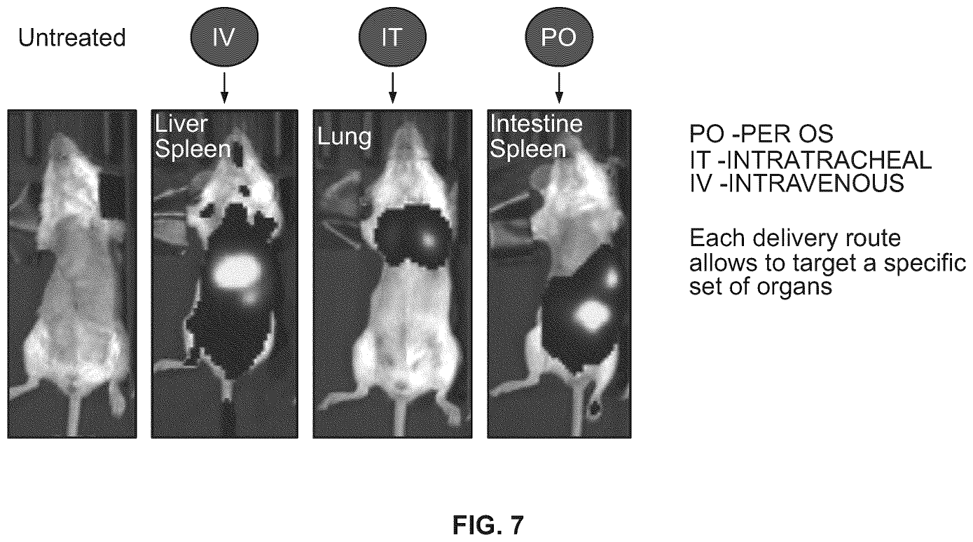

Production, purification, characterization, labelling and administration (by different routes) of MEVs was as described above. On Day 3 after administration, mice were euthanized and the organs of interest of each mouse were sampled and positioned in a Petri dish in order to perform ex vivo acquisitions. The following organs were sampled: liver, spleen, lungs, kidneys, brain, and intestine. The data were analyzed with the IVIS software (Living Image Software for IVIS). FIG. 7 shows representative patterns of biodistribution according to the route of administration, for the Intravenous (IV), Intratracheal (IT) and Per os (PO) routes.

### Pharmacokinetics Results by Each Route of Administration

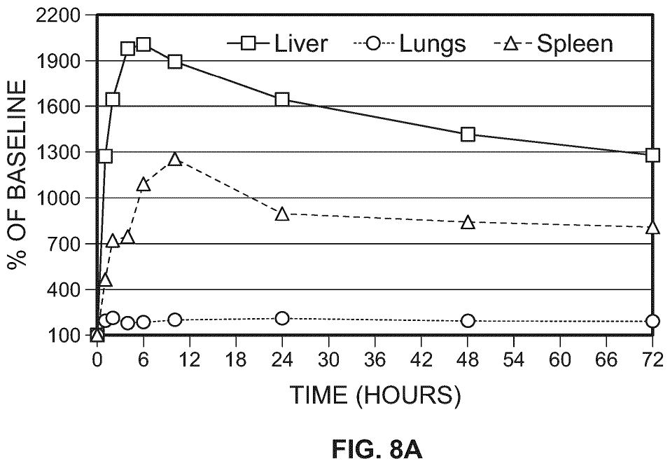

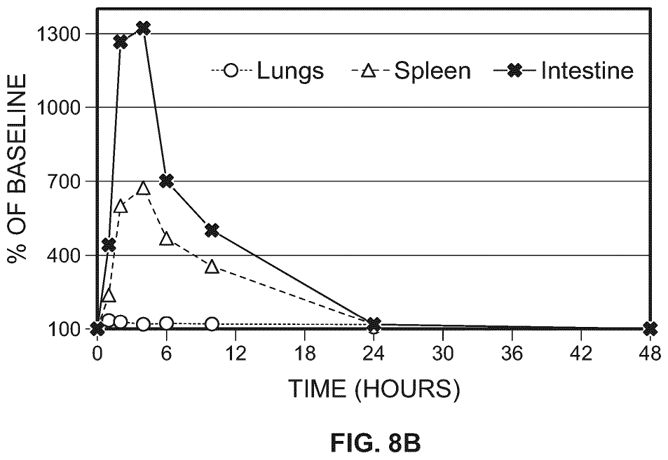

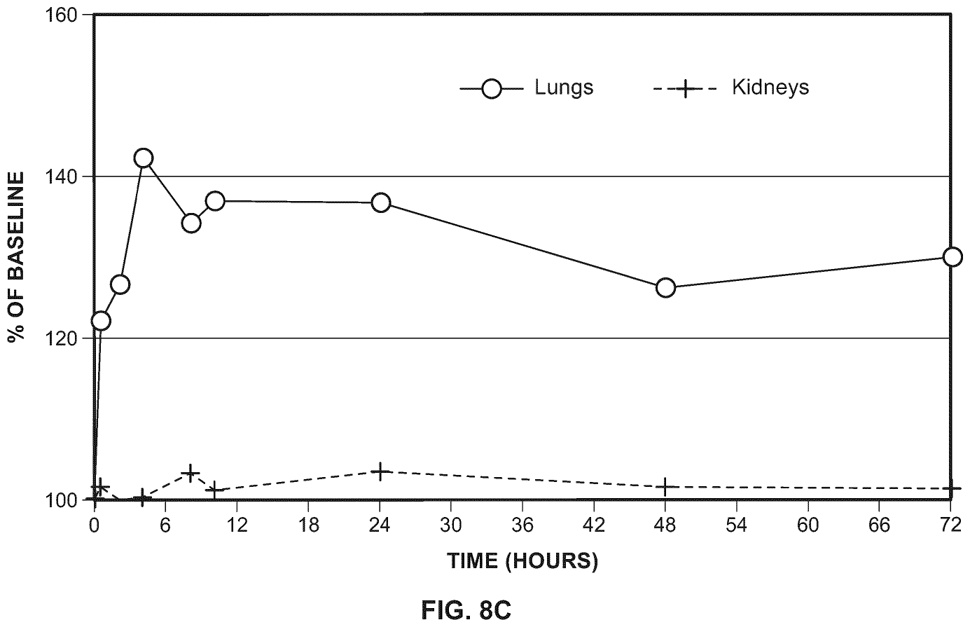

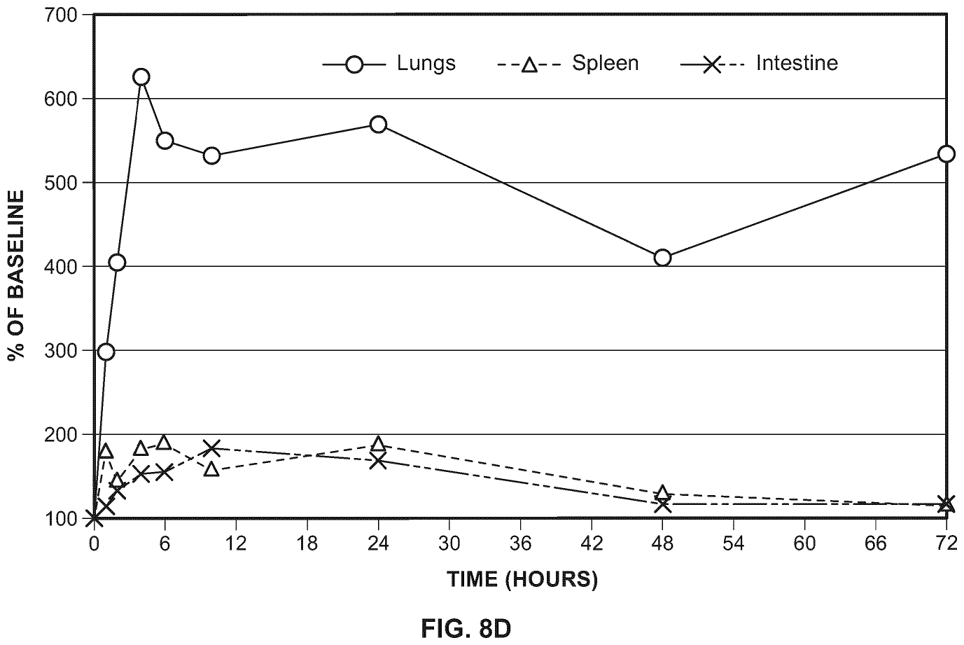

The pharmacokinetic curves demonstrating the accumulation as a percent of baseline over time (hours) after intravenous, Per os, intranasal, and intratracheal administration are set forth in FIGS. 8A-8D, respectively.

a. Intravenous Administration (FIG. 8A)
Organs targeted by unmodified MEVs—liver and spleen
PK parameters:
   Peak/plateau time: 2-24 h (liver), 4-24 h (spleen)
   % at t=day 3 compared to peak/plateau approximately 62% (liver), approx. 61% (spleen)
Longer presence or duration compared to mammalian EVs (see, e.g., Kang et al., Biodistribution of extracellular vesicles following administration into animals: A systematic review. J Extracell Vesicles. 2021; 10:e12085. (doi.org 10.10021jev2.12085)).
No visible signs of toxicity
   b. Per Os (Oral) Administration (FIG. 8B)
Organs targeted by following oral administration: intestine and spleen
Oral availability:
   MEVs are orally available; they resist the passage through the stomach, reach the intestine and subsequently appear in the spleen.
   Mammalian EVs are not orally available, with the exception of bovine milk EVs.
   Feasibility of MEV-based oral vaccines due to ability to reach intestinal tissue with per os route.
PK parameters:
   Peak/plateau time: 1-6 h (intestine), 1-10 h (spleen)
   % at t=10 h compared to peak/plateau: approx. 33% (intestine), approx. 44% (spleen)
   % at t=48 h compared to peak/plateau: 0% (intestine), 0% (spleen)
No visible signs of toxicity
   c. Distribution in Organs Following Intravenous and Oral Administration.

Liver: Following intravenous administration, the liver is the primary target organ. Following oral administration, the liver is marginally targeted, if at all. This indicates that MEV clearance by liver following per os delivery is low or none.

Spleen: The pharmacokinetics profile depends on whether administration is intravenous or oral (per os). Intravenous administration is longer lasting. At day 3, 50-70% of the peak is still remaining. Oral administration is shorter lasting; at 48 hours, 0% remains.

d. Intranasal Administration (FIG. 8C) Organs Targeted by the Unmodified MEVs are the Lungs
Preliminary PK parameters:
   Peak/plateau time: 4-28 h
   % at t=day 3 compared to peak/plateau: 70-80%
No visible signs of toxicity e. Intratracheal administration (FIG. 8D)
Organs targeted by the MEVs are the lungs
   PK parameters:
   Peak/plateau time: 2-72 h
   % at t=day 3 compared to peak/plateau: approx. 80%
   No visible signs of toxicity

### Example 6B

Biodistribution of MEVs Labelled with PK1126 after Per Os (PO) Administration in BALB/cByJ Mouse. Internalization of Fluorescently Labelled MEVs in the Mouse Intestinal Epithelium (Enterocytes), GALT Cells after Per Os Administration

PKH26 is a lipophilic fluorescent dye used for general membrane labeling. It is used for in vitro studies, as well as long term in vivo experiments due to enhanced stability (in vivo half-life is over 100 days). MEVs were labeled with PKH26 as described above and administered orally to BALB/c mice at a dose of 4×10exp10 MEV particles per animal. Between 0.5 to 24 hours post-administration, the animals were sacrificed, and the intestine was harvested.

### A. Organ Sampling and Processing: Intestine

The jejunum and ileum were gently rinsed with a cold PBS solution. Then, a piece of approximately 0.5 to 1 cm of jejunum and ileum were snap-frozen and lastly, each piece was longitudinally placed on bottom of the cryomold for OCT inclusion (as described below).

### B. Snap-Frozen:

A small stainless-steel bowl was placed in the bottom of a container containing dry ice in pellet form and liquid nitrogen. Then, isopentane/2-methylbutane was slowly added in the container. When, the dry ice pellets stop bubbling vigorously or/and the isopentane start to become opaque, the isopentane/2-methylbutane is at optimal temperature. Organs were gently immerged in isopentane/2-methylbutane and then placed in cassette (one cassette with each section of liver, the spleen and the piece of jejunum and one cassette with the piece of ileum only). Then, cassettes were stored at ≤−75° C. until OCT inclusion.

### C. OCT Inclusion Protocol

A small stainless-steel bowl was placed in the bottom of a container containing dry ice in pellet form and liquid nitrogen. Some pellets of dry ice were also placed directly in the bowl. Then, isopentane/2-methylbutane was slowly added in the container. When, the dry ice pellets stop bubbling vigorously and/or the isopentane starts to become opaque, the isopentane/2-methylbutane is at optimal temperature. The frozen tissue samples were then placed and oriented as mentioned above for each organ in the cryomold. The tissues were gently pushed with forceps to ensure that the bottom of surface of the tissue is placed properly (touching the face of the bottom, center in the mold and properly oriented). The cryomold with frozen tissue samples were placed on the surface of the cold isopentane/2-methylbutane. Optimal cutting temperature compound (OCT compound) was carefully deposited onto the specimen until it is completely covered. After hardening of the OCT compound (between 30 seconds and 1 minute), the OCT embedded block was placed in a bag (such as zip freezer bag). The frozen blocks were temporarily stored in dry ice. Then, slices of 5 μm were performed with a cryostat then glued on untreated slides for the histology and on treated slides for the immunochemistry.

### D. HES Staining

The HES staining allowed the observation of the morphology and the structure of tissues. After fixation in acetone, sections were immersed successively in solutions of Harris hematoxylin, eosin and saffron. After dehydration, sections were mounted between slide and cover slip using Entellan® mounting medium. Cytoplasm appeared in pink and nuclei in violet blue. Extracellular matrix was stained in yellow to pink.

### E. PKH26 Dye

Accumulation of fluorescent labeled MEVs was visualized by a LSM700 laser scanning confocal microscope (from Zeiss) and images were processed with LSM Image Browser. DAPI (4′,6-diamidino-2-phenylindole) [Invitrogen, Ref S36938] was used as nuclear counterstain prior to the imaging. Sections were mounted with aqueous medium with DAPI for fluorescent slide scanner observation (maximum 10 days after collection).

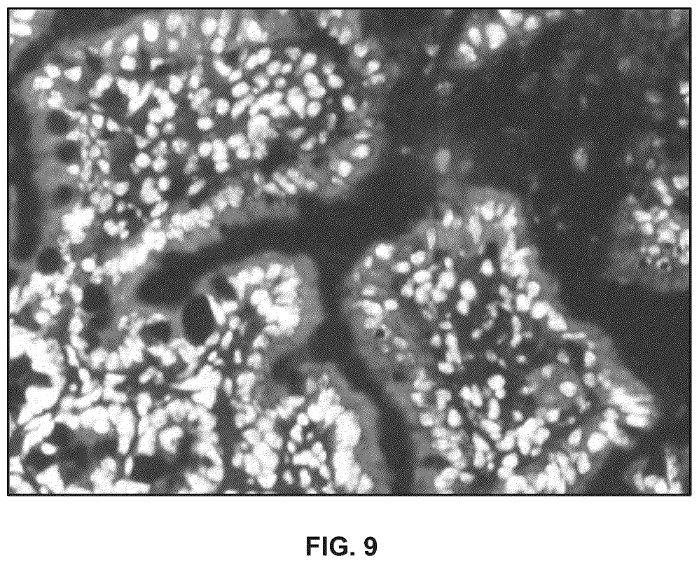

When MEVs are orally administrated to the mice, they follow through the digestive tract. MEVs first reach the stomach, where they resist the stringent conditions of the gastric juice. In about 30 minutes after administration, they reach the intestine, where they stay for several hours. Results are shown in FIGS. 9-11.

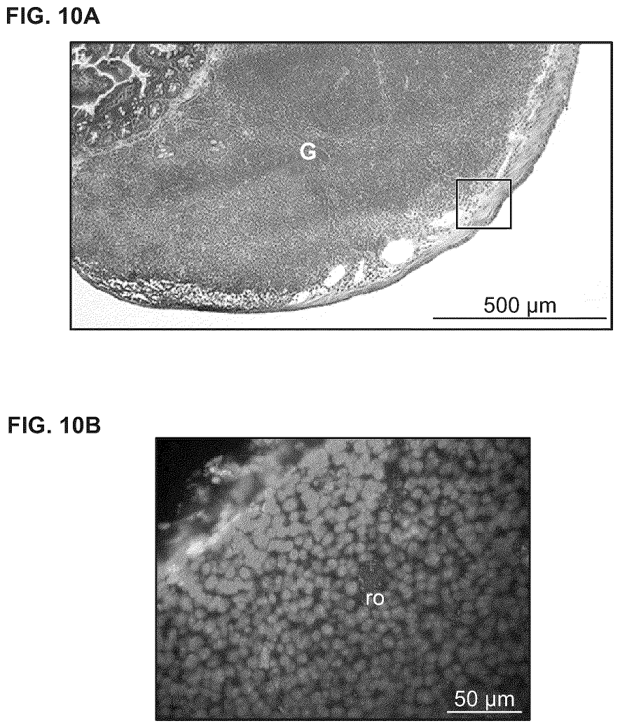

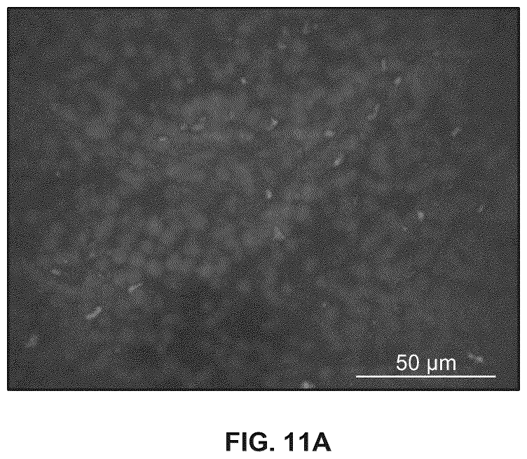

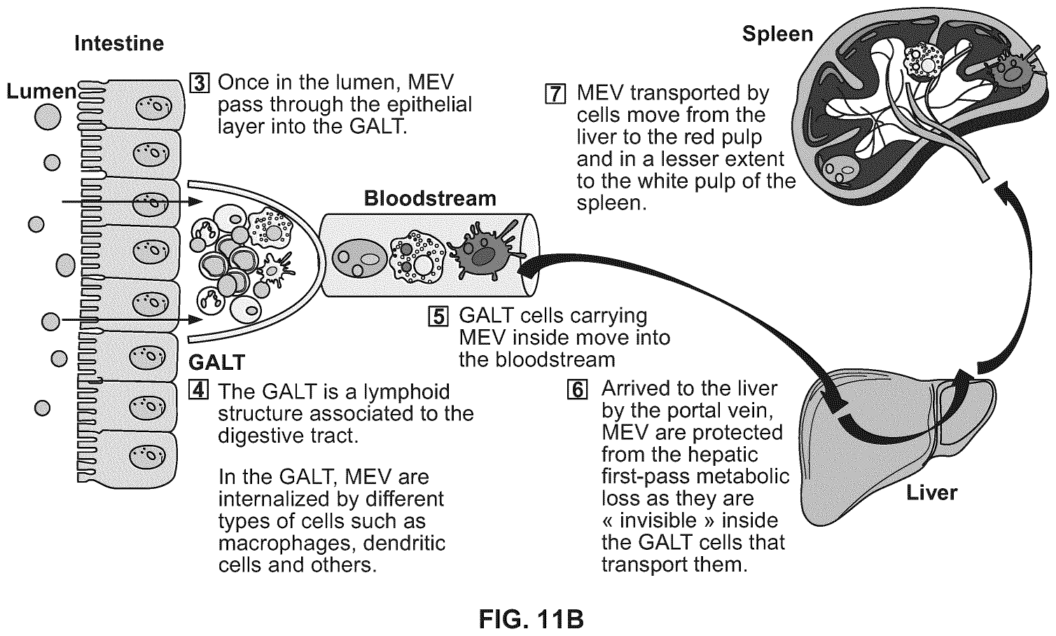

Once in the lumen of the intestine, as predictable from the Example 6, MEVs are internalized by the cells of the intestinal epithelium, or enterocytes (FIG. 9). MEVs also pass through the epithelial layer into the GALT (FIGS. 10A-10B). The GALT is a lymphoid structure associated to the digestive tract (gut-associated lymphoid tissues) located beneath the intestinal epithelium. It is located at specific spots along the intestine. GALT is a dense tissue composed of germinal centers with B and T lymphocytes, plasmocytes and innate immune cells including dendritic cells and macrophages. In the intestine, fluorescence is observed mostly in the GALT. Involvement of the GALT cells are relevant to oral vaccination, as it may be triggering the immune reaction via the antigen-presenting components of the cellular population. Fluorescence appears (FIG. 10B) concentrated in discrete spots around nucleus meaning that MEVs are localized in the plasma of cells occupying all the space in the plasma. This allows revealing of the shape of cells. Cells with MEVs inside correspond to histiocytes (resident macrophages) or dendritic cells, respectively, and are located mainly at the periphery of GALT and in the center of the GALT. No visible MEVs appear in the liver at all times evaluated (30 min to 24 h). A few hours after administration, dendritic/macrophage cells with MEVs inside move from the GALT to the spleen and stay in the spleen for several hours. MEVs primarily reach the red pulp (blood cells and some Th and B cells) are and in a lesser extent the white pulp of the spleen (lymphoid cells) (see FIG. 11A).

In the spleen, fluorescence is visible in several areas corresponding to the white and to the red pulps. The intensity of the fluorescence decreases in a gradient from the red pulps to the white pulps. Fluorescence is detected in spots inside the cell's cytoplasm. Cells with MEVs inside are round or reticular and have similar size; they may respectively be histiocytes (or tissue resident-macrophages) and/or dendritic cells.

Our first conclusion is that labelled-MEVs can be detected in the cytoplasm of mononuclear cells (tissue resident macrophages (histiocytes) and dendritic cells) in the GALT and in the spleen.

Importantly, MEVs reach the spleen directly, via GALT, dendritic cells and macrophages, without going per se into the systemic circulation. Cells can move from the GALT into the bloodstream directly or they can join it after they have first passed through the lymph stream. GALT cells carrying MEVs inside eventually arrived at the liver by the portal vein and/or by mesenteric lymphoid nodes and then to the systemic circulation. In both cases, however, MEVs, are ‘invisible’ to the liver as they have been internalized by GALT cells that naturally transport them to the spleen (see FIG. 11B).

Our second conclusion is that, therefore, the MEVs administered orally are not captured by the liver, as are the MEVs that have been administered intravenously. This is advantageous for MEVs that carry cargo, such as antigens or nucleic acid encoding antigens and/or immune modulators, intended for the immune system.

Our third conclusion, as shown by these results, is that following the oral administration, the ‘absorption’ of MEVs in the intestine (1) does not affect the general morphology of cells or tissues where MEVs have been internalized, including the intestinal epithelium, the GALT, and a few hours later the spleen; and (2) does not induce any inflammatory reaction. In situ IFH analysis of the biodistribution of labelled MEVs (labelled with pKH26 fluorescent dye) following oral administration shows the detection of fluorescence from 8 h to 24 h in all the intestinal epithelium, the GALT, and the spleen. In addition, after oral administration, MEVs are not observed in the liver at all, and they do not seem to travel systemically in a free form (but inside the cells from the GALT that have internalized them in the intestine).

Our fourth conclusion from these results is that MEVs can solve one of the problems facing the development of mucosal vaccines: the delivery of antigens through the mucosa. Therefore, MEVs are a suitable vaccine delivery system (VDS) to elicit a mucosal response by oral administration.

### Example 7

### MEV-Mediated In Vivo Delivery, Expression and Biologic Activity of Luciferase Enzyme and Luciferase-mRNA

A. Intra-Tracheal Administration of MEVs Loaded with Luciferase mRNA or Luciferase Protein

In vivo bioluminescence was used to study the biodistribution and MEV-mediated delivery and expression of luciferase mRNA and luciferase protein. Eight (8) female BALB/cByJ mice were divided into two groups (4 animals per group) for intra-tracheal (IT) administration. Mice in each group were treated either with MEVs loaded with mRNA encoding luciferase (MEV-luc mRNA) or MEVs loaded with luciferase enzyme (MEV-luc protein). MEVs were loaded and characterized as described above. For the IT administration, the animals were maintained under isoflurane-induced anesthesia and placed on a mouse intubation platform. Then, the trachea was backlit with a cold light and 50 μL/mouse of MEV formulations were administered intra-tracheally using a Microsprayer® aerosolizer (Penn-Century). After administration, mice were maintained in the same position on the intubating platform for at least 30 s before being replaced in their cage. A background mouse also was included in the study; this mouse did not receive any MEV administration and served as a control to measure the background level of the bioluminescence acquisition.

### B. Bioluminescence Acquisition

Prior to imaging, the fur of each animal was shaved on the abdomen and thoracic area using an electric clipper. Each mouse was intraperitoneally (IP) injected with 200 μL of luciferin solution at a concentration of 16.5 mg/mL. Bioluminescence acquisitions were performed 10 minutes after the luciferin injection, with mice anesthetized with a mix of isoflurane/oxygen. Animals were positioned in dorsal recumbency (ventral images) to visualize the mice abdomen and thorax and particularly the lungs, liver, intestine, and spleen. Bioluminescence acquisition in vivo was performed with the IVIS® LUMINA X5 optical imaging system (PerkinElmer). The bioluminescence was measured at 6 timepoints: 1 h before MEV administration and 6, 30, 48, 54 and 72 h post-administration. The bioluminescence signal was visualized and quantified in the lungs, liver, intestine, and spleen. Animals were euthanized after the last bioluminescence acquisition. At each timepoint, bioluminescence acquisitions were first performed on the background mouse to measure the background flux level corresponding to the auto-bioluminescence of mice and the noise emitted by the camera of the optical imaging system. Bioluminescence acquisitions then were performed on the experimental mice.

C. Results: Delivery of Luciferase mRNA, Protein Expression and Luciferase Activity In Vivo

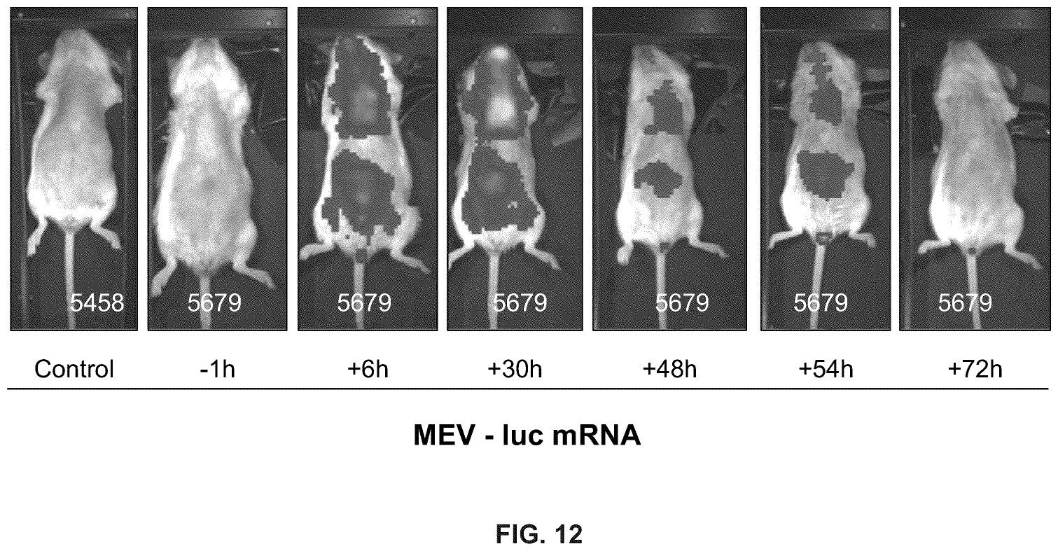

FIG. 12 shows whole-body bioluminescence imaging of a representative animal treated with MEVs loaded with luciferase mRNA. MEV-mediated delivery of mRNA resulted in expression of enzymatically active luciferase in mouse tissues, as evidenced by the luminescence signal detected after the administration of luciferin. The target organs after intratracheal administration include the respiratory system (lungs and naso-buccal epithelium), as well as the gastrointestinal tract (intestinal epithelium) resulting from partial regurgitation of the MEV formulation. Luciferase activity after a single administration of MEVs was detectable starting from 6 hours post-administration and lasted for 2-3 days. The control was a background mouse with no MEV administration.

### D. Results: Luciferase Delivery and Activity In Vivo

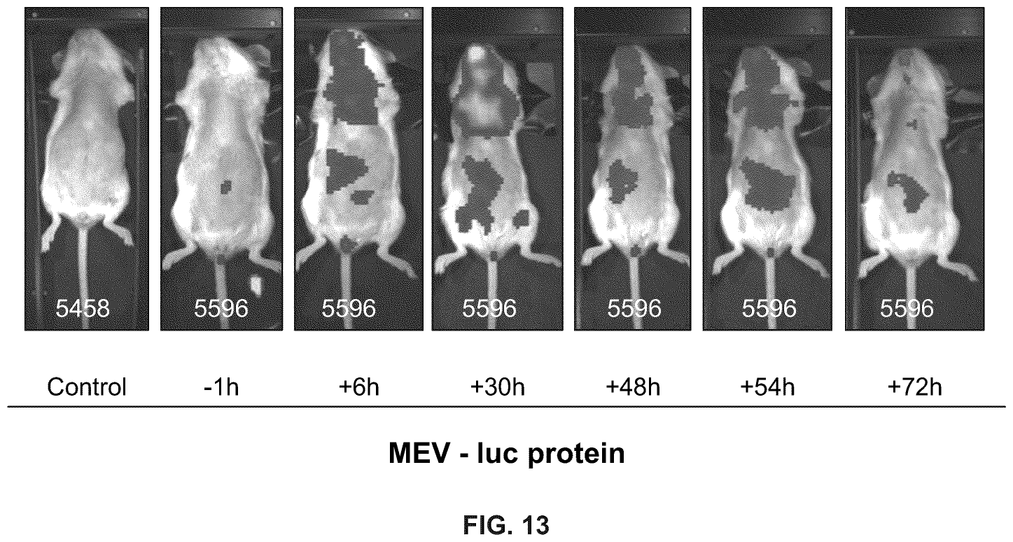

FIG. 13 depicts whole-body bioluminescence imaging of a representative animal treated with MEVs loaded with luciferase enzyme. MEV-mediated delivery of the protein resulted in enzymatic activity of luciferase in mouse tissues, as evidenced by the luminescence signal detected after the administration of luciferin. The target organs after intratracheal administration include the respiratory system (lungs and naso-buccal epithelium), as well as the gastrointestinal tract (intestinal epithelium) resulting from partial regurgitation of the MEV formulation. Luciferase activity after a single administration of MEVs was detectable starting from 6 hours post-administration and lasted for 3-4 days. The control is a background mouse with no MEV administration. The results demonstrate that MEVs are able to deliver intact payload, including cargoes of degradation-sensitive enzymatic activity and extremely labile mRNA molecules. Therefore, MEVs allow for delivery and expression of payloads in the intestinal epithelial cells. This further demonstrates the potential of using MEVs to reach to the intestinal wall and deliver orally formulated vaccines.

Following oral administration of MEVs loaded with the luciferase protein or with luciferase-mRNA reach the intestine and enter the intestinal epithelial cells in vivo. A similar observation has been made in vitro with FHs-74 Int cells and with FHC cells, which are human embryonic epithelial cells from small intestine and from colon, respectively). After penetration the payload is released and either the protein, or the mRNA translate in protein are active and the biological activity of luciferase was well detected. These results shown, also the availability of MEVs as VDS for different modalities of payloads (proteins, mRNAs, and others).

### Example 8

Assessment of the Capacity of Extracellular Vesicles from Microalgae (MEVs) as a Protein Vaccine Delivery System Following Intramuscular (IM) Administration

### A. In Vivo Study in Healthy C57BL/6J Mice.

Animals were divided in groups (6 mice per group) and treated with the following:
Group 1: IM administered with OVA (Ovalbumin) in adjuvant (Complete Freund Adjuvant (CFA) on Day 0 or Incomplete Freund Adjuvant (IFA) on Day 21) on Days 0 and 21,
Group 2: IM administered with MEV-OVA on Days 0 and 21,
Group 3: IM administered with adjuvant only (CFA on Day 0 and IFA on Day 21) on Days 0 and 21,
Group 4: IM administered with MEV only on Days 0 and 21,
Group 5: IM administered with OVA (Ovalbumin) in adjuvant (Complete Freund Adjuvant (CFA) on Day 40 or Incomplete Freund Adjuvant (IFA) on Day 61) on Days 40 and 61.
Group 6: IM administered with MEV-OVA on Days 40 and 61.
Group 7: Control group; mock administration

Mice of groups 1-4 and 7 were euthanized on Day 43 after the first vaccinal administration. Mice of groups 5-6 were euthanized on Day 82 after the first vaccinal administration.

Whole blood was sampled just before euthanasia to collect serum for quantification of anti-OVA antibody by Enzyme-Linked Immunosorbent Assay (ELISA), and to perform flow cytometry as defined below.

Just after euthanasia, spleen, and inguinal and iliac lymph nodes (LNs) were collected and processed to perform flow cytometry analysis to quantify the T cell activation after in vitro stimulation with OVA peptides.

### B. Immunization Material and Mice Treatment:

### 1. Group 1: OVA in Adjuvant

On Day 0

OVA was dissolved in sterile water to obtain a solution concentrated at 0.1 mg/mL. 300 μL of OVA solution was thoroughly mixed with 300 μL of CFA (well homogenized by vortexing) in two 1 mL luer-lock syringes connected through the luer fitting to the 3-way valve. The thick white emulsion of OVA in adjuvant was used to treat the mice of Group 1.

On Day 21:

OVA was dissolved in sterile water to obtain a solution concentrated at 0.1 mg/mL. 300 μL of OVA solution was thoroughly mixed with 300 μL of IFA (well homogenized by vortexing) in two 1 mL luer-lock syringes connected through the luer fitting to the 3-way valve. The thick white emulsion of OVA in adjuvant was used to treat the mice of Group 1.

2. Group 2: MEVs Loaded with OVA

On Days 0, and 21:

MEVs were produced, purified, and characterized as described in Example land 2. MEVs were loaded with ovalbumin as described in the above Examples. MEV-OVA were vigorously mixed by vortexing for about 60 seconds and used to treat the mice of Group 2.

### 3. Group 3: Adjuvants (Complete Freud Adjuvant (CFA) or Incomplete Freud Adjuvant (IFA)

On Day 0:

CFA was well homogenized by vortexing and used to treat the mice of Group 3.

On Day 21:

IFA are well homogenized by vortexing and used to treat the mice of Group 3.

### 4. Group 4: MEVs (Non-Loaded)

On Days 0, and 21:

MEVs were produced, purified, and characterized as described in Example 1 and 2. Non-loaded MEVs were vigorously mixed by vortexing for about 60 seconds and used to treat the mice of Group 4.

### 5. Group 5: OVA in Adjuvant

On Day 0:

OVA was dissolved in sterile water to obtain a solution concentrated at 0.2 mg/mL. 300 μL of OVA solution was thoroughly mixed with 300 μL of CFA (well homogenized by vortexing) in two 1 mL luer-lock syringes connected through the luer fitting to the 3-way valve. The thick white emulsion of OVA in adjuvant was used to treat the mice of Group 5.

On Day 21:

OVA was dissolved in sterile water to obtain a solution concentrated at 0.2 mg/mL. 300 μL of OVA solution was thoroughly mixed with 300 μL of IFA (well homogenized by vortexing) in two 1 mL luer-lock syringes connected through the luer fitting to the 3-way valve. The thick white emulsion of OVA in adjuvant was used to treat the mice of Group 5.

6. Group 6: MEVs Loaded with OVA

On Days 40, and 61:

MEVs were produced, purified, and characterized as described in Examples 1 and 2. MEVs were loaded with ovalbumin as described in the above Examples. MEV-OVA were vigorously mixed by vortexing for about 60 seconds and used to treat the mice of Group 6.

### C. Intramuscular Administration

IM injections were performed into the right quadriceps using a disposable plastic syringe and an appropriate needle.

On Day 0 and Day 21:
Group 1 was IM administered with 100 μL of an emulsion of OVA (50 mg/100 mL) in adjuvant (CFA on Day 0 and IFA on Day 21),
Group 2 was IM administered with 100 μL of MEV-OVA at a concentration of 4¹⁰ particles/mL,
Group 3 was IM administered with 100 μL of adjuvant (CFA on Day 0 and IFA on Day 21),
Group 4 was IM administered with 40 μL of MEV at a concentration of 1·2¹¹ particles/mL,
Group 5 was IM administered with 100 μL of an emulsion of OVA i (100 mg/100 mL) in adjuvant (CFA on Day 0 and IFA on Day 21),
Group 6 was IM administered with 100 μL of MEV-OVA at a concentration of 4¹¹ particles/mL.

### D. Blood Sampling and Serum Collection

According to the experimental schedule, at 43 or 82 days after first IM administration in groups 1-4 or groups 5-6 respectively, whole blood (WB) was collected by retro-orbital sinus or intracardiac puncture under anesthesia.
1—Whole Blood sampling for flow cytometry acquisitions:
About 300 μL of WB were collected and deposited in microtube previously heparinized (60 μL of heparin) to avoid coagulation.
2—WB sampling for serum collection:
Minimum of 300 μL of WB were sampled and deposited in a Serum Separation Tube (SST) in order to obtain a minimum of 150 μL of serum. The SST were kept at room temperature until clotting and centrifuged at 3000 g for 10 minutes at room temperature. Serum aliquots were stored at <−75° C. until processing for quantification of anti-OVA antibodies by ELISA.

### E. Animal Euthanasia and Organs Sampling and Processing

According to the experimental schedule, at 43 or 82 days after first IM administration groups 1-4 and groups 5-6 respectively, animals were euthanized under anesthesia by cervical dislocation and the selected organs were collected.
Spleen (free of fat) was cut into small pieces (about 1-2 mm³ per piece) and deposited together in 1 mL of Complete Culture Medium (CCM composed of RPMI1640 medium supplemented with 10% Fetal Bovine Serum (FBS), 1% L-Glutamine and 1% Penicillin-Streptomycin).
Right inguinal and iliac lymph nodes (side of the IM injection site) were also cut into small pieces (about 1-2 mm³ per piece) and deposited together in 0.5 mL of CCM.

The spleen pieces with 1 mL of CCM were transferred in a BD™ Medimachine Medicon of 50 μm (pre-rinsed with Cell Wash buffer (BD Biosciences)). The Medicon was inserted in the BD™ Medimachine System, and one cycle of 30 seconds was performed to disaggregate the spleen tissues. The cell suspension obtained was aspirated with a syringe through the syringe port of Medicon. The Medicon was rinsed 2 times with 1 mL of Cell Wash buffer and the cell suspension obtained was also aspirated. Then, the obtained cell suspension was filtered using a BD™ Medimachine Filcon of 100 μm and deposited in a 5 mL tube. The cells were counted using an automatic cell counter. Finally, the cell suspension was split in 2 tubes: 2×10⁶ cells for T cell activation protocol and 1×10⁶ cells for flow cytometry staining. The tubes were centrifuged at 300 g for 7 minutes at RT. The supernatant was completely removed. Cell pellet of tube 1 (for T cell activation protocol) was resuspended in 100 μL of RPMI complete medium (RPMI, 1% P/S, 10% FBS, 1% Hepes 1M, 1% Sodium pyruvate 100 mM, 1% NEAA 100×) and cell pellet of tube 2 (for flow cytometry staining) was resuspended in 100 μL of Cell Wash. Cell suspensions were stored at +5° C.±3° C. until staining for flow cytometry or T cell activation protocol.

The LNs pieces with 0.5 mL of CCM were transferred in a BD™ Medimachine Medicon of 50 μm (pre-rinsed with Cell Wash buffer). The Medicon was inserted in the BD™ Medimachine System, and one cycle of 30 seconds was performed to disaggregate the LNs tissues. The Medicon was rinsed once with 0.5 mL of Cell Wash buffer and the cell suspension obtained was aspirated with a syringe through the syringe port of Medicon. Then, the obtained cell suspension was filtered using a BD™ Medimachine Filcon of 100 μm and deposited in a 5 mL tube. The cells were counted using an automatic cell counter. Finally, the cell suspension was split in 2 tubes: 2×10⁶ cells for T cell activation protocol and 1×10⁶ cells for flow cytometry staining. The tubes were centrifuged at 300 g for 7 minutes at RT. The supernatant was completely removed. Cell pellet of tube 1 (for T cell activation protocol) was resuspended in 100 μL of RPMI complete medium (RPMI, 1% P/S, 10% FBS, 1% Hepes 1M, 1% Sodium pyruvate 100 mM, 1% NEAA 100×) and cell pellet of tube 2 (for flow cytometry staining) was resuspended in 100 μL of Cell Wash. Cell suspensions were stored at +5° C.±3° C. until staining for flow cytometry or T cell activation protocol.

### F. Quantification of Antibody Anti-OVA by ELISA

Immunoglobulins (Ig) quantification was performed by an indirect ELISA according to the dedicated kit from Chondrex: Kit #3011 for Total IgG, Kit #3013 for IgG1, Kit #3018 for IgA. The same protocol was used for the three different kits. Briefly, 100 μL of diluted OVA solution were added in each well and then the plate was incubated at +5±3° C. overnight. The plate was washed with 1× wash buffer at least three times and 100 μL of blocking Buffer were added to each well and incubated at RT for 1 hour. The plate was washed a second time and 100 μL of diluted standard [range from to 12.5 ng/mL to 0.2 ng/mL] and serum sample (1/50 dilution) were added in duplicate. The plate was incubated at RT for 2 hours and washed as described before. Then, the secondary antibody was added in each well (100 μL) and the incubation was performed at RT for 1 hour. After incubation, the plate was washed and 100 μL of TMB solution (previously diluted) were added to each well. Reaction with this chromogen solution was done during 25 min at RT and stopped by the addition of 25 μL of 2N sulfuric acid. Finally, the plate was gently mixed, and absorbance was read at 450 nm with a GloMax® plate reader (Promega, Madison, WI). The average of duplicate readings for each standard control were used to plot a standard curve, which was used to quantify the samples in ng/mL of blood.

G. Stimulation Protocol: Interferon Gamma (IFN-g) and Tumor Growth Factor Alpha (TNF-α) Production in CD4+ and CD8+ Populations from Peripheral Blood, LNs and Spleen.

After resuspension of cell pellet of spleen, LNs and blood in 100 μL of RPMI complete medium, the cell suspension was placed in a 96-well plate and incubated between +35° C. and +40° C. with 1 to 6% of CO₂ until peptide stimulation performed as described below. Briefly, 100 μL of 2× peptide cocktail (2 μg/mL of brefeldin A, 2 μl/mL of SIINFEKL (SEQ ID NO:189) peptide and 5 μl/mL of SQAVHAAHAEINEAGR peptide (SEQ ID NO:190) in RPMI complete medium: RPMI, 1% P/S, 10% FBS, 1% Hepes 1M, 1% Sodium pyruvate 100 mM, 1% NEAA 100×) were added to each experimental well. The 96-well plate was incubated between +35° C. and +40° C. with 1 to 6% of CO₂ overnight (for 17 hours). After incubation, cells were centrifuged at 300 g for 5 minutes at RT and supernatant was discarded. Then cell pellets were resuspended in 200 μL of Stain Buffer and transferred in 5 mL tubes until staining for flow cytometry analysis as described below.

H. Flow Cytometry on Cells Activated with Peptides

### Staining of Cells Suspension:

After stimulation protocol, cells were centrifuged at 300 g for 5 minutes at RT and supernatant was removed. Cell pellets were resuspended in 54 μL of Mix Fc Block and incubated for 10 minutes at +5±3° C., followed by adding 52.5 μL of the Mix Extra of antibodies (panel detailed in Table 24) and incubation for 30 minutes at RT in dark. 300 μL of Staining buffer were added, tubes were centrifuged at 300 g for 5 minutes at RT; supernatant was discarded, and cells resuspended in 400 μL of staining buffer. Tubes were centrifuged again at 300 g for 5 minutes at RT; supernatant was discarded, cells were resuspended in 200 μL of Cytofix/Cytoperm™ solution (BD) and incubated 20 minutes at +5±3° C. 200 μL of 1× Perm/Wash™ buffer (BD) diluted in water (1/50) were added, tubes were centrifuged again at 300 g for 5 minutes at RT, supernatant was discarded, and cells resuspended in 400 μL of Perm/Wash™ buffer. Tubes were centrifuged again at 300 g for 5 minutes at RT and supernatant was discarded, cells were resuspended in 56 μL of the Mix Intra of antibodies and incubated 30 minutes at +5±3° C. in dark. 300 μL of Perm/Wash™ buffer were added, tubes were centrifuged at 300 g for 5 minutes at RT and supernatant was discarded and cells resuspended in 400 μL of Perm/Wash™ buffer. Finally, tubes were centrifuged again at 300 g for 5 minutes at RT and supernatant was discarded, cells were then resuspended in 300 μL of Stain Buffer for flow cytometry acquisition.

### Preparation of Mix:

Mix Fc Block: 50 μL of Stain Buffer
3 μL of mouse Fc Block
1 μL of Golgi Stop

Mix Extra: Extracellular staining: 50 μL of Brilliant Buffer with:
2.0 μL mCD8a/PE,
0.3 μL mCD4/PE-Cy7,
0.25 μL Live Dead

Mix Intra: Intracellular staining: 50 μL of Brilliant Buffer with:
2.5 μL mTNFa/BV421,
3.5 μL mIFNg/BV650.

Samples were stored at +5±3° C. prior to acquisition.

TABLE 7
Panel of antibodies used in flow cytometry experiments (cells activated with peptides)

| | | | | | | Volume/ | |
|---|---|---|---|---|---|---|---|
| Lasers | Filters | Dye | Description | Clone | Source | sample (μl) | |
| Violet | 448/45 | BV421 | mTNFa | MP6-XT22 | BD 563387 | 2.5 | μl |
| (405 nm) | 606/36 | BV650 | mIFNg | XMG1.2 | BD 563854 | 3.5 | μl |
| Blue | 560 LP | PE | mCD8a | 5H10-1 | BD 567630 | 2.0 | μl |
| (488 nm) | (586/42) | | | | | | |
| | 752 LP | PE-CY7 | mCD4 | RM4-5 | BD 552775 | 0.3 | μl |
| | (783/56) | | | | | | |
| Red | 752 LP | Live/dead | LIVE/DEAD ™ | N/A | Invitrogen | 0.25 | μl |
| (640 nm) | (783/56) | | Fixable Near-IR | | | | |
| | | | Dead Cell | | | | |
| | | | Stain Kit | | | | |

### I. WB, Spleen and LN Cells Staining and Flow Cytometry Acquisitions

The flow cytometry acquisitions were performed with a BD FACSLyric™ flow cytometer (BD Biosciences). A quality control was performed before each acquisition session using CS&T Beads (BD Biosciences).

### Staining of Cells Suspension:

A red blood cell (RBC) lysis was performed before staining for the blood samples using the BD FACS™ Lyse Wash Assistant (LWA) (Duo-lyse-VXC protocol). Briefly, 100 μL of WB were incubated sequentially with 900 μL and then with 700 μL of BD Pharm Lyse™ lysing buffer solution, to lyse the RBCs. Samples were washed with BD® CellWASH buffer (BD Biosciences) to obtain a cell suspension of about 350 μL. Then, staining of WB, spleen, small intestine and LNs suspension cells was performed according to the following protocol:

Cells (about 1×10⁶ cells in 100 μL of CellWASH buffer or WB cell suspension obtained after RBC lysis) were incubated for 5 minutes with 54 μL of Mix Fc Block in the dark at +5±3° C. Next, cells were incubated for 30 minutes in the dark at +5±3° C. with 68 μL of mix of antibodies Mix Extra (panel detailed in Table 25). Cells were washed with BD© CellWASH buffer (BD Biosciences) and were resuspended with about 350 L±50 μL of CellWASH buffer using the LWA (Wash only-VXC protocol).

### Staining Preparation of Mix:

Mix Fc Block: 50 μL of Stain Buffer
3 μL of mouse Fc Block
1 μL of Golgi Stop

Mix Extra: Extracellular staining: 50 μL of Brilliant Buffer buffer with all antibodies as described in Table 25.

Samples were stored at +5±3° C. prior to acquisition.

TABLE 8
Panel of antibodies used in flow cytometry experiments (WB, Spleen and LN cells)

| | | | | | Volume/ | |
|---|---|---|---|---|---|---|
| Lasers | Filters | Dye | Description | Clone | sample μl | |
| Violet | 715/50 | BV711 | mcD62L | MEL-14 | 1.25 | ul |
| (405 nm) | 755 LP | BV786 | mcD49d | Clone R1-2 | 3.5 | μl |
| Blue | 560LP (586/42) | PE | mcD8a | 5H10-1 | 2.0 | μl |
| (488 nm) | 665 LP (700/54) | BY700 | mcD44 | IM7 | 3.5 | μl |
| | 752 LP (783/56) | PE-CY7 | mcD4 | RM4-5 | 0.3 | μl |
| Red | 660/10 | AF 647 | mcD11a | M17/4 | 3.5 | μl |
| (640 nm) | 705 LP (720/30) | R718 | mcD127 | A7R34 | 3.5 | μl |
| | 752 LP (783/56) | Live/Dead ™ | Live/Dead ™ | N/A | 0.25 | μl |
| | | stain | stain Kit* | | | |

*Live/Dead ™Fixable Near-IR Dead Cell Stain Kit

### J. Flow Cytometry Analysis

Samples were acquired on the day of staining on BD FACS Lyric™ flow cytometer. About 100,000 events were recorded or the totality of the tube. Flow cytometry data were analyzed with BD FACSuite™ software. The following parameters (number of events and percentages) were analyzed in whole blood, spleen, and lymph nodes suspension cells non-activated with peptides:
Viable cells
Among viable cells:
CD4+/CD8− cells
CD8+/CD4− cells
CD4+/CD8+ cells
CD4−/CD8− cells
Among CD4+/CD8− viable cells and among CD8+/CD4− viable cells
CD49+ cells
CD49− cells
CD11a+ cells
CD11a− cells
CD44+ cells
CD62L+ cells
CD44 low/CD62L high cells
CD44 low/CD62L low cells
CD44 high/CD62L low cells
CD44 high/CD62L high cells
CD127+ cells
Among CD4+/CD8+ viable cells and among CD4−/CD8− viable cells
CD44+/CD62L+/CD49d+ cells
CD44+/CD62L+/CD49d− cells
CD49+/CD11a+ cells
CD49+/CD11a− cells

The following parameters (number of events and percentages) were analyzed in whole blood, spleen, intestine, and lymph nodes suspension cells activated with peptides:
Viable cells
Among viable cells:
CD4+/CD8− viable cells
CD8+/CD4− viable cells
Among CD4+/CD8− viable cells
IFNγ+ cells
IFNγ Mean Fluorescence Intensity (MFI)
TNFα+ cells
TNFα MFI

Overall, only the data obtained on a parent population with more than 100 events were analyzed

### K. Results:

The administration of MEVs loaded with OVA elicited a humoral immune response against OVA, when administered by the IM route twice at day 0 and day 21. MEVs loaded with OVA not only induce IgG antibodies against OVA, but also induce a strong isotype switching from IgG to IgA antibodies, indicating the mediation of a cellular immune responsive to the isotype switching (see, FIG. 14B). Control groups were mice immunized with OVA/adjuvant or only with adjuvant as shown in FIG. 14A.

FIGS. 18, 19A-B, and 20A-B show the results of the immunization with MEVs loaded with OVA by IM administration, in terms of the CD44/CD62L cell populations. FIG. 18 shows the gating used in the experiment. FIG. 19 shows the results in spleen; FIG. 19A shows the population of CD4+/CD44/CD62L cells, and FIG. 19B the population of CD8+/CD44/CD62L cells. The results show an increase of the CD44+/CD62L+ population for CD4+ and for CD8+ cells. These results indicate an increase in the T central memory cells after the immunization with MEVs loaded with OVA by IM administration. FIGS. 20A and 20B show the outcome for the same populations that occur in the inguinal lymph nodes.

FIGS. 21A-B, 22A-B, and 23A-B show the outcome after immunization with MEVs loaded with OVA by IM administration in the cellular immune response in terms of the CD44(hi)/CD49d cell populations. FIGS. 21A-B show the gating used in the experiment. FIGS. 22A-B show the outcome in spleen; FIG. 22A shows the CD4+/CD44+/CD62L+/CD49− cell populations, and FIG. 22B shows the CD8+/CD44+/CD62L+/CD49− cell populations. The results demonstrate an increase of the CD44+/CD62L+/CD49− cells for both CD4+ and CD8+. These results indicate an increase in the T virtual central memory activated cells after the immunization with MEVs loaded with OVA by IM administration. FIGS. 23A and 23B show the results for the same populations found in the inguinal lymph nodes.

FIGS. 24A-B, 25A-B, and 26A-B show the outcome after immunization with MEVs loaded with OVA by IM administration in cellular immune response, in terms of CD44(hi)/CD11a(hi) cell populations. FIGS. 24A-B show the gating used in the experiment. FIG. 25A-B show the results in spleen; FIG. 25A shows the CD4+/CD44(hi)/CD11a(hi) cell population, and FIG. 25B shows the CD8+/CD44(hi)/CD11a(hi) cell population.

The results demonstrate an increase of the CD44(hi)/CD11a(hi) cell populations for CD4+ and for CD8+. These results indicate an increase in the antigen specific T cells after the immunization with MEVs loaded with OVA by IM administration. FIGS. 26A and 26B show the results for the same populations found in the inguinal lymph nodes.

### L. Conclusions

The immunization by MEVs loaded with an immunogenic payload after IM administration elicits a payload-specific humoral and cellular immune responses. The observation of the isotype class switching (from IgG to IgA antibodies) by antigen-loaded MEVs is in accord with the observation that antigen-loaded MEVs elicit an antigen-specific cellular response.

### Example 9

Assessment of the Capacity of Extracellular Vesicles from Microalgae (MEVs) as a
Protein Vaccine Delivery System after Oral (PO) Administration.

### A. In Vivo Study in Healthy C57BL/6J Mice.

Animals were divided in groups (6 mice per group) and treated with the following:
Group 1: PO administered with MEV-OVA on Days 0, 7, 14, and 21,
Group 2: PO administered with MEV only on Days 0, 7, 14, and 21,
Group 3: PO administered with MEV only on Days 0, 7, 14, and 21, and with MEV-OVA on Days 40, 47, 54, and 61.

Mice in groups 1-2 were euthanized on Day 43 after the first vaccinal administration. Mice in group 3 were euthanized on Day 82 after the first vaccinal administration.

Whole blood was sampled just before euthanasia to collect serum for quantification of anti-OVA antibody by Enzyme-Linked Immunosorbent Assay (ELISA), and to perform flow cytometry as defined below.

Just after euthanasia, spleen is collected and processed to perform flow cytometry analysis to quantify the T cell activation after in vitro stimulation with OVA peptides.

### B. Immunization Material and Mice Treatment:

1. Group 1: MEVs Loaded with OVA

On Days 0, 7, 14 and 21:

MEVs were produced, purified, and characterized as described in Examples 1 and 2. MEVs were loaded with ovalbumin as described in Example 2. MEV-OVA were vigorously mixed by vortexing for about 60 seconds and used to treat the mice of Group 1.

### 2. Group 2: MEV (Non-Loaded)

On Days 0, 7, 14, and 21:

MEVs were produced, purified, and characterized as described in Examples 1 and 2. Non-loaded MEVs were vigorously mixed by vortexing for about 60 seconds and used to treat the mice of Group 2.

3. Group 3: MEVs-Non Loaded+MEVs Loaded with OVA

On Days 0, 7, 14, and 21:

MEVs were produced, purified, and characterized as described in Examples 1 and 2. Non-loaded MEVs were vigorously mixed by vortexing for about 60 seconds and used to treat the mice of Group 3.

On Days 40, 47, 54, and 61:

MEVs were produced, purified, and characterized as described in Examples 1 and 2. MEVs were loaded with ovalbumin as described in Example 2. MEV-OVA were vigorously mixed by vortexing for about 60 seconds and used to treat the mice of Group 3.

### C. Oral Administration

PO (per os) administration was performed using a disposable plastic syringe and an appropriate needle.

On Day 0, 7, 14, and Day 21:
Group 1 was PO administered with 100 μL of MEV-OVA at a concentration of 4¹⁰ particles/mL,
Group 2 was PO administered with 100 μL of non-loaded MEVs at a concentration of 1·2¹¹ particles/mL,
Group 3 was PO administered with 100 μL of non-loaded MEVs at a concentration of 1·2¹¹ particles/mL,
Group 3 was PO administered with 100 μL of non-loaded MEVs at a concentration of 1·2¹¹ particles/mL,

On Day 40, 47, 54, and 61
Group 3 was PO administered with 100 μL of MEV-OVA at a concentration of 4¹¹ particles/mL.

### D. Blood Sampling and Serum Collection

According to the experimental schedule, at 43 or 82 days after first PO administration in groups 1-2 or group 3 respectively, whole blood (WB) was collected by retro-orbital sinus or intracardiac puncture under anesthesia. Whole Blood sampling for flow cytometry acquisitions and for serum collection was performed as described in Example 6.

### E. Animal Euthanasia and Organs Sampling and Processing

According to the experimental schedule, at 43 or 82 days after first PO administration groups 1-2 and group 3 respectively, animals were euthanized under anesthesia and the spleen was collected and processed as described in Example 6.

### F. Quantification of Antibody Anti-OVA by ELISA

Immunoglobulin (Ig) quantification was performed by an indirect ELISA as described in Example 6.

G. Interferon Gamma (IFN-g) and Tumor Growth Factor Alpha (TNF-α) Production in CD4+ and CD8+ Populations from Peripheral Blood and Spleen.

The stimulation protocol was used as described in Example 6.

H. Flow Cytometry on Cells Activated with Peptides

The cells were stained as described in Example 6

### I. WB and Spleen Cells Staining and Flow Cytometry Acquisitions

The flow cytometry acquisitions were performed as described in Example 6.

### J. Flow Cytometry Analysis

Samples were acquired as described in Example 6.

The administration (at days 0, 7, 14, and 21) in mice, by the PO route, of MEVs loaded with OVA elicits a humoral immune response against OVA. Moreover, MEVs loaded with OVA not only induce IgG but also IgA antibodies against OVA, showing a strong isotype class switching IgG to IgA antibodies, thus indicating a cellular immune response, as shown in FIG. 14C. The control mice were immunized with OVA/adjuvant or only with adjuvant; results shown in FIG. 14A.

The immunization with MEVs loaded with OVA by PO administration induce a cellular immune response, involving the CD44/CD62L cell population, and showing an increase in the T virtual central memory activated on TCD4+ and TCD8+ cells.

The immunization with MEVs loaded with OVA by PO administration induce a cellular immune response, involving the CD44(hi)/CD49d cell populations, and showing an increase in the T central memory on TCD4+ and TCD8+ cells.

The immunization with MEVs loaded with OVA by PO administration induces a cellular immune response, involving the CD44(hi)/CD11a(hi) cell populations, and showing an increase on the T central memory on TCD4+ and TCD8+ cells.

### K. Conclusions

The administration of MEVs loaded with an immunogenic payload by the PO route elicits a humoral immune response, and a cellular immune response. The observation of the isotype class switching (from IgG to IgA antibodies) by antigen-loaded MEVs is in full agreement with the observation that antigen-loaded MEVs elicit an antigen-specific cellular response. The observations above support the notion that by PO administration, MEVs elicit a mucosal immune response against their payload.

The use of MEVs by PO administration as a vaccine delivery system (VDS) overcomes the general observation that oral/intestinal delivery of antigens is rather tolerogenic (creates tolerance, rather than immune response, against the antigen). Results presented here show the MEVs are a vaccination delivery system that is non-tolerogenic, as they elicit strong humoral and cellular immune responses against the antigenic payload.

The repeat treatment with non-loaded MEVs by PO administration does not preclude the appearance of the immune response against OVA when the same animals are subsequently administered with OVA-loaded MEVs (FIG. 14C). This observation demonstrates the absence of a neutralizing reaction against MEVs, and the absence of a tolerogenic response to MEVs (see above).

As shown in the FIG. 14C, orally administered MEVs elicit humoral immune response against an antigenic payload. It is surprising that the oral delivery of protein antigen loaded into MEVs results in generation of antigen-specific antibodies. Oral delivery is the most desirable and patient-accepted route of administration, with over 60% of commercialized small molecule drug products using the oral route. What is more, oral route of vaccine administration provides the advantage of stimulating mucosal immunity (as discussed in Examples 6). The vaccine-generated antibodies specific to MEV cargo include both IgG and IgA immunoglobulins. Hence, the immune response following per os MEV administration is characterized by significant isotype class switching to IgA antibodies, which indicates MEV potential for triggering mucosal immunity. MEV-based formulations for oral delivery may therefore offer added value for vaccine development.

### Example 10

Assessment of the Capacity of Extracellular Vesicles from Microalgae (MEVs) as a Peptide Vaccine Delivery System after Intramuscular (IM) Administration.

### A. In Vivo Study in Healthy C57BL/6J Mice.

Animals are divided in groups (6 mice per group) and treated with the following:
Group 1: IM administered with MEV-OVApep-CD8 on Days 0 and 21,
Group 2: IM administered with MEV-OVApep-CD4 on Days 0 and 21,
Group 3: IM administered with MEV-OVApep-CD4 and MEV-OVApep-CD8 on Days 0 and 21,
Group 4: IM administered with MEV only on Days 0 and 21,

Mice are euthanized on Day 43.

Whole blood is sampled just before euthanasia to collect serum for quantification of anti-OVA antibody by Enzyme-Linked Immunosorbent Assay (ELISA), and to perform flow cytometry as defined below.

Just after euthanasia, spleen, and inguinal and iliac lymph nodes (LNs) are collected and processed to perform flow cytometry analysis to quantify the T cell activation after in vitro stimulation with OVA peptides.

### B. Immunization Material and Mice Treatment:

1. Group 1: MEVs loaded with OVA peptide CD8

On Days 0, and 21:

MEVs are produced, purified, and characterized as described in Examples 1 and 2. MEVs are loaded with dominant epitope of ovalbumin OVA₂₅₇₋₂₆₄ (SIINFEKL; SEQ ID NO:189) as described in Example 2. MEV-OVApep-CD8 are vigorously mixed by vortexing for about 60 seconds and used to treat the mice of Group 1.
2. Group 2: MEVs loaded with OVA peptide CD4

On Days 0, and 21:

MEVs are produced, purified, and characterized as described in Examples 1 and
2. MEVs are loaded with dominant epitope of ovalbumin OVA₃₂₃₋₃₃₉ (SQAVHAAHAEINEAGR; SEQ ID NO:190) as described in Example 2.

MEV-OVApep-CD4 are vigorously mixed by vortexing for about 60 seconds and used to treat the mice of Group 2.
3. Group 3: MEVs loaded with OVA peptide CD4 and OVA peptide CD8

On Days 0, and 21:
MEVs are produced, purified, and characterized as described in Examples 1 and 2. MEVs are loaded with dominant epitope of ovalbumin OVA₃₂₃₋₃₃₉ and ovalbumin OVA₂₅₇₋₂₆₄ as described in Example 2. MEV-OVApep-CD4 are vigorously mixed by vortexing for about 60 seconds and used to treat the mice of Group 3.
4. Group 4: MEVs (non-loaded)

On Days 0, and 21:

MEVs are produced, purified, and characterized as described in Examples 1 and 2. Non-loaded MEVs are vigorously mixed by vortexing for about 60 seconds and used to treat the mice of Group 4.

### C. Intramuscular Administration

IM injections are performed into the right quadriceps using a disposable plastic syringe and an appropriate needle.

On Day 0 and Day 21:
Group 1, Group 2 and Group 3 is IM administered with 100 μL of MEV-OVApep at a concentration of 4¹ particles/mL,
Group 4 is IM administered with 40 μL of MEV at a concentration of 1·2¹¹ particles/mL,

### D. Blood Sampling and Serum Collection

According to the experimental schedule, at 43 days after first IM administration, whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first IM administration, animals are euthanized under anesthesia and the selected organs are collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood, LNs and Spleen.

The stimulation protocol is used as described in Example 6.

H. Flow Cytometry on Cells Activated with Peptides

The cells are stained as described in Example 6.

### I. WB, LNs and Spleen Cells Staining and Flow Cytometry Acquisitions

The flow cytometry acquisitions are performed as described in Example 6.

### J. Flow Cytometry Analysis

Samples are acquired as described in Example 6.

### Example 11

Assessment of the Capacity of Extracellular Vesicles from Microalgae (MEVs) as a Peptide Delivery System after Oral (PO) Administration.

### A. In Vivo Study in Healthy C57BL/6J Mice.

Animals are divided into 2 groups (6 mice per group) and treated with the following:
Group 1: IM administered with MEV-OVApep-CD8 on Days 0, 7, 14, and 21,
Group 2: IM administered with MEV-OVApep-CD4 on Days 0, 7, 14, and 21,
Group 3: IM administered with MEV-OVApep-CD4 and MEV-OVApep-CD8 on Days 0, 7, 14, and 21,
Group 4: IM administered with MEV only on Days 0, 7, 14, and 21,

Mice are euthanized on Day 43.

Whole blood is sampled just before euthanasia to collect serum for quantification of anti-OVA antibody by Enzyme-Linked Immunosorbent Assay (ELISA), and to perform flow cytometry as defined below.

Just after euthanasia, spleen is collected and processed to perform flow cytometry analysis to quantify the T cell activation after in vitro stimulation with OVA peptides.

### B. Immunization Material and Mice Treatment:

1. Group 1: MEVs loaded with OVA peptide-CD8
   On Days 0, 7, 14 and 21:
   a. MEVs are produced, purified, and characterized as described in Examples 1 and 2.
   b. MEVs are loaded with dominant epitope of ovalbumin OVA257-264 (SIINFEKL, (SEQ ID NO: 189) as described in Example 2. MEV-OVApep-CD8 are vigorously mixed by vortexing for about 60 seconds and used to treat the mice of Group 1.
2. Group 2: MEVs loaded with OVA peptide CD4
   On Days 0, 7, 14 and 21:
   a. MEVs are produced, purified, and characterized as described in Examples 1 and 2.
   b. MEVs are loaded with dominant epitope of ovalbumin OVA₃₂₃₋₃₃₉ (SQAVHAAHAEINEAGR, (SEQ ID NO:190) as described in Example 2. MEV-OVApep are vigorously mixed by vortexing for about 60 seconds and used to treat the mice of Group 2.
3. Group 3: MEVs loaded with OVA peptide-CD4 and OVA peptide-CD8
   On Days 0, 7, 14 and 21:
   a. MEVs are produced, purified, and characterized as described in Examples 1 and 2.
   b. MEVs are loaded with dominant epitope of ovalbumin OVA₃₂₃₋₃₃₉ and ovalbumin OVA 257-264 as described in Example 2. MEV-OVApep are vigorously mixed by vortexing for about 60 seconds and used to treat the mice of Group 3.
4. Group 4: MEV (non-loaded)
   On Days 0, 7, 14, and 21:
   a. MEVs are produced, purified, and characterized as described in Examples 1 and 2.
   b. Non-loaded MEVs are vigorously mixed by vortexing for about 60 seconds and used to treat the mice of Group 4.

### C. Oral Administration

PO (per os) administration is performed using a disposable plastic syringe and an appropriate needle.

On Day 0, 7, 14, and Day 21:
Group 1, Group 2, and Group3 is PO administered with 100 μL of MEV-OVA-peptides at a concentration of 4¹⁰ particles/mL,
Group 2 is PO administered with 100 μL of non-loaded MEVs at a concentration of 1·2¹¹ particles/mL.

### D. Blood Sampling and Serum Collection

According to the experimental schedule, at 43 days after first PO administration whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first PO administration animals are euthanized under anesthesia and the spleen is collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood and Spleen.

The stimulation protocol is used as described in Example 6.

H. Flow Cytometry on Cells Activated with Peptides

The cells are stained as described in Example 6.

### I. WB and Spleen Cells Staining and Flow Cytometry Acquisitions

The flow cytometry acquisitions are performed as described in Example 6.

### J. Flow Cytometry Analysis

Samples are acquired as described in Example 6.

### Example 12

Assessment of the Capacity of Extracellular Vesicles from Microalgae (MEVs) as a mRNA Vaccine Delivery System after Intramuscular (IM) Administration.

### A. In Vivo Study in Healthy C57BL/6J Mice.

Animals are divided into 2 groups (6 mice per group) and treated with the following:
Group 1: IM administered with MEV-OVA mRNA (OVA encoding mRNA) on Days 0 and 21,
Group 2: IM administered with MEVs only on Days 0 and 21,
Mice are euthanized on Day 43.

Whole blood is sampled just before euthanasia to collect serum for quantification of anti-OVA antibody by Enzyme-Linked Immunosorbent Assay (ELISA), and to perform flow cytometry as defined below.

Just after euthanasia, spleen, and inguinal and iliac lymph nodes (LNs) are collected and processed to perform flow cytometry analysis to quantify the T cell activation after in vitro stimulation with OVA peptides.

### B. Immunization Material and Mice Treatment:

1. Group 1: MEVs Loaded with OVA mRNA

On Days 0, and 21:

MEVs are produced, purified, and characterized as described in Examples 1 and 2. MEVs are loaded with ovalbumin mRNA (OVA mRNA) as described in Example 2. MEV-OVA mRNA are vigorously mixed by vortexing for about 60 seconds and used to treat the mice of Group 1.

### 2. Group 2: MEVs (Non-Loaded)

On Days 0, and 21:

MEVs are produced, purified, and characterized as described in Examples 1 and 2. Non-loaded MEVs are vigorously mixed by vortexing for about 60 seconds and used to treat the mice of Group 2.

### C. Intramuscular Administration

IM injections are performed into the right quadriceps using a disposable plastic syringe and an appropriate needle.

On Day 0 and Day 21:
Group 1 is IM administered with 100 μL of MEV-OVA mRNA at a concentration of 4¹⁰ particles/mL,
Group 2 is IM administered with 40 μL of MEVs at a concentration of 1·2¹¹ particles/mL,

### D. Blood Sampling and Serum Collection

According to the experimental schedule, at 43 days after first IM administration, whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first IM administration, animals are euthanized under anesthesia and the selected organs are collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood, LNs and Spleen.

The stimulation protocol is used as described in Example 6.

H. Flow Cytometry on Cells Activated with Peptides

The cells are stained as described in Example 6.

### I. WB, LNs and Spleen Cells Staining and Flow Cytometry Acquisitions

The flow cytometry acquisitions are performed as described in Example 6.

### J. Flow Cytometry Analysis

Samples are acquired as described in Example 6.

### Example 13

Assessment of the Capacity of Extracellular Vesicles from Microalgae (MEVs) as a mRNA Delivery System after Oral (PO) Administration.

### A. In Vivo Study in Healthy C57BL/6J Mice.

Animals are divided into groups (6 mice per group) and treated with the following:
Group 1: PO administered with MEV-OVA mRNA on Days 0, 7, 14, and 21,
Group 2: PO administered with MEV only on Days 0, 7, 14 and 21.

Mice are euthanized on Day 43.

Whole blood is sampled just before euthanasia to collect serum for quantification of anti-OVA antibody by Enzyme-Linked Immunosorbent Assay (ELISA), and to perform flow cytometry as defined below.

Immediately after euthanasia, spleen is collected and processed to perform flow cytometry analysis to quantify the T cell activation after in vitro stimulation with OVA peptides.

### B. Immunization Material and Mice Treatment:

1. Group 1: MEVs Loaded with OVA mRNA

On Days 0, 7, 14 and 21:

MEVs are produced, purified, and characterized as described in Examples 1 and 2. MEVs are loaded with mRNA for ovalbumin (OVA mRNA) as described in the Examples above.

MEV-OVA mRNA is vigorously mixed by vortexing for about 60 seconds and used to treat the mice of Group 1.

### 2. Group 2: MEV (Non-Loaded)

On Days 0, 7, 14, and 21:

MEVs are produced, purified, and characterized as described in Examples 1 and 2. Non-loaded MEVs are vigorously mixed by vortexing for about 60 seconds and used to treat the mice of Group 2.

### C. Oral Administration

PO (per os) administration is performed using a disposable plastic syringe and an appropriate needle.

On Day 0, 7, 14, and Day 21:
Group 1 is PO administered with 100 μL of MEV-OVA mRNA at a concentration of 4¹⁰ particles/mL,
Group 2 is PO administered with 100 μL of non-loaded MEVs at a concentration of 1·2¹¹ particles/mL.

### D. Blood Sampling and Serum Collection

According to the experimental schedule, at 43 days after first PO administration whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first PO administration animals are euthanized under anesthesia and the spleen is collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood and Spleen.

The stimulation protocol is used as described in Example 6.

H. Flow Cytometry on Cells Activated with Peptides

The cells are stained as described in Example 6.

### I. WB and Spleen Cells Staining and Flow Cytometry Acquisitions

The flow cytometry acquisitions are performed as described in Example 6.

### J. Flow Cytometry Analysis

Samples are acquired as described in Example 6.

### Example 14

Assessment of the Capacity of Extracellular Vesicles from Microalgae (MEVs) as a DNA Vaccine Delivery System after Intramuscular (IM) Administration

### A. In Vivo Study in Healthy C57BL/6J Mice.

Animals are divided into 2 groups (6 mice per group) and treated with the following:
Group 1: IM administered with MEV-pOVA on Days 0 and 21,
Group 2: IM administered with MEV only on Days 0 and 21,

Mice are euthanized on Day 43.

Whole blood is sampled just before euthanasia to collect serum for quantification of anti-OVA antibody by Enzyme-Linked Immunosorbent Assay (ELISA), and to perform flow cytometry as described below. Immediately after euthanasia, spleen, and inguinal and iliac lymph nodes (LNs) are collected and processed to perform flow cytometry analysis to quantify the T cell activation after in vitro stimulation with OVA peptides.

### B. Immunization Material and Mice Treatment:

1. Group 1: MEVs Loaded with pOVA

On Days 0, and 21:

MEVs are produced, purified, and characterized as described in Examples 1 and 2. MEVs are loaded with OVA-encoding expression plasmid DNA (pOVA, cat. no. 31598, Addgene) as described in Example 2. MEV-pOVA are vigorously mixed by vortexing for about 60 seconds and used to treat the mice of Group 1.

### 2. Group 2: MEVs (Non-Loaded)

On Days 0, and 21:

MEVs are produced, purified, and characterized as described in Examples 1 and 2. Non-loaded MEVs are vigorously mixed by vortexing for about 60 seconds and used to treat the mice of Group 2.

### C. Intramuscular Administration

IM injections are performed into the right quadriceps using a disposable plastic syringe and an appropriate needle.

On Day 0 and Day 21:
Group 1 is IM administered with 100 μL of MEV-pOVA at a concentration of 4¹⁰ particles/mL,
Group 2 is IM administered with 40 μL of MEVs at a concentration of 1·2¹¹ particles/mL,

### D. Blood Sampling and Serum Collection

According to the experimental schedule, at 43 days after first IM administration, whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first IM administration, animals are euthanized under anesthesia and the selected organs are collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood, LNs and Spleen.

The stimulation protocol is used as described in Example 6.

H. Flow Cytometry on Cells Activated with Peptides

The cells are stained as described in Example 6.

### I. WB, LNs and Spleen Cells Staining and Flow Cytometry Acquisitions

The flow cytometry acquisitions are performed as described in Example 6.

### J. Flow Cytometry Analysis

Samples are acquired as described in Example 6.

### Example 15

Assessment of the Capacity of Extracellular Vesicles from Microalgae (MEVs) as a DNA Delivery System after Oral (PO) Administration.

### A. In Vivo Study in Healthy C57BL/6J Mice.

Animals are divided into 2 groups (6 mice per group) and treated with the following:
Group 1: PO administered with MEV-pOVA on Days 0, 7, 14, and 21,
Group 2: PO administered with MEVs only on Days 0, 7, 14, and 21.

Mice are euthanized on Day 43.

Whole blood is sampled just before euthanasia to collect serum for quantification of anti-OVA antibody by Enzyme-Linked Immunosorbent Assay (ELISA), and to perform flow cytometry as defined below.

Just after euthanasia, spleen is collected and processed to perform flow cytometry analysis to quantify the T cell activation after in vitro stimulation with OVA peptides.

### B. Immunization Material and Mice Treatment:

1. Group 1: MEVs Loaded with pOVA

On Days 0, 7, 14, and 21:

MEVs are produced, purified, and characterized as described in Examples 1 and 2. MEVs are loaded with OVA-encoding expression plasmid DNA (pOVA, cat. no. 31598, Addgene) as described in Example 2. MEV-pOVA are vigorously mixed by vortexing for about 60 seconds and used to treat the mice of Group 1.

### 2. Group 2: MEV (Non-Loaded)

On Days 0, 7, 14, and 21:

MEVs are produced, purified, and characterized as described in Examples 1 and 2. Non-loaded MEVs are vigorously mixed by vortexing for about 60 seconds and used to treat the mice of Group 2.

### C. Oral Administration

PO (per os) administration is performed using a disposable plastic syringe and an appropriate needle.

On Day 0, 7, 14, and Day 21:
Group 1 is PO administered with 100 μL of MEV-pOVA at a concentration of 4¹ particles/mL,
Group 2 is PO administered with 100 μL of non-loaded MEVs at a concentration of 1·2¹¹ particles/mL.

### D. Blood Sampling and Serum Collection

According to the experimental schedule, at 43 days after first PO administration whole blood (WB) is collected by retro-orbital sinus or intracardiac puncture under anesthesia. Whole Blood sampling for flow cytometry acquisitions and for serum collection is performed as described in Example 6.

### E. Animal Euthanasia and Organs Sampling and Processing

According to the experimental schedule, at 43 days after first PO administration animals are euthanized under anesthesia and the spleen is collected and processed as described in Example 6.

### F. Quantification of Antibody Anti-OVA by ELISA

Immunoglobulin (Ig) quantification is performed by an indirect ELISA as described in Example 6.

G. Interferon Gamma (IFN-g) and Tumor Growth Factor Alpha (TNF-α) Production in CD4+ and CD8+ Populations from Peripheral Blood and Spleen.

The stimulation protocol is used as described in Example 6.

H. Flow Cytometry on Cells Activated with Peptides

The cells are stained as described in Example 6.

I. WB and Spleen cells staining and flow cytometry acquisitions

The flow cytometry acquisitions are performed as described in Example 6.

J. Flow cytometry analysis

Samples are acquired as described in Example 6.

### Example 16

Assessment of Anti-Tumor Efficacy of MEV-Based Vaccine Delivery Systems after Intramuscular (IM) and Oral (PO) Administration.

### A. Animal Model

C57BL/6J mice are used for the in vivo tumor growth studies. Briefly, B16-OVA cells and E.G7-OVA cells are harvested at the exponential growth phase and washed with PBS. The animals are divided into groups and injected subcutaneously into the right flank with B16-OVA cells (2×10⁵ in 100 μL) or E.G7-OVA cells (2×10⁶ in 100 μL). Tumor size is measured daily using electronic calipers and is expressed as a volume (mm³) using the volume equation 0.5×(a×b²), in which a is the long diameter and b is the short diameter.

### B. Prophylactic Vaccination Scheme

The animals (12 mice per group) are treated with MEV-based OVA vaccine (see Examples 3-10) either with intramuscular injection (IM subgroup) or per os administration (PO subgroup) starting from day 1. Tumor challenge with either B16-OVA cells (2×10⁵ in 100 μL) or E.G7-OVA cells (2×10⁶ in 100 μL) is performed on day 27 as described above. The control groups receive the same amount of MEVs non-loaded with any cargo and administered with the same route. Tumor size is measured until the endpoint.

### C. Therapeutic Vaccination Scheme

The animals (12 mice per group) are injected with either B16-OVA cells (2×10⁵ in 100 μL) or E.G7-OVA cells (2×10⁶ in 100 μL) on day 0 as described above. MEV-based OVA vaccine (see Examples 3-10) is administered on days 3, 7, 10, and 17, either with intramuscular injection (IM subgroup) or per os administration (PO subgroup). The control groups receive the same amount of MEVs non-loaded with any cargo and administered with the same route. Tumor size is measured until the endpoint.

### Example 17

### Preparation of Freeze-Dried MEV Formulations for Intramuscular (IM) and Oral (PO) Administration.

After isolation, purification, and loading (as described above), MEVs are dialyzed with 20 mM potassium phosphate buffer in Slide-A-Lyzer MINI dialysis cassettes (20K MWCO; Thermo Fisher Scientific). After dialysis, MEVs are filtered through a 0.2 μm PES membrane syringe filter (VWR International) and mixed 1:1 with stabilizer stock solutions to match the following final buffer (A or B), surfactant (A or B) and cryoprotectant (A or B) concentrations:
buffer A: 10 mM sodium phosphate (Sigma-Aldrich), pH 7.4;
buffer B: 10 mM potassium phosphate (Sigma-Aldrich), pH 7.4;
surfactant A: 0.02% polyvinylpyrrolidone (Kollidon 17 PF, BASF);
surfactant B: 0.02% poloxamer 188 (Kolliphor P188, BASF)
cryoprotectant A: 5% polyvinylpyrrolidone (Kollidon 17 PF, BASF);
cryoprotectant B: 5% sucrose (Sigma-Aldrich).

2R glass vials (Fiolax clear, Schott) with igloo rubber stoppers (B2-TR coating, West Pharmaceutical Services) are cleaned with Highly Purified Water (HPW) and dried for 8 h at 60° C. The samples are lyophilized in 2R vials with 200 μL fill volume. Lyophilization is performed on a pilot-scale freeze-dryer (LyoStar 3). After an equilibration step at −5° C. for 15 min, the samples are frozen at −1° C. min⁻¹ to −50° C. and held for 120 min. Primary drying is performed at −20° C. and 40 mTorr with manometric end point determination. Secondary drying is performed at 20° C. and mTorr for 8 hours. Samples are stoppered under slight vacuum at 450 Torr nitrogen, and vials are crimped with aluminum seals. The lyophilizates are stored at room temperature (25° C.) until use. For reconstitution, 190 μL of HPW is added to each vial. The vials are shaken gently to ensure wetting of the complete lyophilizate.

Upon reconstitution of the freeze-dried formulations, MEVs are evaluated for shape, size and homogeneity characteristics as described in Example 2. The cargo content is quantified and characterized as described in Example 4. The reconstituted formulations are used for intramuscular and oral immunization studies as described in Examples 6-13.

### Example 17A

### Inhibition or Stimulation of Toll Like Receptors (TLR) Using MEV-Mediated Delivery in Macrophage Cells In Vitro

Toll-like receptors have a crucial role in the detection of microbial infection in mammals and insects. In mammals, the TLRs recognize conserved products unique to microbial metabolism. This specificity allows the Toll proteins to detect the presence of infection and to induce activation of inflammatory and antimicrobial innate immune responses. Recognition of microbial products by Toll-like receptors expressed on dendritic cells triggers functional maturation of dendritic cells and leads to initiation of antigen-specific adaptive immune responses.

Toll-like receptors (TLRs) are the important mediators of inflammatory pathways in the gut and the TLRs thereby play a major role in mediating the immune responses towards a wide variety of pathogen-derived ligands and link adaptive immunity with the innate immunity. MEVs have the capacity to efficiently penetrate cells and deliver their payload inside the cells. This ability of MEVs provides a way to modulate intracellular TLRs and to thereby stimulate intracellular pathways driven by TLRs. MEVs loaded with agonists or antagonists of TLRs can be used to target intracellular TLRs and deliver the payload in the intracellular compartments where TLRs are located (see, FIGS. 17A and 17B, which show MEVs and their interactions with TLRS and ligands).

### A. Cell Cultures:

### THP1-Derived Macrophage Culture

The ATCC® human monocyte THP-1 cell line is cultured in RPMI-1640 medium supplemented with 10% FBS, 2 mM L-glutamine, 100 U/mL penicillin, 100 μg/mL streptomycin, and 0.05 mM 2-mercaptoethanol. THP-1 monocytes are differentiated into M0 macrophages (M0-M). The cells are seeded at 1×10⁵ cells/mL and incubated at 37° C. with 5% CO₂ for 48 h in the presence of 50 ng/mL of PMA; then, the conditioned medium with PMA are removed and replaced with fresh medium for 3 days for cell recovery. Macrophage maturations are assessed by flow cytometry. After five days of culture, the cells are harvested and stained for CD68, a marker of mature macrophages, using the antihuman monoclonal antibody (mAb) anti CD68 PE (clone Y1/82A) and its isotype control PE Mouse IgG2b, K Isotype Ctrl Antibody (clone MPC-11). The expression of CD68 in THP-1-derived macrophages are assessed using a flow cytometer. At least 30,000 cells are acquired, and the two-fold increase in mean fluorescence intensity (MFI) of CD68 compared with the isotype control are considered successful maturation.

### RAW 264.7 Macrophage Culture

RAW 264.7 cells are obtained from ATCC®. Cells are maintained in D10 and passaged every three days when more than 80% confluent. In preparation for phagocytosis assays, cells are harvested and centrifuged at room temperature for 7 min at 1100 rpm using an IEC clinical benchtop centrifuge, re-suspended in R10, counted, and viability assessed using trypan blue dye exclusion (Sigma-Aldrich). Viability in all experiments is >90%.

### B. Reagents

TLR ligands are dsRNA: (polyadenylic-polyuridilyc acid (polyA:U)); (polyinosinic:polycytidylic acid (Poly I:C)); antivirals (as R848, R837, CL075, and CL264); taxol; flagellin; the flagellin-mimetic peptide flp22 (peptide from flagellin); or unmethylated CpG motif oligonucleotides (ODN class A, class B, and class C).

For phagocytosis assays, macrophages are cultured in sterile tissue culture medium (R10), composed of RPMI 1640 medium (Life Technologies, Burlington, ON), supplemented with 10% heat-inactivated fetal calf serum (Life Technologies, Burlington, ON), 100 U/mL penicillin (Life Technologies, Burlington, ON), 100 μg/mL streptomycin (Life Technologies, Burlington, ON), 25 mM N-2-hydroxyethylpiperazine-NV-2-ethanesulphonic acid (HEPES, Life Technologies, Burlington, ON) and 10 mM L-glutamine (Life Technologies, Burlington, ON). Dulbecco's Modified Eagles Medium (DMEM) (Life Technologies, Burlington, ON), supplemented in the same way as the RPMI, to form D10, are used for growth of RAW 264.7 macrophages.

MEVs were labeled with pKH26 as described in Example 3 or MEVs were loaded with different payloads as describe below.

### C. Loading of MEVs

MEVs are loaded with different ligands of TLRs as described herein (see, also, International PCT application No PCT/EP2022/07037). The TLR ligands included: dsRNA (polyadenylic-polyuridilyc acid (poly(A:U)); Polyinosinic:polycytidylic acid (Poly (I:C) HMW or Poly(I:C) LMW); antivirals (as R848, R837, CL075, and CL264); taxol; flagellin; the flagellin-mimetic peptide flp22 (peptide from flagellin); or unmethylated CpG motif oligonucleotides (ODN class A, class B, and class C), which are TLR3 (dsRNA (polyadenylic-polyuridilyc acid (poly(A:U)); Polyinosinic:polycytidylic acid (Poly (I:C)), TLR4 (taxol); TLR5 (flagellin; flagellin-mimetic peptide flp22 (peptide from flagellin)), TLR7 and TLR8 (antivirals such as R848, R837, CL075, and CL264), and TLR9 (unmethylated CpG motif oligonucleotides) agonists.

### D. MEVs Internalization

The THP-1 or RAW cells were treated at different time points with MEVs labeled with pKH26 or loaded with TLR ligands at different timepoints. 24 hours after the seeding, cells were counted and a mixture of cell culture media with different the different treatments were prepared: TLR ligands or MEVs (non-loaded or loaded with different TLR ligands). The final concentration of MEVs were between 200 and 1,000 loaded-MEVs/cell, the TLR ligands alone is shown in the results, figures, or tables.

Internalization of MEVs by M0 Macrophages: MEVs were labeled with PKH26 Red Fluorescent Cell Linker Kits following the methods described herein (see, also PCT/EP2022/070371 and U.S. provisional application Ser. No. 63/349,006). The labeled MEVs were incubated with THP-1 or RAW cells for 1 h, 3 h, 6 h, 16 h, and 24 h at 37° C. with 5% CO₂. After incubation, the cells are fixed with PFA 4%, permeabilized with 0.1% Triton™ X-100, and stained with Actin Green, USA, 1 drop/mL of PBS) that binds actin with high affinity, and the nuclei are stained with Hoechst, dilution 1:1000). The samples are analyzed by confocal microscopy.

Internalization of MEVs by M0 Macrophages: MEVs loaded with different payloads as described above and following the methods described above. The loaded MEVs were incubated with THP-1 or RAW cells for 1 h, 3 h, 6 h, 16 h, and 24 h at 37° C. with 5% CO₂. After incubation, the cells are fixed with PFA 4%, permeabilized with 0.1% Triton™ X-100, and stained with Actin Green, USA, 1 drop/mL of PBS) that binds actin with high affinity, and the nuclei are stained with Hoechst, dilution 1:1000). The samples are analyzed by confocal microscopy.

### E. Macrophage Phagocytosis Assay

For phagocytosis assays, 1×10⁵ RAW 264.7 macrophages in R10 medium are plated onto sterile 4 well chambered Millicell® EZ SLIDES glass culture slides (Millipore Corporation, Bedford, MA), and macrophages allowed to adhere in a humidified 5% CO₂ incubator for 30 min at 37° C. Zymosan particles are added to the cells at a zymosan to macrophage ratio of 10:1. Phagocytosis is allowed to proceed for 1 h at 37° C. in a humidified 5% CO₂ incubator. In experiments where the effects of TLR ligands are assessed, cells are incubated in the presence of these TLR receptor agonists for 16 h prior to addition of zymosan. For experiments assessing scavenger receptor blockade, inhibitors are added 15 min prior to addition of zymosan. Phagocytosis is terminated by vigorously washing off non-ingested particles with warm R10 three times. Glass slides are air-dried for 15 min, stained using Wright's stain (Sigma-Aldrich), and slides examined by light microscopy at X600 magnification using a Nikon® Eclipse 50i photomicroscope. Phagocytosis is determined from photomicrographs at X600 magnification by counting at least 200 macrophages in 16 different fields of view for each experimental treatment. Phagocytosis is assessed as the mean number of particles per cell (MNP). Phagocytic index (PI) is used to express overall phagocytosis and determined as percentage of phagocytic cells multiplied by MNP (PI=percentage phagocytosis X MNP). Photomicrographs of cells for illustrations are taken either unstained by phase contrast at ×400 magnification using an inverted microscope (Nikon® Eclipse TE2000S), or stained cells captured at ×600 magnification using a Nikon® Eclipse 50i photomicroscope.

### F. Macrophage Cell Surface Receptor Expression

To assess the effect of LPS on macrophage cell surface receptor expression, 1×10⁵ macrophages cells are cultured in R10 alone or pretreated overnight with LPS at 10 ng/mL. Wells are washed with Ca2⁺ and Mg2⁺ free PBS, and then the cells are detached using Accutase™ (BD Bioscience, Mississauga, ON). Cell surface expression of Class A scavenger receptors (SR-A) and the lectin receptor Dectin-1 is detected using FITC-labelled IgG antibodies against the respective surface proteins, and this is compared with fluorescence of appropriate isotype-matched control antibodies. Cells are stained using either anti SR-A IgG (1 μg/mL) (isotype control IgG2) or anti Dectin-1 IgG (1 μg/mL) (isotype control IgG2). All antibodies and controls are obtained from Bio-Rad (Raleigh, NC). Antibodies are added to 100 μl of cells re-suspended in staining buffer composed of PBS, 2% bovine serum albumin and 0.1% sodium azide, to a concentration of 10⁶ cells/mL. During staining, cells are incubated on ice and kept in the dark for 45 min. Thereafter, cells are washed, re-suspended in 200 μl of stain buffer, and analyzed using a BD FACSJazz™ system (BD Biosciences, San Jose, CA) and Flow-Jo software (Tree Star, Ashland, OR). Forward and side scatter are used to discriminate live macrophages prior to analysis of SR-A and Dectin-1 expression.

### G. Cytotoxicity Determination

### MTT (3-[4,5-Dimethylthiazol-2-yl]-2,5 Diphenyl Tetrazolium Bromide) Assay

Cell viability is determined by an MTT assay as previously described. The THP-1 cells are seeded in triplicate in 48-well plates; 24 h post-seeding, the cells are treated with MEVs for 24 and 48 h. The absorbance is measured by an ELISA reader at 540 nm. Values are expressed as a percentage of cell growth versus that in the control (untreated cells).

### H. Western Blotting:

Total proteins from the THP-1 cells or RAW 264.7 treated with loaded MEVs as described above were isolated and analyzed by SDS-PAGE followed by Western Blotting. The primary antibodies used in the experiments are as follows: anti-NF-kB antibody (Novus, NB100-2176, dilution 1:500), anti-phosphorylated-NF-kB antibody p65 (S536) (Cell signaling, 3033, dilution 1:1000), anti-IRF-3 antibody (EPR24184) (ABCAM,ab68481, dilution 1:500), anti-phosphorylated-IRF-3 antibody (Ser396) (E.875.8) (Thermo Fischer Scientific, MA (−14947, dilution 1:1000), anti-PD-L1 antibody (Abcam, Cambridge, UK, ab213524, dilution 1:1000; antipSTAT3 (R&D System, AF4607-SP, dilution 1:500), anti-STAT3 (Novus Biologicals, Denver, CO, USA, NBP2-24463, dilution 1:1000), anti-HSP70 (Novus Biologicals, NB600-1469, dilution 1:1000), anti-HSC70 (Santa Cruz, Dallas, TX, USA, sc-7298, dilution 1:500), anti-Calnexin (Santa Cruz, sc-23954, dilution 1:500), anti-Tubulin (Santa Cruz, sc-398103, dilution 1:1000), anti-P Actin (Santa Cruz, sc-81178, dilution 1:1000), anti-GAPDH (Santa Cruz, sc-47724, dilution 1:1000). After overnight incubation with the primary antibodies at 4° C., the membranes are incubated with HRP-conjugated secondary antibody (Thermo Fisher Scientific) for 1 h, at 4° C.; the chemiluminescent signal is detected using a ChemiDoc™ MP imaging system (Bio-Rad).

### I. Real-Time PCR

The THP-1 and RAW cells are seeded in 12 well-plates at 1×10⁵ cells/mL and differentiated in M0 macrophages, as described above; the cells then are treated with MEVs for 6 and 24 h. At the end of the treatments, total RNA is extracted using QIAGEN® kit. The RNA is reverse transcribed to cDNA using the High-Capacity cDNA Reverse Transcription kit. Then, the cDNA is subjected to quantitative real-time reverse transcriptase polymerase chain reaction (RT-PCR) analysis. The sequences of the primers used are indicated in Table 9, below.

TABLE 9
List of primers used for Real-Time PCR.

| | | SEQ | | SEQ |
|---|---|---|---|---|
| | | ID | | ID |
| Primers | Forward | NO. | Reverse | NO. |
| GAPDH | ATGGGGAAGGTGAAGGTCG | 150 | GGGTCATTGATGGCAACAATAT | 151 |
| PD-L1 | TCACGGTTCCCAAGGACCTA | 152 | AGGTCTTCCTCTCCATGCAC | 153 |
| IL-6 | GGTACATCCTCGACGGCATC | 154 | GTGCCTCTTTGCTGCTTTCAC | 155 |
| | T | | | |
| IL-1β | ACAGATGAAGTGCTCCTTCC | 156 | GTCGGAGATTCGTAGCTGGAT | 157 |
| | A | | | |
| TNF-α | CCAGGCAGTCAGATCATCTTCTC | 158 | AGCTGGTTATCTCTCAGCTCCAC | 159 |

Real-time PCR is performed using Step One Real-time PCR System Thermal Cycling Block (Applied Biosystem) in a 20 μL reaction containing 300 nM of each primer, 2 μL of template cDNA, and 18 μL of 2×SYBR® Green I Master Mix. The PCR is run at 95° C. for 20 s followed by 40 cycles of 95° C. for 3 s and 60° C. for 30 s. GAPDH is used as the endogenous control. Relative changes in gene expression between control and treated samples are determined using the ΔCt method.

### J. ELISA Test

The amount of IL-6 secreted in the culture medium of THP-1 cells treated with MEVs was determined using a human TL-6 ELISA kit (Invitrogen KAC1261). For cells in suspension (THP-1), 90,000 cells were seeded per 0.5 mL of media in a 12-well plate. After 24 hours, cells were counted and a mixture of cell culture media with different TLR ligands or MEVs (non-loaded or loaded with different TLR ligands) were prepared with a final concentration of MEVs between 200 and 1,000 loaded-MEVs/cell. Cells were treated for 48 hrs. then the supernatants were collected. Measurements were carried out as per the manufacturer's protocol (Invitrogen KAC1261).

The amount of IFNα secreted in the culture medium of THP-1 cells treated with MEVs was determined using a human IFNα ELISA kit (PBL-Assay Science, 41135-1). For cells in suspension (THP-1), 90,000 cells were seeded per 0.5 mL of media in a 12-well plate. After 24 hours, cells were counted and a mixture of cell culture media with different TLR ligands or MEVs (non-loaded or loaded with different TLR ligands) were prepared with a final concentration of MEVs between 200 and 1,000 loaded-MEVs/cell. Cells were treated for 48 hrs. then the supernatants were collected. The ELISA was performed according to the manufacturer's instructions.

For RAW cells the cytokines secreted by cells treated with MEVs loaded or non-loaded was determined using Luminex Multiplex Immunoassay (by R&D System, Procarta Plex Mouse Basic kit (EOX010-20440-901) and Procarta Plex Mouse TGF beta1 simplex (EPX01A-20608-901)). The assays were performed according to the manufacturer's instructions. All treatments are described in the figures and tables.

### K. Results:

1. Internalization of MEVs into Human Epithelial Cells In Vitro

The MEVs labeled with PKH26 or loaded with different payloads either agonist of TLRs or proteins as ovalbumin (OVA) or mRNAs coding for OVA were incubated at different time points and were either internalized or phagocyted by human monocytes TPH-1 cells or mice monocytes/macrophages M0 RAW 264.7 cells showing the ability of MEVs to penetrate to cells and to deliver its payloads (oligonucleotides, mRNAs, proteins, dsRNAs).

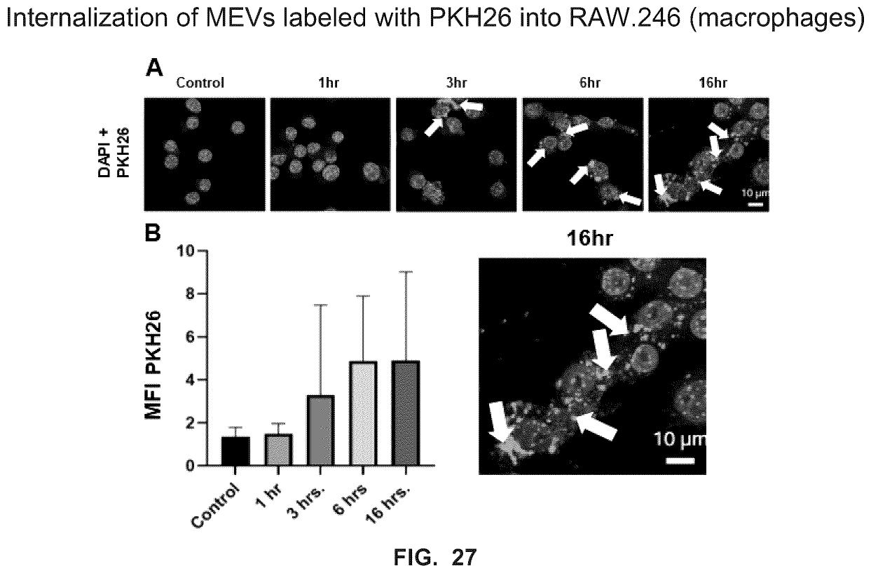

FIG. 27A shows the internalization of MEVs labeled with PKH26 into RAW cells at different timepoints. FIG. 27B shows the kinetics of internalization of MEVs into Raw cells. These results on human cells in vitro confirm previous observations made in vivo in mice that showed the internalization of MEVs labeled with PKH26 into idiocytes (tissular macrophages) and dendritic cells in the GALT. (Figures of example 6B).

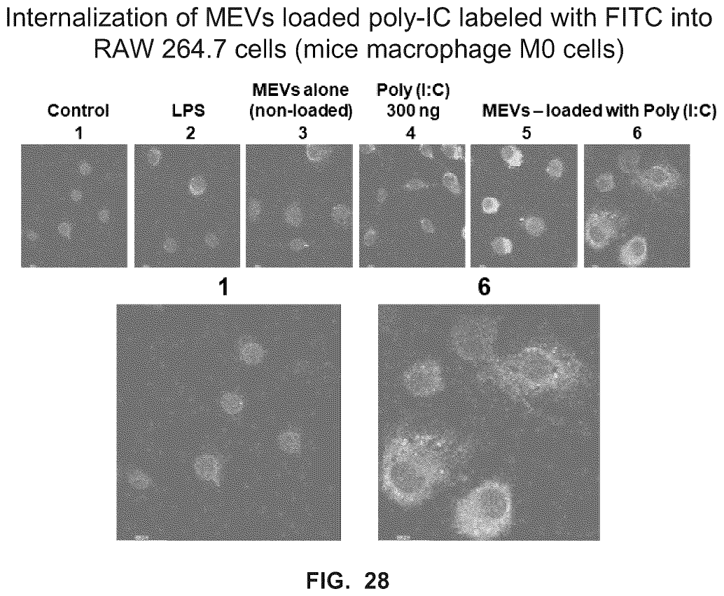

FIG. 28 shows the delivery of the payload into RAW cells in excellent correlation with the MEVs internalization shown in FIGS. 27A-27B.

2. Payload Biological Activity after Delivery by MEVs into Human Epithelial Cells

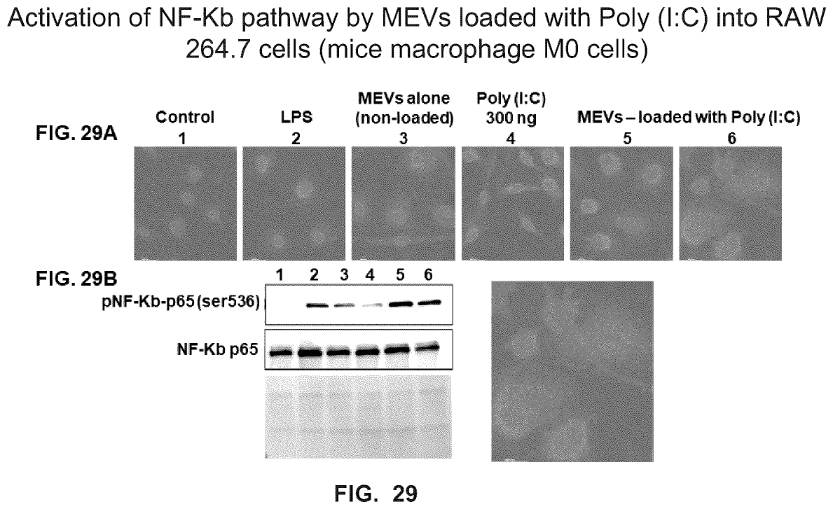

The stimulation of the phosphorylation of intracellular markers as NF-Kb and IRF-3 molecules was observed after the incubation at different timepoints with MEVs loaded with different payloads agonist of TLR, in RAW264.7 cells showing an effective biological activity of the payload when deliver by MEVs into the cells. FIGS. 29A-29B show the results of the activation of NF-Kb intracellular pathway after delivery of TLR-3 agonist by MEVs into RAW264.7 cells.

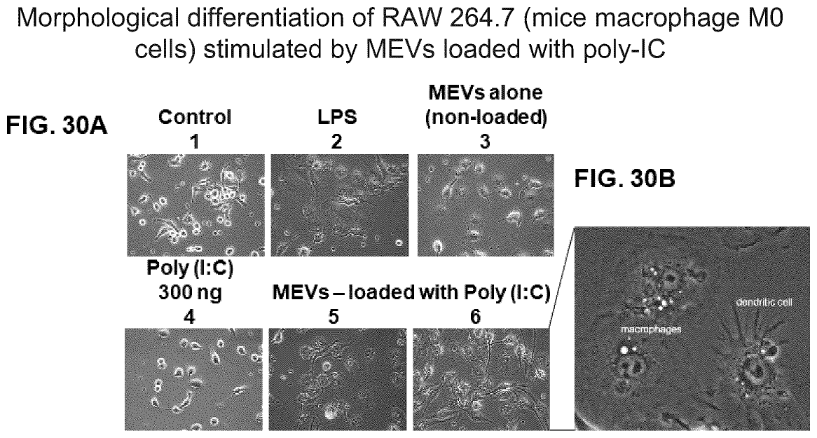

After incubation for 24 hs with MEVs loaded with TLR agonist RAW264.7 cells are morphological differentiated into M1/M2 macrophages and/or dendritic cells. FIGS. 30A-30B show the results.

### L. Conclusions:

MEVs can penetrate macrophage cells (by internalization or phagocytosis) and deliver their payloads. When the payloads are agonist or antagonist of TLR, once delivered, they bind to the TLR thus inducing the activation/deactivation of an intracellular pathway. In particular, for TLRs that are not located in the cellular membrane but in intracellular endosome membranes (such as TLR3, TLR5, TLR7, TLR8, and TLR9), MEVs can reach such endosomes and deliver their payloads into the endosomes thus allowing the payloads to stimulate the endosomal TLRs. The stimulation of TLRs induces intracellular pathways involved in the modulation of inflammatory reaction, modulation of imbalance of cytokines, tissular reparation, and innate and or adaptative immune response.

### Example 17B

### Inhibition or Stimulation of Toll Like Receptors (TLR) Using MEV-Mediated Delivery in Human Epithelial Cells In Vitro

Toll-like receptors have a crucial role in the detection of microbial infection in mammals and insects. In mammals, these receptors have evolved to recognize conserved products unique to microbial metabolism. This specificity allows the Toll proteins to detect the presence of infection and to induce activation of inflammatory and antimicrobial innate immune responses. Recognition of microbial products by Toll-like receptors expressed on dendritic cells triggers functional maturation of dendritic cells and leads to initiation of antigen-specific adaptive immune responses.

In particularly, Toll-like receptors (TLRs) are the important mediators of inflammatory pathways in the gut which play a major role in mediating the immune responses towards a wide variety of pathogen-derived ligands and link adaptive immunity with the innate immunity.

MEVs have the capacity to efficiently penetrate cells and deliver their payload inside those cells. This ability of MEVs opens the possibility to address the intracellular TLRs and stimulate intracellular pathways driven by TLRs. MEVs loaded with agonists or antagonists of TLRs can be used to target intracellular TLRs and deliver the payload in the intracellular compartments where TLRs are located, as described in FIGS. 17A and 17B.

### A. Cell Cultures

BEAS-2B human bronchial epithelial cells were obtained from the American Type Culture Collection (ATCC) (CRL-9609). The cells were cultured in BEBM Basal Medium (CC-3171, Lonza) supplemented with BEGM SingleQuots Supplement Pack (CC-4175, Lonza) and 100 U/ml of penicillin, and 100 g/ml of streptomycin (15140122, Gibco) at 37° C. under 5% CO₂. Non-transformed human small intestinal epithelial FHs-74 Int cells were also obtained from ATCC (CCL-241). The cells were maintained in Hybri-Care medium (ATCC-46-X) supplemented with 10% heat-inactivated fetal bovine, and 100 μg/ml of streptomycin (15140122, Gibco), and 30 ng/ml EGF (SRP3027-500 μG, SIGMA) at 37° C. under 5% CO₂. Non-transformed human large intestinal epithelial FHC cells were also obtained from ATCC (CRL-1831). The cells were maintained in DMEM/F-12 media (31330038, Gibco) supplemented with 10% heat-inactivated fetal bovine, 100 U/ml of penicillin −100 μg/ml of streptomycin (15140122, Gibco), 20 ng/ml EGF (SRP3027-500 μG, SIGMA), 10 ng/ml Cholera Toxin (C8052-1MG, SIGMA), 1 mM HEPES (H0887, SIGMA), 5 μg/ml Insulin (I9278), 5 μg/ml Transferrin (T8158, SIGMA), 100 ng/ml Hydrocortisone (352450050, Thermo Scientific) at 37° C. under 5% CO₂.

### B. Reagents

TLR ligands are dsRNA: (polyadenylic-polyuridilyc acid (polyA:U)); (polyinosinic:polycytidylic acid (Poly I:C)); antivirals (as R848, R837, CL075, and CL264); taxol; flagellin; the flagellin-mimetic peptide flp22 (peptide from flagellin); or unmethylated CpG motif oligonucleotides (ODN class A, class B, and class C).

For phagocytosis assays, macrophages are cultured in sterile tissue culture medium (R10), composed of RPMI 1640 medium (Life Technologies, Burlington, ON), supplemented with 10% heat-inactivated fetal calf serum (Life Technologies, Burlington, ON), 100 U/mL penicillin (Life Technologies, Burlington, ON), 100 μg/mL streptomycin (Life Technologies, Burlington, ON), 25 mM N-2-hydroxyethylpiperazine-NV-2-ethanesulphonic acid (HEPES, Life Technologies, Burlington, ON) and 10 mM L-glutamine (Life Technologies, Burlington, ON). Dulbecco's Modified Eagles Medium (DMEM) (Life Technologies, Burlington, ON), supplemented in the same way as the RPMI, to form D10, are used for growth of RAW 264.7 macrophages.

MEVs labeled with pKH26 as described in Example 3 or MEVs loaded with different payloads as describe below.

### C. Loading of MEVs

MEVs are loaded with different ligands of TLRs as described herein (see, also, International PCT application No PCT/EP2022/07037). The TLR ligands included: dsRNA (polyadenylic-polyuridilyc acid (poly(A:U)); Polyinosinic:polycytidylic acid (Poly (I:C) HMW or Poly(I:C) LMW); antivirals (as R848, R837, CL075, and CL264); taxol; flagellin; the flagellin-mimetic peptide flp22 (peptide from flagellin); or unmethylated CpG motif oligonucleotides (ODN class A, class B, and class C), which are TLR3 (dsRNA (polyadenylic-polyuridilyc acid (poly(A:U)); Polyinosinic:polycytidylic acid (Poly (I:C)), TLR4 (taxol); TLR5 (flagellin; flagellin-mimetic peptide flp22 (peptide from flagellin)), TLR7 and TLR8 (antivirals such as R848, R837, CL075, and CL264), and TLR9 (unmethylated CpG motif oligonucleotides) agonists.

### D. MEVs Internalization

The human epithelial cells were treated at different time points with MEVs labeled with PKH26 or loaded with TLR ligands at different timepoints. 24 hours the seeding, cells were counted and a mixture of cell culture media with different the different treatments were prepared: TLR ligands or MEVs (non-loaded or loaded with different TLR ligands). The final concentration of MEVs were between 200 and 1,000 loaded-MEV/cell, the TLR ligands alone is shown in the results figures or tables.

Internalization of MEVs by human epithelial cells by MEVs were labeled with PKH26 Red Fluorescent Cell Linker Kits following the methods described herein (see, also PCT/EP2022/070371 and U.S. provisional application Ser. No. 63/349,006). The labeled MEVs were incubated with human epithelial cells for 1 h, 3 h, 6 h, 16 h, and 24 h at 37° C. with 5% CO₂. After incubation, the cells are fixed with PFA 4%, permeabilized with 0.1% Triton™ X-100, and stained with Actin Green, USA, 1 drop/mL of PBS) that binds actin with high affinity, and the nuclei are stained with Hoechst, dilution 1:1000). The samples are analyzed by confocal microscopy.

Internalization of MEVs by human epithelial cells by MEVs loaded with different payloads as described above and following the methods described above. The loaded MEVs were incubated with human epithelial cells for 1 h, 3 h, 6 h, 16 h, and 24 h at 37° C. with 5% CO₂. After incubation, the cells are fixed with PFA 4%, permeabilized with 0.1% Triton™ X-100, and stained with Actin Green, USA, 1 drop/mL of PBS) that binds actin with high affinity, and the nuclei are stained with Hoechst, dilution 1:1000.

### E. Immunofluorescence

Cells were grown on coverslips to reach 70-80% confluency then directly with 4% paraformaldehyde (PFA) in PBS for 20 min at room temperature. Cells were permeabilized by with 0.2% Triton X100-PBS for 10 min then transferred into 0.1% Tween-PBS for 5 min. Coverslips were then incubated with primary antibodies in 0.1% Tween-5% BSA-PBS for 1-2 h, washed with 0.1% Tween-PBS, then incubated with secondary antibodies in Tween 0.1%-BSA 5%-PBS for 1 h. All the incubations were carried out in darkness in a humidified chamber at room temperature. Finally, coverslips are washed again with 0.1% Tween-PBS, incubated with Hoechst (SIGMA-Aldrich, 94403) to label DNA for 5 min, and then mounted on glass slides with Prolong (Life Technologies). Cells were analyzed by fluorescence microscopy. Antibodies against pNF-kB p65 (S536) (Cell signaling, 3033) were used.

### F. Cytotoxicity Determination

MTT (3-[4,5-Dimethylthiazol-2-yl]-2,5 Diphenyl Tetrazolium Bromide) Assay Cell viability is determined by an MTT assay as previously described. The human epithelial cells are seeded in triplicate in 48-well plates; 24 h post-seeding, the cells are treated with MEVs for 24 and 48 h. The absorbance is measured by an ELISA reader at 540 nm. Values are expressed as a percentage of cell growth versus that in the control (untreated cells).

### G. Western Blotting:

Total proteins from human epithelial cells treated with loaded MEVs as described above were isolated and analyzed by SDS-PAGE followed by Western Blotting. The primary antibodies used in the experiments are as follows: anti-NF-kB antibody (Novus, NB100-2176, dilution 1:500), anti-phosphorylated-NF-kB antibody p65 (S536) (Cell signaling, 3033, dilution 1:1000), anti-IRF-3 antibody (EPR24184) (ABCAM,ab68481, dilution 1:500), anti-phosphorylated-IRF-3 antibody (Ser396) (E.875.8) (Thermo Fischer Scientific, MA (−14947, dilution 1:1000), anti-PD-L1 antibody (Abcam, Cambridge, UK, ab213524, dilution 1:1000; antipSTAT3 (R&D System, AF4607-SP, dilution 1:500), anti-STAT3 (Novus Biologicals, Denver, CO, USA, NBP2-24463, dilution 1:1000), anti-HSP70 (Novus Biologicals, NB600-1469, dilution 1:1000), anti-HSC70 (Santa Cruz, Dallas, TX, USA, sc-7298, dilution 1:500), anti-Calnexin (Santa Cruz, sc-23954, dilution 1:500), anti-Tubulin (Santa Cruz, sc-398103, dilution 1:1000), anti-P Actin (Santa Cruz, sc-81178, dilution 1:1000), anti-GAPDH (Santa Cruz, sc-47724, dilution 1:1000). After overnight incubation with the primary antibodies at 4° C., the membranes are incubated with HRP-conjugated secondary antibody (Thermo Fisher Scientific) for 1 h, at 4° C.; the chemiluminescent signal is detected using a ChemiDoc™ MP imaging system (Bio-Rad).

### H. Real-Time PCR

The human epithelial cells are seeded in 12 well-plates at 1×10⁵ cells/mL and differentiated in M0 macrophages, as described above; the cells then are treated with MEVs for 6 and 24 h. At the end of the treatments, total RNA is extracted using QIAGEN® kit. The RNA is reverse transcribed to cDNA using the High-Capacity cDNA Reverse Transcription kit. Then, the cDNA is subjected to quantitative real-time reverse transcriptase polymerase chain reaction (RT-PCR) analysis. The sequences of the primers used are indicated in Table 9, above.

Real-time PCR is performed using Step One Real-time PCR System Thermal Cycling Block (Applied Biosystem) in a 20 μL reaction containing 300 nM of each primer, 2 μL of template cDNA, and 18 μL of 2×SYBR® Green I Master Mix. The PCR is run at 95° C. for 20 s followed by 40 cycles of 95° C. for 3 s and 60° C. for 30 s. GAPDH is used as the endogenous control. Relative changes in gene expression between control and treated samples are determined using the ΔCt method.

### L. ELISA Test

The amount of IL-6 secreted in the culture medium of epithelial cells treated with MEVs was determined using a human IL-6 ELISA kit (Invitrogen KAC1261). For adherent cells (FHs-74 and FHC), stimulations were carried out in 12-well plates with cells seeded at 40,000 cells/well. After 24 hours, cells were counted and a mixture of cell culture media with different TLR ligands or MEVs (non-loaded or loaded with different TLR ligands) were prepared with a final concentration of MEVs between 200 and 1,000 loaded-MEV/cell. Cells were treated for 48 hrs. then the supernatants were collected. Measurements were carried out as per the manufacturer's protocol (Invitrogen KAC1261).

The amount of IFNα secreted in the culture medium of THP-1 cells treated with MEVs was determined using a human IFNα ELISA kit (PBL-Assay Science, 41135-1). For adherent cells (FHs-74 and FHC), stimulations were carried out in 12-well plates with cells seeded at 40,000 cells/well. After 24 hours, cells were counted and a mixture of cell culture media with different TLR ligands or MEVs (non-loaded or loaded with different TLR ligands) were prepared with a final concentration of MEVs between 200 and 1,000 loaded-MEV/cell. Cells were treated for 48 hrs. then the supernatants were collected. The ELISA was performed according to the manufacturer's instructions.

### J. Results:

1. Internalization of MEVs into Human Epithelial Cells In Vitro

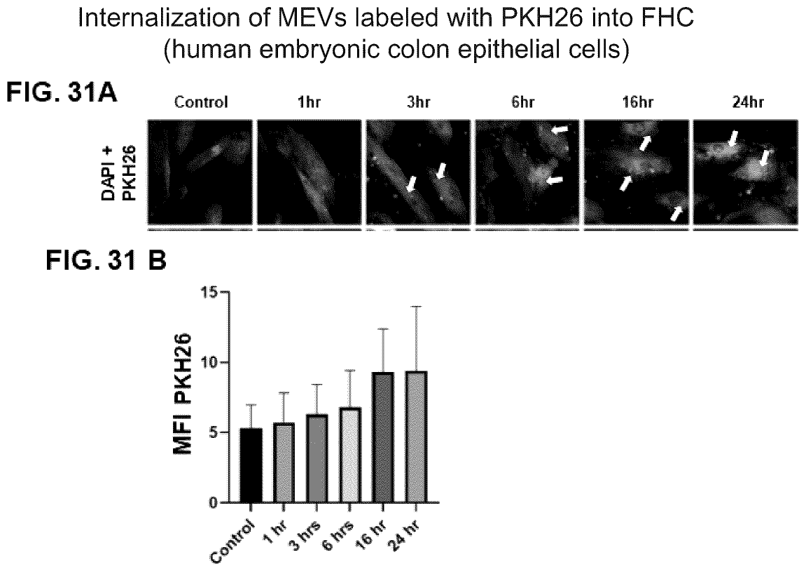

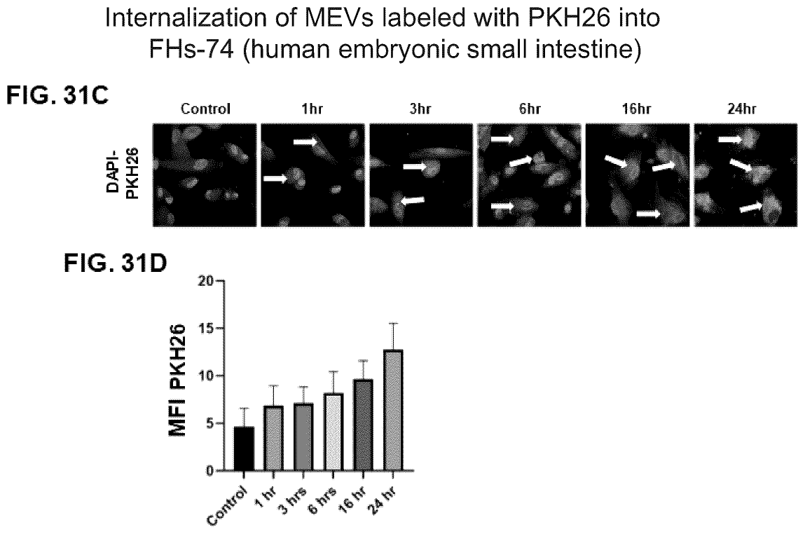

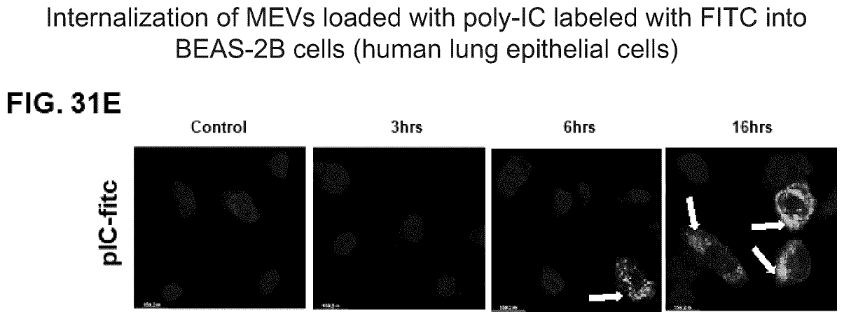

The MEVs labeled with PKH26 or loaded with different payloads either agonist of TLRs or proteins as ovalbumin (OVA) or mRNAs coding for OVA were incubated at different time point and internalized into epithelial cells from small intestine, colon, and lung, showing the ability of MEVs to penetrate to epithelial cells and to deliver its payloads (oligonucleotides, mRNAs, proteins, dsRNAs). FIGS. 31A-E show these results in FHs-74, FHC, and BEAS-2B cells small intestinal, colon and lung epithelial cells, respectively. These results confirm on human cells in vitro, the observation previously made in vivo in mice, for both respiratory and intestinal epithelial (see Figures of Examples 6 and 7, for MEVs internalization and payload delivery, respectively).

2. Payload Biological Activity after Delivery by MEVs into Human Epithelial Cells

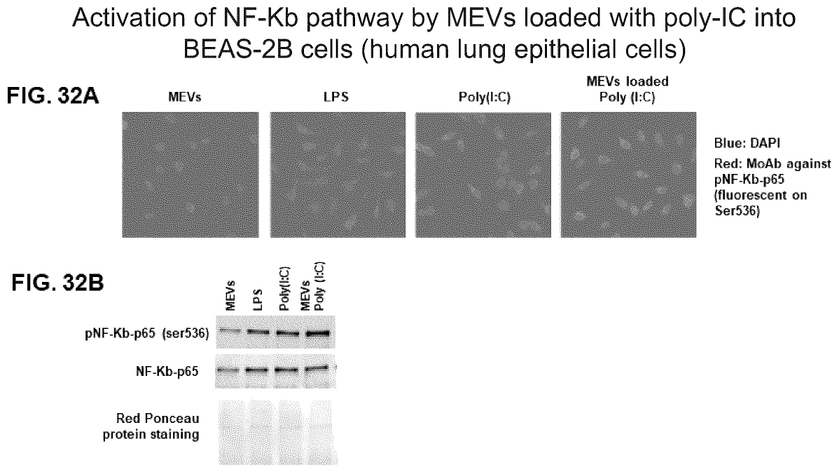

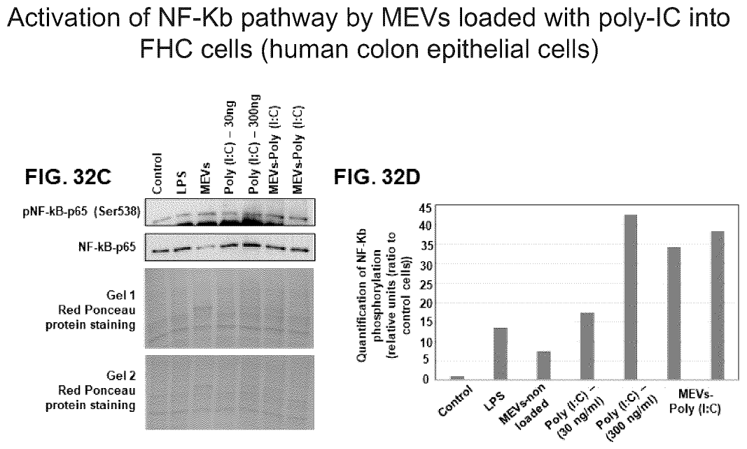

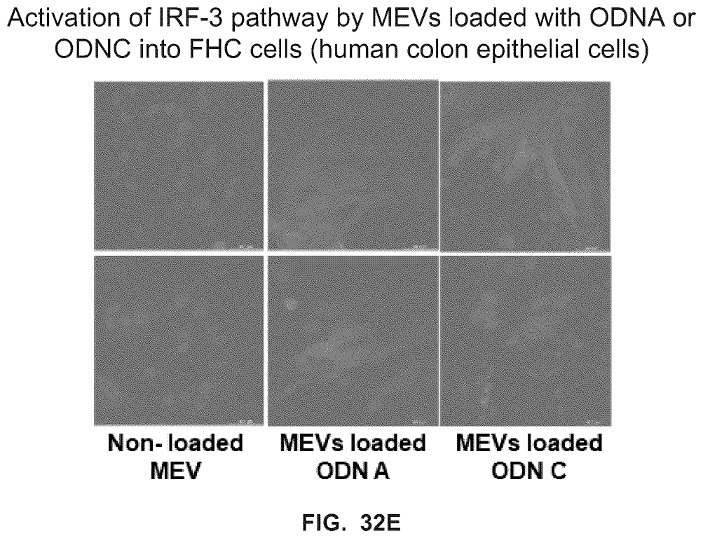

The stimulation of the phosphorylation of intracellular markers as NF-Kb and IRF-3 molecules was observed after the incubation at different timepoints with MEVs loaded with different payloads agonist of TLR, in human epithelial cells. These results (FIGS. 32 A-E) confirm, human cells in vitro, the observations made in vivo in mice, for respiratory and for intestinal epithelial (Figures of Example 7 for payload delivery, expression, and biological activity).

### K. Conclusions:

MEVs can penetrate epithelial cells, including intestinal and respiratory epithelial cells, and deliver their payloads. When the payloads are agonists or antagonists of a TLR, the payloads delivered by MEVs bind to the TLRs activating or disactivating an intracellular pathway. For TLRs, such as TLR3, TLR5, TLR7, TLR8, and TLR9, that are not located in the cellular membrane but are located in intracellular endosome membranes, MEVs reach such endosomes and deliver their payloads into the endosomes, thus allowing the payloads to stimulate (or inhibit, depending upon payload) the endosomal TLRs. The stimulation of TLRs induces intracellular pathways involved in the modulation of inflammatory reaction, modulation of imbalance of cytokines, tissular reparation, and innate and or adaptative immune response. Inhibition by antagonists inhibits such pathways.

### Example 18

### Evaluation of Toxicity of MEVs in Mice

Experiments were performed to assess the signs of MEV toxicity in vivo using Balb/C mouse model after oral and intratracheal administration.

### A. Analysis of MEV Toxicity In Vivo:

Toxicity was evaluated in several ways: clinical signs, body weights, hematological analysis, biochemical analysis, histological analysis on main organs (liver, spleen, kidney, lung and brain). The MEV samples were stored at −80° C. and thawed just prior to in vivo administrations. After thawing, each sample was mixed by vigorous vortexing for 1-2 minutes. Male Balb/C mice at 5 weeks of age and with a mass about 20 g each, were used. Animals were acclimatized. Animals were housed in polyethylene cages (<5 animals/cage), in a controlled environment with 12:12 light-dark at the temperature of 24±1° C. (mean±SD) and fed once daily with an adapted pelleted feed. Water was offered ad libitum. The animals were randomly assigned to experimental groups and acclimatized for at least 7 days before the initiation of the designed study. The experimental groups are described in Table 10, below.

The experiment was designed to determine the eventual toxicity of the test compound after its administration in mice through exemplary routes including oral, and respiratory tract (intratracheal) routes. As described above, male Balb/C mice, with a mass about 20 g each, were used. Animals were acclimatized. After a test item is administered, all mice are closely monitored for 10 days.

TABLE 10
Experimental groups in MEV toxicity study.

| | | Route of | |
|---|---|---|---|
| Group no. | Group type | administration | MEV dosage |
| 1 | Vehicle control | PO (oral) | — |
| | (PBS) | | |
| 2 | Experimental | PO (oral) | 4 × 10exp11 per animal |
| 3 | Experimental | PO (oral) | 1 × 10exp12 per animal |
| 4 | Vehicle control | IT (intratracheal) | — |
| | (PBS) | | |
| 5 | Experimental | IT (intratracheal) | 4 × 10exp11 per animal |

### Clinical Examination

Daily clinical examination of all animals included observation of behavior and signs of suffering (cachexia, weakening, difficulty for moving or feeding, etc.); test item toxicity (hunching, convulsions), and other such parameters. Determination of body weight once a week for each animal, a body weight curve was designed (Mean+SD). Observation of acute reactions was done after administration of the tested compounds.

### Clinical Pathology Investigations

After the end of the in-life phase, all animals were euthanized. Clinical pathology investigations were performed at experiment termination.

### Blood Collection

The blood samples were collected from mice by intracardiac puncture into different vials. Aliquots of blood were collected for various clinical pathology investigations into tubes containing anticoagulants: for hematology analysis with K2 EDTA and for biochemistry analysis with lithium heparin.

### Clinical Chemistry

Plasma was separated after centrifugation of whole blood samples, 45000 rpm for 15 minutes and analyzed for the following parameters at the end of treatment for all animals. Clinical chemistry analysis parameters are indicated as: Alanine Aminotransferase (ALT), Aspartate Aminotransferase (AST), and Gamma-glutamyl transferase (GGT) in units per liter (U/L); Urea in g/L and Creatinine in mg/L.

### Hematology

The following hematological parameters were determined at the end of treatment of all animals: Hemoglobin (HGB) in g/L; Hematocrit (HCT) in L/L; Mean Corpuscular Volume (MCV) in fL; Eosinophils (EO) and MHCH (BASO) as 10⁹/L.

When animals are euthanatized, an autopsy is performed, and a careful macroscopic evaluation is performed. If any organs look abnormal, a detailed description and analysis is performed.

### Histological Analysis

Histological analysis is performed for the 5 following organs: Lung, Spleen, Liver, Kidney, and Brain. The organs are collected, weighed, macroscopically observed, fixed in 4% Paraformaldehyde (PFA) and paraffin-embedded, cut into 5-7 μM sections and then observed under a fluorescence microscope.

### Histopathological Scoring

Each H&E (hematoxylin- and eosin-stained) section was thoroughly examined histologically, and lesions observed were recorded in an Excel spreadsheet. Severity of lesions were graded (minimal, mild, moderate, or severe). Their distribution also was characterized, for example as focal, multifocal, focally extensive or diffuse, and by their localization.

### B. Toxicity Study Results: Clinical Examinations

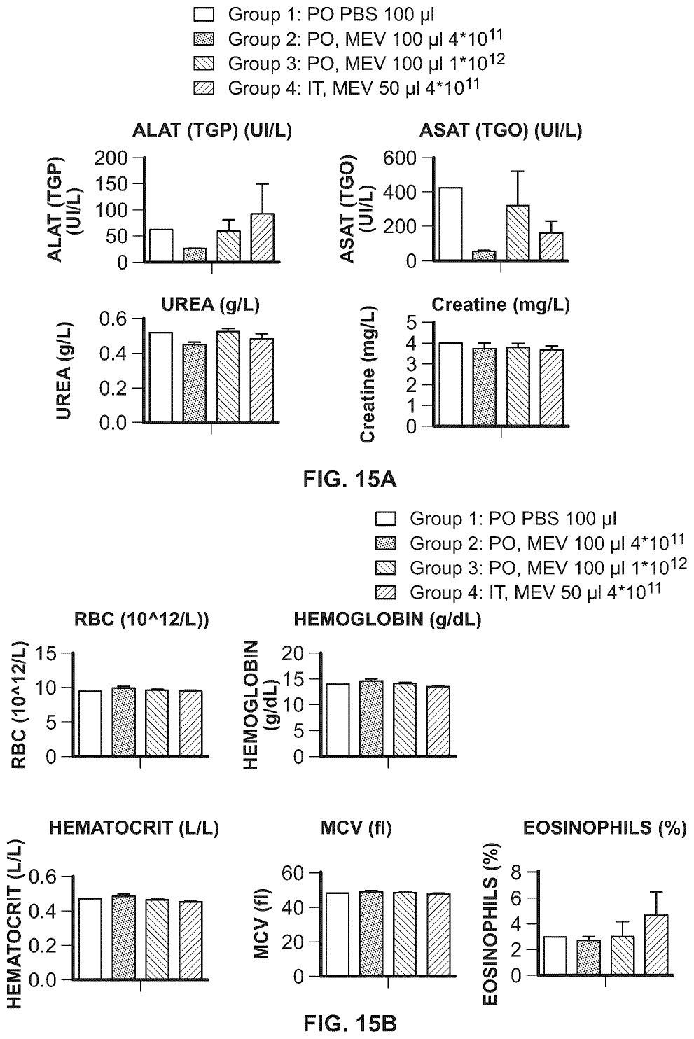

The results of assessment of toxicity of MEVs in a mouse model after oral (PO) or intratracheal (IT) administration at different doses in 4 groups of mice for each parameter are shown in FIGS. 15A and 15B, which shows clinical chemistry and hematology in mice after administration (PO, IT) of MEV, respectively.

### Clinical Chemistry

As shown below, two parameters Urea and Creatinine did not change in all treated groups compared to control groups. The enzymes Alanine Aminotransferase (ALT) and Aspartate Aminotransferase (AST) shown random non-significant deviations. The parameter Gamma-glutamyl transferase value is detected at low level (<6) in all groups indicating a protocol error and are not presented.

The results of the clinical chemistry parameters do not indicate any toxicity at all tested doses in the mice either by oral or intratracheal administration.

### Hematology

The measured parameters were not significantly changed compared to controls, indicating that the MEVs do not result in toxicity. MEV toxicity was evaluated by (1) chemistry parameters: ALAT, ASAT, urea and creatine; and (2) by hematology parameters in four groups of mice: Group 1 mice were administered 100 μl of PBS by PO delivery; Group 2 mice were administered 100 μl of 4*10¹¹ MEWV mouse by PO delivery (bar with black and white tiles); Group 3 mice were administered 100 μl of 4*10¹² MEV/mouse by PO delivery (bars with vertical lines); Group 4 mice were administered 100 μl of 4*10¹¹ MEV/mouse by IT delivery. The results present blood parameters that can be altered when there is one or more of blood-, kidney-, and liver-related toxicity. Hematocrit, hemoglobin, eosinophil levels, RBC count, and volume (MCV) are hematologic parameters; urea and creatine are biochemical markers of kidney injury; ALAT (or ALT) and ASAT (or AST) are biochemical markers of liver injury. Alanine transaminase (ALT), also called alanine aminotransferase (ALAT), is a transaminase enzyme that occurs in plasma and in various body tissues, but most commonly in the liver. Elevated levels of ALT can be related to liver-related problems, such as hepatitis and/or liver damage. Aspartate transaminase (AST), also known as aspartate aminotransferase (ASAT), is another transaminase enzyme important in amino acid metabolism. Beyond liver toxicity, AST can be elevated also in diseases affecting other organs. Serum ALT level, serum AST level, and their ratio (AST/ALT ratio) are used clinically as biomarkers for liver health. In the instant study, no statistically significant differences between experimental groups in ALT and AST levels were observed (see, FIG. 15A).

Eosinophils (eosinophiles) are a variety of white blood cells and one of the immune system components responsible for combating multicellular parasites and certain infections in vertebrates. Eosinophils usually account for less than 7% of the circulating leukocytes. Beyond causes related to infection or parasitic infestation, elevated eosinophil levels (also known as eosinophilia) can be also a sign of allergic or atopic reactions. In the instant study no statistically significant differences between experimental groups in eosinophil levels were observed.

### Histology

The main organs were collected, fixed in 4% PFA, paraffin-embedded, cut into sections and stained with H&E.

### Lung

Histopathological changes observed in lung sections are primarily limited to minimal to mild focal to multifocal alveolar hemorrhages in 8/10 lung sections examined in animals receiving intratracheal negative control material and test item. Changes were relatively subtle and limited in some of the alveolar spaces. This observation was observed at comparable incidence and severity in negative control and treated animals.

### Kidney

Presence of a few basophilic tubules were observed in 1 animal treated PO with 4×10¹¹ MEV dose. This finding was minimal in severity and often is observed spontaneously at low incidence and severity in laboratory rodent animals. It is therefore considered as incidental in origin in the present study.

### Spleen, Liver, and Brain

No histopathological changes were observed in spleen, brain, or liver in all sections examined.

### Summary

The pulmonary changes observed in most animals receiving the experimental material intra-tracheally were considered not related to treatment. There was no treatment-related change observed in all organs examined, indicating that MEVs are not toxic to animals under the experimental conditions (see, Table 11).

TABLE 11
Histopathology results after administration
orally or intratracheally of MEVs

| Group | 1 | 2 | 3 | 4 | 5 |
|---|---|---|---|---|---|
| Organ | Brain | | | | |
| Histopathological Findings | | | | | |
| Number examined | 3 | 6 | 5 | 4 | 6 |
| Unremarkable | 3 | 6 | 5 | 4 | 6 |
| Organ | Spleen | | | | |
| Histopathological Findings | | | | | |
| Number examined | 3 | 6 | 6 | 4 | 6 |
| Unremarkable | 3 | 6 | 6 | 4 | 6 |
| Organ | Kidney | | | | |
| Histopathological Findings | | | | | |
| Number examined | 3 | 6 | 6 | 4 | 6 |
| Unremarkable | 3 | 5 | 6 | 4 | 6 |
| Basophilic tubules | | | | | |
| Minimal | 0 | 1 | 0 | 0 | 0 |
| Organ | Liver | | | | |
| Histopathological Findings | | | | | |
| Number examined | 3 | 6 | 6 | 4 | 6 |
| Unremarkable | 3 | 6 | 6 | 4 | 6 |
| Organ | Lung | | | | |
| Histopathological Findings | | | | | |
| Number examined | 3 | 6 | 6 | 4 | 6 |
| Unremarkable | 3 | 6 | 6 | 1 | 1 |
| Hemorrhage, alveolar | | | | | |
| Minimal | 0 | 0 | 0 | 2 | 4 |
| Mild | 0 | 0 | 0 | 1 | 1 |

### C. Conclusion

Regarding the clinical aspects, all the animals, at each time of the study, exhibited normal behaviors, body weight, water and food consumption. The clinical findings indicate general tolerance in the mouse model of the test MEV products. Under the experimental conditions, both negative controls (PBS) and MEV at concentration of 4*10¹¹ MEV/mouse or 4*10¹² MEV/mouse by oral administration and at concentration of 4*10¹¹ MEV/mouse by IT administration did not induce any abnormal, toxic effect on animals.

### Example 19

MEVs Loaded with TLR9 Agonists for the Treatment of Inflammatory Bowel Disease (IBD)

IBD is characterized by an acute or chronic relapsing-remitting inflammation of the gastrointestinal tract. IBD encompasses chronic inflammatory disorders as Crohn's disease (CD) and Ulcerative Colitis (UC), both characterized by a perturbed homeostasis between commensal bacteria and mucosal immunity driving to a mucosal inflammation.

Toll like receptors (TLRs) control not only innate immunity but also regulate adaptative immunity, such T cell activation, by playing a pleiotropic role in the induction of intestinal inflammation (Corridon D., et al. Front Med (Lausanne) 2018; 16:32. Cario E. Inflamm Bowel Disease 2010; 16:1583-97.) The intracellular TLR9 recognizes unmethylated dinucleotide CpG which acts as an agonist for such a pleiotropic role. TLR9 is expressed in macrophages, dendritic cells and B cells, but also in epithelial cells from intestinal and respiratory track. TLR9 deficient mice develop more severe UC compared to wildtype mice (Lee J., et al. Nat Cell Biol 2006; 1:327-36).

MEVs loaded with CpG oligonucleotides, such as ONDA or ONDX, appear to be an excellent system to treat IBD, given the ability of MEVs to pass through the stomach and to deliver its payload in the intestinal epithelium and in GALT cells as demonstrated in example 6.

In mice, the disease can be induced by the treatment with Dextran Sodium Sulfate (DSS) to create a disease model for the evaluation of experimental therapies. Thus, the treatment of mice suffering from DSS-induced acute experimental colitis is a widely used animal model for the evaluation of therapies against the IBD disease. In our case, acute colitis was induced in mice by DSS 3% in drinking water for eight days. Mice were treated with MEVs loaded with agonists of TLR9 (ODNA or ODNX) for 4 days and euthanized on D8 for acute inflammation analysis. All handling of animals was conducted carefully to reduce stress to the minimum. All the experiments were performed in compliance with the guidelines of the French Ministry of Agriculture for experiments with laboratory animals.

### A. Loading of MEVs

Isolated and purified MEVs were loaded with specific payloads as described in Example 3 using the best method for loading for the specific payload type. For the example, 10χExp12 MEVs were loaded with 10 μg Oligonucleotides (CpG oligonucleotide) agonist of TLR9. The molecules used were ONDA (Invivogen, vaccine grade ODN 1585 a class A CpG oligonucleotide agonist of TLR9) and ONDX (or cobitolimob, class B CpG oligonucleotide agonist of TLR9—synthetized by Eurogentec).

ODNX sequence:
SEQ ID NO: 193
5′-GGAACAGTTCGTCCATGGC-3′
ODNA sequence:
SEQ ID NO: 194
5′-ggGGTCAACGTTGAgggggg-3′ (bases in capital are
phosphodiesters, and those in small case are phosphonothioates..

### B. Experimental Design and Mice Treatment Schedule:

Animals were randomized before treatment group's allocation based on body weight. The disease was induced by administration of DSS in drinking water (3%, w/v, MW: 40000, TdB Consultancy AB) in C57BL/6 female mice at 9 weeks of age. Mice were administered with test compounds either at D3 and D6 (M1, M3 and M5 intrarectal), or daily from D3 to D6 (M2 and M4, per os). Experimental groups were as follows: seven animals per group; a total of 49 mice.

### Group 1: Normal Mice (Overall Positive Control)

Mice of this group are not treated with DDS (no DDS in water), and not treated with any test compound.

### Group 2: IBD-Mice (Negative Control)

Mice of this group are treated with 3% DDS for 7 days in drinking water (3%, w/v, MW: 40000, TdB Consultancy AB), treated with PBS solution. Administration: 100 μL by p.o.

### Group 3: IBD Treated Mice (Treatment Positive Control)

Mice of this group are treated with 3% DDS for 7 days in drinking water (3%, w/v, MW: 40000, TdB Consultancy AB), treated with ODNX dissolved in sterile water 1 mg/mL solution. Administration 100 μL by intrarectal (i.r.).

### C4. Group 4: MEVs (ONDX-Loaded)

Mice of this group are treated with 3% DDS for 7 days in drinking water (3%, w/v, MW: 40000, TdB Consultancy AB), treated MEVs loaded-ODNX, 10×Exp12 MEVs/mL solution. Administration 100 μL by p.o.

### C5. Group 5: MEVs (ONDX-Loaded)

Mice of this group are treated with 3% DDS for 7 days in drinking water (3%, w/v, MW: 40000, TdB Consultancy AB), treated MEVs loaded-ODNX, 10×Exp12 MEVs/mL solution. Administration 100 μL by i.r.

### C6. Group 6: MEVs (ODNA-Loaded)

Mice of this group are treated with 3% DDS for 7 days in drinking water (3%, w/v, MW: 40000, TdB Consultancy AB), treated MEVs loaded-ODNA, 10×Exp12 MEVs/mL solution. Administration 100 μL by p.o.

### Group 7: IBD Treated Mice (MEVs Treatment Positive Control)

Mice of this group are treated with 3% DDS for 7 days in drinking water (3%, w/v, MW: 40000, TdB Consultancy AB), treated with ODNA, dissolved in sterile water 1 mg/mL solution. Administration 100 μL by i.r.

All groups and treatments are summarized in the following table.

TABLE 12
Experimental design and mice treatments

| Group | Mice | Disease | | | Dose and |
|---|---|---|---|---|---|
| # | number | induction | Treatment | Route | frequency |
| 1 | 7 | Water | Untreated | N/A | N/A |
| 2 | 7 | 3% DSS | Vehicle (PBS) | p.o. | 100 μL/mouse, |
| | | | | | daily from |
| | | | | | D 3 to D 7 |
| 3 | 7 | 3% DSS | M1: ODNX | i.r. | 100 μg in 100 |
| | | | | | μl/mouse, |
| | | | | | D 3 and D 6 |
| 4 | 7 | 3% DSS | M2: | p.o. | 100 μl/mouse, |
| | | | MEVs + ODNX | | daily from |
| | | | | | D 3 to D 7 |
| 5 | 7 | 3% DSS | M3: | i.r. | 100 μl/mouse, |
| | | | MEVs + ODNX | | D 3 and D 6 |
| 6 | 7 | 3% DSS | M4: | p.o. | 100 μl/mouse, |
| | | | MEVs + ODNA | | daily from |
| | | | | | D 3 to D 7 |
| 7 | 7 | 3% DSS | M5: ODNA | i.r. | 50 μg in 100 |
| | | | | | μl/mouse, |
| | | | | | D 3 and D 6 |

### C. IBD Analysis

Mice were euthanized at D8; samples were harvested to determine the extent of intestinal inflammation using various quantitative and semi quantitative methods including body weight evolution, clinical score (Disease Activity index; DAI, Table 13, below), large intestine measurement, cytokine analysis in colon and/or ileum tissue (TNFα, TGFβ, IFNα, IFNγ, IL-6 and IL-17A) and intestinal and colon histopathology for inflammation analysis, as follows and as summarized in Table 14, below.

In-Life Samples (all groups)
Bodyweight (daily)
Disease Activity Index (DAI) (diarrhea and blood in stool)

TABLE 13
Disease Activity Index scoring criteria

| | Description | Score |
|---|---|---|
| Diarrhea | Normal stool | 0 |
| | Light diarrhea | 2 |
| | Severe diarrhea | 4 |
| Blood in stool | No | 0 |
| | Light bleeding | 2 |
| | Severe bleeding | 4 |
| Weight loss | <1% | 0 |
| | 1-5% | 1 |
| | 5-10% | 2 |
| | 10-15% | 3 |
| | >15% | 4 |
| | Final score | /12 |

### Terminal Samples:

Colon collection (for all groups)
   Length measurement
   TNFα, TGFβ, IFNα, IFNγ, IL-6 and IL-17A levels
   Histological analysis (H&E staining)
Ileum collection (for all groups)
   Length measurement
   TNFα, TGFβ, IFNα, IFNγ, IL-6 and IL-17A levels for groups 1, 2, 4, 6
   Histological analysis (H&E staining)

TABLE 14
Intestinal Inflammation Analysis Summary

| Organ or | Time- | Sample | |
|---|---|---|---|
| Parameter | Point | Condition | Analysis |
| Body | Daily | N/A | Body weight evolution |
| weight | (all | | |
| | groups) | | |
| Small | D 8 (all | Organ | Total length |
| intestine | groups) | measurement | |
| Small | D 8 | Tissue | Cytokine analysis (TNFα, TGFβ, |
| intestine | (groups | homogenate | IFNα, IFNγ, IL-6 and IL-17A) |
| | 1, 2, 4, | | |
| | 6) | | |
| Small | D 8 (all | FFPE | Histological analysis (HE) |
| intestine | groups) | | |
| Colon | D 8 (all | Organ | Total length |
| | groups) | measurement | |
| Colon | D 8 (all | Tissue | Cytokine analysis (TNFα, TGFβ, |
| | groups) | homogenate | IFNα, IFNγ, IL-6 and IL-17A) |
| Colon | D 8 (all | FFPE* | Histological analysis (HE) |
| | groups) | | |

*FFPE Archived Formalin-Fixed Paraffin-Embedded (FFPE)

### D. Gut Tissue Harvesting

Ileum and large intestines were harvested and measured before processing. The samples dedicated to histological analysis were embedded in paraffin 48-72 h after formalin fixation (FFPE) and processed for H&E staining, and the samples dedicated to multiplex analysis were snap-frozen for homogenization.

### E. Cytokine Measurement in Ileum/Colon Homogenates

TNFα, TGFβ, IFNα, IFNγ, IL-6 and IL-17A concentrations in colon and/or ileum homogenates were determined by Multiplex Immunoassay (Luminex Lx200, Millipore) according to the manufacturer's instructions. Results are reported as pg/mL.

F. Ileum and Colon histology

1.5-cm piece of ileum and 1-cm piece of colon tissue were fixed in 4% buffered formaldehyde, paraffin embedded, sectioned, and then stained with Hematoxylin and Eosin (H&E) staining. Histopathological changes were individually scored by two independent technicians and validated by a pathologist. Each mouse was scored individually for each of the parameters as a combined score (Table 15), as follows, from Pelin Arda-Pirincci & Guliz Aykol-Celik. Galectin-1 reduces the severity of dextran sulfate sodium (DSS)-induced ulcerative colitis by suppressing

TABLE 15
Histological colitis damage scoring system

| Feature graded | Grade | Description |
|---|---|---|
| Severity of inflammation | 0 | None |
| | 1 | Slight |
| | 2 | Moderate |
| | 3 | Severe |
| Spread of inflammation | 0 | None |
| | 1 | Mucosa |
| | 2 | Mucosa and submucosa |
| | 3 | Transmural |
| Crypt damage | 0 | None |
| | 1 | Basal ⅓ damaged |
| | 2 | Basal ⅔ damaged |
| | 3 | Only surface epithelium intact |
| | 4 | Entire crypt and epithelium lost |
| Percent involvement | 1 | 1-25% |
| | 2 | 26-50% |
| | 3 | 51-75% |
| | 4 | 76-100% |

inflammatory and oxidative stress response. Bosn J Basic Med Sci. 2020; 20(3):319-328.). The total score is between 0 and 14.

### G. Treatment Results:

### 1. Disease Activity Index (DAI) Results:

The life samples were recovered every day: i. body weight, ii. diarrhea, and iii. blood in stool and used to determine the disease activity index (DAI). The results are summarized in Table 33, for each animal in each group for seven consecutive days.

### 2. Cytokines Measurements by Multiplex Assay

Six cytokines were measured after mice euthanasia (D8) in small intestine (ileum) and colon homogenates. No differences between samples were detected either for IL-6, or IL-17, or IFNα, or IFNγ, or TNFα, or TGFβ from colon homogenates of groups 1, 2, 3, 5, and 7. The analysis of samples from ileum (small intestine) showed quantifiable differences of the cytokine's level between samples from groups 1, 2, 4, 6. Table 34 summarizes these results. As the doses evaluated, the treatment with MEVs loaded with ONDA or with ONDX shows the decrease of inflammatory cytokines such as IFNγ, IL-6, IL-17, and TNFα. As far as the levels of non-inflammatory cytokines are concerned, no difference was detected on the level of IFNα, and an increase was shown in the level of TGFβ. In both cases the decrease and increase on the level of inflammatory and non-inflammatory cytokines is higher when mice were treated with MEVs loaded with ONDA as compared to normal animal (without disease induction—positive control)_or IBD mice (with disease induction, and vehicle (PBS as treatment)-negative control).

TABLE 16
ODNA-loaded MEVs improved the disease activity index in the IBD model.

| | Number | Cumulative score (DAI) | | | | | | | |
|---|---|---|---|---|---|---|---|---|---|
| Group | of mice | D 0 | D 1 | D 2 | D 3 | D 4 | D 5 | D 6 | D 7 |
| GR1: | 1 | 0 | 1 | 1 | 1 | 1 | 1 | 1 | 1 |
| normal mice | 2 | 0 | 0 | 1 | 1 | 1 | 1 | 1 | 0 |
| (positive | 3 | 0 | 0 | 0 | 1 | 1 | 1 | 0 | 0 |
| control) | 4 | 0 | 0 | 0 | 0 | 0 | 0 | 0 | 0 |
| | 5 | 0 | 0 | 0 | 0 | 0 | 0 | 0 | 0 |
| | 6 | 0 | 0 | 1 | 1 | 1 | 0 | 0 | 0 |
| | 7 | 0 | 1 | 1 | 1 | 1 | 0 | 1 | 0 |
| | Mean | 0.00 | 0.29 | 0.57 | 0.71 | 0.71 | 0.43 | 0.43 | 0.14 |
| | sem | 0.00 | 0.18 | 0.20 | 0.18 | 0.18 | 0.20 | 0.20 | 0.14 |
| | N | 7 | 7 | 7 | 7 | 7 | 7 | 7 | 7 |
| GR2: DSS + | 8 | 0 | 0 | 0 | 0 | 1 | 2 | 3 | 12 |
| Vehicle (PBS) | 9 | 0 | 0 | 0 | 0 | 0 | 3 | 6 | 6 |
| (D 3 to D 6, | 10 | 0 | 0 | 0 | 0 | 5 | 6 | 4 | 10 |
| p.o.) | 11 | 0 | 0 | 0 | 0 | 2 | 4 | 6 | 4 |
| (negative | 12 | 0 | 0 | 0 | 1 | 1 | 3 | 6 | 7 |
| control) | 13 | 0 | 0 | 0 | 4 | 4 | 6 | 9 | 7 |
| | 14 | 0 | 0 | 0 | 0 | 5 | 6 | 5 | 9 |
| | Mean | 0.00 | 0.00 | 0.00 | 0.71 | 2.57 | 4.29 | 5.57 | 7.86 |
| | sem | 0.00 | 0.00 | 0.00 | 0.57 | 0.78 | 0.64 | 0.72 | 1.01 |
| | N | 7 | 7 | 7 | 7 | 7 | 7 | 7 | 7 |
| GR3: DSS + | 15 | 0 | 0 | 0 | 0 | 5 | 4 | 7 | 10 |
| ODNX | 16 | 0 | 0 | 0 | 0 | 3 | 4 | 6 | 7 |
| (D 3 and D 6, | 17 | 0 | 0 | 0 | 3 | 3 | 5 | 4 | 5 |
| 100 μg/mouse, | 18 | 0 | 0 | 0 | 0 | 0 | 4 | 5 | 6 |
| i.r.) (treatment | 19 | 0 | 0 | 0 | 0 | 0 | 1 | 3 | 6 |
| positive | 20 | 0 | 0 | 1 | 1 | 2 | 5 | 5 | 8 |
| control) | 21 | 0 | 0 | 0 | 0 | 4 | 6 | 10 | 10 |
| | Mean | 0.00 | 0.00 | 0.14 | 0.57 | 2.43 | 4.14 | 5.71 | 7.43 |
| | sem | 0.00 | 0.00 | 0.14 | 0.43 | 0.72 | 0.59 | 0.87 | 0.75 |
| | N | 7 | 7 | 7 | 7 | 7 | 7 | 7 | 7 |
| GR4: DSS + | 22 | 0 | 0 | 0 | 0 | 0 | 3 | 2 | 9 |
| MEVs + ODNX | 23 | 0 | 0 | 0 | 0 | 4 | 6 | 4 | 9 |
| (D 3 to D 6, | 24 | 0 | 0 | 0 | 1 | 2 | 4 | 5 | 7 |
| 1 × 10¹¹/mL, | 25 | 0 | 1 | 2 | 2 | 2 | 6 | 5 | 6 |
| p.o.) | 26 | 0 | 0 | 0 | 0 | 4 | 3 | 4 | 7 |
| | 27 | 0 | 1 | 1 | 1 | 1 | 6 | 6 | 9 |
| | 28 | 0 | 0 | 0 | 0 | 0 | 3 | 6 | 5 |
| | Mean | 0.00 | 0.29 | 0.43 | 0.57 | 1.86 | 4.43 | 4.57 | 7.43 |
| | sem | 0.00 | 0.18 | 0.30 | 0.30 | 0.63 | 0.57 | 0.53 | 0.61 |
| | N | 7 | 7 | 7 | 7 | 7 | 7 | 7 | 7 |
| GR5: DSS + | 29 | 0 | 0 | 1 | 1 | 6 | 5 | 5 | 6 |
| MEVs + ODNX | 30 | 0 | 0 | 1 | 1 | 3 | 5 | 4 | 6 |
| (D 3 to D 6, | 31 | 0 | 0 | 0 | 0 | 5 | 6 | 7 | 8 |
| 1 × 10¹¹/mL, | 32 | 0 | 0 | 1 | 0 | 3 | 4 | 8 | 9 |
| i.r.) | 33 | 0 | 0 | 0 | 0 | 2 | 7 | 5 | 8 |
| | 34 | 0 | 1 | 1 | 0 | 4 | 4 | 8 | |
| | 35 | 0 | 0 | 0 | 0 | 3 | 4 | 5 | 7 |
| | Mean | 0.00 | 0.14 | 0.57 | 0.29 | 3.71 | 5.00 | 6.00 | 7.33 |
| | sem | 0.00 | 0.14 | 0.20 | 0.18 | 0.52 | 0.44 | 0.62 | 0.49 |
| | N | 7 | 7 | 7 | 7 | 7 | 7 | 7 | 6 |
| GR6: DSS + | 36 | 0 | 1 | 1 | 1 | 4 | 2 | 4 | 6 |
| MEVs + ODNA | 37 | 0 | 0 | 0 | 2 | 2 | 6 | 6 | 6 |
| (D 3 to D 6, | 38 | 0 | 0 | 0 | 2 | 2 | 3 | 5 | 5 |
| 1 × 10¹¹/mL, | 39 | 0 | 1 | 0 | 0 | 2 | 5 | 5 | 6 |
| p.o.) | 40 | 0 | 0 | 0 | 0 | 1 | 6 | 5 | 5 |
| | 41 | 0 | 0 | 0 | 0 | 1 | 4 | 5 | 6 |
| | 42 | 0 | 0 | 0 | 1 | 1 | 2 | 4 | 4 |
| | Mean | 0.00 | 0.29 | 0.14 | 0.86 | 1.86 | 4.00 | 4.86 | 5.43 |
| | sem | 0.00 | 0.18 | 0.14 | 0.34 | 0.40 | 0.65 | 0.26 | 0.30 |
| | N | 7 | 7 | 7 | 7 | 7 | 7 | 7 | 7 |
| GR7: DSS + | 43 | 0 | 0 | 0 | 0 | 2 | 2 | 7 | 8 |
| ODNA. | 44 | 0 | 0 | 0 | 0 | 5 | 8 | 7 | 8 |
| (D 3 and D 6, | 45 | 0 | 0 | 0 | 0 | 3 | 4 | 7 | 7 |
| 50 μg/mouse, | 46 | 0 | 1 | 1 | 1 | 1 | 3 | 6 | 6 |
| i.r.) | 47 | 0 | 0 | 0 | 0 | 1 | 4 | 7 | 8 |
| | 48 | 0 | 0 | 0 | 0 | 0 | 3 | 3 | 3 |
| | 49 | 0 | 0 | 0 | 0 | 0 | 5 | 6 | 7 |
| | Mean | 0.00 | 0.14 | 0.14 | 0.14 | 1.71 | 4.14 | 6.14 | 6.71 |
| | sem | 0.00 | 0.14 | 0.14 | 0.14 | 0.68 | 0.74 | 0.55 | 0.68 |
| | N | 7 | 7 | 7 | 7 | 7 | 7 | 7 | 7 |

TABLE 17
Treatment with MEVs loaded with the TLR9 agonist
decreases inflammatory cytokines in IBD model.

| | Number | IFNγ | IFNα | TNFα | IL-6 | IL-17A | TGFβ |
|---|---|---|---|---|---|---|---|
| Group | of mice | (pg/mL) | (pg/mL) | (pg/mL) | (pg/mL) | (pg/mL) | (pg/mL) |
| GR1: | 1 | 3.92 | 17.57 | 22.26 | 33.17 | 19.28 | 88.55 |
| normal | 2 | 6.77 | 19.09 | 19.50 | 77.89 | 17.53 | 50.35 |
| mice | 3 | 4.78 | 19.57 | 32.66 | 43.92 | 15.44 | 9.95 |
| | 4 | 5.74 | 15.04 | 21.85 | 77.25 | 3.71 | 59.19 |
| | 5 | 6.01 | 19.57 | 29.16 | 79.81 | 11.22 | 51.39 |
| | 6 | 6.81 | 28.27 | 33.67 | 167.79 | 37.51 | 16.29 |
| | 7 | 7.67 | 16.48 | 13.29 | 58.95 | 13.33 | 34.67 |
| | Mean | 5.96 | 19.37 | 24.63 | 76.97 | 16.86 | 44.34 |
| | sem | 0.49 | 1.62 | 2.82 | 16.61 | 3.94 | 10.15 |
| | N | 7 | 7 | 7 | 7 | 7 | 7 |
| GR2: DSS + | 8 | 2.96 | 13.80 | 17.06 | 81.72 | 8.92 | 63.66 |
| Vehicle | 9 | 6.95 | 20.97 | 21.58 | 66.89 | 55.49 | 28.52 |
| (PBS) | 10 | 1.93 | 17.57 | 17.64 | 48.09 | 13.51 | 67.07 |
| (D 3 to D 6, | 11 | 2.31 | 23.96 | 13.21 | 181.62 | 62.91 | 9.95 |
| p.o.) | 12 | 8.44 | 26.65 | 26.70 | 46.71 | 9.45 | 49.29 |
| (negative | 13 | 4.65 | 23.75 | 44.64 | 131.40 | 33.41 | 53.43 |
| control) | 14 | 10.26 | 30.48 | 20.13 | 33.17 | 9.81 | 58.26 |
| | Mean | 5.36 | 22.45 | 22.99 | 84.23 | 27.64 | 47.17 |
| | sem | 1.23 | 2.11 | 3.94 | 20.32 | 8.80 | 7.82 |
| | N | 7 | 7 | 7 | 7 | 7 | 7 |
| GR4: DSS + | 22 | 2.66 | 22.72 | 14.17 | 81.72 | 27.92 | 77.29 |
| MEVs + ODNX | 23 | 2.33 | 27.38 | 25.06 | 64.26 | 17.88 | 46.00 |
| (D 3 to D 6, | 24 | 3.53 | 12.45 | 14.56 | 45.32 | 5.52 | 68.72 |
| 1 × 10¹¹/mL, | 25 | 1.20 | 11.34 | 7.52 | 16.92 | 2.24 | 71.93 |
| p.o.) | 26 | 5.61 | 30.15 | 22.39 | 89.93 | 14.04 | 40.03 |
| | 27 | 1.75 | 13.47 | 7.29 | 10.00 | 2.61 | 61.90 |
| | 28 | 5.03 | 19.09 | 13.04 | 44.62 | 4.25 | 40.03 |
| | Mean | 3.16 | 19.51 | 14.86 | 50.40 | 10.64 | 57.99 |
| | sem | 0.62 | 2.84 | 2.56 | 11.49 | 3.67 | 5.95 |
| | N | 7 | 7 | 7 | 7 | 7 | 7 |
| GR6: DSS + | 36 | 1.29 | 10.55 | 4.03 | 1.80 | 5.52 | 116.00 |
| MEVs + ODNA | 37 | 2.23 | 26.28 | 23.80 | 58.95 | 5.16 | 44.86 |
| (D 3 to D 6, | 38 | 2.10 | 19.81 | 10.07 | 39.68 | 15.09 | 49.29 |
| 1 × 10¹¹/mL, | 39 | 3.40 | 27.92 | 25.59 | 60.29 | 4.80 | 47.12 |
| p.o.) | 40 | 1.22 | 12.09 | 1.44 | 6.19 | 2.24 | 189.41 |
| | 41 | 0.66 | 10.55 | 5.05 | 7.17 | 0.74 | 54.42 |
| | 42 | 4.52 | 14.12 | 18.72 | 24.90 | 6.96 | 44.86 |
| | Mean | 2.20 | 17.33 | 12.67 | 28.43 | 5.79 | 77.99 |
| | sem | 0.51 | 2.79 | 3.76 | 9.44 | 1.74 | 20.92 |
| | N | 7 | 7 | 7 | 7 | 7 | 7 |

### H. Conclusion

Treatment with MIEVs loaded with TLR agonists modulates the level of cytokines in vivo as demonstrated and indicated by the results shown and described herein. Treatment with MEVs loaded with different agonists of TLR9 results in an improved disease activity index (DAI) score in the IBD mice model. Different payloads in the MEVs induced a different degree of improvement or modulation of the immune system, distinctive of each individual payload molecule.

Oral administration of MEVs for delivery of TLR modulators (agonists or antagonists) provides modulation of the imbalance in in the level of cytokines generated as IBD diseases (ulcerative colitis, and Crohn's disease) progress.

These results show that the use of MEVs by respiratory administration (nebulization or other), topical administration (eyes, other tissues), intranasal administration (to the CNS) to delivery agonists and antagonists of TLRS provides for modulation of imbalance in the levels of cytokines generated during the progress of inflammatory diseases or states of diseases.

### I. Other Exemplary TLR9 Ligands for Delivery in MEVs for Targeting TLR9 (See Tables 18A and 18B)

TABLE 18A

| Seq | | |
|---|---|---|
| ID NO: | Oligo | Sequence |
| 195 | S3 1 | AGGCGTTTTT |
| 196 | S3 2 | TGGTTTTTTTCGTTTTTTTTTTTT |
| 197 | S3 3 | TCGTTTTTTTCGTTTTTTTTTTTT |
| | S3 4 | TCCCCC |
| | S3 5 | TCGTT |
| 198 | CpG 3 | TGGTTCGTTTTTTT |
| 199 | CpG 4 | TGGTTTCGTTTTTTTTT |
| 200 | CpG 5 | TGGTTTTCGTTTTTTTTT |
| 201 | CpG 6 | TGGTTTTTCGTTTTTTTTTT |
| 202 | CpG 7 | TGGTTTTTTCGTTTTTTTTTTT |
| 203 | CpG 8 | TCGTCGTTTTTT |
| 204 | CpG 9 | TCGTTCGTTTTTTT |
| 205 | CpG 10 | TCGTTTCGTTTTTTTT |
| 206 | CpG 11 | TCGTTTTCGTTTTTTTTT |
| 207 | CpG 12 | TCGTTTTTCGTTTTTTTTTT |
| 208 | CpG 13 | TCGTTTTTTCGTTTTTTTTTTT |
| 209 | 53 | TGGTGGTTTTGTCGTTTTGTGGTT |
| 210 | 57 | TGGTGGTTTTGTGGTTTTGTGGTT |
| 211 | 57-2 | TGGTGGTTTTGTGGTTTTGTGG |
| 212 | 57-4 | TGGTGGTTTTGTGGTTTTGT |
| 213 | 57-6 | TGGTGGTTTTGTGGTTTT |
| 214 | 57-8 | TGGTGGTTTTGTGGTT |
| 215 | 2-57 | GTGGTTTTGTGGTTTTGTGGTT |
| 216 | 4-57 | GGTTTTGTGGTTTTGTGGTT |
| 217 | 6-57 | TTTTGTGGTTTTGTGGTT |
| 218 | 8-57 | TTGTGGTTTTGTGGTT |
| 219 | 57-A | TAATAATTTTATAATTTTATAATT |
| 220 | 57-A1 | TAATGGTTTTGTGGTTTTGTGGTT |
| 221 | 57-A2 | TGGTGGTTTTGTGGTTTTGTAATT |
| 222 | 57-A3 | TGGTGGTTTTGTAATTTTGTGGTT |
| 223 | 57-8A | TAATAATTTTATAATT |
| 224 | 57-8A1 | TAATGGTTTTGTGGTT |
| 225 | 57-8A2 | TGGTGGTTTTGTAATT |
| 226 | 57-8A3 | TGGTAATTTTGTGGTT |
| 227 | CpG-B | TCGTCGTTTTGTCGTTTTGTCGTT |
| 228 | CpG-C | TCGTCGTTTTCGGCGCGCGCCG |
| 229 | CpG 8(PO) | tcgtcgtttttt |
| 230 | 57(PO) | tggtggttttgtggttttgtggtt |

TABLE 18B

| SEQ | | |
|---|---|---|
| ID NO: | Oligo | Sequence |
| 231 | 18 | CTCTTGATTTCTCAGC |
| 232 | 95 | GACATAGTTAATTGCC |
| 233 | 5 | TCTCATTTATTTCGGC |
| 234 | 549148 | GGCTACTACGCCGTCA |
| 235 | 549139 | AGCGCGCCTGAAGGTT |
| 236 | 465131 | TGCCACCGTAGACACG |
| 237 | 469117 | GTCTGTGCATCTCTCC |
| 238 | 508031 | TGAGGTCCTGCACTGG |
| 239 | 482050 | ATCATGGCTGCAGCTT |
| 240 | 18(2PO) | CtcTTGATTTGTCagC |
| 241 | 05(2PO) | GacATAGTTAATTgcC |
| 242 | 95(2PO) | TctCATTTATTTCggC |
| 243 | H154 | CCTCAAGCTTGAGGGG |
| 244 | 1502 | GAGCAAGCTGGACCTTCCAT |
| 245 | 4000 | TTAGGGTTAGGGTTAGGG |
| 246 | G-ODN | CTCCTATTGGGGGTTTCCTAT |
| 247 | IRS954 | TGCTCCTGGAGGGGTTGT |
| 248 | IRS869 | TCCTGGAGGGGTTGT |
| 249 | INH | CCTGGATGGGAACTTACCGCTGCA |
| 250 | 2114R | TGAAGGGGAGGTCCT |
| 251 | 4352 | TCCTTCCTGGAGGGGAAG |
| 252 | 4191 | TCCTATCCTGGAGGGGAAG |
| 253 | 4351 | (TCCTA)2TCCTGGAGGGGAAG |
| 254 | 4348 | TCGTATCCTGGAGGGGAAG |
| 255 | 4348 | TAATATCCTGGAGGGGAAG |
| 256 | 4347 | CCTATCCTGGAGGGGAAG |
| 257 | 4350 | TCCTATAATGGAGGGGAAG |
| 258 | ODN | TGGGGGACGATCGTCGGGGGG |
| | 2216 | |

Uppercase indicates phosphonothioates (PS) bonds.
Lowercase indicates phosphodiester (PO) bonds.
2′cET modifications are in bold.

Since modifications will be apparent to those of skill in the art, it is intended that the invention(s) only are limited by the scope of the appended claims.

What is claimed:

1. A microalgae extracellular vesicle (MEV), comprising cargo selected from:
a) an agonist or an antagonist of a Toll-like receptor (TLR) and/or nucleic acid that is or that encodes a TLR agonist or a TLR antagonist; and/or
b) a modulator of an intracellular receptor involved in an immune response, whereby modulation of the TLR or intracellular receptor triggers a downstream signaling pathway mediating regulation of immune mediators; and/or
c) an antigen or immunogen and/or an immunomodulator, or nucleic acid encoding the antigen, immunogen, and/or immunomodulator, wherein the immunomodulator is an agent that acts on the immune system directly or indirectly,
wherein the microalgae is a species of the family Chlorellaceae.

2. The microalgae extracellular vesicle (MEV) of claim 1, wherein:
the cargo comprises a ligand that interacts with an endosomal receptor in an endosomal vesicle, whereby the cargo binds to the receptor and effects a biological response from within the cell;
the receptor is a toll-like receptor; and
the cargo comprises an agonist or an antagonist of a toll-like receptor (TLR) and/or comprises nucleic acid that is or that encodes a TLR agonist or a TLR antagonist, whereby modulation of the TLR receptor triggers a downstream signaling pathway mediating regulation of immune mediators.

3. A composition, comprising an MEV of claim 1, wherein the composition is formulated for administration by a route whereby the MEVs traffic to a cell, tissue, or organ of the immune system.

4. A vaccine composition, comprising a microalgae extracellular vesicle (MEV) of claim 1, wherein the composition does not comprise an exogenous adjuvant.

5. A vaccine composition, comprising a microalgae extracellular vesicle (MEV), of claim 1, wherein:
the vaccine is for treating, preventing, or reducing the severity of a disease, disorder, or condition; the immunomodulator is an agent that acts on the immune system directly or indirectly;
the composition is formulated for administration by a route whereby the MEVs traffic to a cell, tissue, or organ of the immune system; and
the composition does not comprise an exogenous adjuvant;
the vaccine is for treating, preventing, or reducing the severity of a disease, disorder, or condition;
the cargo comprises an agent that acts on the immune system directly or indirectly;
the composition is formulated for administration by a route whereby the MEVs traffic to a cell, tissue, or organ of the immune system; and
the composition does not comprise an exogenous adjuvant.

6. A composition of claim 3, comprising a nucleic acid molecule of any of SEQ ID Nos. 193-228.

7. The composition of claim 3, wherein:
the cargo comprises an agonist or antagonist of a TLR or nucleic acid encoding an agonist or antagonist of a TLR; and
the TLR is TLR9 or TLR3.

8. A vaccine composition, comprising a microalgae extracellular vesicle (MEV), wherein:
the microalgae is a species of the family Chlorellaceae;
the MEV comprise cargo that comprises one or more of antigen, immunogen, and/or an immunomodulator, or comprises nucleic acid encoding the antigen, immunogen, and/or immunomodulator;
the vaccine composition is for treating, preventing, or reducing the severity of a disease, disorder, or condition;
the immunomodulator is an agent that acts on the immune system directly or indirectly;
the composition is formulated for administration by a route whereby the MEVs traffic to a cell, tissue, or organ of the immune system.

9. The vaccine composition of claim 8, wherein the composition does not comprise an exogenous adjuvant.

10. The vaccine composition of claim 8, wherein the antigen comprises an antigen from a pathogen or from a tumor or cancer.

11. The vaccine composition of claim 8, wherein the immunomodulator is a cytokine or chemokine or receptor agonist or antagonist, or a receptor, or ligand that modulate an immune response.

12. The vaccine composition of claim 10, wherein the antigen from a pathogen and the pathogen is a bacterium, a virus, a parasite, or a fungal pathogen.

13. The vaccine composition of claim 8, wherein cargo in the MEVs comprises an antigen or nucleic acid encoding an antigen or an immunogenic protein, polypeptide, peptide from a pathogen.

14. The vaccine composition of claim 12, wherein the pathogen is a bacterial pathogen or a virus.

15. The vaccine composition of claim 14, wherein the pathogen is selected from one or more of: Enterobacterales, Vibrionales, Legionellales, Pseudomonadales, Pasteurellales, Staphylococcus, Streptococcus, Bacillus, Listeria, and Nocardia.

16. The vaccine composition of claim 15, wherein the pathogen is selected from one or more Shigella, Salmonella, Escherichia coli, Vibrio cholerae, Legionella neumophila, Pseudomonas aeruginosa, P syringae, P gingivalis, Acimetobacter, Moraxella, Haemophilus influenzae, Mannheimia, Actinobacillus, Staphylococcus aureus, Streptococcus pneumonia, Listeria Clostridium, and Nocardia species.

17. The vaccine composition of claim 15, wherein the pathogen is virus that is a hepatitis viruses, herpesviruses, varicella zoster virus (VZV), Epstein-Barr virus (EBV), human immunodeficiency virus (HIV), human T-cell leukemia virus (HTLV), Respiratory Syncytial Virus (RSV), measles virus, influenza virus, and coronaviruses.

18. The vaccine composition of claim 17, wherein the coronavirus is selected from among Severe Acute Respiratory Syndrome coronavirus (SARS-CoV), Middle East Respiratory Syndrome coronavirus (MERS-CoV), and Severe Acute Respiratory Syndrome coronavirus 2 (SARS-CoV-2), and Rhinovirus.

19. The vaccine composition of claim 8, wherein the cargo in the MEVs comprises a bacterial or viral antigen or nucleic acid encoding the antigen or an immunogenic portion thereof or epitope thereof selected from one or more of:
heat-labile enterotoxin B subunit of Escherichia coli;
Cholera toxin B (CTB) subunit of Vibrio cholerae extracellular capsule protein F1/immune-modulator V fusion protein of Yersinia pestis;
Outer membrane protein receptor for ferrichrome of Shigella flexneri;
Outer membrane protein OprF of Pseudomonas aeruginosa;
N-terminal portion of the Candida albicans agglutinin-like protein 3 (Als3p) of Staphylococcus aureus;
27-kDa outer membrane protein (T2544) of Salmonella enterica serovar Typhi;
Hepatitis B surface antigen (HBsAg) of Hepatitis B virus (HBV);
E1-E2 genome polyprotein of Hepatitis C virus (HCV) genotype 1a;
Inner capsid protein VP6 of Human rotavirus A;
Outer capsid glycoprotein VP of Rotavirus A;
Capsid protein of Norwalk virus (NV);
spike protein Si fragment of SARS-coronavirus (CoV);
B5R antigenic ectodomain of vaccinia virus;
envelope protein (E) of Japanese encephalitis virus (JEV);
VP4N20 antigenic peptide of Coxsackievirus 16 (CV-A16);
VP4N20 antigenic peptide of Enterovirus 71 (EV71);
Minor capsid protein L of Human papillomavirus type 16 (HPV-16);
Envelope glycoprotein D of Human herpesvirus 1 (HHV-1);
envelope domain III protein of Zika virus;
Major surface glycoprotein G of Human respiratory syncytial virus A (RSV strain A2);
domain III fragment of dengue 2 envelope protein (D2EIII) of Dengue virus type 2;
Merozoite surface protein 4 (MSP4) of Plasmodium falciparum;
Merozoite surface protein 5 (MSP5) od Plasmodium falciparum;
Trans-sialidase Trypanosoma cruzi A2 protein of Leishmania infantum; and
N-terminal portion of the Candida albicans agglutinin-like protein 3 (Als3p) of Candida albicans.

20. The vaccine composition of claim 19, wherein the sequence of the antigen is set forth in any of SEQ ID Nos:160-186 or is an immunogenic, antigenic, or epitope portion thereof.

21. A composition, comprising an MEV of claim 1, wherein the cargo comprises an immune modulator or nucleic acid encoding the immune modulator selected from among one or more of a cytokine, chemokine, co-stimulatory molecule, TNF superfamily of ligands or receptors, Toll-like receptor (TLR) agonist or antagonist, or immune checkpoint inhibitor, or a type I interferon or interferon-γ.

22. The composition of claim 21, wherein the immune modulator is an antibody or antigen-binding fragment thereof that specifically binds to and inhibits one or more of CTLA-4, PD-1, PD-L1, PD-L2, the PD-1/PDL1 pathway, the PD-1/PDL2 pathway, HER2, EGFR, TIM-3, LAG-3, BTLA-4, HHLA-2, CD28, and other checkpoints or immune suppressors, or tumor antigens.

23. The composition of claim 3, wherein the composition is formulated for oral administration, intramuscular administration, inhalation into the lungs or nose, mucosal administration, or local administration, or subcutaneous administration.

24. A composition comprising the MEV of claim 1, wherein the cargo comprises mRNA or DNA encoding the antigen or portion thereof, or the immunomodulator.

25. A composition, comprising an MEV of claim 1, wherein the MEV cargo comprises a TLR antagonist or agonist, and/or nucleic acid encoding a TLR agonist or antagonist.

26. The composition of claim 25, wherein the TLR antagonist or agonist is one or more of dsRNA (polyadenylic-polyuridilyc acid (poly(A:U)); polyino-sinic:polycytidylic acid (Poly (I:C) High Molecular (HMW) or Poly(I:C) Low Molecular Weight ((LMW); an antivirals shat is R848, R837, CL075, or CL264); taxol; flagellin; the flagellin-mimetic peptide flp22; unmethylated CpG motif oligo-nucleotides, CpG oligodeoxynucleotide(ODN) of class A, class B, and/or class C, dsRNA polyadenylic-polyuridilyc acid (poly(A:U); polyinosinic:polycytidylic acid (Poly (I:C)), taxol; flagellin; flagellin-mimetic peptide flp22, antivirals R848, R837, CL075, and/or CL264).

27. The vaccine of claim 25, wherein:
a) the TLR and TLR agonist is selected from one or more of:
TLR1 and the agonist is a triacyl lipopeptide Pam3CSK4;
TLR2 and that agonist Zymosan, Porin, Modulin, Lipoproteins, Lipoteichoic acid, Diacyl lipopeptides, Atypical LPS, Peptidoglycan, Triacyl lipopeptides
TLR3 and an agonist that is a dsRNA;
TLR4 and an that is Mannan, Taxol, and/or lipopolysaccharide (LPS);
TLR5 ant the agonis it a bacterial flagellin, profilin, HMGB1, and/or a Small molecule agonist CBLB502;
TLR6 and the agonist is Zymosan, Porin, Modulin, Lipoproteins, Lipoteichoic acid, Diacyl lipopeptides (Pam2CSK4), Atypical LPS, and/or a peptidoglycan;
TLR7 and the agonist is imidazoquinoline, loxoribine, a ssRNA, bropirimine, and/or resiquimod;
TLR8 and the agonist is a ssRNA, and/or a small synthetic compound;
TLR9 and the agonist is CpG DNA;
TLR10 and the agonist is a diacyl and/or triacyl lipopeptide; and
TLR11 and the agonist is a profilin-like protein, or a non-pathogenic bacterium; and/or
b) the TLR and antagonist is selected from one or more of:
TLR1 and the antagonist is CU-T12-9 and/or MMG-11;
TLR2 and the antagonist is AT1-AT8, CU-CPT22, CU-T12-9, MMG-11, NPT1220-312, Phloretin, and/or a sulfoglycolipid;
TLR3 and the antagonist is CU-CPT4a, monoclonal antibody NT04685, and/or monoclonal antibody CNT05429;
TLR4 and the antagonist is Norbinaltorphimine, T4Ics, T5342126, and/or Simvastatin;
TLR5 and the antagonist is TH1020;
TLR6 and the antagonist is Simvastatin;
TLR7 and the antagonist is chloroquine, hydroxychloroquine, and/or quinacrine;
TLR8 and the antagonist is CU-CPT8m, and/or CU-CPT9a; and
TLR9 and the antagonist is NPT1220-312, chloroquine, hydroxychloroquine, quinacrine; and/or suppressive or inhibitory oligonucleotides.

28. The composition of claim 3, wherein the cargo comprises an immunomodulator that suppresses the inflammatory response.

29. The composition of claim 3, wherein the cargo comprises an immunomodulator that suppresses an inflammatory response but does not suppress and anti-cancer immune response.

30. The composition of claim 3, wherein the MEVs are formulated for oral administration and the disease, disorder, or condition involves the gastrointestinal tract or the immune system or the white spleen.

31. The composition of claim 3, wherein the microalgae is a species of Chlorella selected from among Chlorella ellipsoidea, Chlorella pyrenoidosa, Chlorella sorokiniana, Chlorella vulgaris, and Chlorella variabilis.

32. The MEV of claim 28, wherein the Chlorella is Chlorella vulgaris.

33. A method of treatment, prevention, or reduction of the severity of a disease, disorder, or condition, comprising administering a vaccine composition comprising the MEV of claim 1 to a subject.

34. A method of treatment, prevention, or reduction of the severity of an enteric disease, disorder, or condition, comprising administering a vaccine composition comprising the MEV of claim 13 to a subject.

35. The method of claim 33, wherein the vaccine composition is administered orally or intramuscularly.

36. The method of claim 33, wherein the composition does not comprise an exogenous adjuvant.

37. The method of claim 33, wherein the vaccine is administered a plurality of times.

38. The method of claim 33, wherein the vaccine elicits a protective humoral response that comprises serum IgG and IgA and/or mucosal IgG and IgA.

39. The method of claim 33, wherein the vaccine composition is formulated for oral administration and the disease, disorder, or condition is a disease, disorder, or condition involving a pathogen or is cancer or an immune system disorder.

40. The method of claim 33, wherein the disease, disorder, or condition is one more of cancer; an autoimmune disease; an inflammatory disease, disorder or condition, or a disease, disorder, or condition in which inflammation plays a role in the etiology of the disease, disorder, or condition.
